(19) United States
(12) Patent Application Publication
(10) Pub. No.: US 2024/0024330 A1
(43) Pub. Date: Jan. 25, 2024

(54) CRF RECEPTOR ANTAGONISTS AND METHODS OF USE

(71) Applicant: Neurocrine Biosciences, Inc., San Diego, CA (US)

(72) Inventors: Gordon Raphael Loewen, San Diego, CA (US); Evan Smith, San Diego, CA (US); Robert H. Farber, San Diego, CA (US); Jean L. Chan, San Diego, CA (US)

(21) Appl. No.: 18/042,748

(22) PCT Filed: Aug. 25, 2021
(86) PCT No.: PCT/US2021/047550

§ 371 Date: Feb. 23, 2023

Related U.S. Application Data
(60) Provisional application No. 63/070,343, filed on Aug. 26, 2020.

(51) Int. Cl. A61K 31/5377; A61K 47/22; A61K 47/10; A61K 9/16; A61K 9/20; A61K 9/10; A61K 9/48; A61P 5/00

(52) U.S. Cl. CPC A61K 31/5377; A61K 47/22; A61K 47/10; A61K 9/1652; A61K 9/1611; A61K 9/1635; A61P 5/00; A61K 9/2009; A61K 9/2013; A61K 9/2027; A61K 9/10; A61K 9/4866; A61K 9/2054

(57) ABSTRACT

Provided are methods related to treating congenital adrenal hyperplasia in a subject in need thereof comprising administering to the subject a compound of Formula (I), or a pharmaceutically acceptable salt thereof.

### BACKGROUND

### Technical Field

The present disclosure generally relates to compounds of Formula (I), or pharmaceutically acceptable salts thereof, and compositions comprising compounds of Formula (I), or pharmaceutically acceptable salts thereof, that are useful in treating various diseases and/or disorders that would benefit from the same such as congenital adrenal hyperplasia (CAH).

### Description of the Related Technology

Classic congenital adrenal hyperplasia (CAH) is a disease that includes a group of autosomal recessive disorders that result in an enzyme deficiency that alters the production of adrenal steroids due to 21-hydroxylase deficiency, a condition that results in little or no cortisol biosynthesis.

One clinical manifestation of the absence of cortisol that occurs in congenital adrenal hyperplasia (CAH) is the lack of feedback inhibition of pituitary adrenocorticotropic hormone (ACTH) secretion. Increased ACTH levels cause adrenal hyperplasia and the enzyme block causes a shunting of cortisol precursor steroids to alternate pathways. Most notably, the shunting to androgens leads to virilization and other developmental complications in females, and the elevated ACTH levels are associated with the formation of testicular adrenal rest tumors in males. In addition, since the same enzyme (21-hydroxylase) is used in the pathway for the biosynthesis of the mineralocorticoids, a number of these patients suffer from aldosterone deficiency which may result in dehydration and death due to salt-wasting.

While survival is properly ensured through steroid replacement strategies based on physiologic dosing of glucocorticoids (e.g., hydrocortisone) and mineralocorticoids (e.g., fludrocortisone), these doses are often inadequate to suppress the overproduction of ACTH, progestogens, and androgens (e.g., 17-hydroxyprogesterone [17-OHP], androstenedione, and testosterone). The uncontrolled symptoms of androgen excess, indeed, have a substantial impact on the day-to-day functioning and development of these patients. The glucocorticoid doses required to treat the androgen excess are typically well above the normal physiologic doses used for cortisol replacement alone (as in patients with Addison's disease). This increased exposure to glucocorticoids may lead to iatrogenic Cushing's syndrome, increased cardiovascular risk factors, glucose intolerance, and decreased bone mineral density in CAH patients (Elnecave et al., J Pediatr Endocrinol Metab. 2008 December; 21(12):1155-62; King et al., J Clin Endocrinol Metab. 2006 March; 91(3):865-9; Migeon and Wisniewski, Endocrinol Metab Clin North Am. 2001 March; 30(1):193-206).

Corticotropin-releasing factor is a hypothalamic hormone released directly into the hypophyseal portal vasculature and acts on specific CRF₁ receptors on corticotropes in the anterior pituitary to stimulate the release of ACTH. Blockade of these receptors has been shown to decrease the release of ACTH in both animals and humans. Therefore, compounds that block CRF₁ receptors have the potential to directly inhibit the excessive ACTH release that occurs in CAH and thereby allow for normalization of androgen production while using lower, more physiologic doses of hydrocortisone. The compound of Formula (I), or a pharmaceutically acceptable salt thereof, may provide an important therapeutic approach to treat subjects with CAH.

### BRIEF SUMMARY

Provided herein are compounds that are CRF₁ receptor antagonists. In particular provided herein are compounds of Formula (I), or pharmaceutically acceptable salts thereof, and compositions comprising compounds of Formula (I), or pharmaceutically acceptable salts thereof, and methods related to treating congenital adrenal hyperplasia in a subject.

Some embodiments provide a compound of Formula (I):

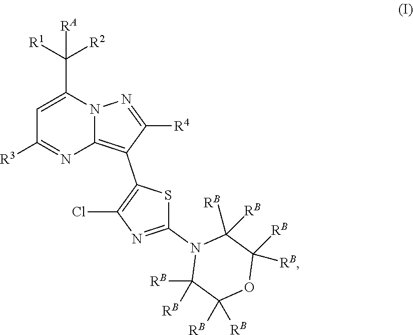

or a pharmaceutically acceptable salt thereof, wherein:
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium; and each R^{B} is independently hydrogen or deuterium. In some embodiments, at least one of R^{A} or R^{B} is deuterium.

Some embodiments provide a compound of Formula (IA):

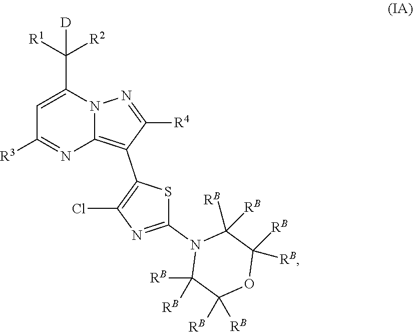

or a pharmaceutically acceptable salt thereof, wherein:
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium; and each R^{B} is independently hydrogen or deuterium.

Some embodiments provide a compound of Formula (IB):

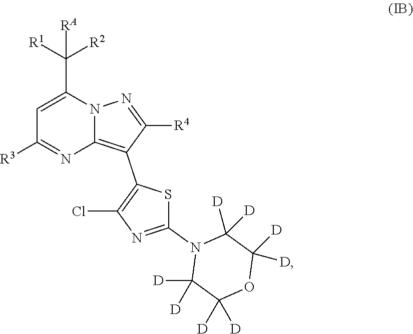

or a pharmaceutically acceptable salt thereof, wherein:
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium.

In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen.

Some embodiments provide a pharmaceutical composition comprising a compound of Formulae (I), (IA) or (IB), or a pharmaceutically acceptable salt thereof; and at least one pharmaceutically acceptable excipient.

Some embodiments provide a pharmaceutical composition comprising a compound as disclosed and described herein, or a pharmaceutically acceptable salt thereof, and at least one pharmaceutically acceptable excipient.

Some embodiments provide a compound of Formulae (I), (IA) or (IB), or a pharmaceutically acceptable salt thereof, or a compound as disclosed and described herein, or a pharmaceutically acceptable salt thereof, for use in treating congenital adrenal hyperplasia in a subject, wherein the compound or preparation is administered in an amount sufficient to reduce the level of one or more biomarkers selected from (a) 17-hydroxyprogesterone (17-OHP); (b) adrenocorticotropic hormone (ACTH); and (c) androstenedione in the subject

Some embodiments provide a method of treating congenital adrenal hyperplasia in a subject in need thereof comprising administering a compound of Formulae (I), (IA) or (IB), or a pharmaceutically acceptable salt thereof, or a compound as disclosed and described herein, or a pharmaceutically acceptable salt thereof, in an amount sufficient to reduce the level of one or more biomarkers selected from (a) 17-hydroxyprogesterone (17-OHP); (b) adrenocorticotropic hormone (ACTH); and (c) androstenedione in the subject.

Some embodiments provide a method for reducing the severity of one or more symptoms selected from hirsutism, precocious puberty, fertility problems, acne, and growth impairment in a subject having classic congenital adrenal hyperplasia, comprising administering a compound of Formulae (I), (IA) or (IB), or a pharmaceutically acceptable salt thereof, or a compound as disclosed and described herein, or a pharmaceutically acceptable salt thereof, in an amount sufficient to reduce the level of androstenedione in the subject.

Some embodiments provide a method of reducing the level of one or more biomarkers of congenital adrenal hyperplasia in a subject having congenital adrenal hyperplasia comprising administering to the subject a compound of Formulae (I), (IA) or (IB), or a pharmaceutically acceptable salt thereof, or a compound as disclosed and described herein, or a pharmaceutically acceptable salt thereof.

Some embodiments provide a method of reducing the dosage of corticosteroid administered to a subject having congenital adrenal hyperplasia for controlling congenital adrenal hyperplasia comprising administering to the subject a compound of Formulae (I), (IA) or (IB), or a pharmaceutically acceptable salt thereof, or a compound as disclosed and described herein, or a pharmaceutically acceptable salt thereof.

Some embodiments provide a method of reducing the severity of one or more side effects of glucocorticoid treatment in a subject having congenital adrenal hyperplasia comprising administering to the subject a compound of Formulae (I), (IA) or (IB), or a pharmaceutically acceptable salt thereof, or a compound as disclosed and described herein, or a pharmaceutically acceptable salt thereof, wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, reactivation of latent infections.

Some embodiments provide a method of treating congenital adrenal hyperplasia in a subject comprising i) measuring the level of one or more biomarkers selected from (a) 17-hydroxyprogesterone (17-OHP); (b) adrenocorticotropic hormone (ACTH); and (c) androstenedione in a biological sample obtained from the subject; ii) analyzing the level of the one or more biomarkers to determine if the level of the one or more biomarkers is elevated compared to a healthy subject not having congenital adrenal hyperplasia; and iii) administering to the subject a compound of Formulae (I), (IA) or (IB), or a pharmaceutically acceptable salt thereof, or a compound as disclosed and described herein, or a pharmaceutically acceptable salt thereof, if the subject is determined to have elevated levels of the one or more biomarkers.

Some embodiments provide a compound of Formulae (I), (IA) or (IB), or a pharmaceutically acceptable salt thereof, or a compound as disclosed and described herein, or a pharmaceutically acceptable salt thereof, for use in treating congenital adrenal hyperplasia in a subject, wherein the compound, or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject.

Some embodiments provide a compound of Formulae (I), (IA) or (IB), or a pharmaceutically acceptable salt thereof, or a compound as disclosed and described herein, or a pharmaceutically acceptable salt thereof, for use in treating in reducing the severity of one or more symptoms selected from hirsutism, precocious puberty, fertility problems, acne, and growth impairment in a subject having classic congenital adrenal hyperplasia, wherein the compound is administered in an amount sufficient to reduce the level of androstenedione in the subject.

Some embodiments provide a compound of Formulae (I), (IA) or (IB), or a pharmaceutically acceptable salt thereof, or a compound as disclosed and described herein, or a pharmaceutically acceptable salt thereof, for use in reducing the level of one or more biomarkers of congenital adrenal hyperplasia in a subject having congenital adrenal hyperplasia.

Some embodiments provide a compound of Formulae (I), (IA) or (IB), or a pharmaceutically acceptable salt thereof, or a compound as disclosed and described herein, or a pharmaceutically acceptable salt thereof, for use in reducing the dosage of corticosteroid administered to a subject having congenital adrenal hyperplasia.

Some embodiments provide a compound of Formulae (I), (IA) or (IB), or a pharmaceutically acceptable salt thereof, or a compound as disclosed and described herein, or a pharmaceutically acceptable salt thereof, for use in reducing the severity of one or more side effects of glucocorticoid treatment in a subject having congenital adrenal hyperplasia, wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, reactivation of latent infections.

Some embodiments provide a compound of Formulae (I), (IA) or (IB), or a pharmaceutically acceptable salt thereof, or a compound as disclosed and described herein, or a pharmaceutically acceptable salt thereof, for use in treating congenital adrenal hyperplasia in a subject comprising i) measuring the level of one or more biomarkers selected from (a) 17-hydroxyprogesterone (17-OHP); (b) adrenocorticotropic hormone (ACTH); and (c) androstenedione in a biological sample obtained from the subject; ii) analyzing the level of the one or more biomarkers to determine if the level of the one or more biomarkers is elevated compared to a healthy subject not having congenital adrenal hyperplasia; and iii) administering to the subject the compound if the subject is determined to have elevated levels of the one or more biomarkers.

### DETAILED DESCRIPTION

As described herein, a compound of Formula (IA):

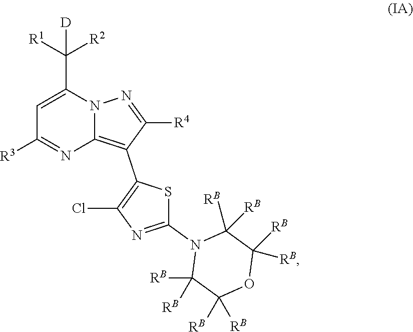

or a pharmaceutically acceptable salt thereof, wherein.
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium; and each R^{B} is independently hydrogen or deuterium for treating CAH in a subject having CAH is provided.

Newborn screening for CAH is performed by immunoassay to measure 17-OHP levels in heel-stick capillary blood specimens obtained within the first 72 hours of life. The blood sample is analyzed for 17-OHP by commercially available dissociation-enhanced lanthanide fluoroimmunoassay (DELFIA; PerkinElmer, Waltham Massachusetts) (White et al., J. Pediatr. 163:10-12 (2013)). Second-tier screening tests utilizing biochemical and molecular genetic testing methods, performed between 8 and 14 days of life, are employed by nine states in the United States and strongly recommended by an additional 5 states. The biochemical method includes immunoassay with organic solvent extraction or liquid chromatography followed by tandem mass spectrometry to measure steroid ratios of 17-01P, androstenedione, and 21-deoxycortisol to cortisol (see, e.g., Speiser et al., Int. J. Pediatr. Endocrinol. 2010:494173, 2010). The genetic screen looks for CYP21A2 mutations that are associated with CAH. While not widely employed in the U.S., the addition of a second screening could potentially improve the sensitivity of the overall screening process, where sensitivity of the first screen alone is approximately 72%.

In absence of results from the newborn screening, female infants with classical CAH are typically identified due to the presence of ambiguous genitalia. Males have normal genitalia at birth and therefore are not diagnosed unless newborn screening is conducted or other medical complications come to attention. Infants who are not initially diagnosed with CAH and suffer from the salt-wasting form of the disease are later diagnosed in the setting of poor weight gain, vomiting, hyperkalemia and hyponatremia within the first few weeks of life.

Treatment of CAH is based on normalization of hormone and steroid levels using a variety of medications from diagnosis in infancy through adulthood. Glucocorticoids are the current standard treatment in CAH and are used both to correct the endogenous cortisol deficiency and for reducing the elevated ACTH levels from the pituitary, which drives increased androgen production. Unlike the treatment of Addison's disease (adrenal insufficiency), in which cortisol replacement is sufficient, the treatment of CAH must also reduce ACTH production, to control the subsequent androgen excess as well. Thus, the goals of glucocorticoid treatment include cortisol replacement and suppression of ACTH to prevent virilization and menstrual disturbances in women and to inhibit testicular adrenal rest tumors in men. Mineralocorticoid replacement is needed to achieve normal plasma renin activity for maintenance of regular blood pressure, electrolyte balance, and volume status in those patients with the salt-wasting form of CAH.

The regimen of glucocorticoid treatment must support normal physiology and also ensure that sufficient cortisol is available during events that may elicit a strong stress response (e.g., intercurrent illness, exercise, hypotension). Careful monitoring is also necessary to avoid the development of iatrogenic Cushing's syndrome due to glucocorticoid overtreatment in an effort to adequately suppress androgen production, or Addisonian syndrome due to under-treatment.

Overtreatment with mineralocorticoids may cause hypertension while under-treatment may lead to low blood pressure, salt loss, fatigue and increased requirements for glucocorticoids. Typical laboratory tests for monitoring treatment efficacy include measurement of plasma concentrations of 17-OHP, androstenedione, testosterone, renin activity, and electrolytes.

Adult patients with CAH have an increased prevalence of risk factors for cardiovascular disease including obesity, hypertension, and insulin resistance (see, e.g., Kim et al., Semin. Reprod. Med. 27(4):316-21 (2009)). A study of a large cohort of pediatric and adult CAH patients (n=244) demonstrated that patients are prescribed a variety of glucocorticoid treatment regimens yet frequently suffer from poor hormonal control and the aforementioned adverse outcomes (see, e.g., Finkielstain et al., J. Clin. Endocrinol Metab. 97(12):4429-38 (2012)).

Treatment of CAH includes efforts to normalize the cortisol deficiency with glucocorticoids (usually hydrocortisone in children but often more potent agents with narrow therapeutic indices, such as dexamethasone, in adults) and, if necessary for salt-wasting, mineralocorticoids (usually fludrocortisone). The glucocorticoid doses required to achieve sufficient suppression of excess androgens, however, are usually well above the normal physiologic dose used for cortisol replacement alone as in patients with Addison's disease. This increased exposure to glucocorticoids can lead to iatrogenic Cushing's syndrome, increased cardiovascular risk factors, glucose intolerance, and decreased bone mineral density in CAH patients (see, e.g., Elnecave et al., J. Pediatr. Endocrinol. Metab. 21:1155-62 (2008); King et al., J. Clin. Endocrinol. Metab. 91(3):8656-59 (2006); Migeon et al., Endocrinol. Metab. Clin. North Am. 30:193-206 (2001)). Recently, best practices for the clinical management of congenital adrenal hyperplasia were published in the Journal of Clinical Endocrinology andMetabolism (Speiser, P. W., et al. J. Clin. Endocrinol. Metab. November 2018, 103(11): 1-46). This article is incorporated by reference in its entirety.

Corticotropin-releasing factor (CRF) was isolated from ovine hypothalami and identified as a 41-amino acid peptide. CRF has been found to produce profound alterations in endocrine, nervous, and immune system function. CRF is believed to be the major physiological regulator of the basal and stress-induced release of adrenocorticotropic hormone (“ACTH”), 8-endorphin, and other pro-opiomelanocortin (“POMC”)-derived peptides from the anterior pituitary (see, e.g., Vale et al., Science 213:1394-1397, 1981). Secretion of CRF causes release of ACTH from corticotrophs in the anterior pituitary via binding to the CRF₁ receptor, a member of the class B family of G-protein coupled receptors.

Due to the physiological significance of CRY, the development of biologically-active small molecules having significant CRF₁ receptor binding activity and which are capable of antagonizing the CRF₁ receptor remains a desirable goal and has been the subject of ongoing research and development for the treatment of anxiety, depression, irritable bowel syndrome, post-traumatic stress disorder, and substance abuse.

The pituitary hormone ACTH, under the control of hypothalamic corticotropin-releasing factor (CRF), stimulates uptake of cholesterol and drives the synthesis of pregnenolone initiating steroidogenesis in the adrenal gland. The adrenal cortex is comprised of three zones, which produce distinct classes of hormones many of which are driven by ACTH mobilizing cholesterol through this pathway. Deficiencies in these enzymes as a result of mutation or deletion cause the substrate concentrations to increase. In the most common form of CAH resulting from mutations or deletions in the 21-hydroxylase gene (CYP21A2), potent androgens are produced by the adrenal because of the accumulation of the steroid precursors, progesterone and 17-hydroxyprogesterone (17-OHP). Plasma levels of 17-OHP can reach 10-1000 times the normal concentration in these cases. These increases result in the overproduction of androgens, specifically androstenedione, testosterone, and dihydroxytestosterone causing virilization in females. In addition, 21-hydroxylase deficiency in CAH causes insufficient biosynthesis of glucocorticoids and mineralocorticoids, specifically cortisol and aldosterone. Cortisol is a critical negative feedback regulator of hypothalamic CRF secretion and pituitary ACTH release. The lack of glucocorticoid synthesis and release eliminates the restraint on the hypothalamus and pituitary, which causes ACTH levels to increase. The excessive ACTH stimulation causes hypertrophy of the zona fasciculata and zona reticularis resulting in adrenal hyperplasia.

### Definitions

For clarity and consistency, the following definitions will be used throughout this patent document.

As used herein, “about” means±20% of the stated value, and includes more specifically values of ±10%, +5%, +2% and +1% of the stated value.

As used herein, “administration” or “administering” refers to providing a compound described herein or other therapy to a subject in a form that may be introduced into that subject's body in a therapeutically useful form and therapeutically useful amount, including, but not limited to: oral dosage forms, such as, tablets, capsules, syrups, liquid formulations (e.g. solutions and suspensions), and the like; injectable dosage forms, such as, IV, IM, IP, and the like; transdermal dosage forms, including creams, jellies, powders, and patches; buccal dosage forms; inhalation powders, sprays, suspensions, and the like; and rectal suppositories.

A health care practitioner may directly provide a compound described herein to a subject in the form of a sample or may indirectly provide a compound to a subject by providing an oral or written prescription for the compound. Also, for example, a subject may obtain a compound by themselves without the involvement of a health care practitioner. When the compound is administered to the subject, the body is transformed by the compound in some way. When a compound described herein is provided in combination with one or more other agents, “administration” is understood to include the compound and other agents are administered at the same time or at different times. When the agents of a combination are administered at the same time, they may be administered together in a single composition or they may be administered separately. The preferred method of administration may vary depending on various factors, e.g., the components of the pharmaceutical formulation, the site of the disease, and the severity of the disease.

As used herein, “time of day window” refers to a period of time defined by a window start time and a window stop time. These times all refer to local times where a sample was taken. The phrase “same time of day window” when referring to samples taken from the subject mean, e.g., that a sample taken at 8:15 a.m. and a sample taken at 9:15 a.m. are considered to be taken in the same time of day window of, e.g., 2 a.m. to 10 a.m. or 6 a.m. to 10 a.m.

The term “composition” refers to a compound or crystalline form thereof, including but not limited to, salts, and solvates of a compound described herein, in combination with at least one additional component, such as, a composition obtained/prepared during synthesis, preformulation, in-process testing (e.g., TLC, HPLC, NMR samples), and the like.

The term, “compound,” as used herein is meant to include all stereoisomers, geometric isomers, tautomers and isotopes of the structures depicted. The term is also meant to refer to compounds described herein, regardless of how they are prepared, e.g., synthetically, through biological process (e.g., metabolism or enzyme conversion), or a combination thereof. The term can further refer to a single molecule or a plurality of molecules. All compounds, and pharmaceutically acceptable salts thereof, may be found together with other substances such as water and other solvents (e.g., hydrates and solvates) or may be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound. In some embodiments, the compounds described herein, or salts thereof, are substantially isolated. By “substantially isolated” is meant that the compound is at least partially or substantially separated from the environment in which it was formed or detected. Partial separation may include, e.g., a composition enriched in the compounds described herein. Substantial separation may include compositions containing at least about 50%, at least about 60%, at least about 70%, at least about 80%, at least about 90%, at least about 95%, at least about 97%, or at least about 99% by weight of the compounds described herein, or salts thereof.

The term “hydrate” as used herein refers to a compound described herein or a salt thereof that further includes a stoichiometric or non-stoichiometric amount of water bound by non-covalent intermolecular forces.

The term “in need thereof” when referring to treatment are used interchangeably to mean a judgment made by a caregiver (e.g. physician, nurse, nurse practitioner, etc. in the case of humans; veterinarian in the case of animals, including non-human mammals) that a subject or animal requires or will benefit from treatment. This judgment is made based on a variety of factors that are in the realm of a caregiver's expertise, but that includes the knowledge that the subject or animal is ill, or will become ill, as the result of a disease, condition or disorder that is treatable by the compound described herein. Accordingly, the compound described herein may be used in a protective or preventive manner; or compound described herein may be used to alleviate, inhibit, or ameliorate the disease, condition, or disorder.

The term “subject” refers to any animal, including mammals, such as, mice, rats, other rodents, rabbits, dogs, cats, swine, cattle, sheep, horses, primates, and humans. In the context of a clinical trial or screening or activity experiment the subject may be a healthy volunteer or healthy participant without an underlying CRF₁ mediated disorder or condition or a volunteer or participant that has received a diagnosis for a disorder or condition in need of medical treatment as determined by a health care professional. In the context outside of a clinical trial a subject under the care of a health care professional who has received a diagnosis for a disorder or condition is typically described as a subject and may also be referred to as a patient.

The term “pediatric subject” refers to a subject under the age of 21 years at the time of diagnosis or treatment. The term “pediatric” may be further divided into various subpopulations including: neonates (from birth through the first month of life); infants (1 month up to two years of age); children (two years of age up to 12 years of age); and adolescents (12 years of age through 21 years of age (up to, but not including, the twenty-second birthday)) see e.g., Berhman et al., Textbook of Pediatrics, 15th Ed. Philadelphia: W.B. Saunders Company, 1996; Rudolph et al., Rudolph's Pediatrics, 21st Ed. New York: McGraw-Hill, 2002; and Avery et al., Pediatric Medicine, 2nd Ed. Baltimore: Williams & Wilkins; 1994.

The phrase “pharmaceutically acceptable” refers to compounds (and salts thereof), compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutical composition” refers to a specific composition comprising at least one drug substance; including but not limited to, salts, solvates, and hydrates of compounds described herein, whereby the composition is amenable to investigation for a specified, efficacious outcome in a mammal (for example, without limitation, a human). Those of ordinary skill in the art will understand and appreciate the techniques appropriate for determining whether a drug substance has a desired efficacious outcome based upon the needs of the artisan.

The terms “prevent,” and “preventing,” refer to the prevention, elimination or reduction of the occurrence or onset of one or more symptoms associated with a particular disorder. For example, the terms “prevent,” and “preventing” may refer to the administration of therapy on a prophylactic or preventative basis to a subject who may ultimately manifest at least one symptom of a disorder but who has not yet done so. Such subjects may be identified on the basis of risk factors that are known to correlate with the subsequent occurrence of the disease, such as the presence of a biomarker.

The term “solvate” as used herein refers to a solid-state form of a compound described herein, or a pharmaceutically acceptable salt thereof which includes a stoichiometric or non-stoichiometric amount of a solvent bound by non-covalent intermolecular forces. When the solvent is water, the solvate is a hydrate.

The terms “treat” or “treatment” as used herein refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “therapeutically effective amount” refers to the amount of the compound described herein, or a pharmaceutically acceptable salt thereof, or an amount of a pharmaceutical composition comprising the compound described herein or a pharmaceutically acceptable salt thereof, that elicits the biological or medicinal response in a tissue, system, animal, or human that is being sought by a subject, researcher, veterinarian, medical doctor, or other clinician or caregiver, which may include one or more of the following:
(1) preventing the disorder, for example, preventing a disease, condition, or disorder in a subject who may be predisposed to the disease, condition, or disorder but does not yet experience or display the relevant pathology or symptomatology;
(2) inhibiting the disorder, for example, inhibiting a disease, condition, or disorder in a subject who is experiencing or displaying the relevant pathology or symptomatology (i.e., arresting further development of the pathology and/or symptomatology); and
(3) ameliorating the disorder, for example, ameliorating a disease, condition, or disorder in a subject who is experiencing or displaying the relevant pathology or symptomatology (i.e., reversing the pathology and/or symptomatology).

As used herein, the term “contacting” refers to the bringing together of indicated moieties in an in vitro system or an in vivo system. For example, “contacting” CRF₁ receptor with a compound provided herein includes the administration of a compound provided herein (or a pharmaceutically acceptable salt thereof) to a subject, such as a human, having a CRF₁ receptor protein, as well as, for example, introducing a compound provided herein into a sample containing a cellular or purified preparation containing the CRF₁ receptor protein.

Methods

The present disclosure relates to methods of treating congenital adrenal hyperplasia (CAH). The methods include administering to a subject a therapeutically effective amount of a compound of Formula (I), or pharmaceutically acceptable salt thereof.

In some embodiments, the compound of Formula (I), or a pharmaceutically acceptable salt thereof, is administered at a frequency of not less than twice daily; and the amount of the compound of Formula (I), or a pharmaceutically acceptable salt thereof, in the first administration is less than the amount of the compound of Formula (I), or a pharmaceutically acceptable salt thereof, in the second and any subsequent administrations. In some embodiments, the compound of Formula (I), or a pharmaceutically acceptable salt thereof, is administered at a frequency of twice daily. In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:100, 1:1.1 to 1:50, 1:1.1 to 1:10, 1:1.1 to 1:5, 1:1.1 to 1:3, 1:1.1 to 1:2.5 or 1:1.1 to 1:2. In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:1.5, about 1:2, about 1:2.5, about 1:3, about 1:3.5 or about 1:4. In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is 1:1.5, 1:2, 1:2.5, 1:3, 1:3.5 or 1:4. In some embodiments, the amount of the compound of Formula (I), or a pharmaceutically acceptable salt thereof, administered daily is less than or equal to about 1000 mg based on the weight of the free base. In some embodiments, the amount of the compound of Formula (I), or a pharmaceutically acceptable salt thereof, administered daily is from about 50 mg to about 1000 mg, about 100 mg to about 1000 mg, about 100 mg to about 1000 mg, about 100 mg to about 500 mg, about 200 mg to about 500 mg, or about 200 mg to about 450 mg based on the weight of the free base. In some embodiments, the amount of the compound of Formula (I), or a pharmaceutically acceptable salt thereof, administered daily is about 200 mg based on the weight of the free base. In some embodiments, the amount of the compound of Formula (I), or a pharmaceutically acceptable salt thereof, administered daily is about 250 mg based on the weight of the free base. In some embodiments, the amount of the compound of Formula (I), or a pharmaceutically acceptable salt thereof, administered daily is about 400 mg based on the weight of the free base. In some embodiments, the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base. In some embodiments, the first administration of the compound of Formula (I), or a pharmaceutically acceptable salt thereof is about 150 mg based on the weight of the free base and the second administration of the compound of Formula (I), or a pharmaceutically acceptable salt thereof is about 250 mg based on the weight of the free base. In some embodiments, the amount of the compound of Formula (I), or a pharmaceutically acceptable salt thereof, administered daily is about 300 mg based on the weight of the free base. In some embodiments, the amount of the compound of Formula (I), or a pharmaceutically acceptable salt thereof, administered daily is about 450 mg based on the weight of the free base. In some embodiments, the first administration of the compound of Formula (I), or a pharmaceutically acceptable salt thereof is about 100 mg based on the weight of the free base and the second administration of the compound of Formula (I), or a pharmaceutically acceptable salt thereof is about 200 mg based on the weight of the free base. In some embodiments, the first administration of the compound of Formula (I), or a pharmaceutically acceptable salt thereof is about 200 mg based on the weight of the free base and the second administration of the compound of Formula (I), or a pharmaceutically acceptable salt thereof is about 250 mg based on the weight of the free base. In some embodiments, the amount of the compound of Formula (I), or a pharmaceutically acceptable salt thereof, administered daily is about 350 mg based on the weight of the free base. In some embodiments, the amount of the compound of Formula (I), or a pharmaceutically acceptable salt thereof, administered daily is about 500 mg based on the weight of the free base. In some embodiments, the first administration of the compound of Formula (I), or a pharmaceutically acceptable salt thereof is about 150 mg based on the weight of the free base and the second administration of the compound of Formula (I), or a pharmaceutically acceptable salt thereof is about 200 mg based on the weight of the free base. In some embodiments, the first administration of the compound of Formula (I), or a pharmaceutically acceptable salt thereof is about 200 mg based on the weight of the free base and the second administration of the compound of Formula (I), or a pharmaceutically acceptable salt thereof is about 300 mg based on the weight of the free base. In some embodiments, the subject weighs greater than or equal to about 55 kg. In some embodiments, the amount of the compound of Formula (I), or a pharmaceutically acceptable salt thereof, administered daily is about 100 mg based on the weight of the free base. In some embodiments, the subject weighs from about 10 kg to about 20 kg. In some embodiments, the amount of the compound of Formula (I), or a pharmaceutically acceptable salt thereof, administered daily is about 250 mg based on the weight of the free base. In some embodiments, the amount of the compound of Formula (I), or a pharmaceutically acceptable salt thereof, administered daily is about 300 mg based on the weight of the free base. In some embodiments, the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base. In some embodiments, the first administration of the compound of Formula (I), or a pharmaceutically acceptable salt thereof is about 100 mg based on the weight of the free base and the second administration of the compound of Formula (I), or a pharmaceutically acceptable salt thereof is about 200 mg based on the weight of the free base. In some embodiments, the subject weighs from about 20 kg to about 55 kg.

Provided herein is a method of treating congenital adrenal hyperplasia (CAH) comprising administering a compound of Formula (I), or a pharmaceutically acceptable salt thereof to normalize or partially normalize levels of biomarkers associated with congenital adrenal hyperplasia. In some embodiments, normalizing or partially normalizing levels of biomarkers comprises reducing levels of elevated biomarkers or increasing levels of depressed biomarkers as compared to subject without CAH. Provided herein is a method of treating congenital adrenal hyperplasia in a subject in need thereof comprising administering a compound of Formula (I), or a pharmaceutically acceptable salt thereof, in an amount sufficient to reduce the level of one or more biomarkers associated with congenital adrenal hyperplasia. In some embodiments, the biomarkers are selected from (a) 17-hydroxyprogesterone (17-OHP); (b) adrenocorticotropic hormone (ACTH); and (c) androstenedione in the subject.

Some embodiments provide a method of treating congenital adrenal hyperplasia in a subject in need thereof comprising administering to the subject a compound of Formulae (I), (IA) or (IB), or a pharmaceutically acceptable salt thereof, or a compound as disclosed and described herein, or a pharmaceutically acceptable salt thereof, wherein the compound of Formulae (I), (IA) or (IB), or a pharmaceutically acceptable salt thereof, or a compound as disclosed and described herein, or a pharmaceutically acceptable salt thereof, is administered at a frequency of not less than twice daily; and wherein the amount of the compound of Formulae (I), (IA) or (IB), or a pharmaceutically acceptable salt thereof, or a compound as disclosed and described herein, or a pharmaceutically acceptable salt thereof, administered daily is greater than 400 mg based on the weight of the free base. Some embodiments provide a method of treating congenital adrenal hyperplasia in a subject in need thereof comprising administering to the subject a compound of Formulae (I), (IA) or (IB), or a pharmaceutically acceptable salt thereof, or a compound as disclosed and described herein, or a pharmaceutically acceptable salt thereof, wherein the compound of Formulae (I), (IA) or (IB), or a pharmaceutically acceptable salt thereof, or a compound as disclosed and described herein, or a pharmaceutically acceptable salt thereof, is administered at a frequency of not less than twice daily; and wherein the amount of the compound of Formulae (I), (IA) or (IB), or a pharmaceutically acceptable salt thereof, or a compound as disclosed and described herein, or a pharmaceutically acceptable salt thereof, administered daily is less than 500 mg based on the weight of the free base (e.g. less than or equal to 400 mg based on the weight of the free base). In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the compound of Formulae (I), (IA) or (IB), or a pharmaceutically acceptable salt thereof, or a compound as disclosed and described herein, or a pharmaceutically acceptable salt thereof, is administered at a frequency of twice daily. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 100 mg to about 375 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 100 mg to about 350 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 100 mg to about 300 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 100 mg to about 275 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 100 mg to about 250 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 250 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is about 100 mg and the second administration of the compound of Formulae (I), (IA) or (IB), or a pharmaceutically acceptable salt thereof, or a compound as disclosed and described herein, or a pharmaceutically acceptable salt thereof is about 150 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 405 mg to about 1000 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 405 mg to about 600 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 405 mg to about 500 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 405 mg to about 475 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is from 405 mg to about 450 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 300 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is 100 200 mg and the second administration of the compound of Formulae (I), (IA) or (IB), or a pharmaceutically acceptable salt thereof, or a compound as disclosed and described herein, or a pharmaceutically acceptable salt thereof is about 200 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 450 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is about 200 mg and the second administration of the compound of Formulae (I), (IA) or (IB), or a pharmaceutically acceptable salt thereof, or a compound as disclosed and described herein, or a pharmaceutically acceptable salt thereof is about 250 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 225 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is about 100 mg and the second administration of the compound of Formulae (I), (IA) or (IB), or a pharmaceutically acceptable salt thereof, or a compound as disclosed and described herein, or a pharmaceutically acceptable salt thereof is about 125 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 425 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is about 200 mg and the second administration of the compound of Formulae (I), (IA) or (IB), or a pharmaceutically acceptable salt thereof, or a compound as disclosed and described herein, or a pharmaceutically acceptable salt thereof is about 225 mg based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject.

In some embodiments, the reduction in level of any of the biomarkers (e.g., any of 17-OHP, ACTH, and androstenedione) is determined by comparing the level of the biomarker as measured during the circadian release on a day prior to administering the compound of Formula (I), or a pharmaceutically acceptable salt thereof and the level of the biomarker as measured during the circadian release on the day after administering the compound of Formula (I), or a pharmaceutically acceptable salt thereof. A day prior to administering the compound of Formula (I) applies to a subject that has not previously been administered the compound of Formula (I) within at least the past 24 hours.

In some embodiments, the circadian release of biomarkers associated with CAH occurs between the hours of 2 a.m. and 10 a.m. In other embodiments, the circadian release of biomarkers associated with CAH occurs between the hours of 6 a.m. and 10 a.m.

In some embodiments of any of the methods disclosed herein, the compound of Formula (I), or a pharmaceutically acceptable salt, is administered to the subject at nighttime or administration prior to sleep (i.e., bedtime administration). In some embodiments, the compound of Formula (I), or a pharmaceutically acceptable salt thereof, is administered three to eight hours prior to the circadian release of the biomarker. In some embodiments, the compound of Formula (I), or a pharmaceutically acceptable salt thereof, is administered six to eight hours prior to the circadian release of the biomarker. Administration prior to the circadian release may be adapted for shift workers (e.g., those who work at night and sleep during the day), in which case administration will not necessarily occur at nighttime. Administration is therefore dependent upon the expected circadian release of the biomarker, and can vary depending upon the individual's (i.e., subject, patient) particular work and sleep patterns.

In some embodiments of the methods provided herein, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some embodiments, the level of 17-hydroxyprogesterone is reduced by at least 25%. In some embodiments, the level of 17-hydroxyprogesterone is reduced by at least 50%. In some embodiments of the methods provided herein, the level of 17-hydroxyprogesterone is reduced by an amount of from 10% to 60%, 15% to 60%, 20% to 60%, 25% to 60%, 30% to 60%, 35% to 60%, 40% to 60%, or 50% to 60% from pre-administration levels. In some embodiments of the methods provided herein, the level of 17-hydroxyprogesterone is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some embodiments, the level of 17-hydroxyprogesterone is reduced to a level within the range of 17-hydroxyprogesterone expected for a subject without CAH, i.e., less than 1,000 ng/dL or less than 200 ng/dL.

In some embodiments of the methods provided herein, the level of adrenocorticotropic hormone is reduced by at least 10%, at least 15%, at least 20%, at least 25%, at least 30%, at least 35%, at least 40%, at least 50%, at least 55% or at least 60% from pre-administration levels. In some embodiments, the level of adrenocorticotropic hormone is reduced by at least 25%. In some embodiments, the level of adrenocorticotropic hormone is reduced by at least 40%. In some embodiments, the level of adrenocorticotropic hormone is reduced by at least 50%. In some embodiments of the methods provided herein, the level of adrenocorticotropic hormone is reduced by an amount of from 10% to 60%, 15% to 60%, 20% to 60%, 25% to 60%, 30% to 60%, 35% to 60%, 40% to 60%, or 50% to 60% from pre-administration levels.

In some embodiments of the methods provided herein, the level of adrenocorticotropic hormone is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels.

In some embodiments, the level of adrenocorticotropic hormone is reduced to a level within the range of adrenocorticotropic hormone expected for a subject without CAH.

In some embodiments of the methods provided herein, the level of androstenedione is reduced by at least 10%, at least 15%, at least 20%, at least 25%, at least 30%, at least 35%, at least 40%, at least 50%, at least 55% or at least 60% from pre-administration levels. In some embodiments, the level of androstenedione is reduced by at least 25%. In some embodiments, the level of androstenedione is reduced by at least 30%. In some embodiments, the level of androstenedione is reduced by at least 50%. In some embodiments of the methods provided herein, the level of androstenedione is reduced by an amount of from 10% to 60%, 15% to 60%, 20% to 60%, 25% to 60%, 30% to 60%, 35% to 60%, 40% to 60%, or 50% to 60% from pre-administration levels.

In some embodiments of the methods provided herein, the level of androstenedione is reduced by an amount of from about 10% to about 90%, about 15% to about 90%, about 20% to about 90%, about 25% to about 90%, about 30% to about 90%, about 35% to about 90%, about 40% to about 90%, about 50% to about 90%, about 55% to about 90%, or about 60% to about 90% from pre-administration levels. In some embodiments of the methods provided herein, the level of 17-hydroxyprogesterone is reduced by at least 50% and the level of androstenedione is reduced by at least 50% from pre-administration levels.

In some embodiments, the level of androstenedione is reduced to a level within the range of androstenedione expected for a subject without CAH, i.e., less than 200 ng/dL.

Also provided herein is a method for reducing the severity of one or more symptoms selected from hirsutism, precocious puberty, fertility problems, acne, and growth impairment in a subject having classic congenital adrenal hyperplasia, comprising administering a compound of Formula (I), or a pharmaceutically acceptable salt thereof. In some embodiments, the compound of Formula (I), or a pharmaceutically acceptable salt thereof is administered at a frequency of not less than twice daily. In some embodiments, the amount of the compound of Formula (I), or a pharmaceutically acceptable salt thereof, in the first administration is less than the amount of the compound of Formula (I), or a pharmaceutically acceptable salt thereof, in the second and any subsequent administrations. In some embodiments, the compound of Formula (I), or a pharmaceutically acceptable salt thereof, is administered in an amount sufficient to reduce one or more biomarker of CAH in a subject, e.g., reduce the androstenedione in the subject. Growth impairment can refer to, e.g., accelerated height velocity, accelerated weight velocity, and/or accelerated bone age. In some embodiments, the level of androstenedione is reduced by at least 30% from pre-administration levels. In some embodiments, the level of androstenedione is reduced by at least 40% from pre-administration levels. In some embodiments, the level of androstenedione is reduced by at least 50% from pre-administration levels. In some embodiments of the methods provided herein, the level of androstenedione is reduced by an amount of from 10% to 60%, 15% to 60%, 20% to 60%, 25% to 60%, 30% to 60%, 35% to 60%, 40% to 60%, or 50% to 60% from pre-administration levels.

Provided herein is a method for reducing the level of one or more biomarkers of congenital adrenal hyperplasia in a subject having congenital adrenal hyperplasia comprising administering to the subject a compound of Formula (I), or a pharmaceutically acceptable salt thereof. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the one or more biomarkers of congenital adrenal hyperplasia are selected from (a) 17-hydroxyprogesterone (17-OHP); (b) adrenocorticotropic hormone (ACTH); and (c) androstenedione. In some embodiments, the compound of Formula (I), or a pharmaceutically acceptable salt thereof, is administered at a frequency of not less than twice daily. In some embodiments, the amount of the compound of Formula (I), or a pharmaceutically acceptable salt thereof, in the first administration is less than the amount of the compound of Formula (I), or a pharmaceutically acceptable salt thereof, in the second and any subsequent administrations.

Provided herein is a method for reducing the dosage of corticosteroid administered to a subject having congenital adrenal hyperplasia for controlling congenital adrenal hyperplasia comprising administering to the subject a compound of Formula (I), or a pharmaceutically acceptable salt thereof. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the compound of Formula (I), or a pharmaceutically acceptable salt thereof, is administered at a frequency of not less than twice daily. In some embodiments, the amount of the compound of Formula (I), or a pharmaceutically acceptable salt thereof, in the first administration is less than the amount of the compound of Formula (I), or a pharmaceutically acceptable salt thereof, in the second and any subsequent administrations. In some embodiments, the corticosteroid is a glucocorticoid.

Also provided herein is a method of reducing the severity of one or more side effects of glucocorticoid treatment in a subject having congenital adrenal hyperplasia comprising administering to the subject a compound of Formula (I), or a pharmaceutically acceptable salt thereof. The long-term effects of glucocorticoid treatment are well documented in the art (see, e.g., Oray, M. et al. (2016): Long-term effect of glucocorticoids, Expert Opinion on Drug Safety. DOI: 10.1517/14740338.2016.1140743). Such side effects are associated with every biological system, e.g., musculoskeletal (e.g., osteoporosis, avascular necrosis of bone, and myopathy), endocrine and metabolic (e.g., hyperglycemia, diabetes mellitus, dyslipidemia, weight gain, Cushing syndrome, Cushingoid features, growth suppression, adrenal suppression), gastrointestinal (e.g., gastritis, peptic ulcer, gastrointestinal bleeding, visceral perforation, hepatic steatosis, pancreatitis), cardiovascular (e.g., hypertension, coronary heart disease, ischemic heart disease, heart failure), dermatologic (e.g., dermatoprosis, skin atrophy, ecchymosis, purpura, erosions, striae, delayed wound healing, easy bruising, acne, hirsutism, and hair loss), neuropsychiatric (e.g., mood changes, depression, euphoria, mood lability, irritability, akathisia, anxiety, cognitive impairment, psychosis, dementia, and delirium), ophthalmologic (e.g., cataract, glaucoma, ptosis, mydriasis, opportunistic ocular infections, and central serous chorioretinopathy), and immunologic (e.g., suppression of cell-mediated immunity, predisposition to infections, and reactivation of latent infections). In some embodiments, the compound of Formula (I), or a pharmaceutically acceptable salt thereof, is administered at a frequency of not less than twice daily. In some embodiments, the amount of the compound of Formula (I), or a pharmaceutically acceptable salt thereof, in the first administration is less than the amount of the compound of Formula (I), or a pharmaceutically acceptable salt thereof, in the second and any subsequent administrations. In some embodiments, the compound or pharmaceutical composition is administered at an amount sufficient to reduce the level of 17-hydroxyprogesterone (17-OHP) by at least 50% as compared to the level prior to administration. In some embodiments, the compound or pharmaceutical composition is administered at an amount sufficient to reduce the level of androstenedione by at least 30% as compared to the level prior to administration. In some embodiments, the compound or preparation is administered at an amount sufficient to (a) reduce the level of 17-hydroxyprogesterone (17-OHP) by at least 50% as compared to the level prior to administration; and (b) reduce the level of androstenedione by at least 30% as compared to the level prior to administration.

Accordingly, in some embodiments, the side effects of glucocorticoid treatment are selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, reactivation of latent infections, and any combination thereof.

Provided herein is a method of treating congenital adrenal hyperplasia in a subject comprising:
(i) measuring the level of one or more biomarkers selected from (a) 17-hydroxyprogesterone (17-OHP); (b) adrenocorticotropic hormone (ACTH); and (c) androstenedione in a biological sample obtained from the subject;
(ii) analyzing the level of the one or more biomarkers to determine if the level of the one or more biomarkers is elevated compared to a healthy subject not having congenital adrenal hyperplasia; and
(iii) administering to the subject a compound of Formula (I), or a pharmaceutically acceptable salt thereof if the subject is determined to have elevated levels of the one or more biomarkers.

In some embodiments, the method further comprises (iv) measuring the level of the one or more biomarkers after administering a compound of Formula (I), or a pharmaceutically acceptable salt thereof, in a biological sample obtained from the subject to determine whether the subject has reduced levels of the one or more biomarkers as compared with the measurement of step (i). In some embodiments, the method further comprises (v) continuing the administration of the compound of Formula (I), or a pharmaceutically acceptable salt thereof if the subject has reduced levels of the one or more biomarkers.

In some embodiments, steps (i) and (iv) are performed on biological samples taken from the subject in a similar manner and within a same time of day window. In some embodiments, steps (i) and (iv) are performed on biological samples taken from the subject within the time of day window from 2 a.m. to 10 a.m. In some embodiments, steps (i) and (iv) are performed on biological samples taken from the subject within the time of day window from 6 a.m. to 10 a.m. In some embodiments, steps (i) and (iv) comprise measuring the levels of at least two biomarkers selected from (a) 17-hydroxyprogesterone (17-OHP); (b) adrenocorticotropic hormone (ACTH); and (c) androstenedione. In some embodiments, steps (i) and (iv) comprise measuring the levels of (a) 17-hydroxyprogesterone (17-OHP); (b) adrenocorticotropic hormone (ACTH); and (c) androstenedione. In some embodiments, step (i) comprises measuring the level of 17-hydroxyprogesterone (17-01P), wherein the level of 17-hydroxyprogesterone (17-01P) is elevated when it is greater than or equal to 1,000 ng/dL. In some embodiments, step (i) comprises measuring the level of androstenedione, wherein the level of androstenedione is elevated when it is greater than 200 ng/dL.

In some embodiments of the methods of the present disclosure, the compound of Formula (I) is administered at an amount equivalent to from about 25 mg to about 150 mg of the compound of Formula (I) free base. In some embodiments, compound of Formula (I), or a pharmaceutically acceptable salt thereof, is administered at an amount equivalent to about 50 mg or about 100 mg of the compound of Formula (I) free base.

In some embodiments of the methods disclosed herein, the compound of Formula (I) is administered in the free base form.

In some embodiments of the methods disclosed herein, the compound of Formula (I) is administered twice daily (i.e., as a first and second administration). In some embodiments, the amount of the compound of Formula (I), or a pharmaceutically acceptable salt in the first administration is less than the amount of the compound of Formula (I), or a pharmaceutically acceptable salt thereof, in the second administration.

Also provided herein is method of treating congenital adrenal hyperplasia in a subject, the method comprising:
(a) selecting a subject who has a glucocorticoid dose of greater than 11 mg/m²/day after a time period of being administered a compound of Formula (I), or a pharmaceutically acceptable thereof, at an amount of about 200 mg twice daily based on the weight of the free base; and
(b) administering to the subject the compound of Formula (I), or a pharmaceutically acceptable salt thereof, at a frequency of twice daily;
wherein the amount of the compound of Formula (I), or a pharmaceutically acceptable salt thereof, in the first administration is less than the amount of the compound of Formula (I), or a pharmaceutically acceptable salt thereof, in the second administration. In some embodiments, the amount of the compound of Formula (I), or a pharmaceutically acceptable salt thereof, administered daily is greater than or equal to about 400 mg based on the weight of the free base.

In some embodiments, the amount of the compound of Formula (I), or a pharmaceutically acceptable salt thereof, administered daily in step (b) is greater than or equal to about 200 mg based on the weight of the free base.

In some embodiments, the glucocorticoid dose of step (a) is measured in hydrocortisone equivalents (which may be adjusted for body surface area (BSA)).

In some embodiments, the time period of administration in step (a) is at least about 4 weeks. In some embodiments, the time period of administration in step (a) is at least about 24 weeks. In some embodiments, the time period of administration in step (a) is at least about 6 months. In some embodiments, the time period of administration in step (a) is at least about one year.

Also provided herein is a method of treating CAH in a pediatric subject. The methods include administering to a pediatric subject a therapeutically effective amount of a compound of Formula (I), or pharmaceutically acceptable salt thereof. In some embodiments, the pediatric subject weighs greater than or equal to about 55 kg and a therapeutically effective amount of the compound of Formula (I), or a pharmaceutically acceptable salt thereof is about 100 mg administered twice daily (i.e., a total daily amount of about 200 mg based on the free base). In some embodiments, the pediatric subject weighs from about 10 kg to about 20 kg and a therapeutically effective amount of the compound of Formula (I), or a pharmaceutically acceptable salt thereof is about 25 mg administered twice daily (i.e., a total daily amount of about 50 mg based on the free base). In some embodiments, the pediatric subject weighs from about 20 kg to about 55 kg and a therapeutically effective amount of the compound of Formula (I), or a pharmaceutically acceptable salt thereof is about 50 mg administered twice daily (i.e., a total daily amount of about 100 mg, based on the free base). In some embodiments, the pediatric subject is a neonate. In some embodiments, the pediatric subject is an infant. In some embodiments, the pediatric subject is a child. In some embodiments, the pediatric subject is an adolescent.

In some embodiments of the methods of the present disclosure, the compound of Formula (I), or a pharmaceutically acceptable salt thereof, is administered to the subject in a fed state. The term “fed state,” as used herein, refers to administration of the compound of Formula (I), or a pharmaceutically acceptable salt thereof from about 1 hour before consumption of food or a nutritional composition to about 1 hour after consumption of food or a nutritional composition. The term “fasted state,” as used herein, refers to a gap of at least two hours between consumption of food or a nutritional composition and administration of the compound of Formula (I), or a pharmaceutically acceptable salt thereof. In some embodiments, the compound of Formula (I), or a pharmaceutically acceptable salt thereof is administered to the subject with food or a nutritional composition, such as a nutritional supplement or formula, a meal replacement beverage, a liquid dietary supplement, or a high caloric liquid meal. In some embodiments, the compound of Formula (I), or a pharmaceutically acceptable salt thereof is administered to the subject within about 1 hour before the subject has consumed food or a nutritional composition. In some embodiments, the compound of Formula (I), or a pharmaceutically acceptable salt thereof is administered to the subject within about 1 hour after the subject has consumed food or a nutritional composition. Examples of suitable nutritional compositions include, but are not limited to, infant formulas, dietary supplements, dietary substitutes, and rehydration compositions. In some embodiments, the food is a product containing concentrated calories and protein. In some embodiments, the nutritional composition is a composition utilized for enteral and parenteral supplementation for infants, specialty infant formulas, supplements for the elderly, and supplements for those with gastrointestinal difficulties and/or malabsorption. Adult and pediatric nutritional formulas are well known in the art and are commercially available (e.g., Similac®, Ensure®, Jevity® and Alimentum® from Ross Products Division, Abbott Laboratories, Columbus, Ohio).

In some embodiments, the nutritional composition is in liquid form. The energy density of the nutritional compositions, when in liquid form, can range from about 0.6 Kcal to about 3 Kcal per mL. In some embodiments, the nutritional composition is in solid or powdered form. When in solid or powdered form, the nutritional supplements can contain from about 1.2 to more than 9 Kcals per gram, such as about 3 to 7 Kcals per gram.

In some embodiments, the nutritional composition is a meal replacement bar. Examples include PowerBar®, Glucerna® bars, Choice DM® bars, Ensure® bars, and Boost® bars. In some embodiments, the nutritional composition is a nutrition shake or meal replacement beverage. Commercially available examples include the Ensure® branded adult products (such as Ensure® Original, Ensure® Plus, Ensure® Enlive, Ensure® High Protein, Ensure® Clear, and Ensure® Light), Glucerna®, Choice DM®, Slim Fast®, Pediasure®, Glytrol®, and Resource®. In some embodiments, the nutritional composition is Ensure® Plus. In some embodiments, the nutritional composition is vanilla-flavored Ensure® Plus. Ensure Plus® is a high calorie liquid dietary supplement that contains 1500 calories per liter with a caloric distribution of 14.7% protein, 32% fat and 53.3% carbohydrate.

In some embodiments of the disclosed methods, the compound of Formula (I), or a pharmaceutically acceptable salt thereof is administered to the subject with 8 fluid ounces (237 mL) of Ensure® Plus. In some embodiments, the Ensure® Plus is vanilla-flavored.

In some embodiments of the methods, the compound of Formula (I), or a pharmaceutically acceptable salt thereof is administered to the subject after administration of the nutritional composition. In some embodiments, the compound of Formula (I), or a pharmaceutically acceptable salt thereof is administered to the subject before administration of the nutritional composition. In some embodiments, the compound of Formula (I), or a pharmaceutically acceptable salt thereof, is administered to the subject at the same time as administration of the nutritional composition.

In some embodiments, the compound of Formula (I), or a pharmaceutically acceptable salt thereof is administered to the subject, followed by administration of the nutritional composition. In some embodiments, the nutritional composition is administered about 1 minute, about 5 minutes, about 10 minutes, about 15 minutes, about 20 minutes, about 25 minutes, about 30 minutes, about 35 minutes, about 40 minutes, about 45 minutes, or about 60 minutes, or within a range defined by any of the preceding values after administration of the compound of Formula (I), or a pharmaceutically acceptable salt thereof. In some embodiments, the nutritional composition is administered 1 minute, 5 minutes, 10 minutes, 15 minutes, 20 minutes, 25 minutes, 30 minutes, 35 minutes, 40 minutes, 45 minutes, or 60 minutes, or within a range defined by any of the preceding values after administration of the compound of Formula (I), or a pharmaceutically acceptable salt thereof. In some embodiments, the nutritional composition is administered within 30 minutes of administering the compound of Formula (I), or a pharmaceutically acceptable salt thereof. In some embodiments of the disclosed methods, the compound of Formula (I), or a pharmaceutically acceptable salt thereof, is administered to the subject with 8 fluid ounces (237 mL) of Ensure® Plus. In some embodiments, the Ensure® Plus is vanilla-flavored.

In some embodiments, the nutritional composition is administered to the subject, followed by administration of the compound of Formula (I), or a pharmaceutically acceptable salt thereof. In some embodiments, compound of Formula (I), or a pharmaceutically acceptable salt thereof is administered about 1 minute, about 5 minutes, about 10 minutes, about 15 minutes, about 20 minutes, about 25 minutes, about 30 minutes, about 35 minutes, about 40 minutes, about 45 minutes, or about 60 minutes, or within a range defined by any of the preceding values after administration of the nutritional composition. In some embodiments, the compound of Formula (I), or a pharmaceutically acceptable salt thereof, is administered 1 minute, 5 minutes, 10 minutes, 15 minutes, 20 minutes, 25 minutes, 30 minutes, 35 minutes, 40 minutes, 45 minutes, or 60 minutes, or within a range defined by any of the preceding values after administration of the nutritional composition. In some embodiments, the compound of Formula (I), or a pharmaceutically acceptable salt thereof, is administered within 30 minutes of administering the nutritional composition. In some embodiments of the disclosed methods, the compound of Formula (I), or a pharmaceutically acceptable salt thereof, is administered to the subject with 8 fluid ounces (237 mL) of Ensure® Plus. In some embodiments, the Ensure® Plus is vanilla-flavored.

In some embodiments of the methods, a food effect is observed between administration of the compound of Formula (I), or a pharmaceutically acceptable salt thereof in a fed state versus a fasted state. The term “food effect,” as used herein, refers to the relative difference in AUC (area under the curve AUC₍₀₋ₜ₎ and/or AUC_{(0-∞)}) or Cₘₐₓ (maximum plasma concentration or peak plasma concentration) of an active substance, when the compound of Formula (I), or a pharmaceutically acceptable salt thereof, is administered orally to a subject, concomitantly with food or in a fed state as compared to the same values when the same compound of Formula (I), or a pharmaceutically acceptable salt thereof is administered in a fasted state. The food effect (F) is calculated as:

F%=[(X_{fasted}−X_{fed})/X_{fasted}]×100

where X_{fed} and X_{fasted} are the values of AUC (AUC₍₀₋ₜ₎ and/or AUC_{(0-∞)}) or Cₘₐₓ in the fed and fasted state, respectively. In some embodiments, an increased, or positive, food effect is observed when the compound of Formula (I), or a pharmaceutically acceptable salt thereof is administered to a subject in a fed state. In some embodiments, administration of the compound of Formula (I), or a pharmaceutically acceptable salt thereof, results in an increased, or positive, food effect, whereby an increased Cₘₐₓ and/or AUC are observed when administered orally in the fed state as compared to the fasting state.

In some embodiments of the methods, the ratio of the AUC in the fed state to the AUC in the fasted state is about 5 to about 10, such as about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 8 to about 10. In some embodiments, the ratio of the AUC in the fed state to the AUC in the fasted state is about 5, about 6, about 7, about 8, about 9, or about 10, or within a range defined by any of the preceding values. In some embodiments of the methods, the ratio of the AUC in the fed state to the AUC in the fasted state is about 10 to about 20.

In some embodiments of the methods, the ratio of the AUC in the fed state to the AUC in the fasted state is 5 to 10, such as 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 8 to 10. In some embodiments, the ratio of the AUC in the fed state to the AUC in the fasted state is 5, 6, 7, 8, 9, or 10, or within a range defined by any of the preceding values.

In some embodiments of the methods, the ratio of the Cₘₐₓ in the fed state to the Cₘₐₓ in the fasted state is about 5 to about 10, such as about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 8 to about 10. In some embodiments, the ratio of the Cₘₐₓ in the fed state to the Cₘₐₓ in the fasted state is about 5, about 6, about 7, about 8, about 9, or about 10, or within a range defined by any of the preceding values. In some embodiments, the mean Cₘₐₓ of the compound of Formula (I), or pharmaceutically acceptable salt thereof, is about 1.5 to about 3 times higher in the fed stated compared to the fasted state. In some embodiments of the methods, the ratio of the Cₘₐₓ in the fed state to the Cₘₐₓ in the fasted state is 5 to 10, such as 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 8 to 10. In some embodiments, the ratio of the Cₘₐₓ in the fed state to the Cₘₐₓ in the fasted state is 5, 6, 7, 8, 9, or 10, or within a range defined by any of the preceding values. In some embodiments, the mean Cₘₐₓ of the compound of Formula (I), or pharmaceutically acceptable salt thereof, is 1.5 to 3 times higher in the fed stated compared to the fasted state. In some embodiments, the mean Cₘₐₓ of the compound of Formula (I), or pharmaceutically acceptable salt thereof, is about 2 times higher in the fed stated compared to the fasted state. In some embodiments of the methods, the ratio of the Cₘₐₓ in the fed state to the Cₘₐₓ in the fasted state is about 10 to about 20.

In some embodiments, the compound of Formula (I), or a pharmaceutically acceptable salt thereof, is administered to the subject with a meal. In some embodiments, the meal is a high fat, high caloric meal. In some embodiments, the meal is a low fat, low caloric meal. In some embodiments, the compound of Formula (I), or a pharmaceutically acceptable salt thereof, is administered within approximately 5 minutes after the start of the meal. In some embodiments, the meal is an evening meal. In some embodiments, the meal is a morning meal. In some embodiments, the meal is completed within about 30 minutes after administration of the compound of Formula (I), or a pharmaceutically acceptable salt thereof.

In some embodiments of the methods disclosed herein (e.g., when the compound of Formula (I) is administered at a frequency of twice daily), the first administration of the compound of Formula (I), or a pharmaceutically acceptable salt thereof is with a morning meal. In some embodiments of the methods disclosed herein, the second administration of the compound of Formula (I), or a pharmaceutically acceptable salt thereof is with an evening meal. In some embodiments of the methods disclosed herein, the first administration of the compound of Formula (I), or a pharmaceutically acceptable salt thereof, is with a morning meal and the second administration of the compound of Formula (I), or a pharmaceutically acceptable salt thereof is with an evening meal. In some embodiments of the methods disclosed herein (e.g., when the compound of Formula (I) is administered at a frequency of twice daily), there are about 6 to about 14 hours between the morning and evening meals. In some embodiments, there are about 8 to about 14 hours between the morning and evening meals. In some embodiments, there are about 11 to about 13 hours between the morning and evening meals. In some embodiments, there are about 12 hours between the morning and evening meals.

In some embodiments, the fed state is with a high fat meal. In some embodiments, the fed state is with a low fat meal. The FDA has provided draft guidelines regarding high fat and low fat meals (“Assessing the Effects of Food on Drugs in INDs and NDAs—Clinical Pharmacology Considerations Guidance for Industry,” U.S. Department of Health and Human Services, Food and Drug Administration, Center for Drug Evaluation and Research (CDER), February 2019, Clinical Pharmacology). Table 1 shows test meal definitions provided by the FDA guidance.

TABLE 1

| Meal | Total | Fat | | |
|---|---|---|---|---|
| Type | Kcal | Kcal | Grams | Percent |
| High-Fat | 800-1000 | 500-600 | 55-65 | 50 |
| Low-Fat | 400-500 | 100-125 | 11-14 | 25 |

The composition of a high fat meal provided by the FDA guidance is depicted in Table 2.

TABLE 2
Composition of a High Fat Meal*

| | |
|---|---|
| Total Calories | 800-1000 |
| Calories from Protein | 150 |
| Calories from Carbohydrates | 250 |
| Calories from Fat | 500-600 |
| An Example of a High Fat | Two eggs fried in butter |
| Breakfast | Two strips of bacon |
| | Two slices of toast with butter |
| | Four ounces of hash brown potatoes |
| | Eight ounces of whole milk |

*50 percent of calories are derived from fat. Substitutions can be made to this meal, if the content, volume, and viscosity are maintained.

The composition of a low fat meal provided by the FDA guidance is depicted in Table 3.

TABLE 3
Composition of a Low Fat Meal

| | |
|---|---|
| Total Calories | 400-500 |
| Fat (g) | 250 |
| Percent Calories from Fat | 25 |
| An Example of a Low Fat | Eight ounces milk (1 percent fat) |
| Breakfast* | One boiled egg |
| | One packet flavored instant oatmeal |
| | made with water |

*This low-fat breakfast contains 387 calories and has 10 grams of fat.

In some embodiments, a high fat meal contains 800-1000 total Kcal and 500-600 fat Kcal. In some embodiments, a low fat meal contains 400-500 total Kcal and 100-125 fat Kcal.

In some embodiments of the methods provided herein, the subject is a pediatric subject.

Also provided herein is a method of treating congenital adrenal hyperplasia (CAH), in a subject in need thereof, comprising administering to the subject a pharmaceutical composition of the present disclosure, wherein the pharmaceutical composition comprises a therapeutically effective amount of the compound of Formula (I), or a pharmaceutically acceptable salt thereof. In some embodiments, the pharmaceutical composition is a lipidic semi-solid formulation. In some embodiments, the pharmaceutical composition is a liquid formulation. In some embodiments, the pharmaceutical composition is administered to the subject in a fed state.

Also provided herein is a pharmaceutical composition of the present disclosure for use in a method of treating congenital adrenal hyperplasia (CAH) in a subject. In some embodiments, the subject is in a fed state.

In some embodiments, the pharmaceutical composition is administered to the subject with a nutritional composition. In some embodiments, the nutritional composition is a liquid dietary supplement comprising about 1000 to about 2000 calories per liter with a fat content greater than about 30%. In some embodiments, the nutritional composition is a liquid dietary supplement comprising 1500 calories per liter with a caloric distribution of 14.7% protein, 32% fat and 53.3% carbohydrate. In some embodiments, the nutritional composition is administered in an amount of about 6 to about 12 fluid ounces. In some embodiments, the nutritional composition is administered in an amount of about 8 fluid ounces. In some embodiments, the nutritional composition is administered within 30 minutes of administration of the pharmaceutical composition.

In some embodiments, the pharmaceutical composition exhibits a positive food effect. In some embodiments, the positive food effect is measured in terms of Cₘₐₓ, AUC, or a combination thereof of a compound of Formula (I) when comparing oral administration of the pharmaceutical composition in the fed and fasting states. In some embodiments, the ratio of the AUC of the compound of Formula (I) in the fed state to the AUC of the compound of Formula (I) in the fasted state is about 5 to about 10. In some embodiments, the ratio of the Cₘₐₓ of the compound of Formula (I) in the fed state to the Cₘₐₓ of the compound of Formula (I) in the fasted state is about 5 to about 10. In some embodiments, the ratio of the AUC of the compound of Formula (I) in the fed state to the AUC of the compound of Formula (I) in the fasted state is about 10 to about 20. In some embodiments, the ratio of the Cₘₐₓ of the compound of Formula (I) in the fed state to the Cₘₐₓ of the compound of Formula (I) in the fasted state is about 10 to about 20. In some embodiments, the ratio of the AUC of the compound of Formula (I) in the fed state to the AUC of the compound of Formula (I) in the fasted state is about 1 to about 4 or about 5 to about 10. In some embodiments, the ratio of the Cₘₐₓ of the compound of Formula (I) in the fed state to the Cₘₐₓ of the compound of Formula (I) in the fasted state is about 1 to about 4 or about 5 to about 10. In some embodiments, the ratio of the AUC of the compound of Formula (I) in the fed state to the AUC of the compound of Formula (I) in the fasted state is about 1 to about 4. In some embodiments, the ratio of the Cₘₐₓ of the compound of Formula (I) in the fed state to the Cₘₐₓ of the compound of Formula (I) in the fasted state is about 1 to about 4. In some embodiments, the ratio of the AUC of the compound of Formula (I) in the fed state to the AUC of the compound of Formula (I) in the fasted state is about 1.5 to about 3. In some embodiments, the ratio of the Cₘₐₓ of the compound of Formula (I) in the fed state to the Cₘₐₓ of the compound of Formula (I) in the fasted state is about 1.5 to about 3. In some embodiments, the ratio of the AUC of the compound of Formula (I) in the fed state to the AUC of the compound of Formula (I) in the fasted state is 1 to 4 or 5 to 10. In some embodiments, the ratio of the Cₘₐₓ of the compound of Formula (I) in the fed state to the Cₘₐₓ of the compound of Formula (I) in the fasted state is 1 to 4 or 5 to 10. In some embodiments, the ratio of the AUC of the compound of Formula (I) in the fed state to the AUC of the compound of Formula (I) in the fasted state is 1 to 4. In some embodiments, the ratio of the Cₘₐₓ of the compound of Formula (I) in the fed state to the Cₘₐₓ of the compound of Formula (I) in the fasted state is 1 to 4. In some embodiments, the ratio of the AUC of the compound of Formula (I) in the fed state to the AUC of the compound of Formula (I) in the fasted state is 1.5 to 3. In some embodiments, the ratio of the Cₘₐₓ of the compound of Formula (I) in the fed state to the Cₘₐₓ of the compound of Formula (I) in the fasted state is 1.5 to 3.

In some embodiments, the subject is a pediatric subject.

In some embodiments, the pharmaceutical composition is formulated for oral administration and exhibits a positive food effect when administered orally. In some embodiments, the compound of Formula (I) has a ratio of the AUC in the fed state to the AUC in the fasted state of about 5 to about 10. In some embodiments, the compound of Formula (I) has a ratio of the Cₘₐₓ in the fed state to the Cₘₐₓ in the fasted state of about 5 to about 10. In some embodiments, the compound of Formula (I) has a ratio of the AUC in the fed state to the AUC in the fasted state of about 10 to about 20. In some embodiments, the compound of Formula (I) has a ratio of the Cₘₐₓ in the fed state to the Cₘₐₓ in the fasted state of about 10 to about 20. In some embodiments, the compound of Formula (I) has a ratio of the AUC in the fed state to the AUC in the fasted state of about 1 to about 4 or about 5 to about 10. In some embodiments, the compound of Formula (I) has a ratio of the Cₘₐₓ in the fed state to the Cₘₐₓ in the fasted state of about 1 to about 4 or about 5 to about 10. In some embodiments, the compound of Formula (I) has a ratio of the AUC in the fed state to the AUC in the fasted state of about 1 to about 4. In some embodiments, the compound of Formula (I) has a ratio of the Cₘₐₓ in the fed state to the Cₘₐₓ in the fasted state of about 1 to about 4. In some embodiments, the compound of Formula (I) has a ratio of the AUC in the fed state to the AUC in the fasted state of about 1.5 to about 3. In some embodiments, the compound of Formula (I) has a ratio of the Cₘₐₓ in the fed state to the Cₘₐₓ in the fasted state of about 1.5 to about 3. In some embodiments, the compound of Formula (I) has a ratio of the AUC in the fed state to the AUC in the fasted state of 1 to 4 or 5 to 10. In some embodiments, the compound of Formula (I) has a ratio of the Cₘₐₓ in the fed state to the Cₘₐₓ in the fasted state of 1 to 4 or 5 to 10. In some embodiments, the compound of Formula (I) has a ratio of the AUC in the fed state to the AUC in the fasted state of 1 to 4. In some embodiments, the compound of Formula (I) has a ratio of the Cₘₐₓ in the fed state to the Cₘₐₓ in the fasted state of 1 to 4. In some embodiments, the compound of Formula (I) has a ratio of the AUC in the fed state to the AUC in the fasted state of 1.5 to 3. In some embodiments, the compound of Formula (I) has a ratio of the Cₘₐₓ in the fed state to the Cₘₐₓ in the fasted state of 1.5 to 3.

In some embodiments, the pharmaceutical composition is administered to the subject with a meal. In some embodiments, the meal is a high fat meal. In some embodiments, the meal is a low fat meal. In some embodiments, the pharmaceutical composition is administered within about 5 minutes after the start of the meal. In some embodiments, the meal is an evening meal. In some embodiments, the meal is a morning meal.

In some embodiments, administering the pharmaceutical composition exhibits a positive food effect. In some embodiments, the positive food effect is measured in terms of Cₘₐₓ, AUC, or combinations thereof of the compound of Formula (I) when comparing oral administration of the pharmaceutical composition in the fed and fasting states. In some embodiments, the ratio of the AUC of the compound of Formula (I) in the fed state to the AUC of the compound of Formula (I) in the fasted state is about 5 to about 10. In some embodiments, the ratio of the Cₘₐₓ of the compound of Formula (I) in the fed state to the Cₘₐₓ of the compound of Formula (I) in the fasted state is about 5 to about 10. In some embodiments, the ratio of the AUC of the compound of Formula (I) in the fed state to the AUC of the compound of Formula (I) in the fasted state is about 10 to about 20. In some embodiments, the ratio of the Cₘₐₓ of the compound of Formula (I) in the fed state to the Cₘₐₓ of the compound of Formula (I) in the fasted state is about 10 to about 20. In some embodiments, the ratio of the AUC of the compound of Formula (I) in the fed state to the AUC of the compound of Formula (I) in the fasted state is about 1 to about 4 or about 5 to about 10. In some embodiments, the ratio of the Cₘₐₓ of the compound of Formula (I) in the fed state to the Cₘₐₓ of the compound of Formula (I) in the fasted state is about 1 to about 4 or about 5 to about 10. In some embodiments, the ratio of the AUC of the compound of Formula (I) in the fed state to the AUC of the compound of Formula (I) in the fasted state is about 1 to about 4. In some embodiments, the ratio of the Cₘₐₓ of the compound of Formula (I) in the fed state to the Cₘₐₓ of the compound of Formula (I) in the fasted state is about 1 to about 4. In some embodiments, the ratio of the AUC of the compound of Formula (I) in the fed state to the AUC of the compound of Formula (I) in the fasted state is about 1.5 to about 3. In some embodiments, the ratio of the Cₘₐₓ of the compound of Formula (I) in the fed state to the Cₘₐₓ of the compound of Formula (I) in the fasted state is about 1.5 to about 3. In some embodiments, the ratio of the AUC of the compound of Formula (I) in the fed state to the AUC of the compound of Formula (I) in the fasted state is 1 to 4 or 5 to 10. In some embodiments, the ratio of the Cₘₐₓ of the compound of Formula (I) in the fed state to the Cₘₐₓ of the compound of Formula (I) in the fasted state is 1 to 4 or 5 to 10. In some embodiments, the ratio of the AUC of the compound of Formula (I) in the fed state to the AUC of the compound of Formula (I) in the fasted state is 1 to 4. In some embodiments, the ratio of the Cₘₐₓ of the compound of Formula (I) in the fed state to the Cₘₐₓ of the compound of Formula (I) in the fasted state is 1 to 4. In some embodiments, the ratio of the AUC of the compound of Formula (I) in the fed state to the AUC of the compound of Formula (I) in the fasted state is 1.5 to 3. In some embodiments, the ratio of the Cₘₐₓ of the compound of Formula (I) in the fed state to the Cₘₐₓ of the compound of Formula (I) in the fasted state is 1.5 to 3.

For the avoidance of doubt, also provided herein is the corresponding compound of Formula (I), or a pharmaceutically acceptable salt thereof, or corresponding pharmaceutical composition comprising a compound of Formula (I), or a pharmaceutically acceptable salt thereof, for use in the corresponding methods, as described herein.

For the avoidance of doubt, also provided herein is use of the corresponding compound of Formula (I), or a pharmaceutically acceptable salt thereof, in the manufacture of a medicament for use in the corresponding methods, as described herein.

For the avoidance of doubt, also provided herein is use of the corresponding pharmaceutical composition comprising a compound of Formula (I), or a pharmaceutically acceptable salt thereof, in the manufacture of a medicament for use in the corresponding methods, as described herein.

Reduction in Glucocorticoid Burden, Adrenal Androgens and Precursors

Glucocorticoids are a class of corticosteroids, which are a class of steroid hormones. Glucocorticoids are corticosteroids that bind to the glucocorticoid receptor that is present in almost every vertebrate animal cell. In some embodiments, the subject is concurrently receiving a dose of a glucocorticoid. In some embodiments, the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate. In some embodiments, the glucocorticoid is cortisol (hydrocortisone). In some embodiments, the glucocorticoid is cortisone. In some embodiments, the glucocorticoid is prednisone. In some embodiments, the glucocorticoid is dexamethasone.

In some embodiments, the glucocorticoid dose is measured in hydrocortisone equivalents. In some embodiments, the glucocorticoid dose is measured as a multiple of the upper limit of normal of physiologic dosing in hydrocortisone equivalents. Any glucocorticoid can be given in a dose that provides approximately the same glucocorticoid effects as normal cortisol production; this is referred to as physiologic, replacement, or maintenance dosing.

In some embodiments, the glucocorticoid dose is a physiologic dose as measured after a time period of administration of the compound of Formula (I), or a pharmaceutically acceptable salt thereof. In some embodiments, the glucocorticoid dose is a physiologic dose of about 4 to about 12 mg/m²/day as measured after a time period of administration of the compound of Formula (I), or a pharmaceutically acceptable salt thereof. In some embodiments, the glucocorticoid dose is a physiologic dose of about 4 to about 9 mg/m²/day as measured after a time period of administration of the compound of Formula (I), or a pharmaceutically acceptable salt thereof. In some embodiments, the glucocorticoid dose is a physiologic dose that is less than about 8 mg/m²/day as measured after a time period of administration of the compound of Formula (I), or a pharmaceutically acceptable salt thereof.

In some embodiments, the glucocorticoid dose is a physiologic dose as measured after a time period of administration of the pharmaceutical composition comprising the compound of Formula (I), or a pharmaceutically acceptable salt thereof. In some embodiments, the glucocorticoid dose is a physiologic dose of about 4 to about 12 mg/m²/day as measured after a time period of administration of the pharmaceutical composition comprising the compound of Formula (I), or a pharmaceutically acceptable salt thereof. In some embodiments, the glucocorticoid dose is a physiologic dose of about 4 to about 9 mg/m²/day as measured after a time period of administration of the pharmaceutical composition comprising the compound of Formula (I), or a pharmaceutically acceptable salt thereof. In some embodiments, the glucocorticoid dose is a physiologic dose that is less than about 8 mg/m²/day as measured after a time period of administration of the pharmaceutical composition comprising the compound of Formula (I), or a pharmaceutically acceptable salt thereof.

In some embodiments, the glucocorticoid dose concurrently given to the subject is a normal physiological dose of hydrocortisone equivalents. In some embodiments, the glucocorticoid dose concurrently given to the subject is determined after a time period of administration of the compound of Formula (I), or a pharmaceutically acceptable salt thereof. In some embodiments, the glucocorticoid dose concurrently given to the subject is determined after a time period of administration of the pharmaceutical composition comprising the compound of Formula (I), or a pharmaceutically acceptable salt thereof. In some embodiments, a normal physiological dose of hydrocortisone equivalents is about 2 to about 16 mg/m²/day. In some embodiments, a normal physiological dose of hydrocortisone equivalents is about 4 to about 12 mg/m²/day. In some embodiments, a normal physiological dose of hydrocortisone equivalents is about 5 to about 11 mg/m²/day. In some embodiments, a normal physiological dose of hydrocortisone equivalents is about 6 to about 10 mg/m²/day. In some embodiments, a normal physiological dose of hydrocortisone equivalents is about 7 to about 9 mg/m²/day. In some embodiments, a normal physiological dose of hydrocortisone equivalents is about 4 to about 9 mg/m²/day. In some embodiments, a normal physiological dose of hydrocortisone equivalents is about 8 mg/m²/day. In some embodiments, a normal physiological dose of hydrocortisone equivalents is about 12 mg/m²/day. In some embodiments, a normal physiological dose of hydrocortisone equivalents is less than about 8 mg/m²/day. In some embodiments, a normal physiological dose of hydrocortisone equivalents is about 2, about 3, about 4, about 5, about 6, about 7, about 8, about 9, about 10, about 11, about 12, about 13, about 14, about 15 or about 16 mg/m²/day, or within a range defined by any of the preceding values.

In some embodiments, the glucocorticoid dose concurrently given to the subject is at the upper limit of normal of a normal physiological dose of hydrocortisone equivalents. In some embodiments, the glucocorticoid dose concurrently given to the subject is determined after a time period of administration of the compound of Formula (I), or a pharmaceutically acceptable salt thereof. In some embodiments, the glucocorticoid dose concurrently given to the subject is determined after a time period of administration of the pharmaceutical composition comprising the compound of Formula (I), or a pharmaceutically acceptable salt thereof. In some embodiments, the upper limit of normal is 1.5 times the normal physiological dose. In some embodiments, the upper limit of normal is about 1.5 times the normal physiological dose. In some embodiments, the upper limit of normal is about 1.5 times the normal physiological dose. In some embodiments, the upper limit of normal is about 2 times the normal physiological dose. In some embodiments, the upper limit of normal is about 2.5 times the normal physiological dose. In some embodiments, the upper limit of normal is about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 times the normal physiological dose, or within a range defined by any of the preceding values.

In some embodiments, the glucocorticoid dose of the subject is reduced by about 10% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 30% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 40% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 60% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 70% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by less than about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by about 20% to about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof. In some embodiments, the glucocorticoid dose of the subject is reduced by greater than about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

In some embodiments, the glucocorticoid dose of the subject is reduced by about 10% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 20% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 30% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 40% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 50% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 60% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 70% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by less than about 20% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by about 20% to about 50% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof. In some embodiments, the glucocorticoid dose of the subject is reduced by greater than about 50% after a time period of administration of the pharmaceutical composition comprising the compound of Formula (I), or a pharmaceutically acceptable salt thereof, wherein the reduction of the glucocorticoid dose is relative to the glucocorticoid dose prior to administration of the pharmaceutical composition comprising the compound of Formula (I), or a pharmaceutically acceptable salt thereof.

In some embodiments, the glucocorticoid dose of the subject is reduced within a range defined by any of the preceding values.

In some embodiments, the level of 17-hydroxyprogesterone is reduced by at least 25% after a time period of administration of the compound of Formula (I), or a pharmaceutically acceptable salt thereof, wherein the reduction of the level of 17-hydroxyprogesterone is relative to the level of 17-hydroxyprogesterone prior to administration of the compound of Formula (I), or a pharmaceutically acceptable salt thereof. In some embodiments, the level of 17-hydroxyprogesterone is reduced by at least 50% after a time period of administration of the compound of Formula (I), or a pharmaceutically acceptable salt thereof, wherein the reduction of the level of 17-hydroxyprogesterone is relative to the level of 17-hydroxyprogesterone prior to administration of the compound of Formula (I), or a pharmaceutically acceptable salt thereof. In some embodiments, the level of 17-hydroxyprogesterone is less than 1.5 times the upper limit of normal after a time period of administration of the compound of Formula (I), or a pharmaceutically acceptable salt thereof. In some embodiments, the level of 17-hydroxyprogesterone is within normal limits after a time period of administration of the compound of Formula (I), or a pharmaceutically acceptable salt thereof.

In some embodiments, the level of 17-hydroxyprogesterone is reduced by at least about 25% after a time period of administration of the pharmaceutical composition comprising the compound of Formula (I), or a pharmaceutically acceptable salt thereof, wherein the reduction of the level of 17-hydroxyprogesterone is relative to the level of 17-hydroxyprogesterone prior to administration of the pharmaceutical composition comprising the compound of Formula (I), or a pharmaceutically acceptable salt thereof. In some embodiments, the level of 17-hydroxyprogesterone is reduced by at least about 50% after a time period of administration of the pharmaceutical composition comprising the compound of Formula (I), or a pharmaceutically acceptable salt thereof, wherein the reduction of the level of 17-hydroxyprogesterone is relative to the level of 17-hydroxyprogesterone prior to administration of the pharmaceutical composition comprising the compound of Formula (I), or a pharmaceutically acceptable salt thereof. In some embodiments, the level of 17-hydroxyprogesterone is less than about 1.5 times the upper limit of normal after a time period of administration of the pharmaceutical composition comprising the compound of Formula (I), or a pharmaceutically acceptable salt thereof. In some embodiments, the level of 17-hydroxyprogesterone is within normal limits after a time period of administration of the pharmaceutical composition comprising the compound of Formula (I), or a pharmaceutically acceptable salt thereof.

In some embodiments, the level of 17-hydroxyprogesterone of the subject is reduced within a range defined by any of the preceding values.

In some embodiments, the level of adrenocorticotropic hormone is reduced by at least 25% after a time period of administration of the compound of Formula (I), or a pharmaceutically acceptable salt thereof, wherein the reduction of the level of adrenocorticotropic hormone is relative to the level of adrenocorticotropic hormone prior to administration of the compound of Formula (I), or a pharmaceutically acceptable salt thereof. In some embodiments, the level of adrenocorticotropic hormone is reduced by at least 40% after a time period of administration of the compound of Formula (I), or a pharmaceutically acceptable salt thereof, wherein the reduction of the level of adrenocorticotropic hormone is relative to the level of adrenocorticotropic hormone prior to administration of the compound of Formula (I), or a pharmaceutically acceptable salt thereof. In some embodiments, the level of adrenocorticotropic hormone is reduced by at least 50% after a time period of administration of the compound of Formula (I), or a pharmaceutically acceptable salt thereof, wherein the reduction of the level of adrenocorticotropic hormone is relative to the level of adrenocorticotropic hormone prior to administration of the compound of Formula (I), or a pharmaceutically acceptable salt thereof. In some embodiments, the level of adrenocorticotropic hormone is less than 1.5 times the upper limit of normal after a time period of administration of the compound of Formula (I), or a pharmaceutically acceptable salt thereof. In some embodiments, the level of adrenocorticotropic hormone is within normal limits after a time period of administration of the compound of Formula (I), or a pharmaceutically acceptable salt thereof.

In some embodiments, the level of adrenocorticotropic hormone is reduced by at least about 25% after a time period of administration of the pharmaceutical composition comprising the compound of Formula (I), or a pharmaceutically acceptable salt thereof, wherein the reduction of the level of adrenocorticotropic hormone is relative to the level of adrenocorticotropic hormone prior to administration of the pharmaceutical composition comprising the compound of Formula (I), or a pharmaceutically acceptable salt thereof. In some embodiments, the level of adrenocorticotropic hormone is reduced by at least about 40% after a time period of administration of the pharmaceutical composition comprising the compound of Formula (I), or a pharmaceutically acceptable salt thereof, wherein the reduction of the level of adrenocorticotropic hormone is relative to the level of adrenocorticotropic hormone prior to administration of the pharmaceutical composition comprising the compound of Formula (I), or a pharmaceutically acceptable salt thereof. In some embodiments, the level of adrenocorticotropic hormone is reduced by at least about 50% after a time period of administration of the pharmaceutical composition comprising the compound of Formula (I), or a pharmaceutically acceptable salt thereof, wherein the reduction of the level of adrenocorticotropic hormone is relative to the level of adrenocorticotropic hormone prior to administration of the pharmaceutical composition comprising the compound of Formula (I), or a pharmaceutically acceptable salt thereof. In some embodiments, the level of adrenocorticotropic hormone is less than about 1.5 times the upper limit of normal after a time period of administration of the pharmaceutical composition comprising the compound of Formula (I), or a pharmaceutically acceptable salt thereof. In some embodiments, the level of adrenocorticotropic hormone is within normal limits after a time period of administration of the pharmaceutical composition comprising the compound of Formula (I), or a pharmaceutically acceptable salt thereof.

In some embodiments, the level of adrenocorticotropic hormone of the subject is reduced within a range defined by any of the preceding values.

In some embodiments, the level of androstenedione is reduced by at least 25% after a time period of administration of the compound of Formula (I), or a pharmaceutically acceptable salt thereof, wherein the reduction of the level of androstenedione is relative to the level of androstenedione prior to administration of the compound of Formula (I), or a pharmaceutically acceptable salt thereof. In some embodiments, the level of androstenedione is reduced by at least 30% after a time period of administration of the compound of Formula (I), or a pharmaceutically acceptable salt thereof, wherein the reduction of the level of androstenedione is relative to the level of androstenedione prior to administration of the compound of Formula (I), or a pharmaceutically acceptable salt thereof. In some embodiments, the level of androstenedione is reduced by at least 50% after a time period of administration of the compound of Formula (I), or a pharmaceutically acceptable salt thereof, wherein the reduction of the level of androstenedione is relative to the level of androstenedione prior to administration of the compound of Formula (I), or a pharmaceutically acceptable salt thereof. In some embodiments, the level of androstenedione is less than 1.5 times the upper limit of normal after a time period of administration of the compound of Formula (I), or a pharmaceutically acceptable salt thereof. In some embodiments, the level of androstenedione is within normal limits after a time period of administration of the compound of Formula (I), or a pharmaceutically acceptable salt thereof.

In some embodiments, the level of androstenedione is reduced by at least about 25% after a time period of administration of the pharmaceutical composition comprising the compound of Formula (I), or a pharmaceutically acceptable salt thereof, wherein the reduction of the level of androstenedione is relative to the level of androstenedione prior to administration of the pharmaceutical composition comprising the compound of Formula (I), or a pharmaceutically acceptable salt thereof. In some embodiments, the level of androstenedione is reduced by at least about 30% after a time period of administration of the pharmaceutical composition comprising the compound of Formula (I), or a pharmaceutically acceptable salt thereof, wherein the reduction of the level of androstenedione is relative to the level of androstenedione prior to administration of the pharmaceutical composition comprising the compound of Formula (I), or a pharmaceutically acceptable salt thereof. In some embodiments, the level of androstenedione is reduced by at least about 50% after a time period of administration of the pharmaceutical composition comprising the compound of Formula (I), or a pharmaceutically acceptable salt thereof, wherein the reduction of the level of androstenedione is relative to the level of androstenedione prior to administration of the pharmaceutical composition comprising the compound of Formula (I), or a pharmaceutically acceptable salt thereof. In some embodiments, the level of androstenedione is less than about 1.5 times the upper limit of normal after a time period of administration of the pharmaceutical composition comprising the compound of Formula (I), or a pharmaceutically acceptable salt thereof. In some embodiments, the level of androstenedione is within normal limits after a time period of administration of the pharmaceutical composition comprising the compound of Formula (I), or a pharmaceutically acceptable salt thereof.

In some embodiments, the level of androstenedione of the subject is reduced within a range defined by any of the preceding values.

In some embodiments, the level of testosterone is reduced by at least 25% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof. In some embodiments, the level of testosterone is reduced by at least 30% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof. In some embodiments, the level of testosterone is reduced by at least 50% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof. In some embodiments, the level of testosterone is less than 1.5 times the upper limit of normal after a time period of administration of the compound of Formula (I), or a pharmaceutically acceptable salt thereof. In some embodiments, the level of testosterone is within normal limits after a time period of administration of the compound of Formula (I), or a pharmaceutically acceptable salt thereof.

In some embodiments, the level of testosterone is reduced by at least about 25% after a time period of administration of the pharmaceutical composition comprising the compound of Formula (I), or a pharmaceutically acceptable salt thereof, wherein the reduction of the level of testosterone is relative to the level of testosterone prior to administration of the pharmaceutical composition comprising the compound of Formula (I), or a pharmaceutically acceptable salt thereof. In some embodiments, the level of testosterone is reduced by at least about 30% after a time period of administration of the pharmaceutical composition comprising the compound of Formula (I), or a pharmaceutically acceptable salt thereof, wherein the reduction of the level of testosterone is relative to the level of testosterone prior to administration of the pharmaceutical composition comprising the compound of Formula (I), or a pharmaceutically acceptable salt thereof. In some embodiments, the level of testosterone is reduced by at least about 50% after a time period of administration of the pharmaceutical composition comprising the compound of Formula (I), or a pharmaceutically acceptable salt thereof, wherein the reduction of the level of testosterone is relative to the level of testosterone prior to administration of the pharmaceutical composition comprising the compound of Formula (I), or a pharmaceutically acceptable salt thereof. In some embodiments, the level of testosterone is less than about 1.5 times the upper limit of normal after a time period of administration of the pharmaceutical composition comprising the compound of Formula (I), or a pharmaceutically acceptable salt thereof. In some embodiments, the level of testosterone is within normal limits after a time period of administration of the pharmaceutical composition comprising the compound of Formula (I), or a pharmaceutically acceptable salt thereof.

In some embodiments, the level of testosterone of the subject is reduced within a range defined by any of the preceding values.

In some embodiments, the level of 17-hydroxyprogesterone is reduced by at least 50% and the level of androstenedione is reduced by at least 50% after a time period of administration of the compound of Formula (I), or a pharmaceutically acceptable salt thereof, wherein the reduction of the level of 17-hydroxyprogesterone and the level of androstenedione is relative to the level of 17-hydroxyprogesterone and the level of androstenedione prior to administration of the compound of Formula (I), or a pharmaceutically acceptable salt thereof. In some embodiments, the level of 17-hydroxyprogesterone is less than 1.5 times the upper limit of normal and the level of androstenedione is less than 1.5 times the upper limit of normal after a time period of administration of the compound of Formula (I), or a pharmaceutically acceptable salt thereof. In some embodiments, the level of 17-hydroxyprogesterone is within normal limits and the level of androstenedione is within normal limits after a time period of administration of the compound of Formula (I), or a pharmaceutically acceptable salt thereof.

In some embodiments, the level of 17-hydroxyprogesterone is reduced by at least about 50% and the level of androstenedione is reduced by at least about 50% after a time period of administration of the pharmaceutical composition comprising the compound of Formula (I), or a pharmaceutically acceptable salt thereof, wherein the reduction of the level of 17-hydroxyprogesterone and the level of androstenedione is relative to the level of 17-hydroxyprogesterone and the level of androstenedione prior to administration of the pharmaceutical composition comprising the compound of Formula (I), or a pharmaceutically acceptable salt thereof. In some embodiments, the level of 17-hydroxyprogesterone is less than about 1.5 times the upper limit of normal and the level of androstenedione is less than about 1.5 times the upper limit of normal after a time period of administration of the pharmaceutical composition comprising the compound of Formula (I), or a pharmaceutically acceptable salt thereof. In some embodiments, the level of 17-hydroxyprogesterone is within normal limits and the level of androstenedione is within normal limits after a time period of administration of the pharmaceutical composition comprising the compound of Formula (I), or a pharmaceutically acceptable salt thereof.

In some embodiments, the level of 17-hydroxyprogesterone and androstenedione of the subject is reduced within a range defined by any of the preceding values.

In some embodiments, the subject exhibits a decrease in glucocorticoid burden after a time period of administration of the compound of Formula (I), or a pharmaceutically acceptable salt thereof, wherein the decrease in glucocorticoid burden is relative to the glucocorticoid burden prior to administration of the compound of Formula (I), or a pharmaceutically acceptable salt thereof. In some embodiments, one or more symptoms selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor (TART), control of ovarian adrenal rest tumors (OART) and fertility, is improved after a time period of administration of the compound of Formula (I), or a pharmaceutically acceptable salt thereof, wherein the improvement in the one or more symptoms is relative to the status of the one or more symptoms prior to administration of the compound of Formula (I), or a pharmaceutically acceptable salt thereof.

In some embodiments, the subject exhibits a decrease in glucocorticoid burden after a time period of administration of the pharmaceutical composition comprising the compound of Formula (I), or a pharmaceutically acceptable salt thereof, wherein the decrease in glucocorticoid burden is relative to the glucocorticoid burden prior to administration of the pharmaceutical composition comprising the compound of Formula (I), or a pharmaceutically acceptable salt thereof. In some embodiments, one or more symptoms of glucocorticoid burden selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor (TART), control of ovarian adrenal rest tumor (OART), and fertility, is improved after a time period of administration of the pharmaceutical composition comprising the compound of Formula (I), or a pharmaceutically acceptable salt thereof, wherein the improvement in the one or more symptoms is relative to the status of the one or more symptoms prior to administration of the pharmaceutical composition comprising the compound of Formula (I), or a pharmaceutically acceptable salt thereof.

In some embodiments, the level of one or more adrenal steroids, or a precursor thereof, is reduced by at least 25% after a time period of administration of the compound of Formula (I), or a pharmaceutically acceptable salt thereof, wherein the reduction of the level of the adrenal steroid, or a precursor thereof, is relative to the level of adrenal steroid, or a precursor thereof, prior to administration of the compound of Formula (I), or a pharmaceutically acceptable salt thereof. In some embodiments, the level of adrenal steroid, or a precursor thereof, is reduced by at least 50% after a time period of administration of the compound of Formula (I), or a pharmaceutically acceptable salt thereof, wherein the reduction of the level of adrenal steroid, or a precursor thereof, is relative to the level of adrenal steroid, or a precursor thereof, prior to administration of the compound of Formula (I), or a pharmaceutically acceptable salt thereof. In some embodiments, the level of the adrenal steroid, or a precursor thereof, is less than 1.5 times the upper limit of normal after a time period of administration of the compound of Formula (I), or a pharmaceutically acceptable salt thereof. In some embodiments, the level of the adrenal steroid, or a precursor thereof, is within normal limits after a time period of administration of the compound of Formula (I), or a pharmaceutically acceptable salt thereof.

In some embodiments, the quality of life as measured by the EuroQol 5 Dimensions 5 Levels (EQ-5D-5L) in the subject is improved after a time period of administration of the compound of Formula (I), or a pharmaceutically acceptable salt thereof, wherein the improvement in the EuroQol 5 Dimensions 5 Levels (EQ-5D-5L) is relative to the EuroQol 5 Dimensions 5 Levels (EQ-5D-5L) results prior to administration of the compound of Formula (I), or a pharmaceutically acceptable salt thereof.

In some embodiments, fatigue is reduced in the subject after a time period of administration of the compound of Formula (I), or a pharmaceutically acceptable salt thereof, wherein the reduction in fatigue is relative to the fatigue prior to administration of the compound of Formula (I), or a pharmaceutically acceptable salt thereof.

In some embodiments, sleep is improved in the subject after a time period of administration of the compound of Formula (I), or a pharmaceutically acceptable salt thereof, wherein the improvement in sleep is relative to the sleep prior to administration of the compound of Formula (I), or a pharmaceutically acceptable salt thereof. An improvement in sleep can comprise one or more of reduction in latency to sleep onset, increase in total sleep time, and/or an improvement in sleep quality.

In some embodiments, insulin resistance is reduced in the subject after a time period of administration of the compound of Formula (I), or a pharmaceutically acceptable salt thereof, wherein the reduction of insulin resistance is relative to the insulin resistance prior to administration of the compound of Formula (I), or a pharmaceutically acceptable salt thereof.

In some embodiments, glucose tolerance (e.g., an impaired glucose tolerance) is improved in the subject after a time period of administration of the compound of Formula (I), or a pharmaceutically acceptable salt thereof, wherein the improvement in glucose tolerance is relative to the glucose tolerance prior to administration of the compound of Formula (I), or a pharmaceutically acceptable salt thereof.

In some embodiments, glucose control is increased in the subject after a time period of administration of the compound of Formula (I), or a pharmaceutically acceptable salt thereof, wherein the increase in glucose control is relative to the glucose control prior to administration of the compound of Formula (I), or a pharmaceutically acceptable salt thereof.

In some embodiments, lipid levels reflecting dyslipidemia are improved (e.g., reduced) in the subject after a time period of administration of the compound of Formula (I), or a pharmaceutically acceptable salt thereof, wherein the improvement in lipid levels is relative to the lipid levels prior to administration of the compound of Formula (I), or a pharmaceutically acceptable salt thereof.

In some embodiments, lipid levels reflecting hyperlipidemia are reduced in the subject after a time period of administration of the compound of Formula (I), or a pharmaceutically acceptable salt thereof, wherein the reduction in lipid levels is relative to the lipid levels prior to administration of the compound of Formula (I), or a pharmaceutically acceptable salt thereof.

In some embodiments, bone mineral density is increased in the subject after a time period of administration of the compound of Formula (I), or a pharmaceutically acceptable salt thereof, wherein the increase in bone mineral density is relative to the bone mineral density prior to administration of the compound of Formula (I), or a pharmaceutically acceptable salt thereof.

In some embodiments, bone turnover is improved (e.g., an increase in bone turnover markers consistent with a decrease in bone loss) in the subject after a time period of administration of the compound of Formula (I), or a pharmaceutically acceptable salt thereof, wherein the improvement in bone turnover is relative to the bone turnover prior to administration of the compound of Formula (I), or a pharmaceutically acceptable salt thereof.

In some embodiments, fat mass is decreased in the subject after a time period of administration of the compound of Formula (I), or a pharmaceutically acceptable salt thereof, wherein the decrease in fat mass is relative to the fat mass prior to administration of the compound of Formula (I), or a pharmaceutically acceptable salt thereof.

In some embodiments, body weight is decreased in the subject (e.g., in a subject who is overweight, obese, and/or exhibits central obesity) after a time period of administration of the compound of Formula (I), or a pharmaceutically acceptable salt thereof, wherein the decrease in body weight is relative to the body weight prior to administration of the compound of Formula (I), or a pharmaceutically acceptable salt thereof.

In some embodiments, central obesity is decreased in the subject after a time period of administration of the compound of Formula (I), or a pharmaceutically acceptable salt thereof, wherein the decrease in central obesity is relative to the central obesity prior to administration of the compound of Formula (I), or a pharmaceutically acceptable salt thereof.

In some embodiments, blood pressure is improved in the subject (e.g., a decrease in blood pressure in a subject with hypertension) after a time period of administration of the compound of Formula (I), or a pharmaceutically acceptable salt thereof, wherein the improvement in blood pressure is relative to the blood pressure prior to administration of the compound of Formula (I), or a pharmaceutically acceptable salt thereof.

In some embodiments, the severity of hirsutism is decreased in the subject after a time period of administration of the compound of Formula (I), or a pharmaceutically acceptable salt thereof, wherein the decrease in the severity of hirsutism is relative to the severity of hirsutism prior to administration of the compound of Formula (I), or a pharmaceutically acceptable salt thereof.

In some embodiments, menstrual regularity is improved or restored in the subject after a time period of administration of the compound of Formula (I), or a pharmaceutically acceptable salt thereof, wherein the improvement or restoration of menstrual regularity is relative to the menstrual cycle to administration of the compound of Formula (I), or a pharmaceutically acceptable salt thereof. In some embodiments, an ovulatory menstrual cycle is restored in the subject after a time period of administration of the compound of Formula (I), or a pharmaceutically acceptable salt thereof.

In some embodiments, control of testicular adrenal rest tumor (TART) is improved in the subject after a time period of administration of the compound of Formula (I), or a pharmaceutically acceptable salt thereof, wherein the improvement in control of testicular adrenal rest tumor is relative to the control of testicular adrenal rest tumor prior to administration of the compound of Formula (I), or a pharmaceutically acceptable salt thereof.

In some embodiments, the incidence and/or severity of testicular adrenal rest tumor (TART) is reduced in the subject after a time period of administration of the compound of Formula (I), or a pharmaceutically acceptable salt thereof.

In some embodiments, control of ovarian adrenal rest tumor (OART) is improved in the subject after a time period of administration of the compound of Formula (I), or a pharmaceutically acceptable salt thereof, wherein the improved control of ovarian adrenal rest tumor is relative to the control of ovarian adrenal rest tumor prior to administration of the compound of Formula (I), or a pharmaceutically acceptable salt thereof.

In some embodiments, the incidence and/or severity of ovarian adrenal rest tumor (OART) is reduced in the subject after a time period of administration of the compound of Formula (I), or a pharmaceutically acceptable salt thereof.

In some embodiments, fertility is improved and/or restored in the subject after a time period of administration of the compound of Formula (I), or a pharmaceutically acceptable salt thereof, wherein the improved and/or restored in fertility is relative to the fertility prior to administration of the compound of Formula (I), or a pharmaceutically acceptable salt thereof.

In some embodiments, gonadotropin levels (including, e.g., LH and FSH) are improved and/or normalized in the subject after a time period of administration of the compound of Formula (I), or a pharmaceutically acceptable salt thereof, wherein the improvement and/or normalization in gonadotropin levels is relative to the gonadotropin levels prior to administration of the compound of Formula (I), or a pharmaceutically acceptable salt thereof.

In some embodiments, progesterone levels are decreased in the subject after a time period of administration of the compound of Formula (I), or a pharmaceutically acceptable salt thereof, wherein the decrease in progesterone levels is relative to the progesterone levels prior to administration of the compound of Formula (I), or a pharmaceutically acceptable salt thereof.

In some embodiments, semen quality (e.g., sperm concentration, morphology, motility, vitality, and volume) is improved in the subject after a time period of administration of the compound of Formula (I), or a pharmaceutically acceptable salt thereof, wherein the improvement in semen quality is relative to the semen quality prior to administration of the compound of Formula (I), or a pharmaceutically acceptable salt thereof.

In some embodiments, LH (luteinizing hormone) levels are increased in the subject after a time period of administration of the compound of Formula (I), or a pharmaceutically acceptable salt thereof, wherein the increase in LH levels are relative to the LH levels prior to administration of the compound of Formula (I), or a pharmaceutically acceptable salt thereof.

In some embodiments, the time period of administration is at least about 4 weeks. In some embodiments, the time period of administration is at least about 24 weeks. In some embodiments, the time period of administration is at least about one year. In some embodiments, the time period of administration is at least 4 weeks. In some embodiments, the time period of administration is at least 24 weeks. In some embodiments, the time period of administration is at least one year. In some embodiments, the time period of administration is less than about 1 day. In some embodiments, the time period of administration is about 1, 2, 3, 4, 5, 6 or 7 days, or within a range of any of the preceding values. In some embodiments, the time period of administration is about 1, 2, 3, 4, 5, 6, 7, 8, 9, 10, 11, 12, 13, 14, 15, 16, 17, 18, 19, 20, 21, 22, 23 or 24 weeks, or within a range of any of the preceding values. In some embodiments, the time period of administration is about 1, 2, 3, 4, 5, 6, 7, 8, 9, 10, 11, or 12 months, or within a range of any of the preceding values. It is understood that comparative measurements occur preferably during the morning.

In some embodiments, the subject is a pediatric subject. In some embodiments, the pediatric subject is less than or equal to six years old. In some embodiments, the pediatric subject is greater than six years old and less than eleven years old. In some embodiments, the pediatric subject is greater than ten years old and less than fifteen years old. In some embodiments, the pediatric subject is greater than fourteen years old and less than nineteen years old. In some embodiments, the pediatric subject weighs less than 55 kg. In some embodiments, the pediatric subject weighs from about 20 kg to about 55 kg. In some embodiments, the pediatric subject weighs from about 10 kg to about 20 kg.

In some embodiments, the subject is an adult subject. In some embodiments, the subject is over eighteen years old. In some embodiments, the subject is female. In some embodiments, the subject is male.

In some embodiments, the compound of Formula (I), or a pharmaceutically acceptable salt thereof, is administered as a pharmaceutical composition described herein. In some embodiments, the compound of Formula (I), or a pharmaceutically acceptable salt thereof, is administered as a pharmaceutical composition described in Example 9. In some embodiments, the compound of Formula (I), or a pharmaceutically acceptable salt thereof, is administered as a pharmaceutical composition described in Example 11. In some embodiments, the compound of Formula (I), or a pharmaceutically acceptable salt thereof, is administered as a pharmaceutical composition described in Example 12. In some embodiments, the compound of Formula (I), or a pharmaceutically acceptable salt thereof, is administered as a pharmaceutical composition described in Example 13. In some embodiments, the compound of Formula (I), or a pharmaceutically acceptable salt thereof, is administered as a hydrochloric acid salt or p-toluenesulfonic acid salt.

In some embodiments, the compound of Formula (I), or a pharmaceutically acceptable salt thereof, is administered as a p-toluenesulfonic acid salt described herein.

For the avoidance of doubt, also provided herein is the corresponding compound of Formula (I), or a pharmaceutically acceptable salt thereof, or corresponding pharmaceutical composition comprising a compound of Formula (I), or a pharmaceutically acceptable salt thereof, for use in the corresponding methods, as described herein.

For the avoidance of doubt, also provided herein is use of the corresponding compound of Formula (I), or a pharmaceutically acceptable salt thereof, in the manufacture of a medicament for use in the corresponding methods, as described herein.

For the avoidance of doubt, also provided herein is use of the corresponding pharmaceutical composition comprising a compound of Formula (I), or a pharmaceutically acceptable salt thereof, in the manufacture of a medicament for use in the corresponding methods, as described herein.

### Liquid Formulations

Provided herein (for use in any of the methods disclosed herein) is a pharmaceutical composition in oral solution dosage form comprising:
(a) a compound of a compound of Formula (I):

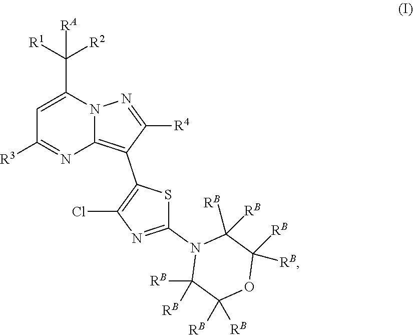

or a pharmaceutically acceptable salt thereof, wherein.
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium; and each R^{B} is independently hydrogen or deuterium;
(b) one or more of a sweetener, an anti-oxidant, and a flavor; and
(c) a liquid vehicle. In some embodiments, at least one of R^{A} or R^{B} is deuterium.

In some embodiments, the pharmaceutical composition comprises about 1 w/v % to about 50 w/v % of the compound of Formula (I), or a pharmaceutically acceptable salt thereof, based on the weight of the free base. In some embodiments, the pharmaceutical composition comprises about 1 w/v % to about 10 w/v % of the compound of Formula (I), or a pharmaceutically acceptable salt thereof, based on the weight of the free base. In some embodiments, the pharmaceutical composition comprises about 5 w/v % of the compound of Formula (I), or a pharmaceutically acceptable salt thereof, based on the weight of the free base. In some embodiments, the pharmaceutical composition comprises about 1, 2, 3, 4, 5, 6, 7, 8, 9, or 10 w/v % of the compound of Formula (I), or a pharmaceutically acceptable salt thereof, based on the weight of the free base, or within a range of any of the preceding values.

In some embodiments, the pharmaceutical composition comprises a sweetener. A sweetener is a formulation component added to improve taste. In some embodiments, the pharmaceutical composition comprises about 0.01 w/v % to about 1.5 w/v % of the sweetener. In some embodiments, the pharmaceutical composition comprises about 0.1 w/v % to about 0.5 w/v % of the sweetener. In some embodiments, the pharmaceutical composition comprises about 0.15 w/v % of the sweetener. In some embodiments, the pharmaceutical composition comprises about 0.1, 0.2, 0.3, 0.4, or 0.5 w/v % of the sweetener, or within a range of any of the preceding values.

In some embodiments, the sweetener is selected from saccharin, sucrose, sucralose, aspartame, dextrose, fructose, maltitol, mannitol, sorbitol, and avantame. In some embodiments, the sweetener is saccharin.

In some embodiments, the pharmaceutical composition comprises an anti-oxidant. An anti-oxidant is a formulation component included to improve stability by preventing oxidation. In some embodiments, the pharmaceutical composition comprises about 0.01 w/v % to about 1.5 w/v % of the anti-oxidant. In some embodiments, the pharmaceutical composition comprises about 0.1 w/v % to about 0.5 w/v % of the anti-oxidant. In some embodiments, the pharmaceutical composition comprises about 0.17 w/v % of the anti-oxidant. In some embodiments, the pharmaceutical composition comprises about 0.1, 0.2, 0.3, 0.4, or 0.5 w/v % of the anti-oxidant, or within a range of any of the preceding values.

In some embodiments, the anti-oxidant is selected from butylated hydroxytoluene, vitamin E TPGS, butylated hydroxyanisole, ascorbic acid, lecithin, tert-butylhydroquinone, and citric acid. In some embodiments, the anti-oxidant is butylated hydroxytoluene.

In some embodiments, the pharmaceutical composition comprises a flavor. A flavor is a formulation component added to mask taste through aromatics. In some embodiments, the pharmaceutical composition comprises about 0.01 w/v % to about 0.5 w/v % of the flavor. In some embodiments, the pharmaceutical composition comprises about 0.05 w/v % to about 0.2 w/v % of the flavor. In some embodiments, the pharmaceutical composition comprises about 0.10 w/v % of the flavor. In some embodiments, the pharmaceutical composition comprises about 0.05, 0.06, 0.07, 0.08, 0.09, 0.10, 0.11, 0.12, 0.13, 0.14, 0.15, 0.16, 0.17, 0.18, 0.19 or 0.2 w/v % of the flavor, or within a range of any of the preceding values.

In some embodiments, the flavor is selected from FONA orange flavor, FONA Juicy Flavor, FONA Grape Flavor, Firmenich SA Lemon Flavor, Firmenich Tetrarome Orange Flavor, IFF Cherry Flavor, and IFF Grape Flavor. In some embodiments, the flavor is FONA orange flavor.

A liquid vehicle is a solvent capable of dissolving or partially dissolving the compound of Formula (I), or a pharmaceutically acceptable salt thereof, for the purposes of delivery as an oral dosing solution. In some embodiments, the pharmaceutical composition comprises about 50 w/v % to about 99.9 w/v % of the liquid vehicle. In some embodiments, the pharmaceutical composition comprises about 90 w/v % to about 99 w/v % of the liquid vehicle. In some embodiments, the pharmaceutical composition comprises about 92 w/v % to about 97 w/v % of the liquid vehicle. In some embodiments, the pharmaceutical composition comprises about 94.6 w/v % of the liquid vehicle. In some embodiments, the pharmaceutical composition comprises about 90, 91, 92, 93, 94, 95, 96, 97, 98 or 99 w/v % of the liquid vehicle, or within a range of any of the preceding values.

In some embodiments, the liquid vehicle is selected from medium-chain triglycerides, propylene glycol dicaprylate/dicaprate, glycerin, propylene glycol, polyethylene glycol, olive oil, soybean oil, corn oil, and transcutol. In some embodiments, the liquid vehicle is medium-chain triglycerides. In some embodiments, the medium-chain triglycerides is Labrafac Lipophile WL1349.

In some embodiments, the pharmaceutical composition further comprises a surfactant. A surfactant is a formulation component added to improve solubility or emulsion properties. In some embodiments, the pharmaceutical composition comprises about 1 w/v % to about 50 w/v % of the surfactant. In some embodiments, the pharmaceutical composition comprises about 10 w/v % to about 30 w/v % of the surfactant. In some embodiments, the pharmaceutical composition comprises about 20 w/v % of the surfactant. In some embodiments, the pharmaceutical composition comprises about 10, 11, 12, 13, 14, 15, 16, 17, 18, 19, 20, 21, 22, 23, 24, 25, 26, 27, 28, 29 or 30 w/v % of the surfactant, or within a range of any of the preceding values.

In some embodiments, the surfactant is selected from oleoyl polyoxyl-6 glycerides, linoleoyl polyoxyl-6 glycerides, Polysorbate 80, Polysorbate 20, vitamin E polyethylene glycol succinate, Gelucire, lauroyl polyoxyl-32 glycerides, sodium lauryl sulfate, Poloxamer, corn oil PEG-6 esters, and hydrogenated palm/palm kernel oil PEG-6 esters. In some embodiments, the surfactant is oleoyl polyoxyl-6 glycerides. In some embodiments, the oleoyl polyoxyl-6 glycerides is LABRAFIL M 1944 CS.

In some embodiments, the pharmaceutical composition comprises about 50 w/v % to about 90 w/v % of the liquid vehicle. In some embodiments, the pharmaceutical composition comprises about 70 w/v % to about 80 w/v % of the liquid vehicle. In some embodiments, the pharmaceutical composition comprises about 75 w/v % of the liquid vehicle. In some embodiments, the pharmaceutical composition comprises about 74.6 w/v % of the liquid vehicle. In some embodiments, the pharmaceutical composition comprises about 70, 71, 72, 73, 74, 75, 76, 77, 78, 79, or 80 w/v % of the liquid vehicle, or within a range of any of the preceding values.

In some embodiments, the liquid vehicle is selected from medium-chain triglycerides, propylene glycol dicaprylate/dicaprate, glycerin, propylene glycol, polyethylene glycol, olive oil, soybean oil, corn oil, and transcutol. In some embodiments, the liquid vehicle is medium-chain triglycerides. In some embodiments, the medium-chain triglycerides is Labrafac Lipophile WL1349.

In some embodiments, the pharmaceutical composition comprises:
(a) the compound of Formula (I), or a pharmaceutically acceptable salt thereof;
(b) a sweetener;
(c) an anti-oxidant;
(d) a flavor; and
(e) a liquid vehicle.

In some embodiments, the pharmaceutical composition further comprises a surfactant.

In some embodiments, the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of a sweetener;
(c) about 0.1 w/v % to about 0.2 w/v % of an anti-oxidant;
(d) about 0.05 w/v % to about 0.2 w/v % of a flavor; and
(e) about 92 w/v % to about 97 w/v % of a liquid vehicle.

In some embodiments, the pharmaceutical composition comprises:
(a) about 5 w/v % of the compound of Formula (I), or a pharmaceutically acceptable salt thereof, based on the weight of the free base;
(b) about 0.15 w/v % of a sweetener;
(c) about 0.17 w/v % of an anti-oxidant;
(d) about 0.1 w/v % of a flavor; and
(e) about 94.6 w/v % of a liquid vehicle.

In some embodiments, the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of a sweetener;
(c) about 0.1 w/v % to about 0.2 w/v % of an anti-oxidant;
(d) about 0.05 w/v % to about 0.2 w/v % of a flavor;
(e) about 15 w/v % to about 25 w/v % of a surfactant; and
(f) about 70 w/v % to about 80 w/v % of a liquid vehicle.

In some embodiments, the pharmaceutical composition comprises:
(a) about 5 w/v % of the compound of Formula (I), or a pharmaceutically acceptable salt thereof, based on the weight of the free base;
(b) about 0.15 w/v % of a sweetener;
(c) about 0.17 w/v % of an anti-oxidant;
(d) about 0.1 w/v % of a flavor;
(e) about 20 w/v % of a surfactant; and
(f) about 75 w/v % of a liquid vehicle.

In some embodiments, the pharmaceutical composition comprises:
(a) the compound of Formula (I), or a pharmaceutically acceptable salt thereof;
(b) saccharin;
(c) butylated hydroxytoluene;
(d) FONA orange flavor; and
(e) medium-chain triglycerides.

In some embodiments, the pharmaceutical composition further comprises oleoyl polyoxyl-6 glycerides.

In some embodiments, the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of saccharin;
(c) about 0.1 w/v % to about 0.2 w/v % of butylated hydroxytoluene;
(d) about 0.05 w/v % to about 0.2 w/v % of FONA orange flavor; and
(e) about 92 w/v % to about 97 w/v % of medium-chain triglycerides.

In some embodiments, the pharmaceutical composition comprises:
(a) about 5 w/v % of the compound of Formula (I), or a pharmaceutically acceptable salt thereof, based on the weight of the free base;
(b) about 0.15 w/v % of saccharin;
(c) about 0.17 w/v % of butylated hydroxytoluene;
(d) about 0.1 w/v % of FONA orange flavor; and
(e) about 94.6 w/v % of medium-chain triglycerides.

In some embodiments, the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of saccharin;
(c) about 0.1 w/v % to about 0.2 w/v % of butylated hydroxytoluene;
(d) about 0.05 w/v % to about 0.2 w/v % of FONA orange flavor;
(e) about 15 w/v % to about 25 w/v % of oleoyl polyoxyl-6 glycerides; and
(f) about 70 w/v % to about 80 w/v % of medium-chain triglycerides.

In some embodiments, the pharmaceutical composition comprises:
(a) about 5 w/v % of the compound of Formula (I), or a pharmaceutically acceptable salt thereof, based on the weight of the free base;
(b) about 0.15 w/v % of saccharin;
(c) about 0.17 w/v % of butylated hydroxytoluene;
(d) about 0.1 w/v % of FONA orange flavor;
(e) about 20 w/v % of oleoyl polyoxyl-6 glycerides; and
(f) about 75 w/v % of medium-chain triglycerides.

In some embodiments, the pharmaceutical composition comprises the compound of Formula (I) as a free base.

In some embodiments, the pharmaceutical composition is formulated in unit dosage form, wherein the compound of Formula (I), or a pharmaceutically acceptable salt thereof, is present in an amount of about 5 mg/mL to about 200 mg/mL, based on the weight of the free base. In some embodiments, the compound of Formula (I), or a pharmaceutically acceptable salt thereof, is present in the unit dosage form in an amount of about 75 mg/mL to about 150 mg/mL, based on the weight of the free base. In some embodiments, the compound of Formula (I), or a pharmaceutically acceptable salt thereof, is present in the unit dosage form in an amount of about 50 mg/mL, based on the weight of the free base. In some embodiments, the compound of Formula (I), or a pharmaceutically acceptable salt thereof, is present in the unit dosage form in an amount of about 100 mg/mL, based on the weight of the free base.

In some embodiments, the liquid pharmaceutical composition has a viscosity between about 1 to about 50 centipoise at about 25° C.

Chemical Groups

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

For compounds of Formula (I), and pharmaceutically acceptable salts thereof, in which a variable appears more than once, each variable may be a different moiety independently selected from the group defining the variable. For example, where a structure is described having two R groups that are simultaneously present on the same compound, the two R groups may represent different moieties independently selected from the group defined for R.

As used herein, an “excipient” refers to a substance that is added to a composition to provide, without limitation, bulk, consistency, stability, binding ability, lubrication, disintegrating ability, etc., to the composition. A “diluent” is a type of excipient and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion, or inhalation. A pharmaceutically acceptable excipient is a physiologically and pharmaceutically suitable non-toxic and inactive material or ingredient that does not interfere with the activity of the drug substance. Pharmaceutically acceptable excipients are well known in the pharmaceutical art and described, for example, in Rowe et al., Handbook of Pharmaceutical Excipients: A Comprehensive Guide to Uses, Properties, and Safety, 5th Ed., 2006, and in Remington: The Science and Practice of Pharmacy (Gennaro, 21st Ed. Mack Pub. Co., Easton, PA (2005)). Preservatives, stabilizers, dyes, buffers, and the like may be provided in the pharmaceutical composition. In addition, antioxidants and suspending agents may also be used. For compositions formulated as liquid solutions, acceptable carriers and/or diluents include saline and sterile water, and may optionally include antioxidants, buffers, bacteriostats and other common additives. In some embodiments, the diluents may be a buffered aqueous solution such as, without limitation, phosphate buffered saline. The compositions may also be formulated as capsules, granules, or tablets which contain, in addition to a compound as disclosed and described herein, diluents, dispersing and surface-active agents, binders, and lubricants. One skilled in this art may further formulate a compound as disclosed and described herein in an appropriate manner, and in accordance with accepted practices, such as those disclosed in Remington, supra.

As used herein, a “dose” or “dosage” refers to the measured quantity of drug substance to be taken at one time by a subject. In certain embodiments, wherein the drug substance is not a free base or free acid, the quantity is the molar equivalent to the corresponding amount of free base or free acid.

As used herein, a “pharmaceutically acceptable salt” refers to salts of a compound having an acidic or basic moiety which are not biologically or otherwise undesirable for use in a pharmaceutical. In many cases, the compounds disclosed herein are capable of forming acid and/or base salts by virtue of the presence of an acidic or basic moiety (e.g. amino and/or carboxyl groups or groups similar thereto). Pharmaceutically acceptable acid addition salts may be formed by combining a compound having a basic moiety with inorganic acids and organic acids. Inorganic acids which may be used to prepare salts include, for example, hydrochloric acid, hydrobromic acid, sulfuric acid, nitric acid, phosphoric acid, and the like. Organic acids which may be used to prepare salts include, for example, methanesulfonic acid, ethanesulfonic acid, p-toluenesulfonic acid, and the like. Pharmaceutically acceptable base addition salts may be formed by combining a compound having an acidic moiety with inorganic and organic bases. Inorganic bases which may be used to prepare salts include, for example, sodium, potassium, lithium, ammonium, calcium, magnesium, iron, zinc, manganese, aluminum hydroxides, carbonates, bicarbonates, phosphates, and the like. In some embodiments, the inorganic base salt is ammonium, potassium, sodium, calcium, and magnesium hydroxides, carbonates, bicarbonates, or phosphates. Organic bases from which may be used to prepare salts include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Generally, such salts may be prepared by reacting the free acid or base forms of these compounds with at least a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN). Lists of suitable salts are found in WO 87/05297; Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977); each of which is incorporated herein by reference in its entirety. A reference for the preparation and selection of pharmaceutical salts of the present disclosure is P. H. Stahl & C. G. Wermuth, Handbook of Pharmaceutical Salts, Verlag Helvetica Chimica Acta, Zurich, 2002 which is incorporated herein by reference in its entirety.

The compounds described herein may be asymmetric (e.g., having one or more stereocenters). All stereoisomers, such as enantiomers and diastereomers, are intended unless otherwise indicated. It is understood that, in any compound described herein having one or more chiral centers, if an absolute stereochemistry is not expressly indicated, then each center may independently be the (R)-configuration, or the (S)-configuration, or a mixture thereof. Thus, the compounds provided herein may be enantiomerically pure, enantiomerically enriched, a racemic mixture, diastereomerically pure, diastereomerically enriched, or a stereoisomeric mixture. Preparation of enantiomerically pure or enantiomerically enriched forms may be accomplished by resolution of racemic mixtures or by using enantiomerically pure or enriched starting materials or by stereoselective or stereospecific synthesis. Stereochemical definitions are available in E. L. Eliel, S. H. Wilen & L. N. Mander, Stereochemistry of Organic Compounds, John Wiley & Sons, Inc., New York, NY, 1994 which is incorporated herein by reference in its entirety. In some embodiments, where the compound described herein is chiral or otherwise includes one or more stereocenters, the compound may be prepared with an enantiomeric excess or diastereomeric excess of greater than about 75%, greater than about 80%, greater than about 85%, greater than about 90%, greater than about 95%, or greater than about 99%.

Resolution of racemic mixtures of compounds may be carried out by any of numerous methods known in the art. An example method includes fractional recrystallizaion using a chiral resolving organic acid with a racemic compound containing a basic group. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Other chiral resolving agents suitable for fractional crystallization methods include stereoisomerically pure forms of methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Similarly, fractional recrystallization using a chiral resolving base may be utilized with a racemic compound containing a basic group.

Resolution of racemic mixtures may also be carried out by elution on a column packed with an optically active resolving agent (e.g., dinitrobenzoylphenylglycine). A suitable elution solvent composition may be determined by one skilled in the art.

In some embodiments, a compound described herein may be prepared having at least about 5%, at least about 10%, at least about 20%, at least about 30%, at least about 40%, at least about 50%, at least about 60%, at least about 70%, at least about 80%, at least about 90%, at least about 95%, at least about 99%, or at least about 99.9% enantiomeric excess, or an enantiomeric excess within a range defined by any of the preceding numbers.

The compounds described herein and their pharmaceutically acceptable salts may be found together with other substances such as water and solvents, for example, in the form of hydrates or solvates. When in the solid-state, the compounds described herein and salts thereof may occur in various forms and may, e.g., take the form of solvates, including hydrates. The compounds may be in any solid-state form, such as a crystalline form, amorphous form, solvated form, etc. and unless clearly indicated otherwise, reference in the specification to compounds and salts thereof should be understood as reading on any solid-state form of the compound.

The compounds described herein may be used in a neutral form, such as, a free acid or free base form. Alternatively, the compounds may be used in the form of pharmaceutically acceptable salts, such as pharmaceutically acceptable addition salts of acids or bases.

In some embodiments, the compounds described herein, or salts thereof, are substantially isolated. The phrase “substantially isolated” refers to the compound that is at least partially or substantially separated from the environment in which it was formed or detected. Partial separation may include, for example, a composition enriched in the compound described herein. Substantial separation may include compositions containing at least about 50%, at least about 60%, at least about 65%, at least about 70%, at least about 75%, at least about 80%, at least about 85%, at least about 90%, at least about 95%, at least about 97%, or at least about 99% by weight of the compound described herein, or salt thereof.

Isotopes

The compounds disclosed and described herein allow atoms at each position of the compound independently to have: 1) an isotopic distribution for a chemical element in proportional amounts to those usually found in nature or 2) an isotopic distribution in proportional amounts different to those usually found in nature unless the context clearly dictates otherwise. A particular chemical element has an atomic number defined by the number of protons within the atom's nucleus. Each atomic number identifies a specific element, but not the isotope; an atom of a given element may have a wide range in its number of neutrons. The number of both protons and neutrons in the nucleus is the atom's mass number, and each isotope of a given element has a different mass number. A compound wherein one or more atoms have an isotopic distribution for a chemical element in proportional amounts different to those usually found in nature is commonly referred to as being an isotopically-labeled compound. Each chemical element as represented in a compound structure may include any isotopic distribution of said element. For example, in a compound structure a hydrogen atom may be explicitly disclosed or understood to be present in the compound. At any position of the compound that a hydrogen atom may be present, the hydrogen atom may be an isotopic distribution of hydrogen, including but not limited to protium (H) and deuterium (2H) in proportional amounts to those usually found in nature and in proportional amounts different to those usually found in nature. Thus, reference herein to a compound encompasses all potential isotopic distributions for each atom unless the context clearly dictates otherwise. Examples of isotopes include isotopes of hydrogen, carbon, nitrogen, oxygen, phosphorous, sulfur, fluorine, chlorine, bromine, and iodine. As one of skill in the art would appreciate, any of the compounds as disclosed and described herein may include radioactive isotopes. Accordingly, also contemplated is use of compounds as disclosed and described herein, wherein one or more atoms have an isotopic distribution different to those usually found in nature, such as having ²H or ³H in greater proportion, or ^{I}C, ¹³C, or ¹⁴C in greater proportion than found in nature. By way of general example, and without limitation, isotopes of hydrogen include protium (H), deuterium (2H), and tritium (3H). Isotopes of carbon include carbon-11 (¹¹C), carbon-12 (¹²C), carbon-13 (¹³C), and carbon-14 (¹⁴C). Isotopes of nitrogen include nitrogen-13 (¹³N), nitrogen-14 (¹⁴N) and nitrogen-15 (¹⁵N). Isotopes of oxygen include oxygen-14 (¹⁴0), oxygen-15 (¹⁵O), oxygen-16 (¹⁶O), oxygen-17 (¹⁷O), and oxygen-18 (¹⁸O). Isotope of fluorine include fluorine-17 (¹⁷F), fluorine-18 (¹⁸F) and fluorine-19 (¹⁹F). Isotopes of phosphorous include phosphorus-31 (³¹P), phosphorus-32 (³²P), phosphorus-33 (³³P), phosphorus-34 (³⁴P), phosphorus-35 (³⁵P) and phosphorus-36 (³⁶P). Isotopes of sulfur include sulfur-32 (³²S), sulfur-33 (³³S), sulfur-34 (³⁴S), sulfur-35 (³⁵S), sulfur-36 (³⁶S) and sulfur-38 (³⁸S). Isotopes of chlorine include chlorine-35 (³⁵Cl), chlorine-36 (³⁶Cl) and chlorine-37 (³⁷Cl). Isotopes of bromine include bromine-75 (⁷⁵Br), bromine-76 (⁷⁶Br), bromine-77 (⁷⁷Br), bromine-79 (⁷⁹Br), bromine-81 (⁸¹Br) and bromine-82 (⁸²Br). Isotopes of iodine include iodine-123 (¹²³I), iodine-124 (¹²⁴I) iodine-125 (¹²⁵I), iodine-131 (¹³¹I) and iodine-135 (¹³⁵I). In some embodiments, atoms at every position of the compound have an isotopic distribution for each chemical element in proportional amounts to those usually found in nature. In some embodiments, an atom in one position of the compound has an isotopic distribution for a chemical element in proportional amounts different to those usually found in nature (remainder atoms having an isotopic distribution for a chemical element in proportional amounts to those usually found in nature). In some embodiments, atoms in at least two positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least three positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least four positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least five positions of the compound independently have an isotopic distribution for a chemical element in proportional amounts different to those usually found in nature (remainder atoms having an isotopic distribution for a chemical element in proportional amounts to those usually found in nature). In some embodiments, atoms in at least six positions of the compound independently have an isotopic distribution for a chemical element in proportional amounts different to those usually found in nature (remainder atoms having an isotopic distribution for a chemical element in proportional amounts to those usually found in nature).

Certain compounds, for example those having incorporated radioactive isotopes such as ³H and ¹⁴C, are also useful in drug or substrate tissue distribution assays. Tritium (³H) and carbon-14 (¹⁴C) isotopes are particularly preferred for their ease of preparation and detectability. Compounds with isotopes such as deuterium (²H) in proportional amounts greater than usually found in nature may afford certain therapeutic advantages resulting from greater metabolic stability, such as, for example, increased in vivo half-life or reduced dosage requirements. Isotopically-labeled compounds may generally be prepared by performing procedures routinely practiced in the chemical art. Methods are readily available to measure such isotope perturbations or enrichments, such as, mass spectrometry, and for isotopes that are radio-isotopes additional methods are available, such as, radio-detectors used in connection with HPLC or GC.

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, protium (H), deuterium (2H), tritium (H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵0O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I) iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound described herein contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen may include ²H as the major isotopic form, as example, or any carbon include be 13C as the major isotopic form, as example, or any nitrogen may include ¹⁵N as the major isotopic form, as example, and any oxygen may include ¹⁸O as the major isotopic form, as example. In certain embodiments, an “isotopic variant” of a compound contains an unnatural proportion of deuterium (²H).

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position.

Synthetic methods for incorporating radio-isotopes into organic compounds are applicable to compound described herein and are well known in the art. These synthetic methods, for example, incorporating activity levels of tritium into target molecules, are as follows: A. Catalytic Reduction with Tritium Gas: This procedure normally yields high specific
activity products and requires halogenated or unsaturated precursors.
B. Reduction with Sodium Borohydride [3H]: This procedure is rather inexpensive and requires precursors containing reducible functional groups such as aldehydes, ketones, lactones, esters and the like.
C. Reduction with Lithium Aluminum Hydride [3H]: This procedure offers products at almost theoretical specific activities. It also requires precursors containing reducible functional groups such as aldehydes, ketones, lactones, esters and the like.
D. Tritium Gas Exposure Labeling: This procedure involves exposing precursors containing exchangeable protons to tritium gas in the presence of a suitable catalyst.
E. N-Methylation using Methyl Iodide [³H]: This procedure is usually employed to prepare O-methyl or N-methyl (³H) products by treating appropriate precursors with high specific activity methyl iodide (³H). This method in general allows for higher specific activity, such as for example, about 70-90 Ci/mmol.

A radiolabeled form of a compound of Formula (I) may be used in a screening assay to identify/evaluate compounds. In general terms, a newly synthesized or identified compound (i.e., test compound) may be evaluated for its ability to reduce binding of a radiolabeled form of a compound of Formula (I) to CRF₁ receptor. The ability of a test compound to compete with a radiolabeled form of a compound of Formula (I) for the binding to CRF₁ receptor directly correlates to its binding affinity.

Certain labeled compounds of the present disclosure bind to CRF₁ receptor. In one embodiment the labeled compound has an IC₅₀ less than about 500 μM. In one embodiment the labeled compound has an IC₅₀ less than about 100 μM. In one embodiment the labeled compound has an IC₅₀ less than about 10 μM. In one embodiment the labeled compound has an IC₅₀ less than about 1 μM. In one embodiment the labeled compound has an IC₅₀ less than about 0.1 μM. In one embodiment the labeled compound has an IC₅₀ less than about 0.01 μM. In one embodiment the labeled compound has an IC₅₀ less than about 0.005 μM.

### Compounds

The present disclosure provides compounds of Formula (I):

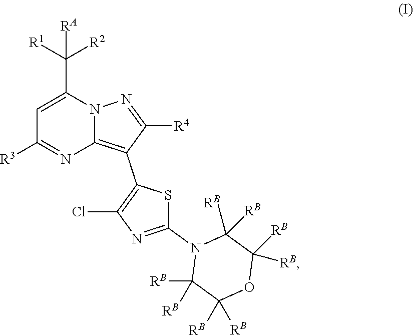

or pharmaceutically acceptable salts thereof, wherein.
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium; and each R^{B} is independently hydrogen or deuterium. In some embodiments, at least one of R^{A} or R^{B} is deuterium.

Some embodiments provide compounds of Formula (IA):

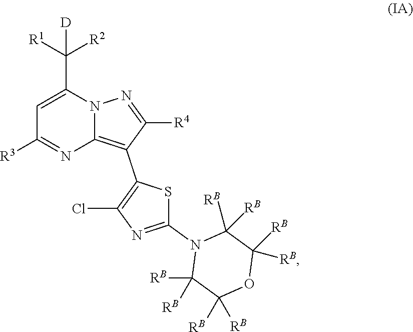

or pharmaceutically acceptable salts thereof, wherein:
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium; and each R^{B} is independently hydrogen or deuterium.

Some embodiments provide compounds of Formula (IB):

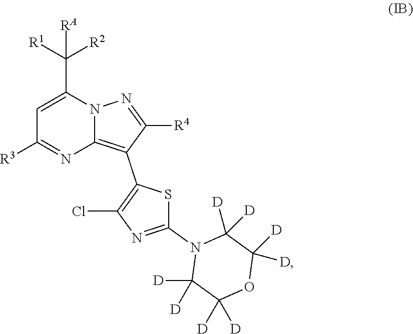

or pharmaceutically acceptable salts thereof, wherein:
R¹ is —C(R^{A})₂C(R^{A})₃; R² is —C(R^{A})₂C(R^{A})₃; R³ is —C(R^{A})₃; R⁴ is —C(R^{A})₃; each R^{A} is independently hydrogen or deuterium.

In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen.

Some embodiments provide a compound of Formulae (I), (IA) or (IB), or a pharmaceutically acceptable salt thereof, or a compound as disclosed and described herein, or a pharmaceutically acceptable salt thereof, for use in treating congenital adrenal hyperplasia in a subject, wherein the compound, or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the reduction in level of any of the biomarkers (e.g., any of 17-OHP, ACTH, and androstenedione) is determined by comparing the level of the biomarker as measured during the circadian release on a day prior to administering the compound of Formula (I), or a pharmaceutically acceptable salt thereof and the level of the biomarker as measured during the circadian release on the day after administering the compound of Formula (I), or a pharmaceutically acceptable salt thereof. A day prior to administering the compound of Formula (I) applies to a subject that has not previously been administered the compound of Formula (I) within at least the past 24 hours. In some embodiments, the circadian release of biomarkers associated with CAH occurs between the hours of 2 a.m. and 10 a.m. In other embodiments, the compound is administered three to eight hours prior to the circadian release of the biomarker. In some embodiments, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some embodiments, the level of 17-hydroxyprogesterone is reduced by at least 25%. In some embodiments, the level of 17-hydroxyprogesterone is reduced by at least 40%. In some embodiments, the level of 17-hydroxyprogesterone is reduced by at least 50%. In some embodiments, the level of 17-hydroxyprogesterone is reduced by an amount of from 10% to 60%, 15% to 60%, 20% to 60%, 25% to 60%, 30% to 60%, 35% to 60%, 40% to 60%, or 50% to 60% from pre-administration levels. In some embodiments, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some embodiments, the level of adrenocorticotropic hormone is reduced by at least 25%. In some embodiments, the level of adrenocorticotropic hormone is reduced by at least 40%. In some embodiments, the level of adrenocorticotropic hormone is reduced by at least 50%. In some embodiments, the level of adrenocorticotropic hormone is reduced by an amount of from 10% to 60%, 15% to 60%, 20% to 60%, 25% to 60%, 30% to 60%, 35% to 60%, 40% to 60%, or 50% to 60% from pre-administration levels. In some embodiments, the level of androstenedione is reduced by at least 25%. In some embodiments, the level of androstenedione is reduced by at least 40%. In some embodiments, the level of androstenedione is reduced by at least 50%. In some embodiments, the level of androstenedione is reduced by an amount of from 10% to 60%, 15% to 60%, 20% to 60%, 25% to 60%, 30% to 60%, 35% to 60%, 40% to 60%, or 50% to 60% from pre-administration levels.

Some embodiments provide a compound of Formulae (I), (IA) or (IB), or a pharmaceutically acceptable salt thereof, or a compound as disclosed and described herein, or a pharmaceutically acceptable salt thereof, for use in reducing the severity of one or more symptoms selected from hirsutism, precocious puberty, fertility problems, acne, and growth impairment in a subject having classic congenital adrenal hyperplasia, wherein the compound is administered in an amount sufficient to reduce the level of androstenedione in the subject. In some embodiments, the growth impairment is selected from one or more of accelerated height velocity, accelerated weight velocity, or accelerated bone age. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the level of androstenedione is reduced by an amount of from 10% to 60%, 15% to 60%, 20% to 60%, 25% to 60%, 30% to 60%, 35% to 60%, 40% to 60%, or 50% to 60% from pre-administration levels. In some embodiments, the level of androstenedione is reduced by at least 25%. In some embodiments, the level of androstenedione is reduced by at least 30%. In some embodiments, the level of androstenedione is reduced by at least 50%. In some embodiments, the level of androstenedione is reduced by an amount of from 10% to 60%, 15% to 60%, 20% to 60%, 25% to 60%, 30% to 60%, 35% to 60%, 40% to 60%, or 50% to 60% from pre-administration levels.

Some embodiments provide a compound of Formulae (I), (IA) or (IB), or a pharmaceutically acceptable salt thereof, or a compound as disclosed and described herein, or a pharmaceutically acceptable salt thereof, for use in reducing the level of one or more biomarkers of congenital adrenal hyperplasia in a subject having congenital adrenal hyperplasia. In some embodiments, the one or more biomarkers of congenital adrenal hyperplasia are selected from (a) 17-hydroxyprogesterone (17-01P); (b) adrenocorticotropic hormone (ACTH); and (c) androstenedione. In some embodiments, at least one of R^{A} or R^{B}is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B}is hydrogen. In some embodiments, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some embodiments, the level of 17-hydroxyprogesterone is reduced by at least 25%. In some embodiments, the level of 17-hydroxyprogesterone is reduced by at least 40%. In some embodiments, the level of 17-hydroxyprogesterone is reduced by at least 50%. In some embodiments, the level of 17-hydroxyprogesterone is reduced by an amount of from 10% to 60%, 15% to 60%, 20% to 60%, 25% to 60%, 30% to 60%, 35% to 60%, 40% to 60%, or 50% to 60% from pre-administration levels. In some embodiments, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some embodiments, the level of adrenocorticotropic hormone is reduced by at least 25%. In some embodiments, the level of adrenocorticotropic hormone is reduced by at least 40%. In some embodiments, the level of adrenocorticotropic hormone is reduced by at least 50%. In some embodiments, the level of adrenocorticotropic hormone is reduced by an amount of from 10% to 60%, 15% to 60%, 20% to 60%, 25% to 60%, 30% to 60%, 35% to 60%, 40% to 60%, or 50% to 60% from pre-administration levels. In some embodiments, the level of androstenedione is reduced by at least 25%. In some embodiments, the level of androstenedione is reduced by at least 40%. In some embodiments, the level of androstenedione is reduced by at least 50%. In some embodiments, the level of androstenedione is reduced by an amount of from 10% to 60%, 15% to 60%, 20% to 60%, 25% to 60%, 30% to 60%, 35% to 60%, 40% to 60%, or 50% to 60% from pre-administration levels.

Some embodiments provide a compound of Formulae (I), (IA) or (IB), or a pharmaceutically acceptable salt thereof, or a compound as disclosed and described herein, or a pharmaceutically acceptable salt thereof, for use in reducing the dosage of corticosteroid administered to a subject having congenital adrenal hyperplasia. In some embodiments, the corticosteroid is a glucocorticoid. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen.

Some embodiments provide a compound of Formulae (I), (IA) or (IB), or a pharmaceutically acceptable salt thereof, or a compound as disclosed and described herein, or a pharmaceutically acceptable salt thereof, for use in reducing the severity of one or more side effects of glucocorticoid treatment in a subject having congenital adrenal hyperplasia, wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, reactivation of latent infections. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the compound is administered at an amount sufficient to reduce the level of 17-hydroxyprogesterone (17-OHP) by at least 50% as compared to the level prior to administration. In some embodiments, the compound is administered at an amount sufficient to reduce the level of androstenedione by at least 30% as compared to the level prior to administration. In some embodiments, the compound is administered at an amount sufficient to (a) reduce the level of 17-hydroxyprogesterone (17-OHP) by at least 50% as compared to the level prior to administration; and (b) reduce the level of androstenedione by at least 30% as compared to the level prior to administration.

Some embodiments provide a compound of Formulae (I), (IA) or (IB), or a pharmaceutically acceptable salt thereof, or a compound as disclosed and described herein, or a pharmaceutically acceptable salt thereof, for use in treating congenital adrenal hyperplasia in a subject comprising
(i) measuring the level of one or more biomarkers selected from (a) 17-hydroxyprogesterone (17-OHP); (b) adrenocorticotropic hormone (ACTH); and (c) androstenedione in a biological sample obtained from the subject;
(ii) analyzing the level of the one or more biomarkers to determine if the level of the one or more biomarkers is elevated compared to a healthy subject not having congenital adrenal hyperplasia; and
(iii) administering to the subject a compound of Formula (I), or a pharmaceutically acceptable salt thereof if the subject is determined to have elevated levels of the one or more biomarkers.

In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the use further comprises (iv) measuring the level of the one or more biomarkers after administering a compound of Formula (I), or a pharmaceutically acceptable salt thereof, in a biological sample obtained from the subject to determine whether the subject has reduced levels of the one or more biomarkers as compared with the measurement of step (i). In some embodiments, the use further comprises (v) continuing the administration of the compound of Formula (I), or a pharmaceutically acceptable salt thereof if the subject has reduced levels of the one or more biomarkers. In some embodiments, steps (i) and (iv) are performed on biological samples taken from the subject in a similar manner and within a same time of day window. In some embodiments, steps (i) and (iv) are performed on biological samples taken from the subject within the time of day window from 2 a.m. to 10 a.m. In some embodiments, steps (i) and (iv) are performed on biological samples taken from the subject within the time of day window from 6 a.m. to 10 a.m. In some embodiments, steps (i) and (iv) comprise measuring the levels of at least two biomarkers selected from (a) 17-hydroxyprogesterone (17-OHP); (b) adrenocorticotropic hormone (ACTH); and (c) androstenedione. In some embodiments, steps (i) and (iv) comprise measuring the levels of (a) 17-hydroxyprogesterone (17-OHP); (b) adrenocorticotropic hormone (ACTH); and (c) androstenedione. In some embodiments, step (i) comprises measuring the level of 17-hydroxyprogesterone (17-01P), wherein the level of 17-hydroxyprogesterone (17-OHP) is elevated when it is greater than or equal to 1,000 ng/dL. In some embodiments, step (i) comprises measuring the level of androstenedione, wherein the level of androstenedione is elevated when it is greater than 200 ng/dL.

Some embodiments provide a compound of Formulae (I), (IA) or (IB), or a pharmaceutically acceptable salt thereof, or a compound as disclosed and described herein, or a pharmaceutically acceptable salt thereof, for use in for use in a method of treating congenital adrenal hyperplasia in a subject, wherein the compound of Formula (I), or a pharmaceutically acceptable salt thereof, is administered at a frequency of not less than twice daily; and wherein the amount of the compound of Formulae (I), (IA) or (IB), or a pharmaceutically acceptable salt thereof, or a compound as disclosed and described herein, or a pharmaceutically acceptable salt thereof, in the first administration is less than the amount of the compound of Formulae (I), (IA) or (IB), or a pharmaceutically acceptable salt thereof, or a compound as disclosed and described herein, or a pharmaceutically acceptable salt thereof, in the second and any subsequent administrations. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the compound of Formulae (I), (IA) or (IB), or a pharmaceutically acceptable salt thereof, or a compound as disclosed and described herein, or a pharmaceutically acceptable salt thereof, is administered at a frequency of twice daily.

In some embodiments, the compound of Formulae (I), (IA) or (IB), or a pharmaceutically acceptable salt thereof, or a compound as disclosed and described herein, or a pharmaceutically acceptable salt thereof, is administered at a frequency of twice daily (i.e., comprising a first administration and a second administration).

In some embodiments, the ratio of the amount of the Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from 1:1.1 to 1:100, 1:1.1 to 1:95, 1:1.1 to 1:90, 1:1.1 to 1:85, about 1:1.1 to 1:80, about 1:1.1 to 1:75, 1:1.1 to 1:70, 1:1.1 to 1:65, 1:1.1 to 1:60, 1:1.1 to 1:55, 1:1.1 to 1:50, 1:1.1 to 1:45, 1:1.1 to 1:40, 1:1.1 to 1:35, 1:1.1 to 1:30, 1:1.1 to 1:25, 1:1.1 to 1:20, about 1:1.1 to 1:15, 1:1.1 to 1:10, 1:1.1 to 1:9, 1:1.1 to 1:8, 1:1.1 to 1:7, 1:1.1 to 1:6, 1:1.1 to 1:5, 1:1.1 to 1:4, 1:1.1 to 1:3.5, 1:1.1 to 1:3, 1:1.1 to 1:2.5, 1:1.1 to 1:2, 1:1.1 to 1:1.5, or 1:1.1 to 1.25.

In some embodiments, the ratio of the amount of the Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:100, about 1:1 to about 1:95, about 1:1 to about 1:90, about 1:1 to about 1:85, about 1:1 to about 1:80, about 1:1 to about 1:75, about 1:1 to about 1:70, about 1:1 to about 1:65, about 1:1 to about 1:60, about 1:1 to about 1:55, about 1:1 to about 1:50, about 1:1 to about 1:45, about 1:1 to about 1:40, about 1:1 to about 1:35, about 1:1 to about 1:30, about 1:1 to about 1:25, about 1:1 to about 1:20, about 1:1 to about 1:15, about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3.5, about 1:1 to about 1:3, about 1:1 to about 1:2.5, about 1:1 to about 1:2, about 1:1 to about 1:1.5 or about 1:1 to about 1.25.

In some embodiments, the ratio of the amount of the Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:100.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:50.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:10.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:5.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:3.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:2.5.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is from about 1:1 to about 1:2.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is about 1:1.5.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is about 1:2.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is about 1:2.5.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration about 1:3.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is about 1:3.5.

In some embodiments, the ratio of the amount of the compound of Formulae (I), (IA) or (IB), or a pharmaceutically acceptable salt thereof, or a compound as disclosed and described herein, or a pharmaceutically acceptable salt thereof, in the first administration to the amount of the compound of Formulae (I), (IA) or (IB), or a pharmaceutically acceptable salt thereof, or a compound as disclosed and described herein, or a pharmaceutically acceptable salt thereof, in the second administration is about 1:4.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is less than or equal to about 1000 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 25 mg to about 1000 mg, about 50 mg to about 1000 mg, about 50 mg to about 950 mg, about 50 mg to about 900 mg, about 50 mg to about 850 mg, about 50 mg to about 800 mg, about 50 mg to about 750 mg, about 50 mg to about 700 mg, about 50 mg to about 650 mg, about 50 mg to about 600 mg, about 50 mg to about 550 mg, about 50 mg to about 500 mg, about 50 mg to about 450 mg, about 50 mg to about 400 mg, about 50 mg to about 350 mg, about 50 mg to about 300 mg, about 75 mg to about 350 mg, or about 75 mg to about 300 mg, wherein the daily amounts are based on the weight of the free base of the compound.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 100 mg to about 1000 mg, about 100 mg to about 950 mg, about 100 mg to about 900 mg, about 100 mg to about 850 mg, about 100 mg to about 800 mg, about 100 mg to about 750 mg, about 100 mg to about 700 mg, about 100 mg to about 650 mg, about 100 mg to about 600 mg, about 100 mg to about 550 mg, about 100 mg to about 500 mg, about 100 mg to about 450 mg, about 100 mg to about 400 mg, about 100 mg to about 350 mg, about 100 mg to about 300 mg, or about 100 mg to about 250, wherein the daily amounts are based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is from about 50 mg to about 1000 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is from about 100 mg to about 1000 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is from about 100 mg to about 500 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is from about 200 mg to about 500 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is from about 200 mg to about 450 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 250 mg based on the weight of the free base. In a further embodiment, the first administration of the compound of Formula (I), or a pharmaceutically acceptable salt thereof is about 100 mg based on the weight of the free base and the second administration of the compound of Formulae (I), (IA) or (IB), or a pharmaceutically acceptable salt thereof, or a compound as disclosed and described herein, or a pharmaceutically acceptable salt thereof, is about 150 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 400 mg based on the weight of the free base. In a further embodiment, the first administration of the compound of Formula (I), or a pharmaceutically acceptable salt thereof is about 150 mg based on the weight of the free base and the second administration of the compound of Formulae (I), (IA) or (IB), or a pharmaceutically acceptable salt thereof, or a compound as disclosed and described herein, or a pharmaceutically acceptable salt thereof, is about 250 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 300 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is about 100 mg and the second administration of the compound of Formulae (I), (IA) or (IB), or a pharmaceutically acceptable salt thereof, or a compound as disclosed and described herein, or a pharmaceutically acceptable salt thereof is about 200 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 450 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is about 200 mg and the second administration of the compound of Formulae (I), (IA) or (IB), or a pharmaceutically acceptable salt thereof, or a compound as disclosed and described herein, or a pharmaceutically acceptable salt thereof is about 250 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 350 mg based on the weight of the free base. In a further embodiments, the first administration of the compound of Formulae (I), (IA) or (IB), or a pharmaceutically acceptable salt thereof, or a compound as disclosed and described herein, or a pharmaceutically acceptable salt thereof is about 150 mg based on the weight of the free base and the second administration of the compound of Formulae (I), (IA) or (IB), or a pharmaceutically acceptable salt thereof, or a compound as disclosed and described herein, or a pharmaceutically acceptable salt thereof is about 200 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 500 mg based on the weight of the free base. In a further embodiments, the first administration of the compound of Formulae (I), (IA) or (IB), or a pharmaceutically acceptable salt thereof, or a compound as disclosed and described herein, or a pharmaceutically acceptable salt thereof is about 200 mg based on the weight of the free base and the second administration of the compound of Formulae (I), (IA) or (IB), or a pharmaceutically acceptable salt thereof, or a compound as disclosed and described herein, or a pharmaceutically acceptable salt thereof is about 300 mg based on the weight of the free base.

In some embodiments, the subject weighs greater than or equal to about 55 kg. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 400 mg or above and the subject weighs greater than or equal to about 55 kg.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 100 mg based on the weight of the free base.

In some embodiments, the subject weighs from about 10 kg to about 20 kg.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 250 mg based on the weight of the free base. In some embodiments, the first administration of the compound of Formulae (I), (IA) or (IB), or a pharmaceutically acceptable salt thereof, or a compound as disclosed and described herein, or a pharmaceutically acceptable salt thereof is about 100 mg based on the weight of the free base and the second administration of the compound of of Formulae (I), (IA) or (IB), or a pharmaceutically acceptable salt thereof, or a compound as disclosed and described herein, or a pharmaceutically acceptable salt thereof is about 150 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 300 mg based on the weight of the free base. In some embodiments, the first administration of the compound of Formulae (I), (IA) or (IB), or a pharmaceutically acceptable salt thereof, or a compound as disclosed and described herein, or a pharmaceutically acceptable salt thereof is about 100 mg based on the weight of the free base and the second administration of the compound of of Formulae (I), (IA) or (IB), or a pharmaceutically acceptable salt thereof, or a compound as disclosed and described herein, or a pharmaceutically acceptable salt thereof is about 200 mg based on the weight of the free base. In some embodiments, the subject weighs from about 20 kg to about 55 kg.

Some embodiments provide a compound of Formulae (I), (IA) or (IB), or a pharmaceutically acceptable salt thereof, or a compound as disclosed and described herein, or a pharmaceutically acceptable salt thereof, for use in for use in a method of treating congenital adrenal hyperplasia in a subject, wherein the compound of Formulae (I), (IA) or (IB), or a pharmaceutically acceptable salt thereof, or a compound as disclosed and described herein, or a pharmaceutically acceptable salt thereof, is administered at a frequency of not less than twice daily; and wherein the amount of the compound of Formulae (I), (IA) or (IB), or a pharmaceutically acceptable salt thereof, or a compound as disclosed and described herein, or a pharmaceutically acceptable salt thereof, administered daily is greater than 400 mg based on the weight of the free base. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the compound of Formulae (I), (IA) or (IB), or a pharmaceutically acceptable salt thereof, or a compound as disclosed and described herein, or a pharmaceutically acceptable salt thereof, is administered at a frequency of twice daily.

In some embodiments, the compound of Formulae (I), (IA) or (IB), or a pharmaceutically acceptable salt thereof, or a compound as disclosed and described herein, or a pharmaceutically acceptable salt thereof, is administered at a frequency of twice daily (i.e., comprising a first administration and a second administration).

In some embodiments, the amount of the Formulae (I), (IA) or (IB), or a pharmaceutically acceptable salt thereof, or a compound as disclosed and described herein, or a pharmaceutically acceptable salt thereof, administered daily is from 405 mg to about 1000 mg based on the weight of the free base. In some embodiments, the amount of the Formulae (I), (IA) or (IB), or a pharmaceutically acceptable salt thereof, or a compound as disclosed and described herein, or a pharmaceutically acceptable salt thereof, administered daily is from 405 mg to about 600 mg based on the weight of the free base. In some embodiments, the amount of the Formulae (I), (IA) or (IB), or a pharmaceutically acceptable salt thereof, or a compound as disclosed and described herein, or a pharmaceutically acceptable salt thereof, administered daily is from 405 mg to about 500 mg based on the weight of the free base. In some embodiments, the amount of the Formulae (I), (IA) or (IB), or a pharmaceutically acceptable salt thereof, or a compound as disclosed and described herein, or a pharmaceutically acceptable salt thereof, administered daily is from 405 mg to about 475 mg based on the weight of the free base. In some embodiments, the amount of the Formulae (I), (IA) or (IB), or a pharmaceutically acceptable salt thereof, or a compound as disclosed and described herein, or a pharmaceutically acceptable salt thereof, administered daily is from 405 mg to about 450 mg based on the weight of the free base. In some embodiments, the amount of the Formulae (I), (IA) or (IB), or a pharmaceutically acceptable salt thereof, or a compound as disclosed and described herein, or a pharmaceutically acceptable salt thereof, administered daily is about 450 mg based on the weight of the free base. In further embodiments, the first administration of the compound of Formulae (I), (IA) or (IB), or a pharmaceutically acceptable salt thereof, or a compound as disclosed and described herein, or a pharmaceutically acceptable salt thereof is about 200 mg and the second administration of the compound of Formulae (I), (IA) or (IB), or a pharmaceutically acceptable salt thereof, or a compound as disclosed and described herein, or a pharmaceutically acceptable salt thereof is about 250 mg based on the weight of the free base.

In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 425 mg based on the weight of the free base. In a further embodiments, the first administration of the compound of Formulae (I), (IA) or (IB), or a pharmaceutically acceptable salt thereof, or a compound as disclosed and described herein, or a pharmaceutically acceptable salt thereof is about 200 mg based on the weight of the free base and the second administration of the compound of Formulae (I), (IA) or (IB), or a pharmaceutically acceptable salt thereof, or a compound as disclosed and described herein, or a pharmaceutically acceptable salt thereof is about 225 mg based on the weight of the free base. In some embodiments, the amount of the compound of Formulae (I), (IA) or (IB), or a pharmaceutically acceptable salt thereof, or a compound as disclosed and described herein, or a pharmaceutically acceptable salt thereof, administered daily is about 225 mg based on the weight of the free base. In a further embodiments, the first administration of the compound of Formulae (I), (IA) or (IB), or a pharmaceutically acceptable salt thereof, or a compound as disclosed and described herein, or a pharmaceutically acceptable salt thereof is about 100 mg based on the weight of the free base and the second administration of the compound of Formulae (I), (IA) or (IB), or a pharmaceutically acceptable salt thereof, or a compound as disclosed and described herein, or a pharmaceutically acceptable salt thereof is about 125 mg based on the weight of the free base.

In some embodiments, the compound of Formulae (I), (IA) or (IB), or a pharmaceutically acceptable salt thereof, or a compound as disclosed and described herein, or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject. In some embodiments, the reduction in level of any of the biomarkers (e.g., any of 17-OHP, ACTH, and androstenedione) is determined by comparing the level of the biomarker as measured during the circadian release on a day prior to administering the compound of Formula (I), or a pharmaceutically acceptable salt thereof and the level of the biomarker as measured during the circadian release on the day after administering the compound of Formula (I), or a pharmaceutically acceptable salt thereof. A day prior to administering the compound of Formula (I) applies to a subject that has not previously been administered the compound of Formula (I) within at least the past 24 hours. In some embodiments, the circadian release of biomarkers associated with CAH occurs between the hours of 2 a.m. and 10 a.m. In other embodiments, the compound is administered three to eight hours prior to the circadian release of the biomarker. In some embodiments, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some embodiments, the level of 17-hydroxyprogesterone is reduced by at least 25%. In some embodiments, the level of 17-hydroxyprogesterone is reduced by at least 40%. In some embodiments, the level of 17-hydroxyprogesterone is reduced by at least 50%. In some embodiments, the level of 17-hydroxyprogesterone is reduced by an amount of from 10% to 60%, 15% to 60%, 20% to 60%, 25% to 60%, 30% to 60%, 35% to 60%, 40% to 60%, or 50% to 60% from pre-administration levels. In some embodiments, the level of 17-hydroxyprogesterone is reduced by at least 10%, at least 15%, at least 20%, at least 25%, at least 30%, at least 35%, at least 40%, at least 50%, at least 55% or at least 60% from pre-administration levels. In some embodiments, the level of adrenocorticotropic hormone is reduced by at least 25%. In some embodiments, the level of adrenocorticotropic hormone is reduced by at least 40%. In some embodiments, the level of adrenocorticotropic hormone is reduced by at least 50%. In some embodiments, the level of adrenocorticotropic hormone is reduced by an amount of from 10% to 60%, 15% to 60%, 20% to 60%, 25% to 60%, 30% to 60%, 35% to 60%, 40% to 60%, or 50% to 60% from pre-administration levels. In some embodiments, the level of androstenedione is reduced by at least 25%. In some embodiments, the level of androstenedione is reduced by at least 40%. In some embodiments, the level of androstenedione is reduced by at least 50%. In some embodiments, the level of androstenedione is reduced by an amount of from 10% to 60%, 15% to 60%, 20% to 60%, 25% to 60%, 30% to 60%, 35% to 60%, 40% to 60%, or 50% to 60% from pre-administration levels.

Some embodiments provide a compound of Formulae (I), (IA) or (IB), or a pharmaceutically acceptable salt thereof, or a compound as disclosed and described herein, or a pharmaceutically acceptable salt thereof, for use in a method of reducing the dosage of corticosteroid administered to a subject having congenital adrenal hyperplasia for controlling congenital adrenal hyperplasia, wherein the compound of Formula (I), or a pharmaceutically acceptable salt thereof, is administered at a frequency of not less than twice daily; and wherein the amount of the compound of Formula (I), or a pharmaceutically acceptable salt thereof, in the first administration is less than the amount of the compound of Formula (I), or a pharmaceutically acceptable salt thereof, in the second and any subsequent administrations. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the corticosteroid is a glucocorticoid.

Some embodiments provide a compound of Formulae (I), (IA) or (IB), or a pharmaceutically acceptable salt thereof, or a compound as disclosed and described herein, or a pharmaceutically acceptable salt thereof, for use in a method of reducing the severity of one or more side effects of glucocorticoid treatment in a subject having congenital adrenal hyperplasia, wherein the compound of Formulae (I), (IA) or (IB), or a pharmaceutically acceptable salt thereof, or a compound as disclosed and described herein, or a pharmaceutically acceptable salt thereof, is administered at a frequency of not less than twice daily; wherein the compound of Formulae (I), (IA) or (IB), or a pharmaceutically acceptable salt thereof, or a compound as disclosed and described herein, or a pharmaceutically acceptable salt thereof, in the first administration is less than the amount of the compound of Formulae (I), (IA) or (IB), or a pharmaceutically acceptable salt thereof, or a compound as disclosed and described herein, or a pharmaceutically acceptable salt thereof, in the second and any subsequent administrations; and wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, reactivation of latent infections. In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the compound is administered at an amount sufficient to reduce the level of 17-hydroxyprogesterone (17-OHP) by at least 50% as compared to the level prior to administration. In some embodiments, the compound is administered at an amount sufficient to reduce the level of androstenedione by at least 30% as compared to the level prior to administration. In some embodiments, the compound is administered at an amount sufficient to (a) reduce the level of 17-hydroxyprogesterone (17-01P) by at least 50% as compared to the level prior to administration; and (b) reduce the level of androstenedione by at least 30% as compared to the level prior to administration.

In some embodiments, the compound of Formulae (I), (IA) or (IB), or a pharmaceutically acceptable salt thereof, or a compound as disclosed and described herein, or a pharmaceutically acceptable salt thereof, is administered twice daily at an amount from about 25 mg to about 250 mg based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is administered twice daily as: (a) a first administration of about 50 mg based on the weight of the free base and a second administration of about 350 mg based on the weight of the free base; or (b) a first administration of about 100 mg based on the weight of the free base and a second administration of about 300 mg based on the weight of the free base; or (c) a first administration of about 150 mg based on the weight of the free base and a second administration of about 250 mg based on the weight of the free base; or (d) a first administration of about 200 mg based on the weight of the free base and a second administration of about 250 mg based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is administered twice daily as: (a) a first administration of about 50 mg based on the weight of the free base and a second administration of about 150 mg based on the weight of the free base; or (b) a first administration of about 100 mg based on the weight of the free base and a second administration of about 200 mg based on the weight of the free base; or (c) a first administration of about 100 mg based on the weight of the free base and a second administration of about 250 mg based on the weight of the free base; or (d) a first administration of about 150 mg based on the weight of the free base and a second administration of about 250 mg based on the weight of the free base. In some embodiments, the compound is administered in the free base form.

In some embodiments, the subject is in a fed state. In some embodiments, the compound of Formulae (I), (IA) or (IB), or a pharmaceutically acceptable salt thereof, or a compound as disclosed and described herein, or a pharmaceutically acceptable salt thereof, is administered to the subject with a nutritional composition. In some embodiments, the nutritional composition is a liquid dietary supplement comprising about 1000 to about 2000 calories per liter with a fat content greater than about 30%. In some embodiments, the nutritional composition is a liquid dietary supplement comprising 1500 calories per liter with a caloric distribution of 14.7% protein, 32% fat and 53.3% carbohydrate. In some embodiments, the nutritional composition is administered in an amount of about 6 to about 12 fluid ounces. In some embodiments, the nutritional composition is administered in an amount of about 6 to about 12 fluid ounces. In some embodiments, the nutritional composition is administered in an amount of about 8 fluid ounces. In some embodiments, the nutritional composition is administered within 30 minutes of each administration of the compound of Formulae (I), (IA) or (IB), or a pharmaceutically acceptable salt thereof, or a compound as disclosed and described herein, or a pharmaceutically acceptable salt thereof. In some embodiments, the compound of Formulae (I), (IA) or (IB), or a pharmaceutically acceptable salt thereof, or a compound as disclosed and described herein, or a pharmaceutically acceptable salt thereof, is administered to the subject with a meal. In some embodiments, the meal is a high fat meal. In some embodiments, the meal is a low fat meal. In some embodiments, the meal is completed within about 30 minutes after administration of the compound of Formulae (I), (IA) or (IB), or a pharmaceutically acceptable salt thereof, or a compound as disclosed and described herein, or a pharmaceutically acceptable salt thereof. In some embodiments, the first administration of the compound of Formulae (I), (IA) or (IB), or a pharmaceutically acceptable salt thereof, or a compound as disclosed and described herein, or a pharmaceutically acceptable salt thereof is with a morning meal. In some embodiments, the second administration of the compound of Formulae (I), (IA) or (IB), or a pharmaceutically acceptable salt thereof, or a compound as disclosed and described herein, or a pharmaceutically acceptable salt thereof is with an evening meal. In some embodiments, the first administration of the compound of Formulae (I), (IA) or (IB), or a pharmaceutically acceptable salt thereof, or a compound as disclosed and described herein, or a pharmaceutically acceptable salt thereof, is with a morning meal and the second administration of the compound of Formulae (I), (IA) or (IB), or a pharmaceutically acceptable salt thereof, or a compound as disclosed and described herein, or a pharmaceutically acceptable salt thereof is with an evening meal. In some embodiments, there are about 6 to about 14 hours, about 8 to about 14 hours or about 11 to about 13 hours between the first and second administrations of the compound of Formulae (I), (IA) or (IB), or a pharmaceutically acceptable salt thereof, or a compound as disclosed and described herein, or a pharmaceutically acceptable salt thereof. In some embodiments, there are about 12 hours between the first and second administrations of the compound of Formulae (I), (IA) or (IB), or a pharmaceutically acceptable salt thereof, or a compound as disclosed and described herein, or a pharmaceutically acceptable salt thereof. In some embodiments, the subject is concurrently receiving a dose of a glucocorticoid. In some embodiments, the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate. In some embodiments, the glucocorticoid is cortisol (hydrocortisone). In some embodiments, the glucocorticoid is cortisone. In some embodiments, the glucocorticoid is prednisone. In some embodiments, the glucocorticoid is measured in hydrocortisone equivalents. In some embodiments, the glucocorticoid dose is measured as a multiple of the upper limit of normal of physiologic dosing in hydrocortisone equivalents. In some embodiments, the glucocorticoid dose is a physiologic dose as measured after a time period of administration of the compound of Formulae (I), (IA) or (IB), or a pharmaceutically acceptable salt thereof, or a compound as disclosed and described herein, or a pharmaceutically acceptable salt thereof. In some embodiments, the glucocorticoid dose is a physiologic dose of about 4 to about 12 mg/m²/day, about 4 to about 9 mg/m²/day, or less than about 8 mg/m²/day as measured after a time period of administration of the compound of Formulae (I), (IA) or (IB), or a pharmaceutically acceptable salt thereof, or a compound as disclosed and described herein, or a pharmaceutically acceptable salt thereof. In some embodiments, the glucocorticoid dose of the subject is reduced by about 10%, about 20%, about 30%, about 40%, about 50%, about 60% or about 70% after a time period of administration of the compound of Formulae (I), (IA) or (IB), or a pharmaceutically acceptable salt thereof, or a compound as disclosed and described herein, or a pharmaceutically acceptable salt thereof, wherein the reduction of the glucocorticoid dose is relative to the glucocorticoid dose prior to administration of the compound of Formulae (I), (IA) or (IB), or a pharmaceutically acceptable salt thereof, or a compound as disclosed and described herein, or a pharmaceutically acceptable salt thereof. In some embodiments, the glucocorticoid dose of the subject is reduced by less than about 20% after a time period of administration of the compound of Formulae (I), (IA) or (IB), or a pharmaceutically acceptable salt thereof, or a compound as disclosed and described herein, or a pharmaceutically acceptable salt thereof, wherein the reduction of the glucocorticoid dose is relative to the glucocorticoid dose prior to administration of the compound of Formulae (I), (IA) or (IB), or a pharmaceutically acceptable salt thereof, or a compound as disclosed and described herein, or a pharmaceutically acceptable salt thereof. In some embodiments, the glucocorticoid dose of the subject is reduced by about 20% to about 50% after a time period of administration of the compound of Formulae (I), (IA) or (IB), or a pharmaceutically acceptable salt thereof, or a compound as disclosed and described herein, or a pharmaceutically acceptable salt thereof, wherein the reduction of the glucocorticoid dose is relative to the glucocorticoid dose prior to administration of the compound Formulae (I), (IA) or (IB), or a pharmaceutically acceptable salt thereof, or a compound as disclosed and described herein, or a pharmaceutically acceptable salt thereof. In some embodiments, the glucocorticoid dose of the subject is reduced by greater than about 50% after a time period of administration of the compound of Formulae (I), (IA) or (IB), or a pharmaceutically acceptable salt thereof, or a compound as disclosed and described herein, or a pharmaceutically acceptable salt thereof, wherein the reduction of the glucocorticoid dose is relative to the glucocorticoid dose prior to administration of the compound of Formulae (I), (IA) or (IB), or a pharmaceutically acceptable salt thereof, or a compound as disclosed and described herein, or a pharmaceutically acceptable salt thereof. In some embodiments, the level of 17-hydroxyprogesterone is less than about 1.5 times the upper limit of normal after a time period of administration of the compound of Formulae (I), (IA) or (IB), or a pharmaceutically acceptable salt thereof, or a compound as disclosed and described herein, or a pharmaceutically acceptable salt thereof. In some embodiments, the level of 17-hydroxyprogesterone is within normal limits after a time period of administration of the compound of Formulae (I), (IA) or (IB), or a pharmaceutically acceptable salt thereof, or a compound as disclosed and described herein, or a pharmaceutically acceptable salt thereof. In some embodiments, the level of adrenocorticotropic hormone is less than about 1.5 times the upper limit of normal after a time period of administration of the compound of Formulae (I), (IA) or (IB), or a pharmaceutically acceptable salt thereof, or a compound as disclosed and described herein, or a pharmaceutically acceptable salt thereof. In some embodiments, the level of adrenocorticotropic hormone is within normal limits after a time period of administration of the compound of Formulae (I), (IA) or (IB), or a pharmaceutically acceptable salt thereof, or a compound as disclosed and described herein, or a pharmaceutically acceptable salt thereof. In some embodiments, the level of androstenedione is less than about 1.5 times the upper limit of normal after a time period of administration of the compound of Formulae (I), (IA) or (IB), or a pharmaceutically acceptable salt thereof, or a compound as disclosed and described herein, or a pharmaceutically acceptable salt thereof. In some embodiments, the level of androstenedione is within normal limits after a time period of administration of the compound of Formulae (I), (IA) or (IB), or a pharmaceutically acceptable salt thereof, or a compound as disclosed and described herein, or a pharmaceutically acceptable salt thereof. In some embodiments, the level of testosterone is reduced by at least about 25%, about 30% or about 50% after a time period of administration of the compound of Formulae (I), (IA) or (IB), or a pharmaceutically acceptable salt thereof, or a compound as disclosed and described herein, or a pharmaceutically acceptable salt thereof, wherein the reduction of the level of testosterone is relative to the level of testosterone prior to administration of the compound of Formulae (I), (IA) or (IB), or a pharmaceutically acceptable salt thereof, or a compound as disclosed and described herein, or a pharmaceutically acceptable salt thereof. In some embodiments, the level of testosterone is less than about 1.5 times the upper limit of normal after a time period of administration of the compound of Formulae (I), (IA) or (IB), or a pharmaceutically acceptable salt thereof, or a compound as disclosed and described herein, or a pharmaceutically acceptable salt thereof. In some embodiments, the level of testosterone is within normal limits after a time period of administration of the compound of Formulae (I), (IA) or (IB), or a pharmaceutically acceptable salt thereof, or a compound as disclosed and described herein, or a pharmaceutically acceptable salt thereof. In some embodiments, the subject exhibits a decrease in glucocorticoid burden after a time period of administration of the compound of Formulae (I), (IA) or (IB), or a pharmaceutically acceptable salt thereof, or a compound as disclosed and described herein, or a pharmaceutically acceptable salt thereof, wherein the decrease in glucocorticoid burden is relative to the glucocorticoid burden prior to administration of the compound of Formulae (I), (IA) or (IB), or a pharmaceutically acceptable salt thereof, or a compound as disclosed and described herein, or a pharmaceutically acceptable salt thereof. In some embodiments, one or more symptoms of glucocorticoid burden selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor, control of ovarian adrenal rest tumor, and fertility, is improved after a time period of administration of the compound of Formulae (I), (IA) or (IB), or a pharmaceutically acceptable salt thereof, or a compound as disclosed and described herein, or a pharmaceutically acceptable salt thereof, wherein the improvement in the one or more symptoms is relative to the status of the one or more symptoms prior to administration of the compound of Formulae (I), (IA) or (IB), or a pharmaceutically acceptable salt thereof, or a compound as disclosed and described herein, or a pharmaceutically acceptable salt thereof. In some embodiments, the time period of administration is at least about 4 weeks. In some embodiments, the time period of administration is at least about 24 weeks. In some embodiments, the time period of administration is at least about one year.

Some embodiments provide a compound of Formulae (I), (IA) or (IB), or a pharmaceutically acceptable salt thereof, or a compound as disclosed and described herein, or a pharmaceutically acceptable salt thereof, for use in a method of treating congenital adrenal hyperplasia in a subject, the method comprising: (a) selecting a subject who has a glucocorticoid dose of greater than 11 mg/m²/day after a time period of being administered a compound of Formulae (I), (IA) or (IB), or a pharmaceutically acceptable salt thereof, or a compound as disclosed and described herein, or a pharmaceutically acceptable salt thereof, at an amount of about 200 mg once or twice daily based on the weight of the free base; and (b) administering to the subject the compound of Formulae (I), (IA) or (IB), or a pharmaceutically acceptable salt thereof, or a compound as disclosed and described herein, or a pharmaceutically acceptable salt thereof, at a frequency of twice daily; wherein the amount of the compound of Formulae (I), (IA) or (IB), or a pharmaceutically acceptable salt thereof, or a compound as disclosed and described herein, or a pharmaceutically acceptable salt thereof, in the first administration is less than the amount of the compound of Formulae (I), (IA) or (IB), or a pharmaceutically acceptable salt thereof, or a compound as disclosed and described herein, or a pharmaceutically acceptable salt thereof, in the second administration; and wherein the amount of the compound of Formulae (I), (IA) or (IB), or a pharmaceutically acceptable salt thereof, or a compound as disclosed and described herein, or a pharmaceutically acceptable salt thereof, administered daily is less than 500 mg based on the weight of the free base (e.g. less than or equal to 400 mg based on the weight of the free base). In some embodiments, at least one of R^{A} or R^{B} is deuterium. In some embodiments, R¹ is —CH₂CD₃, or R² is —CH₂CD₃. In some embodiments, R¹ is —CH₂CD₃, and R² is —CH₂CD₃. In some embodiments, R¹ is —CH₂CH₃, and R² is —CH₂CH₃. In some embodiments, R¹ is —CD₂CD₃, or R² is —CD₂CD₃. In some embodiments, R¹ is —CD₂CD₃, and R² is —CD₂CD₃. In some embodiments, R³ is CD₃; or R⁴ is CD₃. In some embodiments, each R^{A} is hydrogen. In some embodiments, each R^{B} is hydrogen. In some embodiments, the time period of administration is at least about 4 weeks. In some embodiments, the time period of administration is at least about 24 weeks. In some embodiments, the time period of administration is at least about one year. In some embodiments, the subject is female. In some embodiments, the subject is male. In some embodiments, the compound of Formulae (I), (IA) or (IB), or a pharmaceutically acceptable salt thereof, or a compound as disclosed and described herein, or a pharmaceutically acceptable salt thereof is administered as a pharmaceutical composition comprising: (a) the compound of Formulae (I), (IA) or (IB), or a pharmaceutically acceptable salt thereof, or a compound as disclosed and described herein, or a pharmaceutically acceptable salt thereof; and (b) one or more of a lubricant, a diluent, a binder, and a glidant. In some embodiments, the pharmaceutical composition comprises about 70 wt % to about 97 wt % of the compound of Formulae (I), (IA) or (IB), or a pharmaceutically acceptable salt thereof, or a compound as disclosed and described herein, or a pharmaceutically acceptable salt thereof, based on the weight of the free base. In some embodiments, the pharmaceutical composition comprises about 80 wt % to about 96 wt % of the compound of Formulae (I), (IA) or (IB), or a pharmaceutically acceptable salt thereof, or a compound as disclosed and described herein, or a pharmaceutically acceptable salt thereof, based on the weight of the free base. In some embodiments, the pharmaceutical composition comprises about 85 wt % to about 90 wt % of the compound of Formulae (I), (IA) or (IB), or a pharmaceutically acceptable salt thereof, or a compound as disclosed and described herein, or a pharmaceutically acceptable salt thereof, based on the weight of the free base. In some embodiments, the pharmaceutical composition comprises a lubricant. In some embodiments, the pharmaceutical composition comprises a diluent. In some embodiments, the pharmaceutical composition comprises a binder. In some embodiments, the pharmaceutical composition comprises a glidant. In some embodiments, the pharmaceutical composition comprises the compound of Formulae (I), (IA) or (IB), or a pharmaceutically acceptable salt thereof, or a compound as disclosed and described herein, or a pharmaceutically acceptable salt thereof, in crystalline form. In some embodiments, the pharmaceutical composition comprises the compound of Formulae (I), (IA) or (IB), or a compound as disclosed and described herein as a free base. In some embodiments, the pharmaceutical composition is formulated in unit dosage form, wherein the compound of Formulae (I), (IA) or (IB), or a pharmaceutically acceptable salt thereof, or a compound as disclosed and described herein, or a pharmaceutically acceptable salt thereof, is present in an amount of about 25 mg to about 400 mg, based on the weight of the free base. In some embodiments, the pharmaceutical composition is formulated in unit dosage form, wherein the compound of Formulae (I), (IA) or (IB), or a pharmaceutically acceptable salt thereof, or a compound as disclosed and described herein, or a pharmaceutically acceptable salt thereof, is present in an amount of about 50 mg to about 400 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 50 mg, about 100 mg, about 150 mg, about 200 mg, about 250 mg, about 300 mg, about 350 mg, or about 400 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 75 mg, about 125 mg, about 175 mg, about 225 mg, about 275 mg, about 325 mg, or about 375 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 200 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 400 mg, based on the weight of the free base. In some embodiments, the pharmaceutical composition is in the form of a tablet, capsule, sachet, powder, granules, coated particle, coated tablet, enterocoated tablet, enterocoated capsule, melting strip, or melting film. In some embodiments, the pharmaceutical composition is in tablet form. In some embodiments, the pharmaceutical composition is in capsule form. In some embodiments, the tablet form or capsule form is coated. In some embodiments, the compound of Formulae (I), (IA) or (IB), or a pharmaceutically acceptable salt thereof, or a compound as disclosed and described herein, or a pharmaceutically acceptable salt thereof is administered as a pharmaceutical composition in oral solution dosage form comprising: (a) the compound of Formulae (I), (IA) or (IB), or a pharmaceutically acceptable salt thereof, or a compound as disclosed and described herein, or a pharmaceutically acceptable salt thereof; (b) one or more of a sweetener, an anti-oxidant, and a flavor; and (c) a liquid vehicle. In some embodiments, the pharmaceutical composition comprises: (a) the compound of Formulae (I), (IA) or (IB), or a pharmaceutically acceptable salt thereof, or a compound as disclosed and described herein, or a pharmaceutically acceptable salt thereof; (b) a sweetener; (c) an anti-oxidant; (d) a flavor; and (e) a liquid vehicle. In some embodiments, the pharmaceutical composition further comprises a surfactant. In some embodiments, the pharmaceutical composition comprises: (a) the compound of Formulae (I), (IA) or (IB), or a pharmaceutically acceptable salt thereof, or a compound as disclosed and described herein, or a pharmaceutically acceptable salt thereof; (b) saccharin; (c) butylated hydroxytoluene; (d) a FONA orange flavor; and (e) medium-chain triglycerides. In some embodiments, the pharmaceutical composition further comprises a oleoyl polyoxyl-6 glycerides. In some embodiments, the pharmaceutical composition comprises the compound of Formulae (I), (IA) or (IB) or a compound as disclosed and described herein as a free base. In some embodiments, the pharmaceutical composition is formulated in unit dosage form, wherein the compound of Formulae (I), (IA) or (IB), or a pharmaceutically acceptable salt thereof, or a compound as disclosed and described herein, or a pharmaceutically acceptable salt thereof, is present in an amount of about 5 mg/mL to about 200 mg/mL, based on the weight of the free base. In some embodiments, the unit dosage form comprises the compound of Formulae (I), (IA) or (IB), or a pharmaceutically acceptable salt thereof, or a compound as disclosed and described herein, or a pharmaceutically acceptable salt thereof, in an amount of about 50 mg/mL, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is administered in a pharmaceutical composition comprising a spray-dried dispersion comprising: a compound of Formula (I), or a pharmaceutically acceptable salt thereof; and a polymer selected from a neutral polymer, an enteric polymer, and a pyrrolidone polymer; wherein the weight ratio of the compound of Formula (I) to the polymer is from about 1:1 to about 1:9.

In some embodiments, the spray-dried dispersion comprises: a compound of Formulae (I), (IA) or (IB), or a pharmaceutically acceptable salt thereof, or a compound as disclosed and described herein, or a pharmaceutically acceptable salt thereof; and a polymer that is a copolymer of 1-vinyl-2-pyrrolidone and vinyl acetate having the structure:

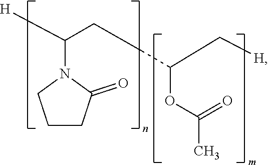

wherein the value of n is about 1 to about 2 times the value of m and the copolymer comprises 1-vinyl-2-pyrrolidone and vinyl acetate at a ratio of about 60:40 by weight; and wherein the weight ratio of the compound of Formula (I) to the copolymer is from about 1:1 to about 1:9.

### Pharmaceutical Compositions, Formulation, and Dosage Forms

The present disclosure further provides for pharmaceutical products such as pharmaceutical compositions, formulations, unit dosage forms, and kits; each comprising a compound of Formula (I), or a pharmaceutically acceptable salt thereof.

The present disclosure also provides for pharmaceutical compositions comprising any of the compounds described herein (e.g., a compound of Formula (I), including specific compounds described herein) or pharmaceutically acceptable salts thereof, and an excipient such as a pharmaceutically acceptable excipient. A pharmaceutically acceptable excipient is a physiologically and pharmaceutically suitable non-toxic and inactive material or ingredient that does not interfere with the activity of the drug substance; an excipient also may be called a carrier. The formulation methods and excipients described herein are exemplary and are in no way limiting. Pharmaceutically acceptable excipients are well known in the pharmaceutical art and described, for example, in Rowe et al., Handbook of Pharmaceutical Excipients: A Comprehensive Guide to Uses, Properties, and Safety, 5th Ed., 2006, and in Remington: The Science and Practice of Pharmacy (Gennaro, 21st Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used.

For compositions formulated as liquid solutions, acceptable carriers and/or diluents include saline and sterile water, and may optionally include antioxidants, buffers, bacteriostats and other common additives. The compositions may also be formulated as pills, capsules, granules, or tablets which contain, in addition to the compounds described herein (e.g., a compound of Formula (I), including specific compounds described herein), diluents, dispersing and surface active agents, binders, and lubricants. One skilled in this art may further formulate the compounds described herein (e.g., a compound of Formula (I), including specific compounds described herein) in an appropriate manner, and in accordance with accepted practices, such as those disclosed in Remington, supra.

Methods of administration include systemic administration of the compounds described herein (e.g., a compound of Formula (I), including specific compounds described herein), preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention may be prepared in aqueous injection solutions which may contain, in addition to the compounds described herein (e.g., a compound of Formula (I), including specific compounds described herein), buffers, antioxidants, bacteriostats, and other additives commonly employed in such solutions.

Pharmaceutical preparations for oral administration may be obtained by any suitable method, typically by uniformly mixing the compound(s) with liquids or finely divided solid carriers, or both, in the required proportions and then, if necessary, processing the mixture, after adding suitable auxiliaries, if desired, forming the resulting mixture into a desired shape to obtain tablets or dragee cores.

Conventional excipients, such as binding agents, fillers, adjuvant, carrier, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that may be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound described herein in a suitable liquid vehicle and filter sterilizing the solution before lyophilization, or simply filling and sealing an appropriate vial or ampule.

Some embodiments provide methods for preparing a pharmaceutical composition comprising the step of admixing a compound of Formula (I), or a pharmaceutically acceptable salt thereof, and a pharmaceutically acceptable carrier.

In making pharmaceutical compositions comprising a compound of Formula (I), or pharmaceutically acceptable salts thereof, the drug substance is typically mixed (i.e., admixed) with an excipient, diluted by an excipient or enclosed within such a carrier in the form of, for example, a capsule, sachet, paper, or other container. When the excipient serves as a diluent, it may be a solid, semi-solid, or liquid material, which acts as a vehicle, carrier, or medium for the drug substance. Thus, the compositions may be in the form of tablets, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

For preparing solid form pharmaceutical compositions such as powders, tablets, capsules, cachets, suppositories and dispersible granules an excipient may be one or more substances which may also act as diluents, flavoring agents, solubilizers, lubricants, suspending agents, binders, preservatives, tablet disintegrating agents, or an encapsulating material. Also included are solid form preparations which are intended to be converted, shortly before use, to liquid form preparations for oral administration. Such liquid forms include solutions, suspensions and emulsions. These preparations may contain, in addition to the drug substance, colorants, flavors, stabilizers, buffers, artificial and natural sweeteners, dispersants, thickeners, solubilizing agents and the like.

For preparing suppositories, a low melting wax, such as an admixture of fatty acid glycerides or cocoa butter, is first melted and the drug substance is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the drug substance such carriers as are known in the art to be appropriate.

Liquid form preparations include solutions, suspensions and emulsions, for example, water or water-propylene glycol solutions. For example, parenteral injection liquid preparations may be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The pharmaceutical compositions may take such forms as suspensions, solutions, or emulsions in oily or aqueous vehicles and may contain formulatory agents such as suspending, stabilizing and/or dispersing agents. Alternatively, the pharmaceutical compositions may be in powder form, obtained by aseptic isolation of sterile solid or by lyophilization from solution, for constitution with a suitable vehicle, e.g., sterile, pyrogen-free water, before use.

The pharmaceutical compositions may be formulated as an aqueous solution, an aqua-alcoholic solution, a solid suspension, an emulsion, a liposomal suspension, or a freeze-dried powder for reconstitution. Such pharmaceutical compositions may be administered directly or as an admixture for further dilution/reconstitution. Route of administration includes intravenous bolus, intravenous infusion, irrigation, and instillation. Suitable solvents include water, alcohols, PEG, propylene glycol, and lipids; pH adjustments using an acid, e.g., HCl or citric acid, may be used to increase solubility and resulting compositions subjected to suitable sterilization procedures know in the art, such as, aseptic filtration. In some embodiments, the pH of the aqueous solution is about 2.0 to about 4.0. In some embodiments, the pH of the aqueous solution is about 2.5 to about 3.5.

Aqueous formulations suitable for oral use may be prepared by dissolving or suspending the drug substance in water and adding suitable colorants, flavors, stabilizing and thickening agents, as desired.

Aqueous suspensions suitable for oral use may be made by dispersing the finely divided drug substance in water with viscous material, such as natural or synthetic gums, resins, methylcellulose, sodium carboxymethylcellulose, or other well-known suspending agents.

For topical administration to the epidermis the compounds described herein, or pharmaceutically acceptable salts thereof may be formulated as gels, ointments, creams or lotions, or as a transdermal patch. Also, formulations suitable for topical administration in the mouth include lozenges comprising drug substance in a flavored base, usually sucrose and acacia or tragacanth; pastilles comprising the drug substance in an inert base such as gelatin and glycerin or sucrose and acacia; and mouthwashes comprising the drug substance in a suitable liquid carrier. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. In some embodiments, topical formulations can contain one or more conventional carriers. In some embodiments, ointments can contain water and one or more hydrophobic carriers selected from, for example, liquid paraffin, polyoxyethylene alkyl ether, propylene glycol, white vaseline, and the like. Carrier compositions of creams may be based on water in combination with glycerol and one or more other components, e.g., glycerinemonostearate, PEG-glycerinemonostearate and cetylstearyl alcohol. Gels may be formulated using isopropyl alcohol and water, suitably in combination with other components such as, for example, glycerol, hydroxyethyl cellulose, and the like.

Solutions or suspensions may be applied directly to the nasal cavity by conventional means, for example with a dropper, pipette or spray. The formulations may be provided in single or multi-dose form. In the latter case of a dropper or pipette, this may be achieved by the subject administering an appropriate, predetermined volume of the solution or suspension. In the case of a spray, this may be achieved for example by means of a metering atomizing spray pump.

Administration to the respiratory tract may also be achieved by means of an aerosol formulation provided in a pressurized pack with a suitable propellant. If the compounds described herein, or pharmaceutically acceptable salts thereof or pharmaceutical compositions comprising them are administered as aerosols, for example as nasal aerosols or by inhalation, this may be carried out, for example, using a spray, a nebulizer, a pump nebulizer, an inhalation apparatus, a metered inhaler or a dry powder inhaler. Pharmaceutical forms for administration of the compounds described herein (or pharmaceutically acceptable salts thereof), as an aerosol may be prepared by processes well known to the person skilled in the art. For their preparation, for example, solutions or dispersions of the compounds described herein (or pharmaceutically acceptable salts thereof), in water, water/alcohol mixtures or suitable saline solutions may be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

Alternatively, the pharmaceutical composition may be provided in the form of a dry powder, for example, a powder mix of the compound in a suitable, powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The compounds of Formula (I), or pharmaceutically acceptable salts thereof may also be administered via a rapid dissolving or a slow release composition, wherein the composition includes a biodegradable rapid dissolving or slow release carrier (such as a polymer carrier and the like). Rapid dissolving or slow release carriers are well known in the art and are used to form complexes that capture therein compounds of Formula (I), or pharmaceutically acceptable salts thereof and either rapidly or slowly degrade/dissolve in a suitable environment (e.g., aqueous, acidic, basic, etc.).

The pharmaceutical preparations are preferably in unit dosage forms. In such form, the preparation is subdivided into unit doses containing appropriate quantities of the drug substance. The unit dosage form may be a packaged preparation, the package containing discrete quantities of preparation, such as packeted tablets, capsules and powders in vials or ampoules. Also, the unit dosage form may be a capsule, tablet, cachet, or lozenge itself, or it may be the appropriate number of any of these in packaged form. In some embodiments, the pharmaceutical preparation is a tablet or capsule for oral administration. In some embodiments, the pharmaceutical preparation is a liquid formulated for intravenous administration.

The compositions may be formulated in a unit dosage form, each dosage containing the drug substance or equivalent mass of the drug substance. The term “unit dosage forms” refers to physically discrete units of a formulation suitable as unitary dosages for human subjects and other mammals, each unit containing a predetermined quantity of drug substance calculated to produce the desired therapeutic effect, in association with a suitable excipient, as described herein.

The compositions described herein may be formulated to provide immediate and/or timed release (also called extended release, sustained release, controlled release, or slow release) of the drug substance after administration to a subject by employing procedures known in the art. For example, the tablets including compounds of Formula (I), or pharmaceutically acceptable salts thereof, may be coated or otherwise compounded to provide a dosage form affording the advantage of prolonged action. For example, the tablet can comprise an inner dosage and an outer dosage component, the latter being in the form of an envelope over the former. The two components may be separated by an enteric layer which serves to resist disintegration in the stomach and permit the inner component to pass intact into the duodenum or to be delayed in release. A variety of materials may be used for such enteric layers or coatings, such materials including a number of polymeric acids and mixtures of polymeric acids with such materials as shellac, cetyl alcohol, and cellulose acetate.

The liquid forms including the drug substance may be incorporated for administration orally or by injection include aqueous solutions, suitably flavored syrups, aqueous or oil suspensions, and flavored emulsions with edible oils such as cottonseed oil, sesame oil, coconut oil, or peanut oil, and similar excipients.

The pharmaceutical compositions described herein may be sterilized by conventional sterilization techniques, or may be sterile filtered. Aqueous solutions may be packaged for use as is, or lyophilized, the lyophilized preparation being combined with a sterile aqueous carrier prior to administration. The pH of the compound preparations is typically between 3 and 11, more preferably from 5 to 9 and most preferably from 7 to 8. It will be understood that use of certain of the foregoing excipients may result in the formation of pharmaceutically acceptable salts.

Compositions for inhalation or insufflation include solutions and suspensions in pharmaceutically acceptable, aqueous or organic solvents, or mixtures thereof, and powders. The liquid or solid compositions may contain suitable excipients as described herein. In some embodiments, the compositions are administered by the oral or nasal respiratory route for local or systemic effect. Compositions may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered orally or nasally from devices which deliver the formulation in an appropriate manner.

The compositions may, if desired, be presented in a pack or dispenser device which may contain one or more-unit dosage forms containing the drug substance. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

For preparing solid compositions such as tablets, the drug substance may be mixed with an excipient to form a solid preformulation composition containing a homogeneous mixture of components. When referring to these preformulation compositions as homogeneous, the drug substance is typically dispersed evenly throughout the composition so that the composition may be readily subdivided into equally effective unit dosage forms such as tablets and capsules. This solid preformulation is then subdivided into unit dosage forms of the type described above containing from, for example, 0.001 mg to 5000 mg of the drug substance or equivalent mass of the drug substance. Representative amounts of the drug substance or equivalent mass of the drug substance in a unit dosage form include, but are not limited to, 0.01 mg, 0.1 mg, 0.2 mg, 0.4 mg, 0.6 mg, 0.8 mg, 1 mg, 2 mg, 4 mg, 6 mg, 8 mg, 10 mg, 12 mg, 14 mg, 16 mg, 18 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 400 mg, 500 mg, 600 mg, 700 mg, 800 mg, 900 mg, 1000 mg, 1500 mg, 2000 mg, 2500 mg, 3000 mg, 3500 mg, 4000 mg, 4500 mg, 5000 mg or an amount within a range defined by any of the preceding amounts.

In some embodiments, the pharmaceutical compositions of the present disclosure are formulated in unit dosage form. In some embodiments, the compound of Formula (I), or pharmaceutically acceptable salt thereof, is present in an amount of about 5 mg to about 200 mg in the unit dosage form. For example, about 5 mg to about 175 mg, about 5 mg to about 150 mg, about 5 mg to about 125 mg, about 5 mg to about 100 mg, about 5 mg to about 75 mg, about 5 mg to about 50 mg, about 5 mg to about 25 mg, about 25 mg to about 200 mg, about 25 mg to about 175 mg, about 25 mg to about 150 mg, about 25 mg to about 125 mg, about 25 mg to about 100 mg, about 25 mg to about 75 mg, about 25 mg to about 50 mg, about 50 mg to about 200 mg, about 50 mg to about 175 mg, about 50 mg to about 150 mg, about 50 mg to about 125 mg, about 50 mg to about 100 mg, about 50 mg to about 75 mg, about 75 mg to about 200 mg, about 75 mg to about 175 mg, about 75 mg to about 150 mg, about 75 mg to about 125 mg, about 75 mg to about 100 mg, about 100 mg to about 200 mg, about 100 mg to about 175 mg, about 100 mg to about 150 mg, about 100 mg to about 125 mg, about 125 mg to about 200 mg, about 125 mg to about 175 mg, about 125 mg to about 150 mg, about 150 mg to about 200 mg, about 150 mg to about 175 mg, or about 175 mg to about 200 mg in the unit dosage form. In some embodiments, the compound of Formula (I), or pharmaceutically acceptable salt thereof, is present in an amount of about 25 mg to about 125 mg in the unit dosage form. In some embodiments, the compound of Formula (I), or pharmaceutically acceptable salt thereof, is present in an amount of about 75 mg to about 150 mg in the unit dosage form. In some embodiments, the compound of Formula (I), or pharmaceutically acceptable salt thereof, is present in an amount of about 5 mg, about 10 mg, about 25 mg, about 35 mg, about 50 mg, about 65 mg, about 75 mg, about 90 mg, about 100 mg, about 125 mg, about 150 mg, about 175 mg, or about 200 mg in the unit dosage form, or within a range defined by any of the preceding values. In some embodiments, the compound of Formula (I), or pharmaceutically acceptable salt thereof, is present in an amount of about 50 mg in the unit dosage form. In some embodiments, the compound of Formula (I), or pharmaceutically acceptable salt thereof, is present in an amount of about 100 mg in the unit dosage form. In some embodiments, the compound of Formula (I), or pharmaceutically acceptable salt thereof, is present in an amount of about 25 mg in the unit dosage form. In some embodiments, the compound of Formula (I), or pharmaceutically acceptable salt thereof, is present in an amount of 5 mg to 250 mg in the unit dosage form. For example, 5 mg to 175 mg, 5 mg to 150 mg, 5 mg to 125 mg, 5 mg to 100 mg, 5 mg to 75 mg, 5 mg to 50 mg, 5 mg to 25 mg, 25 mg to 200 mg, 25 mg to 175 mg, 25 mg to 150 mg, 25 mg to 125 mg, 25 mg to 100 mg, 25 mg to 75 mg, 25 mg to 50 mg, 50 mg to 200 mg, 50 mg to 175 mg, 50 mg to 150 mg, 50 mg to 125 mg, 50 mg to 100 mg, 50 mg to 75 mg, 75 mg to 200 mg, 75 mg to 175 mg, 75 mg to 150 mg, 75 mg to 125 mg, 75 mg to 100 mg, 100 mg to 200 mg, 100 mg to 175 mg, 100 mg to 150 mg, 100 mg to 125 mg, 125 mg to 200 mg, 125 mg to 175 mg, 125 mg to 150 mg, 150 mg to 200 mg, 150 mg to 175 mg, or 175 mg to 200 mg in the unit dosage form. In some embodiments, the compound of Formula (I), or pharmaceutically acceptable salt thereof, is present in an amount of 25 mg to 125 mg in the unit dosage form. In some embodiments, the compound of Formula (I), or pharmaceutically acceptable salt thereof, is present in an amount of 75 mg to 150 mg in the unit dosage form. In some embodiments, the compound of Formula (I), or pharmaceutically acceptable salt thereof, is present in an amount of 5 mg, 10 mg, 25 mg, 35 mg, 50 mg, 65 mg, 75 mg, 90 mg, 100 mg, 125 mg, 150 mg, 175 mg, or 200 mg in the unit dosage form, or within a range defined by any of the preceding values. In some embodiments, the compound of Formula (I), or pharmaceutically acceptable salt thereof, is present in an amount of 50 mg in the unit dosage form. In some embodiments, the compound of Formula (I), or pharmaceutically acceptable salt thereof, is present in an amount of 100 mg in the unit dosage form. In some embodiments, the pharmaceutical compositions of the present disclosure are formulated as a tablet. In some embodiments, the tablet is coated. In some embodiments, the pharmaceutical compositions of the present disclosure are formulated as capsules. In some embodiments, the pharmaceutical compositions are in sachet form. In some embodiments, the pharmaceutical compositions are in granule form.

Kits with unit doses of one or more of the compounds described herein, usually in oral or injectable doses, are provided. Such kits may include a container containing the unit dose, an informational package insert describing the use and attendant benefits of the drugs in treating pathological condition of interest, and optionally an appliance or device for delivery of the composition.

The compounds described herein, or a pharmaceutically acceptable salt thereof, may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The amount of compound or composition administered to a subject will also vary depending upon what is being administered, the purpose of the administration, such as prophylaxis or therapy, the state of the subject, the manner of administration, and the like. In therapeutic applications, compositions may be administered to a subject already suffering from a disease in an amount sufficient to cure or at least partially arrest the symptomology and/or pathology of the disease and its complications. Therapeutically effective doses will depend on the disease condition being treated as well as by the judgment of the attending clinician depending upon factors such as the severity of the disease, the age, weight and general condition of the subject, and the like.

The desired dose may conveniently be presented in a single dose or presented as divided doses administered at appropriate intervals, for example, as two, three, four, or more sub-doses per day. The sub-dose itself may be further divided, e.g., into a number of discrete loosely spaced administrations. The daily dose may be divided, especially when relatively large amounts are administered as deemed appropriate, into several, for example two, three, or four-part administrations. If appropriate, depending on individual behavior, it may be necessary to deviate upward or downward from the daily dose indicated.

It will be apparent to those skilled in the art that the dosage forms described herein may comprise a compound described herein or pharmaceutically acceptable salt thereof.

As described herein optimal doses are generally determined using experimental models and/or clinical trials. The optimal dose of the CRF₁ antagonist may depend upon the body mass, weight, blood volume, or other individual characteristics of the subject. For example, a person skilled in the medical art may consider the subject's condition, that is, stage of the disease, severity of symptoms caused by the disease, general health status, as well as age, gender, and weight, and other factors apparent to a person skilled in the medical art. In general, the amount of a compound described herein, that is present in a dose ranges from about 0.1 mg to about 2 mg per kg weight of the subject. In certain embodiments, a daily dose is about 10-150 mg. The use of the minimum dose that is sufficient to provide effective therapy is usually preferred. Subjects may generally be monitored for therapeutic effectiveness by clinical evaluation and using assays suitable for the condition being treated or prevented, which methods will be familiar to those having ordinary skill in the art and are described herein. The level of a compound that is administered to a subject may be monitored by determining the level of the compound in a biological fluid, for example, in the blood, blood fraction (e.g., plasma, serum), and/or in the urine, and/or other biological sample from the subject. Any method practiced in the art to detect the compound may be used to measure the level of compound during the course of a therapeutic regimen.

Tablets or capsules for oral administration and liquids for oral or intravenous administration are preferred compositions.

The pharmaceutical compositions described herein may be sterilized by conventional sterilization techniques, or may be sterile filtered. Aqueous solutions may be packaged for use as is, or lyophilized, the lyophilized preparation being combined with a sterile aqueous carrier prior to administration. The pH of the compound preparations is typically between 3 and 11, more preferably from 5 to 9 and most preferably from 7 to 8. It will be understood that use of certain of the foregoing excipients may result in the formation of pharmaceutically acceptable salts.

Compositions for inhalation or insufflation include solutions and suspensions in pharmaceutically acceptable, aqueous or organic solvents, or mixtures thereof, and powders. The liquid or solid compositions may contain suitable excipients as described herein. In some embodiments, the compositions are administered by the oral or nasal respiratory route for local or systemic effect. Compositions may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered orally or nasally from devices which deliver the formulation in an appropriate manner.

The compositions may, if desired, be presented in a pack or dispenser device which may contain one or more unit dosage forms containing the drug substance. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that may include a compound described herein formulated in a compatible pharmaceutical carrier may also be prepared, placed in an appropriate container, and labeled for treatment of an indicated condition.

Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

In some embodiments, the pharmaceutical composition includes a filler. In some embodiments, the filler is selected from among binders, diluents, disintegrants, glidants, surfactants, and combinations thereof.

In some embodiments, the filler include saccharides (e.g., sugars, starch, and cellulose), gelatin, calcium carbonate, and synthetic polymers (e.g., polyvinylpyrrolidone, polyethylene glycol, and poloxamers (e.g., Poloxamer 188, a copolymer of polyoxyethylene and polyoxypropylene)). Exemplary fillers include, but are not limited to, glucose, sucrose, lactose, a starch, including modified starches such as sodium starch glycolate (e.g., Explotab®), xylitol, dextrin, saccharose, sorbitol, mannitol (e.g., Parteck® M 200 (mannitol with an average particle size of about 50 μm to about 500 μm) or Parteck® M 100 (mannitol with an average particle size of less than 212 μm)), a cellulose, a polyvinylpyrrolidone, a polyethylene glycol, a polyvinyl alcohol, a polymethacrylate, dibasic calcium phosphate, magnesium stearate, calcium stearate, sodium stearate, stearic acid, hydrogenated vegetable oils, a mineral oil, sodium lauryl sulfate, magnesium lauryl sulfate, glyceryl palmitostearate, sodium benzoate, sodium stearyl fumarate, colloidal silicon dioxide, sodium benzoate, sodium oleate, sodium acetate, aliginic acid, alginates (e.g., sodium alginate), calcium silicate, and ion exchange resins. Exemplary cellulose fillers include microcrystalline cellulose (e.g., Avicel® PH-101 (microcrystalline cellulose with an average particle size of approximately 50 μm) or Avicel® PH 200 (microcrystalline cellulose with an average particle size of approximately 180 μm)), methyl cellulose, ethyl cellulose, hydroxypropyl cellulose, and hydroxypropylmethylcellulose. Exemplary fillers include cross-linked polyvinylpyrrolidone such as with an average particle size of 90 μm to 130 μm) or with an average particle size of 10 μm to 30 μm). Other fillers known to those of skill in the art are also contemplated as being useful when formulated in the pharmaceutical compositions described herein.

In some embodiments, the filler is a binder. Exemplary binders include, but are not limited to, glucose, sucrose, lactose, a starch, including modified starches such as sodium starch glycolate (Explotab®), xylitol, dextrin, saccharose, sorbitol, mannitol (e.g., Parteck® M 200 (mannitol with an average particle size of about 50 μm to about 500 μm), Parteck® M 100 (mannitol with an average particle size of less than 212 μm)), gelatin, gum tragacanth, acacia mucilage, a cellulose, a polyvinylpyrrolidone, a polyethylene glycol, a polyvinyl alcohol, a polymethacrylate, and sodium starch glycolate. Exemplary cellulose fillers include microcrystalline cellulose (e.g., Avicel® PH-101 (microcrystalline cellulose with an average particle size of approximately 50 μm) or Avicel® PH 200 (microcrystalline cellulose with an average particle size of approximately 180 μm)), cellulose ethers, methyl cellulose, ethyl cellulose, croscarmellose sodium, sodium carboxy methyl cellulose starches, hydroxypropyl cellulose, and hydroxypropyl methyl cellulose. Exemplary polyvinylpyrrolidone fillers include cross-linked polyvinylpyrrolidone such as Kollidon® CL (crospovidone with an average particle size of 90 μm to 130 μm) or Kollidon® CL-SF (crospovidone with an average particle size of 10 μm to 30 μm). Other binders known to those of skill in the art are also contemplated as being useful when formulated in the compositions described herein.

In some embodiments, the filler is a diluent. Suitable diluents include, but are not limited to, lactose, mannitol, isomalt, sucrose, dextrose, and sorbitol.

In some embodiments, the filler is a disintegrant. Disintegrants include any agent that promotes breakup of the formulation in an aqueous environment, for example, to promote more rapid release of the active pharmaceutical ingredient (e.g., the compound of Formula (I), or a pharmaceutically acceptable salt thereof). Exemplary disintegrants include, but are not limited to, starch and modified starches, such as corn starch, potato starch, sodium starch glycolate or croscarmellose sodium, alginic acid, alginates, such as sodium alginate, polyvinylpyrrolidone, bentonite, methylcellulose, agar, carboxymethylcellulose, crospovidone, acid-carbonate effervescent systems, such as citric acid with bicarbonate salts, and ion exchange resins. Other disintegrants known to those of skill in the art are also contemplated as being useful when formulated in the compositions described herein.

In some embodiments, the pharmaceutical composition comprises a disintegrant. In some embodiments, the pharmaceutical composition comprises about 1 w/w % to about 30 w/w % of the disintegrant. In some embodiments, the pharmaceutical composition comprises about 5 w/w % to about 15 w/w % of the disintegrant. In some embodiments, the pharmaceutical composition comprises about 10 w/w % of the disintegrant. In some embodiments, the disintegrant is selected from croscarmellose sodium, sodium starch glycolate, crospovidone, and sodium bicarbonate. In some embodiments, the disintegrant is croscarmellose sodium.

In some embodiments, the filler is a glidant. Glidants can be used to improve the flowability of a powder or granules or both. Glidants include, but are not limited to, silicone dioxide, such as colloidal silicon dioxide or hydrated silicon dioxide, magnesium silicate, magnesium aluminometasilicate, talc, starch, calcium silicate, light anhydrous silicic acid, and silicon dioxide aerogels.

In some embodiments, the pharmaceutical composition comprises a glidant. In some embodiments, the pharmaceutical composition comprises about 0.1 w/w % to about 5 w/w % of the glidant. In some embodiments, the pharmaceutical composition comprises about 0.1 w/w % to about 1 w/w % of the glidant. In some embodiments, the pharmaceutical composition comprises about 0.67 w/w % of the glidant. In some embodiments, the glidant is selected from calcium silicate, silicon dioxide, and talc. In some embodiments, the glidant is calcium silicate.

In some embodiments, the filler is a surfactant, wetting agent, solubilizer, or combination thereof. Examples include, but are not limited to, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., Tween®), polyoxyethylene stearates, sodium dodecylsulfate, tyloxapol (a nonionic liquid polymer of the alkyl aryl polyether alcohol type, also known as superinone or triton). Other examples include, but are not limited to, poloxamers such as Pluronic® F68, F127, and F108, which are block copolymers of ethylene oxide and propylene oxide, and polyxamines such as Tetronic® 908 (also known as Poloxamine® 908), which is a tetrafunctional block copolymer derived from sequential addition of propylene oxide and ethylene oxide to ethylenediamine (available from BASF), dextran, lecithin, dialkylesters of sodium sulfosuccinic acid, such as Aerosol® OT, which is a dioctyl ester of sodium sulfosuccinic acid (available from American Cyanimid), Duponol® P, which is a sodium lauryl sulfate (available from DuPont), Triton® X-200, which is an alkyl aryl polyether sulfonate (available from Rohm and Haas), Tween® 20 and Tween® 80, which are polyoxyethylene sorbitan fatty acid esters (available from ICI Specialty Chemicals), Carbowax™ 3550 and 934, which are polyethylene glycols (available from Union Carbide), Crodesta™ F-110, which is a mixture of sucrose stearate and sucrose distearate, and Crodesta™ SL-40 (both available from Croda Inc.), and SA90HCO, which has the chemical formula C₁₈H₃₇—CH₂(CON(CH₃)CH₂(CHOH)₄CH₂OH)₂.

In some embodiments, the pharmaceutical composition comprises a filler. In some embodiments, the pharmaceutical composition comprises about 30 w/w % to about 99 w/w % of the filler. In some embodiments, the pharmaceutical composition comprises about 50 w/w % to about 90 w/w % of the filler. In some embodiments, the pharmaceutical composition comprises about 75.5 w/w % of the filler. In some embodiments, the filler is selected from mannitol, microcrystalline cellulose, lactose, starch, isomalt, silicified microcrystalline cellulose, Dicalcium Phosphate, maltodextrin, and a combination thereof. In some embodiments, the filler is a combination of mannitol and microcrystalline cellulose. In some embodiments, the pharmaceutical composition comprises about 30 w/w % to about 80 w/w % of mannitol. In some embodiments, the pharmaceutical composition comprises about 50 w/w % to about 60 w/w % of mannitol. In some embodiments, the pharmaceutical composition comprises about 56 w/w % of mannitol. In some embodiments, the pharmaceutical composition comprises about 1 w/w % to about 50 w/w % of microcrystalline cellulose. In some embodiments, the pharmaceutical composition comprises about 10 w/w % to about 30 w/w % of microcrystalline cellulose. In some embodiments, the pharmaceutical composition comprises about 20 w/w % of microcrystalline cellulose. In some embodiments, the pharmaceutical composition comprises about 56 w/w % of mannitol and about 20 w/w % of microcrystalline cellulose.

In some embodiments, the pharmaceutical composition includes a lubricant. Lubricants are agents added to pharmaceutical formulations to reduce friction during processing and prevent ingredients from clumping together. Exemplary lubricants include, but are not limited to, talc, starch, magnesium stearate, calcium stearate, sodium stearate, zinc stearate, stearic acid, vegetable stearin, adipic acid, waxy fatty acids, such as glyceryl behenate, a hydrogenated vegetable oil, a mineral oil, a polyethylene glycol, lycopodium, sodium lauryl sulfate, magnesium lauryl sulfate, glyceryl palmitostearate, sodium benzoate, sodium chloride, sterotex, glycerol monostearate, sodium stearyl fumarate, colloidal silicon dioxide, sodium benzoate, sodium oleate, and sodium acetate. Other lubricants known to those of skill in the art are also contemplated as being useful when formulated in the compositions described herein.

In some embodiments, the pharmaceutical composition comprises a lubricant. In some embodiments, the pharmaceutical composition comprises about 0.1 w/w % to about 10 w/w % of the lubricant. In some embodiments, the pharmaceutical composition comprises about 0.1 w/w % to about 1 w/w % of the lubricant. In some embodiments, the pharmaceutical composition comprises about 0.5 w/w % of the lubricant. In some embodiments, the pharmaceutical lubricant is selected from sodium stearyl fumarate, magnesium stearate, stearic acid sodium lauryl sulfate, sodium oleate, glyceryl behenate, and talc. In some embodiments, the lubricant is sodium stearyl fumarate.

In some embodiments, the pharmaceutical composition comprises:
(a) the compound of Formula (I), or a pharmaceutically acceptable salt thereof, and a polymer;
(b) a glidant;
(c) a filler; and
(d) a disintegrant.

In some embodiments, the pharmaceutical composition comprises:
(a) about 1 w/w % to about 20 w/w % of the compound of Formula (I), or a pharmaceutically acceptable salt thereof, and a polymer;
(b) about 0.1 w/w % to about 1 w/w % of a glidant;
(c) about 50 w/w % to about 90 w/w % of a filler; and
(d) about 5 w/w % to about 0.2 w/w % of a disintegrant.

In some embodiments, the pharmaceutical composition comprises:
(a) about 13 w/w % of the compound of Formula (I), or a pharmaceutically acceptable salt thereof, and a polymer;
(b) about 0.67 w/w % of a glidant;
(c) about 75.5 w/w % of a filler; and
(d) about 10 w/w % of a disintegrant.

In some embodiments, the pharmaceutical composition comprises:
(a) the compound of Formula (I), or a pharmaceutically acceptable salt thereof;
(b) calcium silicate;
(c) a combination of mannitol and microcrystalline cellulose; and
(d) croscarmellose sodium.

In some embodiments, the pharmaceutical composition comprises:
(a) about 1 w/w % to about 20 w/w % of the compound of Formula (I), or a pharmaceutically acceptable salt thereof;
(b) about 0.1 w/w % to about 1 w/w % of calcium silicate;
(c) about 50 w/w % to about 60 w/w % of mannitol and about 10 w/w % to about 30 w/w % of microcrystalline cellulose; and
(d) about 5 w/w % to about 0.2 w/w % of croscarmellose sodium.

In some embodiments, the pharmaceutical composition comprises:
(a) about 13 w/w % of the compound of Formula (I), or a pharmaceutically acceptable salt thereof;
(b) about 0.67 w/w % of calcium silicate;
(c) about 56 w/w % of mannitol and about 20 w/w % of microcrystalline cellulose; and
(d) about 10 w/w % of croscarmellose sodium.

Additional excipients can be included in the pharmaceutical formulations of the present disclosure. Further examples of excipients include, but are not limited to, pigments, colorants, flavoring agents, preservatives, and sweeteners. Flavors and colors can be added to improve the taste or appearance of a formulation. Examples of preservatives used in pharmaceutical compositions are aromatic alcohols, such as benzyl or phenol alcohol, antioxidants such as vitamin A, vitamin E, vitamin C, and selenium, amino acids such as cysteine and methionine, citric acid and sodium citrate, or synthetic preservatives such as methyl paraben and propyl paraben. Sweeteners can be added to make the ingredients more palatable, especially in chewable tablets or liquids like syrups.

### Dosing Schedule/Amount

A compound of Formula (I), or a pharmaceutically acceptable salt thereof, may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The amount of compound or composition administered to a subject will also vary depending upon what is being administered, the purpose of the administration, such as prophylaxis or therapy, the state of the subject, the manner of administration, and the like. In therapeutic applications, compositions may be administered to a subject already suffering from a disease in an amount sufficient to cure or at least partially arrest the symptomology and/or pathology of the disease and its complications. Therapeutically effective doses will depend on the disease condition being treated as well as by the judgment of the attending clinician depending upon factors such as the severity of the disease, the age, weight and general condition of the subject, and the like.

Some typical dosage ranges are from 1 μg/kg to 1 g/kg of body weight per day of the drug substance or equivalent mass of the drug substance. In some embodiments, the dose range is from about 0.01 mg/kg to about 100 mg/kg of body weight per day of the drug substance or equivalent mass of the drug substance of the drug substance. Representative amounts in a dose of the drug substance or equivalent mass of the drug substance include, but are not limited to, 0.01 mg/kg, 0.1 mg/kg, 0.2 mg/kg, 0.4 mg/kg, 0.6 mg/kg, 0.8 mg/kg, 1 mg/kg, 2 mg/kg, 4 mg/kg, 6 mg/kg, 8 mg/kg, 10 mg/kg, 12 mg/kg, 14 mg/kg, 16 mg/kg, 18 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, or an amount within a range defined by any of the preceding amounts of body weight per day.

The dosage may be a single one or a series of two or more given in the course of one or more days, as is needed by the subject. In some embodiments, the compounds will be administered for a period of continuous therapy, for example for a week or more, or for months or years. Effective doses may be extrapolated from dose-response curves derived from in vitro or animal model test systems, as well as human clinical trials.

In instances where human dosages for compounds have been established for at least some condition, those same dosages may be used, or dosages that are between 0.1% and 500%, more preferably between about 25% and 250% of the established human dosage. Where no human dosage is established, as will be the case for newly-discovered pharmaceutical compositions, a suitable human dosage may be inferred from ED₅₀ or values, or other appropriate values derived from in vitro or in vivo studies, as qualified by toxicity studies and efficacy studies in animals.

In cases of administration of a pharmaceutically acceptable salt, dosages may be calculated as the free form (e.g. free base or free acid). Often a drug is packaged in a salt form and the dosage form strength refers to the mass of this salt form or the equivalent mass of the corresponding free base or free acid. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but may be estimated from in vitro data. Dosages necessary to achieve the MEC will depend on individual characteristics and route of administration. However, HPLC assays or bioassays may be used to determine plasma concentrations. Dosage intervals may also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

In some embodiments of any of the methods disclosed herein, the compound of Formula (I), or a pharmaceutically acceptable salt thereof, is administered at a frequency of not less than twice daily; and the amount of the compound of Formula (I), or a pharmaceutically acceptable salt thereof, in the first administration is less than the amount of the compound of Formula (I), or a pharmaceutically acceptable salt thereof, in the second and any subsequent administrations.

In some embodiments, the compound of Formula (I), or a pharmaceutically acceptable salt thereof, is administered at a frequency of twice daily (i.e., comprising a first administration and a second administration).

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:100, 1:1.1 to 1:95, 1:1.1 to 1:90, 1:1.1 to 1:85, about 1:1.1 to 1:80, about 1:1.1 to 1:75, 1:1.1 to 1:70, 1:1.1 to 1:65, 1:1.1 to 1:60, 1:1.1 to 1:55, 1:1.1 to 1:50, 1:1.1 to 1:45, 1:1.1 to 1:40, 1:1.1 to 1:35, 1:1.1 to 1:30, 1:1.1 to 1:25, 1:1.1 to 1:20, about 1:1.1 to 1:15, 1:1.1 to 1:10, 1:1.1 to 1:9, 1:1.1 to 1:8, 1:1.1 to 1:7, 1:1.1 to 1:6, 1:1.1 to 1:5, 1:1.1 to 1:4, 1:1.1 to 1:3.5, 1:1.1 to 1:3, 1:1.1 to 1:2.5, 1:1.1 to 1:2, 1:1.1 to 1:1.5, or 1:1.1 to 1.25.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:100, about 1:1 to about 1:95, about 1:1 to about 1:90, about 1:1 to about 1:85, about 1:1 to about 1:80, about 1:1 to about 1:75, about 1:1 to about 1:70, about 1:1 to about 1:65, about 1:1 to about 1:60, about 1:1 to about 1:55, about 1:1 to about 1:50, about 1:1 to about 1:45, about 1:1 to about 1:40, about 1:1 to about 1:35, about 1:1 to about 1:30, about 1:1 to about 1:25, about 1:1 to about 1:20, about 1:1 to about 1:15, about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3.5, about 1:1 to about 1:3, about 1:1 to about 1:2.5, about 1:1 to about 1:2, about 1:1 to about 1:1.5 or about 1:1 to about 1.25.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:100.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:50.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:10.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:3.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:2.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is from about 1:1 to about 1:2.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:1.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:2.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:2.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration about 1:3.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:3.5.

In some embodiments, the ratio of the amount of the compound of Formula (I), or a pharmaceutically acceptable salt thereof, in the first administration to the amount of the compound of Formula (I), or a pharmaceutically acceptable salt thereof, in the second administration is about 1:4.

In some embodiments, the amount of the compound of Formula (I), or a pharmaceutically acceptable salt thereof, administered daily is less than or equal to about 1000 mg based on the weight of the free base.

In some embodiments, the amount of the compound of Formula (I), or a pharmaceutically acceptable salt thereof, administered daily is about 25 mg to about 1000 mg, about 50 mg to about 1000 mg, about 50 mg to about 950 mg, about 50 mg to about 900 mg, about 50 mg to about 850 mg, about 50 mg to about 800 mg, about 50 mg to about 750 mg, about 50 mg to about 700 mg, about 50 mg to about 650 mg, about 50 mg to about 600 mg, about 50 mg to about 550 mg, about 50 mg to about 500 mg, about 50 mg to about 450 mg, about 50 mg to about 400 mg, about 50 mg to about 350 mg, about 50 mg to about 300 mg, about 75 mg to about 350 mg, or about 75 mg to about 300 mg, wherein the daily amounts are based on the weight of the free base of the compound of Formula (I).

In some embodiments, the amount of the compound of Formula (I), or a pharmaceutically acceptable salt thereof, administered daily is about 100 mg to about 1000 mg, about 100 mg to about 950 mg, about 100 mg to about 900 mg, about 100 mg to about 850 mg, about 100 mg to about 800 mg, about 100 mg to about 750 mg, about 100 mg to about 700 mg, about 100 mg to about 650 mg, about 100 mg to about 600 mg, about 100 mg to about 550 mg, about 100 mg to about 500 mg, about 100 mg to about 450 mg, about 100 mg to about 400 mg, about 100 mg to about 350 mg, about 100 mg to about 300 mg, or about 100 mg to about 250, wherein the daily amounts are based on the weight of the free base of the compound of Formula (I).

In some embodiments, the amount of the compound of Formula (I), or a pharmaceutically acceptable salt thereof, administered daily is from about 50 mg to about 1000 mg based on the weight of the free base.

In some embodiments, the amount of the compound of Formula (I), or a pharmaceutically acceptable salt thereof, administered daily is from about 100 mg to about 1000 mg based on the weight of the free base.

In some embodiments, the amount of the compound of Formula (I), or a pharmaceutically acceptable salt thereof, administered daily is from about 100 mg to about 500 mg based on the weight of the free base.

In some embodiments, the amount of the compound of Formula (I), or a pharmaceutically acceptable salt thereof, administered daily is from about 100 mg to about 400 mg based on the weight of the free base.

In some embodiments, the amount of the compound of Formula (I), or a pharmaceutically acceptable salt thereof, administered daily is from about 100 mg to about 300 mg based on the weight of the free base.

In some embodiments, the amount of the compound of Formula (I), or a pharmaceutically acceptable salt thereof, administered daily is about 200 mg based on the weight of the free base. In a further embodiment, the first administration of the compound of Formula (I), or a pharmaceutically acceptable salt thereof is about 50 mg based on the weight of the free base and the second administration of the compound of Formula (I), or a pharmaceutically acceptable salt thereof is about 150 mg based on the weight of the free base.

In some embodiments, the amount of the compound of Formula (I), or a pharmaceutically acceptable salt thereof, administered daily is about 250 mg based on the weight of the free base. In further embodiments, the first administration of the compound of Formula (I), or a pharmaceutically acceptable salt thereof is about 100 mg and the second administration of the compound of Formula (I), or a pharmaceutically acceptable salt thereof is about 150 mg based on the weight of the free base.

In some embodiments, the amount of the compound of Formula (I), or a pharmaceutically acceptable salt thereof, administered daily is about 300 mg based on the weight of the free base. In a further embodiments, the first administration of the compound of Formula (I), or a pharmaceutically acceptable salt thereof is about 100 mg based on the weight of the free base and the second administration of the compound of Formula (I), or a pharmaceutically acceptable salt thereof is about 200 mg based on the weight of the free base.

In some embodiments, the subject weighs greater than or equal to about 55 kg. In some embodiments, the amount of the compound of Formula (I), or a pharmaceutically acceptable salt thereof, administered daily is about 200 mg or above and the subject weighs greater than or equal to about 55 kg.

In some embodiments, the amount of the compound of Formula (I), or a pharmaceutically acceptable salt thereof, administered daily is about 50 mg based on the weight of the free base.

In some embodiments, the subject weighs from about 10 kg to about 20 kg.

In some embodiments, the amount of the compound of Formula (I), or a pharmaceutically acceptable salt thereof, administered daily is about 50 mg based on the weight of the free base and the subject weighs from about 10 kg to about 20 kg.

In some embodiments, the amount of the compound of Formula (I), or a pharmaceutically acceptable salt thereof, administered daily is about 100 mg based on the weight of the free base. In some embodiments, the first administration of the compound of Formula (I), or a pharmaceutically acceptable salt thereof is about 25 mg based on the weight of the free base and the second administration of the compound of Formula (I), or a pharmaceutically acceptable salt thereof is about 75 mg based on the weight of the free base.

In some embodiments, the subject weighs from about 20 kg to about 55 kg and the amount of the compound of Formula (I), or a pharmaceutically acceptable salt thereof, administered daily is about 100 mg based on the weight of the free base.

In some embodiments, the compound of Formula (I), or a pharmaceutically acceptable salt thereof, is administered at a frequency of not less than twice daily, wherein the amount of the compound of Formula (I), or a pharmaceutically acceptable salt thereof, administered daily is greater than 200 mg based on the weight of the free base.

In some embodiments, the amount of the compound of Formula (I), or a pharmaceutically acceptable salt thereof, is administered at a frequency of twice daily.

In some embodiments, the amount of the compound of Formula (I), or a pharmaceutically acceptable salt thereof, administered daily is about 200 mg to about 1000 mg, about 200 mg to about 950 mg, about 200 mg to about 900 mg, about 200 mg to about 850 mg, about 200 mg to about 800 mg, about 200 mg to about 750 mg, about 200 mg to about 700 mg, about 200 mg to about 650 mg, about 200 mg to about 600 mg, about 200 mg to about 550 mg, about 200 mg to about 500 mg, about 200 mg to about 450 mg, about 200 mg to about 400 mg, about 200 mg to about 350 mg, about 200 mg to about 300 mg, or about 200 mg to about 250 mg, wherein the daily amounts are based on the weight of the free base of the compound of Formula (I).

In some embodiments, the amount of the compound of Formula (I), or a pharmaceutically acceptable salt thereof, administered daily is about 225 mg to about 1000 mg, about 225 mg to about 950 mg, about 225 mg to about 900 mg, about 225 mg to about 850 mg, about 225 mg to about 800 mg, about 225 mg to about 750 mg, about 225 mg to about 700 mg, about 225 mg to about 650 mg, about 225 mg to about 600 mg, about 225 mg to about 550 mg, about 225 mg to about 500 mg, about 225 mg to about 450 mg, about 225 mg to about 400 mg, about 225 mg to about 350 mg, about 225 mg to about 300 mg, or about 225 mg to about 250 mg, wherein the daily amounts are based on the weight of the free base of the compound of Formula (I).

In some embodiments, the amount of the compound of Formula (I), or a pharmaceutically acceptable salt thereof, administered daily is from about 200 mg to about 1000 mg based on the weight of the free base.

In some embodiments, the amount of the compound of Formula (I), or a pharmaceutically acceptable salt thereof, administered daily is from about 225 mg to about 1000 mg based on the weight of the free base.

In some embodiments, the amount of the compound of Formula (I), or a pharmaceutically acceptable salt thereof, administered daily is from about 225 mg to about 500 mg based on the weight of the free base.

In some embodiments, the amount of the compound of Formula (I), or a pharmaceutically acceptable salt thereof, administered daily is from about 225 mg to about 400 mg based on the weight of the free base.

In some embodiments, the amount of the compound of Formula (I), or a pharmaceutically acceptable salt thereof, administered daily is from about 225 mg to about 300 mg based on the weight of the free base.

In some embodiments, the amount of the compound of Formula (I), or a pharmaceutically acceptable salt thereof, administered daily is about 250 mg based on the weight of the free base. In a further embodiment, the first administration of the compound of Formula (I), or a pharmaceutically acceptable salt thereof is about 125 mg based on the weight of the free base and the second administration of the compound of Formula (I), or a pharmaceutically acceptable salt thereof is about 125 mg based on the weight of the free base.

In some embodiments, the amount of the compound of Formula (I), or a pharmaceutically acceptable salt thereof, administered daily is about 300 mg based on the weight of the free base. In a further embodiment, the first administration of the compound of Formula (I), or a pharmaceutically acceptable salt thereof is about 150 mg based on the weight of the free base and the second administration of the compound of Formula (I), or a pharmaceutically acceptable salt thereof is about 150 mg based on the weight of the free base.

In some embodiments of the methods disclosed herein (e.g., when the compound of Formulae (I), (IA) or (IB), or a pharmaceutically acceptable salt thereof, or a compound as disclosed and described herein, or a pharmaceutically acceptable salt thereof is administered at a frequency of twice daily), there are about 6 to about 14 hours between the first and second administrations. In some embodiments, there are about 8 to about 14 hours between the first and second administrations. In some embodiments, there are about 11 to about 13 hours between the first and second administrations. In some embodiments, there are about 12 hours between the first and second administrations. In some embodiments of the methods disclosed herein (e.g., when the compound of Formulae (I), (IA) or (IB), or a pharmaceutically acceptable salt thereof, or a compound as disclosed and described herein, or a pharmaceutically acceptable salt thereof is administered at a frequency of twice daily), the compound, or a pharmaceutically acceptable salt thereof, is administered twice daily at an amount from about 25 mg to about 250 mg based on the weight of the free base. In some embodiments, the compound, or a pharmaceutically acceptable salt thereof, is administered twice daily as: (a) a first administration of about 50 mg based on the weight of the free base and a second administration of about 350 mg based on the weight of the free base; or (b) a first administration of about 100 mg based on the weight of the free base and a second administration of about 300 mg based on the weight of the free base; or (c) a first administration of about 150 mg based on the weight of the free base and a second administration of about 250 mg based on the weight of the free base; or (d) a first administration of about 200 mg based on the weight of the free base and a second administration of about 250 mg based on the weight of the free base. In some embodiments, the compound is administered in the free base form.

In some embodiments, the compound, or a pharmaceutically acceptable salt thereof, is administered in a dose of about 25 mg, based on the weight of the free base. In some embodiments, the compound of Formula (I), or a pharmaceutically acceptable salt thereof, is administered in a dose of about 50 mg, based on the weight of the free base. In some embodiments, the compound of Formula (I), or a pharmaceutically acceptable salt thereof, is administered in a dose of about 75 mg, based on the weight of the free base. In some embodiments, the compound of Formula (I), or a pharmaceutically acceptable salt thereof, is administered in a dose of about 100 mg, based on the weight of the free base.

In some embodiments, the compound of Formula (I), or a pharmaceutically acceptable salt thereof, is administered in a dose of about 25 mg, based on the weight of the free base. In some embodiments, the compound of Formula (I), or a pharmaceutically acceptable salt thereof, is administered in a dose of about 50 mg, based on the weight of the free base. In some embodiments, the compound of Formula (I), or a pharmaceutically acceptable salt thereof, is administered in a dose of about 75 mg, based on the weight of the free base. In some embodiments, the compound of Formula (I), or a pharmaceutically acceptable salt thereof, is administered in a dose of about 100 mg, based on the weight of the free base.

In some embodiments, the pharmaceutical composition is administered in a dose of about 25 mg of the compound of Formula (I), or a pharmaceutically acceptable salt thereof, based on the weight of the free base. In some embodiments, the pharmaceutical composition is administered in a dose of about 50 mg of the compound of Formula (I), or a pharmaceutically acceptable salt thereof, based on the weight of the free base. In some embodiments, the pharmaceutical composition is administered in a dose of about 75 mg of the compound of Formula (I), or a pharmaceutically acceptable salt thereof, based on the weight of the free base. In some embodiments, the pharmaceutical composition is administered in a dose of about 100 mg of the compound of Formula (I), or a pharmaceutically acceptable salt thereof, based on the weight of the free base.

In some embodiments, the pharmaceutical composition comprises about 25 mg of the compound of Formula (I), or a pharmaceutically acceptable salt thereof, based on the weight of the free base. In some embodiments, the pharmaceutical composition comprises about 50 mg of the compound of Formula (I), or a pharmaceutically acceptable salt thereof, based on the weight of the free base. In some embodiments, the pharmaceutical composition comprises about 75 mg of the compound of Formula (I), or a pharmaceutically acceptable salt thereof, based on the weight of the free base. In some embodiments, the pharmaceutical composition comprises about 100 mg of the compound of Formula (I), or a pharmaceutically acceptable salt thereof, based on the weight of the free base.

The daily dosage of the compound of Formula (I), or a pharmaceutically acceptable salt thereof, in a pharmaceutical composition as described in the present disclosure can be varied over a wide range from about 1.0 mg to about 10,000 mg per adult human per day, or higher, or any range therein. For oral administration, the compositions can be provided in the form of tablets containing, for example, about 0.01 mg, about 0.05 mg, about 0.1 mg, about 0.5 mg, about 1.0 mg, about 2.5 mg, about 5.0 mg, about 10.0 mg, about 15.0 mg, about 25.0 mg, about 50.0 mg, about 75.0 mg, about 100 mg, about 150 mg, about 200 mg, about 250 or about 500 milligrams of the compound of Formula (I), or pharmaceutically acceptable salt thereof, for the symptomatic adjustment of the dosage to the subject to be treated. In some embodiments, an effective amount of the compound of Formula (I), or pharmaceutically acceptable salt thereof, can be supplied at a dosage level of from about 0.1 mg/kg to about 1000 mg/kg of body weight per day, or any range therein, for example, the range can be from about 0.5 mg/kg to about 500 mg/kg, about 1.0 mg/kg to about 250 mg/kg, about 0.1 mg/kg to about 100 mg/kg, about 0.1 mg/kg to about 50.0 mg/kg of body weight per day, about 0.1 mg/kg to about 15.0 mg/kg of body weight per day, about 0.5 mg/kg to about 7.5 mg/kg of body weight per day, or any amount to range therein. In some embodiments, an effective amount of the compound of Formula (I), or pharmaceutically acceptable salt thereof, can be supplied at a dosage level of from 0.1 mg/kg to 1000 mg/kg of body weight per day, or any range therein, for example, the range can be from 0.5 mg/kg to 500 mg/kg, 1.0 mg/kg to 250 mg/kg, 0.1 mg/kg to 100 mg/kg, 0.1 mg/kg to 50.0 mg/kg of body weight per day, 0.1 mg/kg to 15.0 mg/kg of body weight per day, 0.5 mg/kg to 7.5 mg/kg of body weight per day, or any amount to range therein. A pharmaceutical composition as provided herein can be administered on a regimen of 1 to 4 times per day or in a single daily dose.

In some embodiments, the daily dose of the compound of Formula (I), or a pharmaceutically acceptable salt thereof, is about 25 mg, based on the weight of the free base. In some embodiments, the daily dose of the compound of Formula (I), or a pharmaceutically acceptable salt thereof, is about 50 mg, based on the weight of the free base. In some embodiments, the daily dose of the compound of Formula (I), or a pharmaceutically acceptable salt thereof, is about 75 mg, based on the weight of the free base. In some embodiments, the daily dose of the compound of Formula (I), or a pharmaceutically acceptable salt thereof, is about 100 mg, based on the weight of the free base. In some embodiments, the daily dose of the compound of Formula (I), or a pharmaceutically acceptable salt thereof, is about 150 mg, based on the weight of the free base. In some embodiments, the daily dose of the compound of Formula (I), or a pharmaceutically acceptable salt thereof, is about 200 mg, based on the weight of the free base.

In some embodiments, the compound of Formula (I), or a pharmaceutically acceptable salt thereof, is administered twice daily in a dose of about 25 mg, based on the weight of the free base. In some embodiments, the compound of Formula (I), or a pharmaceutically acceptable salt thereof, is administered twice daily in a dose of about 50 mg, based on the weight of the free base. In some embodiments, the compound of Formula (I), or a pharmaceutically acceptable salt thereof, is administered twice daily in a dose of about 75 mg, based on the weight of the free base. In some embodiments, the compound of Formula (I), or a pharmaceutically acceptable salt thereof, is administered twice daily in a dose of about 100 mg, based on the weight of the free base.

In some embodiments, the daily dose of the pharmaceutical composition is about 25 mg of the compound of Formula (I), or a pharmaceutically acceptable salt thereof, based on the weight of the free base. In some embodiments, the daily dose of the pharmaceutical composition is about 50 mg of the compound of Formula (I), or a pharmaceutically acceptable salt thereof, based on the weight of the free base. In some embodiments, the daily dose of the pharmaceutical composition is about 75 mg of the compound of Formula (I), or a pharmaceutically acceptable salt thereof, based on the weight of the free base. In some embodiments, the daily dose of the pharmaceutical composition is about 100 mg of the compound of Formula (I), or a pharmaceutically acceptable salt thereof, based on the weight of the free base. In some embodiments, the daily dose of the pharmaceutical composition is about 150 mg of the compound of Formula (I), or a pharmaceutically acceptable salt thereof, based on the weight of the free base. In some embodiments, the daily dose of the pharmaceutical composition is about 200 mg of the compound of Formula (I), or a pharmaceutically acceptable salt thereof, based on the weight of the free base.

In some embodiments, the pharmaceutical composition is administered twice daily in a dose of about 25 mg of the compound of Formula (I), or a pharmaceutically acceptable salt thereof, based on the weight of the free base. In some embodiments, the pharmaceutical composition is administered twice daily in a dose of about 50 mg of the compound of Formula (I), or a pharmaceutically acceptable salt thereof, based on the weight of the free base. In some embodiments, the compound of Formula (I), or a pharmaceutically acceptable salt thereof, is administered twice daily in a dose of about 75 mg of the compound of Formula (I), or a pharmaceutically acceptable salt thereof, based on the weight of the free base. In some embodiments, the pharmaceutical composition is administered twice daily in a dose of about 100 mg of the compound of Formula (I), or a pharmaceutically acceptable salt thereof, based on the weight of the free base.

In some embodiments, the method comprises administering a daily dose of the pharmaceutical composition comprising about 25 mg of the compound of Formula (I), or a pharmaceutically acceptable salt thereof, based on the weight of the free base. In some embodiments, the method comprises administering a daily dose of the pharmaceutical composition comprising about 50 mg of the compound of Formula (I), or a pharmaceutically acceptable salt thereof, based on the weight of the free base. In some embodiments, the method comprises administering a daily dose of the pharmaceutical composition comprising about 75 mg of the compound of Formula (I), or a pharmaceutically acceptable salt thereof, based on the weight of the free base. In some embodiments, the method comprises administering a daily dose of the pharmaceutical composition comprising about 100 mg of the compound of Formula (I), or a pharmaceutically acceptable salt thereof, based on the weight of the free base. In some embodiments, the method comprises administering a daily dose of the pharmaceutical composition comprising about 150 mg of the compound of Formula (I), or a pharmaceutically acceptable salt thereof, based on the weight of the free base. In some embodiments, the method comprises administering a daily dose of the pharmaceutical composition comprising about 200 mg of the compound of Formula (I), or a pharmaceutically acceptable salt thereof, based on the weight of the free base.

In some embodiments, the method comprises administering the pharmaceutical composition twice daily in a dose of about 25 mg of the compound of Formula (I), or a pharmaceutically acceptable salt thereof, based on the weight of the free base. In some embodiments, the method comprises administering the pharmaceutical composition twice daily in a dose of about 50 mg of the compound of Formula (I), or a pharmaceutically acceptable salt thereof, based on the weight of the free base. In some embodiments, the method comprises administering the compound of Formula (I), or a pharmaceutically acceptable salt thereof, twice daily in a dose of about 75 mg of the compound of Formula (I), or a pharmaceutically acceptable salt thereof, based on the weight of the free base. In some embodiments, the method comprises administering the pharmaceutical composition twice daily in a dose of about 100 mg of the compound of Formula (I), or a pharmaceutically acceptable salt thereof, based on the weight of the free base.

In some embodiments of the methods disclosed herein, the compound of Formulae (I), (IA) or (IB), or a pharmaceutically acceptable salt thereof, or a compound as disclosed and described herein, or a pharmaceutically acceptable salt thereof is administered as a pharmaceutical composition comprising: (a) the compound of Formulae (I), (IA) or (IB), or a pharmaceutically acceptable salt thereof, or a compound as disclosed and described herein, or a pharmaceutically acceptable salt thereof; and (b) one or more of a lubricant, a diluent, a binder, and a glidant. In some embodiments, the pharmaceutical composition comprises about 70 wt % to about 97 wt % of the compound of Formulae (I), (IA) or (IB), or a pharmaceutically acceptable salt thereof, or a compound as disclosed and described herein, or a pharmaceutically acceptable salt thereof, based on the weight of the free base. In some embodiments, the pharmaceutical composition comprises about 80 wt % to about 96 wt % of the compound of Formulae (I), (IA) or (IB), or a pharmaceutically acceptable salt thereof, or a compound as disclosed and described herein, or a pharmaceutically acceptable salt thereof, based on the weight of the free base. In some embodiments, the pharmaceutical composition comprises about 85 wt % to about 90 wt % of the compound of Formulae (I), (IA) or (IB), or a pharmaceutically acceptable salt thereof, or a compound as disclosed and described herein, or a pharmaceutically acceptable salt thereof, based on the weight of the free base. In some embodiments, the pharmaceutical composition comprises a lubricant. In some embodiments, the pharmaceutical composition comprises a diluent. In some embodiments, the pharmaceutical composition comprises a binder. In some embodiments, the pharmaceutical composition comprises a glidant. In some embodiments, the pharmaceutical composition comprises the compound of Formulae (I), (IA) or (IB), or a pharmaceutically acceptable salt thereof, or a compound as disclosed and described herein, or a pharmaceutically acceptable salt thereof, in crystalline form. In some embodiments, the pharmaceutical composition comprises the compound of Formulae (I), (IA) or (IB), or a compound as disclosed and described herein as a free base. In some embodiments, the pharmaceutical composition is formulated in unit dosage form, wherein the compound of Formulae (I), (IA) or (IB), or a pharmaceutically acceptable salt thereof, or a compound as disclosed and described herein, or a pharmaceutically acceptable salt thereof, is present in an amount of about 25 mg to about 400 mg, based on the weight of the free base. In some embodiments, the pharmaceutical composition is formulated in unit dosage form, wherein the compound of Formulae (I), (IA) or (IB), or a pharmaceutically acceptable salt thereof, or a compound as disclosed and described herein, or a pharmaceutically acceptable salt thereof, is present in an amount of about 50 mg to about 400 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 50 mg, about 100 mg, about 150 mg, about 200 mg, about 250 mg, about 300 mg, about 350 mg, or about 400 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 75 mg, about 125 mg, about 175 mg, about 225 mg, about 275 mg, about 325 mg, or about 375 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 200 mg, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is present in the unit dosage form in an amount of about 400 mg, based on the weight of the free base. In some embodiments, the pharmaceutical composition is in the form of a tablet, capsule, sachet, powder, granules, coated particle, coated tablet, enterocoated tablet, enterocoated capsule, melting strip, or melting film. In some embodiments, the pharmaceutical composition is in tablet form. In some embodiments, the pharmaceutical composition is in capsule form. In some embodiments, the tablet form or capsule form is coated. In some embodiments, the compound of Formulae (I), (IA) or (IB), or a pharmaceutically acceptable salt thereof, or a compound as disclosed and described herein, or a pharmaceutically acceptable salt thereof is administered as a pharmaceutical composition in oral solution dosage form comprising: (a) the compound of Formulae (I), (IA) or (IB), or a pharmaceutically acceptable salt thereof, or a compound as disclosed and described herein, or a pharmaceutically acceptable salt thereof; (b) one or more of a sweetener, an anti-oxidant, and a flavor; and (c) a liquid vehicle. In some embodiments, the pharmaceutical composition comprises: (a) the compound of Formulae (I), (IA) or (IB), or a pharmaceutically acceptable salt thereof, or a compound as disclosed and described herein, or a pharmaceutically acceptable salt thereof; (b) a sweetener; (c) an anti-oxidant; (d) a flavor; and (e) a liquid vehicle. In some embodiments, the pharmaceutical composition further comprises a surfactant. In some embodiments, the pharmaceutical composition comprises: (a) the compound of Formulae (I), (IA) or (IB), or a pharmaceutically acceptable salt thereof, or a compound as disclosed and described herein, or a pharmaceutically acceptable salt thereof; (b) saccharin; (c) butylated hydroxytoluene; (d) a FONA orange flavor; and (e) medium-chain triglycerides. In some embodiments, the pharmaceutical composition further comprises a oleoyl polyoxyl-6 glycerides. In some embodiments, the pharmaceutical composition comprises the compound of Formulae (I), (IA) or (IB) or a compound as disclosed and described herein as a free base. In some embodiments, the pharmaceutical composition is formulated in unit dosage form, wherein the compound of Formulae (I), (IA) or (IB), or a pharmaceutically acceptable salt thereof, or a compound as disclosed and described herein, or a pharmaceutically acceptable salt thereof, is present in an amount of about 5 mg/mL to about 200 mg/mL, based on the weight of the free base. In some embodiments, the unit dosage form comprises the compound of Formulae (I), (IA) or (IB), or a pharmaceutically acceptable salt thereof, or a compound as disclosed and described herein, or a pharmaceutically acceptable salt thereof, in an amount of about 50 mg/mL, based on the weight of the free base. In some embodiments, the compound of Formulae (I), (IA) or (IB), or a pharmaceutically acceptable salt thereof, or a compound as disclosed and described herein, or a pharmaceutically acceptable salt thereof, is administered in a pharmaceutical composition comprising a spray-dried dispersion comprising: a compound of Formula (I), or a pharmaceutically acceptable salt thereof; and a polymer selected from a neutral polymer, an enteric polymer, and a pyrrolidone polymer; wherein the weight ratio of the compound of Formula (I) to the polymer is from about 1:1 to about 1:9.

In some embodiments of the methods disclosed herein, the spray-dried dispersion comprises: a compound of Formulae (I), (IA) or (IB), or a pharmaceutically acceptable salt thereof, or a compound as disclosed and described herein, or a pharmaceutically acceptable salt thereof; and a polymer that is a copolymer of 1-vinyl-2-pyrrolidone and vinyl acetate having the structure:

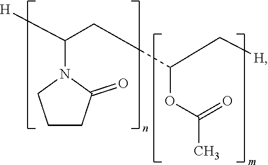

wherein the value of n is about 1 to about 2 times the value of m and the copolymer comprises 1-vinyl-2-pyrrolidone and vinyl acetate at a ratio of about 60:40 by weight; and wherein the weight ratio of the compound of Formula (I) to the copolymer is from about 1:1 to about 1:9. In some embodiments of the methods disclosed herein, the pharmaceutical composition comprises: (a) about 1 w/w % to about 20 w/w % of the spray-dried dispersion of Example 3; (b) about 0.1 w/w % to about 1 w/w % of calcium silicate; (c) about 50 w/w % to about 60 w/w % of mannitol and about 10 w/w % to about 30 w/w % of microcrystalline cellulose; and (d) about 5 w/w % to about 0.2 w/w % of croscarmellose sodium. In some embodiments of the methods disclosed herein, the pharmaceutical composition comprises: (a) about 1 w/w % to about 20 w/w % of the spray-dried dispersion of Example 3; (b) about 0.1 w/w % to about 1 w/w % of calcium silicate; (c) about 50 w/w % to about 60 w/w % of mannitol and about 10 w/w % to about 30 w/w % of microcrystalline cellulose; and (d) about 5 w/w % to about 0.2 w/w % of croscarmellose sodium. In some embodiments of the methods disclosed herein, the pharmaceutical composition comprises: (a) about 13 w/w % of the spray-dried dispersion of Example 3; (b) about 0.67 w/w % of calcium silicate; (c) about 56 w/w % of mannitol and about 20 w/w % of microcrystalline cellulose; and (d) about 10 w/w % of croscarmellose sodium.

Factors associated with the particular subject being treated, including subject age, weight, diet, and time of administration, can result in the need to adjust dosages. In some embodiments, the subject is a human adult. In some embodiments, the subject is a pediatric subject.

One skilled in the art will recognize that both in vivo and in vitro trials using suitable, known, and generally accepted cell and/or animal models are predictive of the ability of a test compound to treat or prevent a given disorder. One skilled in the art will further recognize that human clinical trials including first-in-human, dose ranging and efficacy trials, in healthy subjects and/or those suffering from a given disorder, can be completed according to methods well known in the clinical and medical arts. For example, determining proper dosages for pediatric subjects can be determined using known methods, including weight, age, and models such as Simcyp® Pediatric Simulation modeling (CERTARA, Princeton, N.J.) which can be used to establish a pharmacokinetic approach for dosing that takes into account subject age, ontogeny of the clearance pathways that a compound of Formula (I), or a pharmaceutically acceptable salt thereof, and body surface area (BSA).

In some embodiments, the pharmaceutical compositions of the present disclosure are stable for at least 3 months. In some embodiments, the pharmaceutical compositions are stable for at least 6 months. In some embodiments, the pharmaceutical compositions are stable for at least 9 months. In some embodiments, the pharmaceutical compositions are stable for at least 12 months. For example, the compositions do not exhibit a change (e.g., greater than 5%) in appearance, pH, percent impurities, activity (as measured by in vitro assays), or osmolarity over time, e.g., at least 3 months, 6 months, 9 months, or at least 12 months as compared to the original composition after manufacturing. In some embodiments, the pharmaceutical compositions do not exhibit a significant change, as defined by the International Conference on Harmonisation of Technical Requirements for Registration of Pharmaceuticals for Human Use (ICH), in one or more of appearance, pH, percent impurities, activity (as measured by in vitro assays), or osmolarity over time, e.g., at least 12 months as compared to the original pharmaceutical composition after manufacturing.

Compounds disclosed herein may be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan may be guided by the state of the art to choose an appropriate model, dose, route of administration and/or regime.

It will be apparent to those skilled in the art that the dosage forms described herein may comprise, as the drug substance, either a compound described herein or pharmaceutically acceptable salt, solvate, or hydrate thereof. Moreover, various hydrates and solvates of the compounds described herein and their salts may find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999 which is incorporated herein by reference in its entirety. Accordingly, one aspect of the present invention pertains to methods of administering hydrates and solvates of compounds described herein and/or their pharmaceutical acceptable salts, that may be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Compound Synthesis

Detailed compound synthesis methods are described herein in the Examples. In general, starting components are commercially available chemicals and may be obtained from commercial sources or may be made according to organic synthesis techniques known to those skilled in this art, starting from commercially available chemicals and/or from compounds described in the chemical literature.

In general, the compounds used in the reactions described herein may be made according to organic synthesis techniques known to those skilled in this art, starting from commercially available chemicals and/or from compounds described in the chemical literature. “Commercially available chemicals” may be obtained from standard commercial sources including Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hanover, Germany), Spectrum Quality Product, Inc. (New Brunswick, NJ), TCI America (Portland OR), Trans World Chemicals, Inc. (Rockville MD), and Wako Chemicals USA, Inc. (Richmond VA).

Methods known to one of ordinary skill in the art may be identified through various reference books and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2019) John Wiley & Sons, in over 95 volumes; and “Chemistry of Functional Groups” John Wiley & Sons, in hardcover volumes (86) and electronic volumes (26).

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses according to known methods, where many of the standard chemical supply houses (e.g., those listed above) provide custom synthesis services.

### Protocol A:

General Procedure for Preparation of Compounds of Formula (I)

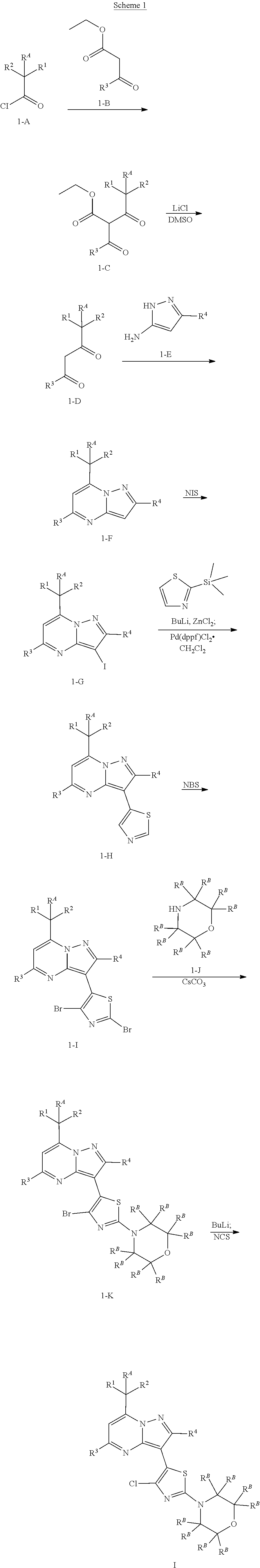

According to Scheme 1, compounds of general formula (I) may be synthesized from compounds of general formula 1-A. In one instance, a compound of general formula 2-A and magnesium chloride in a solvent such a methylene chloride may be combined with a solvent such as pyridine and then reacted with a compound of general formula 1-A to provide a compound of general formula 1-C. A compound of general formula 1-C may be decarboxylated to provide a compound of general Formula 1-D. An example of decarboxylation conditions are: Krapcho decarboxylation conditions where a compound of general formula 1-C may be heated in dimethyl sulfoxide in the presence of LiCl at a temperature of about 130 to 170° C. to provide a compound of general Formula 1-D. Combining a compound of general Formula 1-D with a compound of general formula 1-E in a suitable solvent, such as methanol, ethanol, acetic acid, and the like, at temperatures ranging from 0 to 60° C. affords a compound of general Formula 1-F. A compound of general formula 1-F may be reacted employing standard iodination conditions to provide a compound of general formula 1-G. For example, a compound of general formula 1-F may be reacted with an excess of N-iodosuccinimide in a solvent such as acetonitrile to provide a compound of general formula 1-G. Subsequent coupling of a compound of general formula 1-G with a thiazole zinc halide under Negishi cross-coupling reaction conditions may provide a compound of general formula 1-H. For example, reaction of compounds of general formula 1-G, with commercially available or synthetically accessible thiazole zinc halide in a solvent such as THF, 1,4-dioxane, MTBE and the like, optionally in the presence of a base such as, NaHCO₃, Na₂CO₃, K₂CO₃, K₃PO₄, Cs₂CO₃, and the like, and an organotransition metal catalyst such as Pd(dppf)Cl₂·CH₂Cl₂, Pd₂(dba)₃, Pd(dppf)₂, tetrakis(triphenylphosphine) palladium (0) (Pd(PPh₃)₄), and the like, employing conventional or microwave heating, at temperatures ranging from 80 to 120° C. may provide a compound of general formula 1-H. In one instance, thiazole zinc halide may be prepared by treating 2-trimethylsilanylthiazole dissolved in a solvent, such as THF, 1,4-dioxane, MTBE and the like, under an inert atmosphere at temperatures ranging from about −78° C. to about −100° C. with an organolithium compound such as n-butyl lithium and stirring at temperatures ranging from about −78° C. to about −100° C. for a period of time such as 30 min. to 1 hr and then treating the formed mixture with anhydrous zinc halide such as zinc chloride to afford thiazole zinc halide such as thiazole zinc chloride. In one instance, thiazole zinc chloride may be allowed to warm to rt and then treated with a compound of general formula 1-G and Pd(dppf)Cl₂·CH₂Cl₂ in CH₂Cl₂ under an inert atmosphere where the resulting mixture may refluxed overnight to afford a compound of general formula 1-H. A compound of general formula 1-H may be reacted employing standard bromination conditions to provide a compound of general formula 1-I. For example, a compound of general formula 1-H may be reacted with an excess of N-bromosuccinimide in a solvent such as acetonitrile to provide a compound of general formula 1-I. In one instance, a compound of general formula 1-I may be reacted with a compound of general formula 1-J in the presence of a base such as, NaHCO₃, Na₂CO₃, K₂CO₃, K₃PO₄, Cs₂CO₃, and the like, in an appropriate solvent, such as THF, 1,4-dioxane, MTBE and the like, at temperatures ranging from 80° C. to 120° C. such as 105° C., for a period of 12 to 48 h such as overnight to provide a compound of general formula 1-K. Formation of a compound of general formula I may be accomplished by reacting a compound of general formula 1-K dissolved in a solvent, such as THF, 1,4-dioxane, MTBE and the like, under an inert atmosphere at temperatures ranging from about −78° C. to about −100° C. with an organolithium compound such as n-butyl lithium and stirring at temperatures ranging from about −78° C. to about −100° C. for a period of time such as 30 min. to 1 hr and then treating the formed organolithium compound with a chlorinating reagent such as N-chlorosuccinimide. For example, a compound of general formula 1-K dissolved in THE may be treated with n-butyl lithium at −78° C. and stirred for 30 min. and then treated with N-chlorosuccinimide and allowed to slowly warm to rt with further stirring overnight to provide a compound of general formula I.

### EXAMPLES

The following examples are included to demonstrate embodiments of the disclosure. However, those of skill in the art should, in light of the present disclosure, appreciate that many changes may be made in the specific embodiments which are disclosed and still obtain a like or similar result without departing from the spirit and scope of the disclosure.

### Example 1

### Synthesis of Compound 1

### Scheme 2: Preparation of 4-(4-chloro-5-(2-methyl-5-(methyl-d₃)-7-(pentan-3-yl)pyrazolo[1,5-a]pyrimidin-3-yl)thiazol-2-yl)morpholine (Compound 1)

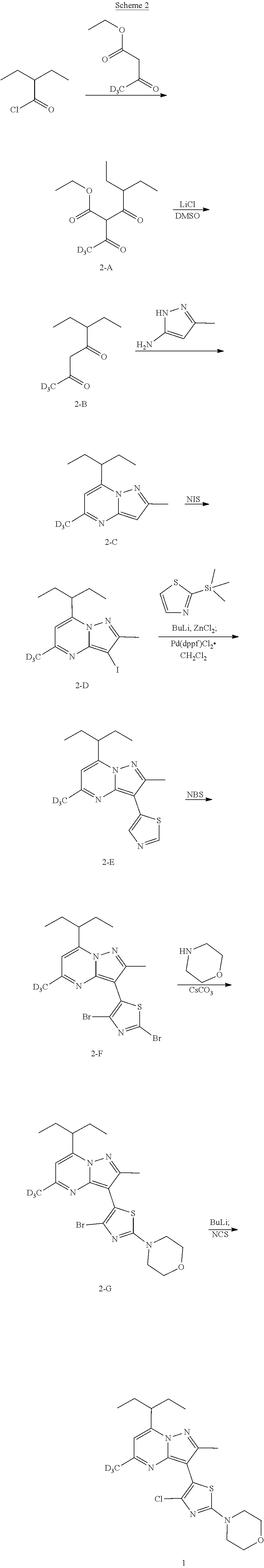

### Step 1: Preparation of Ethyl 2-(acetyl-d₃)-4-ethyl-3-oxohexanoate (Compound 2-A)

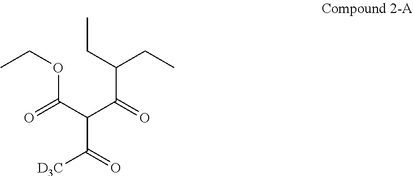 [SMILES: [2H]C([2H])([2H])C(=O)C(C(=O)OCC)C(=O)C(CC)CC]

A mixture of ethyl 3-oxobutanoate-4,4,4-d₃ and magnesium chloride in methylene chloride is stirred at room temperature for one hour. The mixture is cooled in an ice water bath and treated with pyridine dropwise addition. The stirring mixture is treated with di-n-propylacetyl chloride by dropwise addition at 0° C. The mixture is allowed to warm to rt and stir 16 h. The mixture is combined with 1 N HCl and the layers are separated. The organic layer is worked up following standard procedures to afford the title compound.

### Step 2: Preparation of 5-ethylheptane-2,4-dione-1,1,1-d₃ (Compound 2-B)

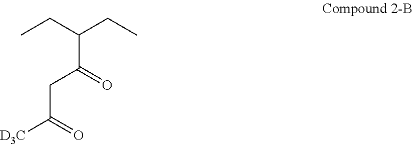

A mixture of ethyl 2-(acetyl-d₃)-4-ethyl-3-oxohexanoate and LiCl in dimethyl sulfoxide and water is heated at 150° C. for 6 to 8 h. The mixture is allowed to cool to rt and extracted with heptane. The organic layer is worked up following standard procedures to afford the title compound.

### Step 3: Preparation of 2-methyl-5-(methyl-d₃)-7-(pentan-3-yl)pyrazolo[1,5-a]pyrimidine (Compound 2-C)

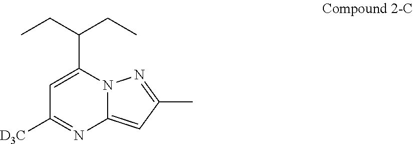 [SMILES: [2H]C([2H])([2H])c1cc(C(CC)CC)n2nc(C)cc2n1]

A mixture of 5-ethylheptane-2,4-dione-1,1,1-d₃ and acetic acid is cooled in an ice bath and treated with 5-amino-3-methylpyrazole and stirred at rt. The excess acetic acid is removed and the remainder is combined with water. The organic layer is worked up following standard procedures to afford the title compound.

### Step 4: Preparation of 3-iodo-2-methyl-5-(methyl-d₃)-7-(pentan-3-yl)pyrazolo[1,5-a]pyrimidine (Compound 2-D)

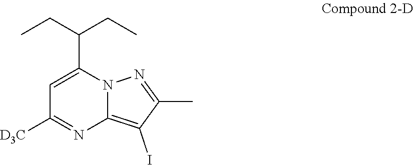 [SMILES: [2H]C([2H])([2H])c1cc(C(CC)CC)n2nc(C)c(I)c2n1]

A mixture of 2-methyl-5-(methyl-d₃)-7-(pentan-3-yl)pyrazolo[1,5-a]pyrimidine and anhydrous acetonitrile is treated with N-iodosuccinimide in portions at 10 minute intervals. The mixture is stirred until disappearance of starting material. The remainder is combined with methylene chloride. The organic layer is worked up following standard procedures to afford the title compound.

### Step 5: Preparation of 5-(2-methyl-5-(methyl-d₃)-7-(pentan-3-yl)pyrazolo[1,5-a]pyrimidin-3-yl)thiazole (Compound 2-E)

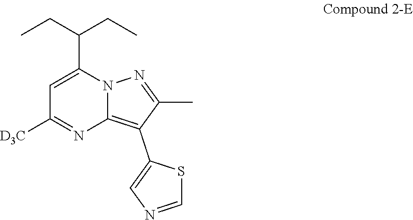 [SMILES: [2H]C([2H])([2H])c1cc(C(CC)CC)n2nc(C)c(-c3cncs3)c2n1]

A mixture of 2-trimethylsilanylthiazole is dissolved in anhydrous THE and cooled to −78° C. The stirring mixture is treated with n-butyl lithium and stirred 30 min at −78° C. Subsequently, anhydrous zinc chloride is added to the stirring mixture in one aliquot and the resulting mixture is stirred 30 min at −78° C. The mixture is then allowed to warm to room temperature stirring 30 min, and 3-iodo-2-methyl-5-(methyl-d₃)-7-(pentan-3-yl)pyrazolo[1,5-a]pyrimidine and dichloro[1,1′-bis(diphenylphosphino)ferrocene]palladium (II) dichloromethane is added. The resulting mixture is refluxed overnight. The mixture is then cooled to rt and combined with saturated sodium bicarbonate, and diluted with ethyl acetate. The aqueous layer is extracted with ethyl acetate. The combined organic layer is worked up following standard procedures to afford the title compound.

### Step 6: Preparation of 2,4-dibromo-5-(2-methyl-5-(methyl-d₃)-7-(pentan-3-yl)pyrazolo[1,5-a]pyrimidin-3-yl)thiazole (Compound 2-F)

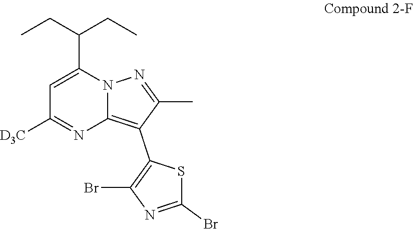 [SMILES: [2H]C([2H])([2H])c1cc(C(CC)CC)n2nc(C)c(-c3sc(Br)nc3Br)c2n1]

A mixture of 5-(2-methyl-5-(methyl-d₃)-7-(pentan-3-yl)pyrazolo[1,5-a]pyrimidin-3-yl)thiazole in acetonitrile is treated with N-bromosuccinimide in one aliquot. The resulting mixture is stirred overnight under an inert atmosphere. The solvent is removed under reduced pressure. The remainder is combined with methylene chloride. The organic layer is worked up following standard procedures to afford the title compound.

### Step 7: Preparation of 4-(4-bromo-5-(2-methyl-5-(methyl-d₃)-7-(pentan-3-yl)pyrazolo[1,5-a]pyrimidin-3-yl)thiazol-2-yl)morpholine (Compound 2-G)

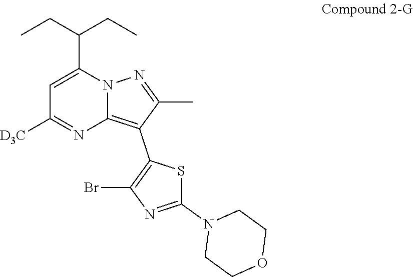 [SMILES: [2H]C([2H])([2H])c1cc(C(CC)CC)n2nc(C)c(-c3sc(N4CCOCC4)nc3Br)c2n1]

A mixture of 2,4-dibromo-5-(2-methyl-5-(methyl-d₃)-7-(pentan-3-yl)pyrazolo[1,5-a]pyrimidin-3-yl)thiazole, morpholine, and cesium carbonate in dioxane is refluxed under an inert atmosphere overnight and then allowed to cool to rt. The mixture is combined with ethyl acetate, washed with water, back extracting the aqueous layer with ethyl acetate. The combined organic is worked up following standard procedures to afford the title compound.

### Step 8: Preparation of 4-(4-chloro-5-(2-methyl-5-(methyl-d₃)-7-(pentan-3-yl)pyrazolo[1,5-a]pyrimidin-3-yl)thiazol-2-yl)morpholine (Compound 1)

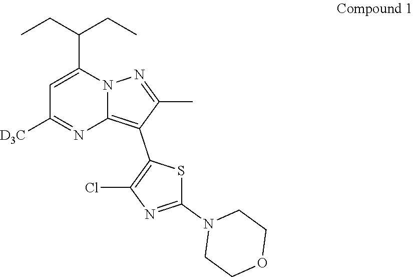 [SMILES: [2H]C([2H])([2H])c1cc(C(CC)CC)n2nc(C)c(-c3sc(N4CCOCC4)nc3Cl)c2n1]

A mixture of 4-(4-bromo-5-(2-methyl-5-(methyl-d₃)-7-(pentan-3-yl)pyrazolo[1,5-a]pyrimidin-3-yl)thiazol-2-yl)morpholine in THE is cooled to −78° C. The mixture is treated with n-butyl lithium and the resulting mixture is stirred at −78° C. for 30 min and then treated with N-chlorosuccinimide stirring for another 30 min. The resulting mixture is allowed to slowly warm to room temperature and stirred overnight. The mixture is combined with a solution of saturated ammonia chloride and extracted with ethyl acetate. The organic is worked up following standard procedures to afford the title compound.

### Example 2

### Synthesis of Compound 2

### Scheme 3: Preparation of 4-(4-chloro-5-(2,5-dimethyl-7-(pentan-3-yl-3-d)pyrazolo[1,5-a]pyrimidin-3-yl)thiazol-2-yl)morpholine (Compound 2)

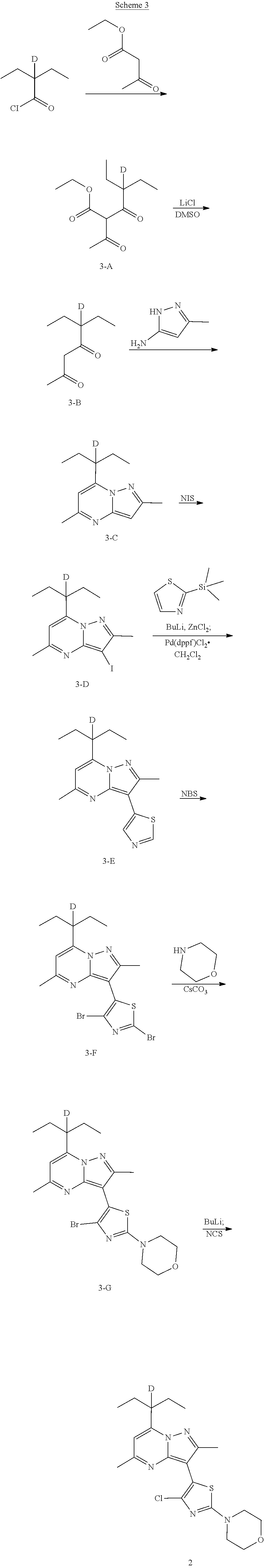

### Step 1: Preparation of Ethyl 2-acetyl-4-ethyl-3-oxohexanoate-4-d (Compound 3-A)

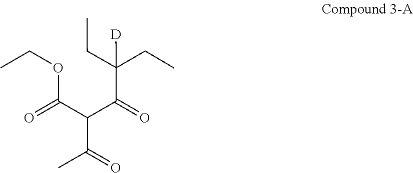 [SMILES: [2H]C(CC)(CC)C(=O)C(C(C)=O)C(=O)OCC]

A mixture of ethyl acetoacetate and magnesium chloride in methylene chloride is stirred at room temperature for one hour. The mixture is cooled in an ice water bath and treated with pyridine dropwise addition. The stirring mixture is treated with 2-ethylbutanoyl-2-d chloride by dropwise addition at 0° C. The mixture is allowed to warm to rt and stir 16 h. The mixture is combined with 1 N HCl and the layers are separated. The organic layer is worked up following standard procedures to afford the title compound.

### Step 2: Preparation of 5-ethylheptane-2,4-dione-5-d (Compound 3-B)

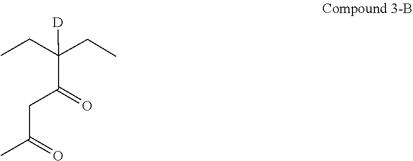

A mixture of ethyl 2-acetyl-4-ethyl-3-oxohexanoate-4-d and LiCl in dimethyl sulfoxide and water is heated at 150° C. for 6 to 8 h. The mixture is allowed to cool to rt and extracted with heptane. The organic layer is worked up following standard procedures to afford the title compound.

### Step 3: Preparation of 2,5-dimethyl-7-(pentan-3-yl-3-d)pyrazolo[1,5-a]pyrimidine (Compound 3-C)

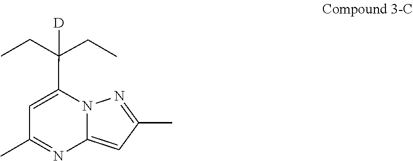

A mixture of 5-ethylheptane-2,4-dione-5-d and acetic acid is cooled in an ice bath and treated with 5-amino-3-methylpyrazole and stirred at rt. The excess acetic acid is removed and the remainder is combined with water. The organic layer is worked up following standard procedures to afford the title compound.

### Step 4: Preparation of 3-iodo-2,5-dimethyl-7-(pentan-3-yl-3-d)pyrazolo[1,5-a]pyrimidine (Compound 3-D)

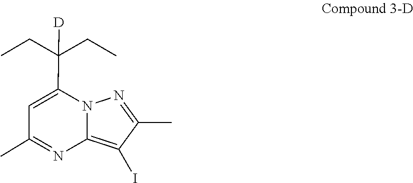

A mixture of 2,5-dimethyl-7-(pentan-3-yl-3-d)pyrazolo[1,5-a]pyrimidine and anhydrous acetonitrile is treated with N-iodosuccinimide in portions at 10 minute intervals. The mixture is stirred until disappearance of starting material. The remainder is combined with methylene chloride. The organic layer is worked up following standard procedures to afford the title compound.

### Step 5: Preparation of 5-(2,5-dimethyl-7-(pentan-3-yl-3-d)pyrazolo[1,5-a]pyrimidin-3-yl)thiazole (Compound 3-E)

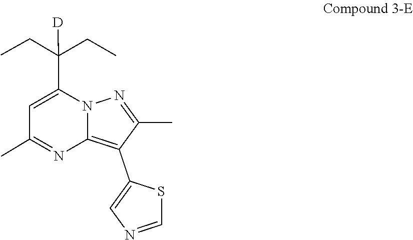 [SMILES: [2H]C(CC)(CC)c1cc(C)nc2c(-c3cncs3)c(C)nn12]

A mixture of 2-trimethylsilanylthiazole is dissolved in anhydrous THE and cooled to −78° C. The stirring mixture is treated with n-butyl lithium and stirred 30 min at −78° C. Subsequently, anhydrous zinc chloride is added to the stirring mixture in one aliquot and the resulting mixture is stirred 30 min at −78° C. The mixture is then allowed to warm to room temperature stirring 30 min, and 3-iodo-2,5-dimethyl-7-(pentan-3-yl-3-d)pyrazolo[1,5-a]pyrimidine and dichloro[1,1′-bis(diphenylphosphino)ferrocene]palladium (II) dichloromethane is added. The resulting mixture is refluxed overnight. The mixture is then cooled to rt and combined with saturated sodium bicarbonate, and diluted with ethyl acetate. The aqueous layer is extracted with ethyl acetate. The combined organic layer is worked up following standard procedures to afford the title compound.

### Step 6: Preparation of 2,4-dibromo-5-(2,5-dimethyl-7-(pentan-3-yl-3-d)pyrazolo[1,5-a]pyrimidin-3-yl)thiazole (Compound 3-F)

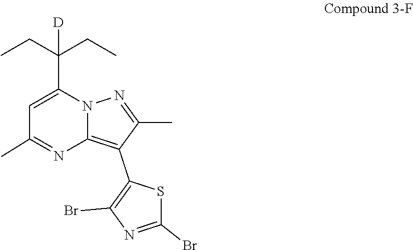 [SMILES: [2H]C(CC)(CC)c1cc(C)nc2c(-c3sc(Br)nc3Br)c(C)nn12]

A mixture of 5-(2,5-dimethyl-7-(pentan-3-yl-3-d)pyrazolo[1,5-a]pyrimidin-3-yl)thiazole in acetonitrile is treated with N-bromosuccinimide in one aliquot. The resulting mixture is stirred overnight under an inert atmosphere. The solvent is removed under reduced pressure. The remainder is combined with methylene chloride. The organic layer is worked up following standard procedures to afford the title compound.

### Step 7: Preparation of 4-(4-bromo-5-(2,5-dimethyl-7-(pentan-3-yl-3-d)pyrazolo[1,5-a]pyrimidin-3-yl)thiazol-2-yl)morpholine (Compound 3-G)

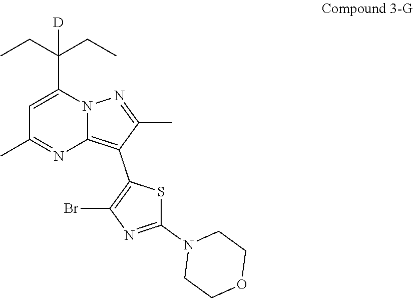 [SMILES: [2H]C(CC)(CC)c1cc(C)nc2c(-c3sc(N4CCOCC4)nc3Br)c(C)nn12]

A mixture of 2,4-dibromo-5-(2,5-dimethyl-7-(pentan-3-yl-3-d)pyrazolo[1,5-a]pyrimidin-3-yl)thiazole, morpholine, and cesium carbonate in dioxane is refluxed under an inert atmosphere overnight and then allowed to cool to rt. The mixture is combined with ethyl acetate, washed with water, back extracting the aqueous layer with ethyl acetate. The combined organic is worked up following standard procedures to afford the title compound.

### Step 8: Preparation of 4-(4-chloro-5-(2,5-dimethyl-7-(pentan-3-yl-3-d)pyrazolo[1,5-a]pyrimidin-3-yl)thiazol-2-yl)morpholine (Compound 2)

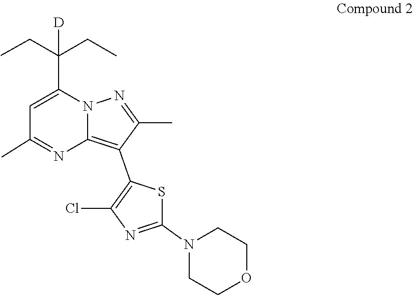 [SMILES: [2H]C(CC)(CC)c1cc(C)nc2c(-c3sc(N4CCOCC4)nc3Cl)c(C)nn12]

A mixture of 4-(4-bromo-5-(2,5-dimethyl-7-(pentan-3-yl-3-d)pyrazolo[1,5-a]pyrimidin-3-yl)thiazol-2-yl)morpholine in THE is cooled to −78° C. The mixture is treated with n-butyl lithium and the resulting mixture is stirred at −78° C. for 30 min and then treated with N-chlorosuccinimide stirring for another 30 min. The resulting mixture is allowed to slowly warm to room temperature and stirred overnight. The mixture is combined with a solution of saturated ammonia chloride and extracted with ethyl acetate. The organic is worked up following standard procedures to afford the title compound.

### Example 3

### Synthesis of Compound 3

### Scheme 4: Preparation of 4-(4-chloro-5-(2,5-dimethyl-7-(pentan-3-yl)pyrazolo[1,5-a]pyrimidin-3-yl)thiazol-2-yl)morpholine-2,2,3,3,5,5,6,6-d₈ (Compound 3)

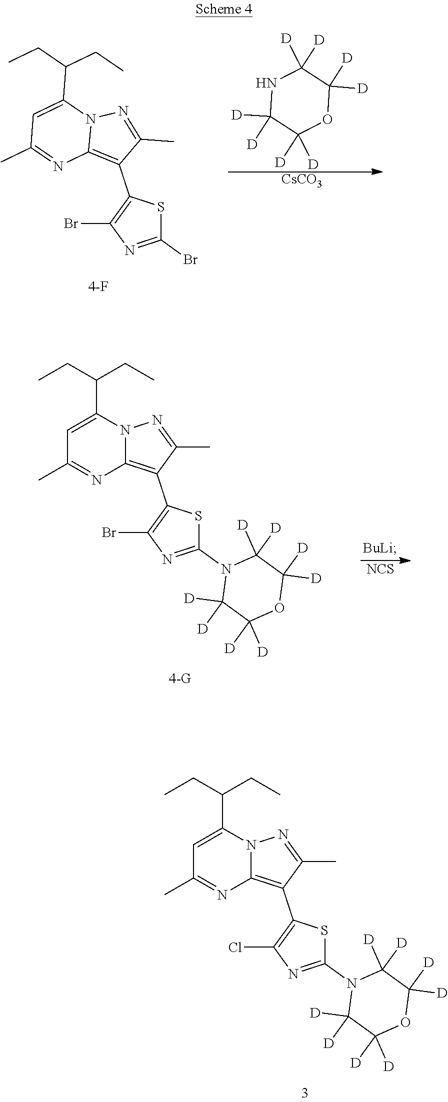

### Step 1: Preparation of 4-(4-bromo-5-(2,5-dimethyl-7-(pentan-3-yl)pyrazolo[1,5-a]pyrimidin-3-yl)thiazol-2-yl)morpholine-2,2,3,3,5,5,6,6-d₈ (Compound 1-F)

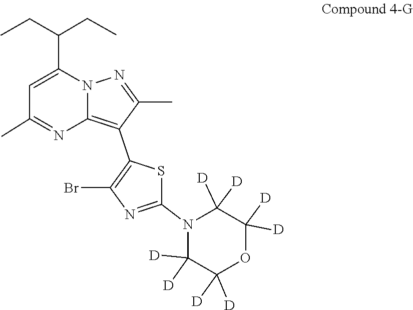 [SMILES: [2H]C1([2H])OC([2H])([2H])C([2H])([2H])N(c2nc(Br)c(-c3c(C)nn4c(C(CC)CC)cc(C)nc34)s2)C1([2H])[2H]]

A mixture of 2,4-dibromo-5-(2,5-dimethyl-7-(pentan-3-yl)pyrazolo[1,5-a]pyrimidin-3-yl)thiazole (prepared as described in U.S. Pat. No. 8,030,304 B2), morpholine-2,2,3,3,5,5,6,6-ds, and cesium carbonate in dioxane is refluxed under an inert atmosphere overnight and then allowed to cool to rt. The mixture is combined with ethyl acetate, washed with water, back extracting the aqueous layer with ethyl acetate. The combined organic is worked up following standard procedures to afford the title compound.

### Step 2: Preparation of 4-(4-chloro-5-(2,5-dimethyl-7-(pentan-3-yl)pyrazolo[1,5-a]pyrimidin-3-yl)thiazol-2-yl)morpholine-2,2,3,3,5,5,6,6-d₈ (Compound 3)

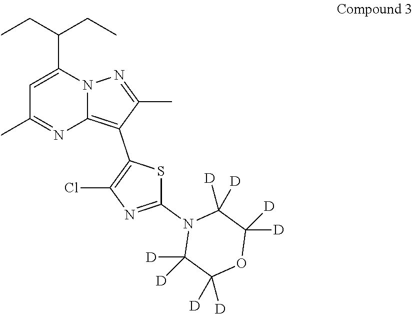 [SMILES: [2H]C1([2H])OC([2H])([2H])C([2H])([2H])N(c2nc(Cl)c(-c3c(C)nn4c(C(CC)CC)cc(C)nc34)s2)C1([2H])[2H]]

A mixture of 4-(4-bromo-5-(2,5-dimethyl-7-(pentan-3-yl)pyrazolo[1,5-a]pyrimidin-3-yl)thiazol-2-yl)morpholine-2,2,3,3,5,5,6,6-ds in THE is cooled to −78° C. The mixture is treated with n-butyl lithium and the resulting mixture is stirred at −78° C. for 30 min and then treated with N-chlorosuccinimide stirring for another 30 min. The resulting mixture is allowed to slowly warm to room temperature and stirred overnight. The mixture is combined with a solution of saturated ammonia chloride and extracted with ethyl acetate. The organic is worked following standard procedures to afford the title compound.

### Example 4

### Liquid Formulation 1 of Compounds of Formula (I)

Table 4 shows liquid formulation 1 of a Compound of Formula (I) free base (e.g. Compound 1, 2 or 3).

TABLE 4

| | | | 50 mg/ml | | |
|---|---|---|---|---|---|
| | | | Oral Solution | | Batch |
| | Quality | | Weight | % | Weight |
| Component | Standard | Function | (mg/mL) | (w/v) | (g) |
| Compounds of Formula (I) FB | In-house | Drug Substance | 50.0 | 5 | 20.03 |
| Saccharin | NF/EP | Sweetener | 1.5 | 0.15 | 0.61 |
| Butylated hydroxytoluene | NF/EP | Anti-oxidant | 1.7 | 0.17 | 0.69 |
| FONA orange flavor | NF | Flavor | 1.0 | 0.1 | 0.41 |
| Labrafac Lipophile WL1349 | NF/EP | Liquid Vehicle | to 1 mL | 94.58 | 358.87 |

| | | | |
|---|---|---|---|
| Total | 1 mL | 100.0 | 380.6 |

### Example 5

### Liquid Formulation 2 of the Compound of Formula (I)

TABLE 5

| | | 50 mg/ml | | |
|---|---|---|---|---|
| | | Oral Solution | | Batch |
| | | Weight | % | Weight |
| Component | Function | (mg/mL) | (w/v) | (g) |
| Compounds of Formula (I) FB | Drug Substance | 50.0 | 5 | 20.17 |
| Saccharin | Sweetener | 1.5 | 0.15 | 0.61 |
| Butylated hydroxytoluene | Anti-oxidant | 1.7 | 0.17 | 0.68 |
| LABRAFIL M 1944 CS | Surfactant | 200.0 | 20 | 80.16 |
| FONA orange flavor | Flavor | 1.0 | 0.1 | 0.40 |
| Labrafac Lipophile WL1349 | Liquid Vehicle | to 1 mL | 74.58 | 278.68 |

| | | | |
|---|---|---|---|
| Total | 1 mL | 100.0 | 380.7 |

### Example 6

Formulation of Extra Granular Component with Compounds of Formula (I)

Compound of Formula (I) free base (e.g. Compound 1, 2 or 3), Povidone and Kolliphor® SLS fine are dry mixed. Water is added to the dry mixture and subjected to a wet granulation at an impellor speed of about 550-560 rpm or 600-610 rpm. The granules are then sifted and dried to afford the extra granular component.

Table 6 show formulation of Extra Granular Component with compounds of Formula (I).

TABLE 6

| | Weight | |
|---|---|---|
| Extra Granular Component | (mg/unit) | % (w/w) |
| Compounds of Formula (I), free base | 50 | 93 |
| Povidone Compendial [plasdoneK29/32] | 3.23 | 6 |
| Kolliphor ® SLS fine | 0.54 | 1 |
| Total | 53.77 | 100 |

### Example 7

### Formulation of Compounds of Formula (I)

The extra granular component of Example 6 is combined with the remaining components listed in Table 7. The resultant mixture is then compressed into tablets containing 50 mg of Compound of Formula (I) free base (e.g. Compound 1, 2 or 3).

TABLE 7

| | 50 mg tablet-1 | | 50 mg tablet-2 | | 50 mg tablet-3 | |
|---|---|---|---|---|---|---|
| | Weight | | Weight | | Weight | |
| Component | (mg/unit) | % (w/w) | (mg/unit) | % (w/w) | (mg/unit) | % (w/w) |
| Extra Granular Component | 53.77 | 95 | 53.77 | 85 | 53.77 | 75 |
| Microcrystalline Cellulose PH102 | — | — | 6.33 | 10 | 14.3 | 20 |
| Croscarmellose Sodium, Compendial | 1.7 | 3 | 1.9 | 3 | 2.15 | 3 |
| Silica Dioxide | 0.57 | 1 | 0.63 | 1 | 0.72 | 1 |
| LIGAMEDMF-2-V Magnesium Stearate | 0.57 | 1 | 0.63 | 1 | 0.72 | 1 |
| Tablet weight | 56.61 | 100 | 63.26 | 100 | 71.66 | 100 |

### Example 8

Formulation of Extra Granular Component with Compounds of Formula (I)

Compound of Formula (I) free base (e.g. Compound 1, 2 or 3), Povidone and Kolliphor® SLS fine and croscarmellose sodium are dry mixed. Water is added to the dry mixture and subjected to a wet granulation at an impellor speed of about 600-610 rpm. The granules are then sifted and dried to afford the extra granular component.

Table 8 show formulation of Extra Granular Component with compounds of Formula (I).

TABLE 8

| | Weight | |
|---|---|---|
| Extra Granular Component | (mg/unit) | % (w/w) |
| Compounds of Formula (I), free base | 50 | 91 |
| Povidone Compendial [plasdoneK29/32] | 3.3 | 6 |
| Kolliphor ® SLS fine | 0.55 | 1 |
| Croscarmellose Sodium, Compendial | 1.1 | 2 |
| Total | 54.95 | 100 |

### Example 9

### Formulation of Compounds of Formula (I)

The extra granular component of Example 8 is combined with the remaining components listed in Table 9. The resultant mixture is then compressed into tablets containing 50 mg of Compound of Formula (I) free base (e.g. Compound 1, 2 or 3).

TABLE 9

| | 50 mg tablet-1 | | 50 mg tablet-2 | | 50 mg tablet-3 | |
|---|---|---|---|---|---|---|
| | Weight | | Weight | | Weight | |
| Component | (mg/unit) | % (w/w) | (mg/unit) | % (w/w) | (mg/unit) | % (w/w) |
| Extra Granular Component | 54.95 | 95 | 54.95 | 85 | 54.95 | 75 |
| Microcrystalline Cellulose-PH102 | — | — | 6.47 | 10 | 14.65 | 20 |
| Croscarmellose Sodium, Compendial | 1.74 | 3 | 1.94 | 3 | 2.20 | 3 |
| Silica Dioxide | 0.58 | 1 | 0.65 | 1 | 0.73 | 1 |
| LIGAMEDMF-2-V Magnesium Stearate | 0.58 | 1 | 0.65 | 1 | 0.73 | 1 |
| Tablet weight | 57.85 | 100 | 64.66 | 100 | 73.26 | 100 |

### Example 10

Formulation of Extra Granular Component with Compounds of Formula (I)

Compound of Formula (I) free base (e.g. Compound 1, 2 or 3), Klucel™ hydroxypropylcellulose, Povidone and Kolliphor® SLS fine and croscarmellose sodium are dry mixed. Water is added to the dry mixture and subjected to a wet granulation at an impellor speed of about 600-610 rpm. The granules are then sifted and dried to afford the extra granular component.

Table 10 show formulation of Extra Granular Component with compounds of Formula (I).

TABLE 10

| | Weight | |
|---|---|---|
| Extra Granular Component | (mg/unit) | % (w/w) |
| Compounds of Formula (I), free base | 50 | 95 |
| Klucel ™ hydroxypropylcellulose | 2.11 | 4 |
| Kolliphor ® SLS fine | 0.55 | 1 |
| Croscarmellose Sodium, Compendial | — | — |
| Total | 52.66 | 100 |

### Example 11

### Formulation of Compounds of Formula (I)

The extra granular component of Example 10 is combined with the remaining components listed in Table 11. The resultant mixture is then compressed into tablets containing 50 mg of Compound of Formula (I) free base (e.g. Compound 1, 2 or 3).

TABLE 11

50 mg tablet

| | Weight | |
|---|---|---|
| Component | (mg/unit) | % (w/w) |
| Extra Granular Component | 52.66 | 75 |
| Microcrystalline Cellulose | 14.04 | 20 |
| PH102 | | |
| Croscarmellose Sodium, | 2.11 | 3 |
| Compendial | | |
| Silica Dioxide | 0.70 | 1 |
| LIGAMEDMF-2-V Magnesium | 0.70 | 1 |
| Stearate | | |
| Tablet weight | 70.21 | 100 |

### Example 12

Formulation of Extra Granular Component with Compounds of Formula (I)

Compound of Formula (I) free base (e.g. Compound 1, 2 or 3), Klucel™ hydroxypropylcellulose, Povidone and Kolliphor® SLS fine and croscarmellose sodium are dry mixed. Water is added to the dry mixture and subjected to a wet granulation at an impellor speed of about 600-610 rpm. The granules are then sifted and dried to afford the extra granular component.

Table 12 show formulation of Extra Granular Component with compounds of Formula (I).

TABLE 12

| | Weight | |
|---|---|---|
| Extra Granular Component | (mg/unit) | % (w/w) |
| Compounds of Formula (I), free base | 50 | 91 |
| Povidone Compendial [plasdoneK29/32] | 3.3 | 6 |
| Kolliphor ® SLS fine | 0.55 | 1 |
| Croscarmellose Sodium, Compendial | 1.1 | 2 |
| Total | 54.95 | 100 |

### Example 13

### Formulation of Compounds of Formula (I)

The extra granular component of Example 12 is combined with the remaining components listed in Table 13. The resultant mixture is then compressed into tablets containing 50 mg of Compound of Formula (I) free base (e.g. Compound 1, 2 or 3).

TABLE 13

| | 50 mg tablet | |
|---|---|---|
| | Weight | |
| Component | (mg/unit) | % (w/w) |
| Extra Granular Component | 54.95 | 85 |
| Microcrystalline Cellulose PH102 | 6.46 | 10 |
| Croscarmellose Sodium, Compendial | 1.94 | 3 |
| Silica Dioxide | 0.64 | 1 |
| LIGAMEDMF-2-V Magnesium Stearate | 0.64 | 1 |
| Tablet weight | 64.63 | 100 |

### Example 14

### Preparation of a 1000 G Batch of a Spray-Dried Dispersion Containing 25% of the Compound of Formula (I) and 75% PVP/VA 64

A 1000 g batch of spray-dried dispersion containing 25% of a compound of Formula (I) (e.g. Compound 1, 2 or 3) and 75% PVP/VA 64 may be prepared as follows. Briefly, acetone (90% (w/w) of the total mixture) is added to a mixing tank followed by the addition of 250 g of a compound of Formula (I) (e.g. Compound 1, 2 or 3) (2.5% (w/w) of the total mixture). The mixture is mixed for 30 minutes in the dark at a temperature range of 15° C. to 27° C. The PVP/VA 64 (750 g, 7.5% (w/w) of the total mixture) is then added and the mixture is stirred for an additional 30 minutes in the dark at a temperature range of 15° C. to 27° C.

The solution is pumped and atomized in a drying chamber. The spray-dried dispersions are prepared in a Pharmaceutical Spray Dryer with 100 kg/hr drying gas capacity (PSD-1). The inlet temperature is set at 75° C. (varied between 60° C.-90° C.). The outlet temperature is set at 35° C. (varied between 32° C.-38° C.). The feed pressure is set at 280 psig (varied between 230-330 psig). The feed rate is set at 110 g/min (varied between 90-130 g/min). The spray dried powder is then dried in a convection tray dryer with a bed depth of ≤2.5 cm at 40° C. (±5° C.) and 15% relative humidity (±10%) for 24 hours under amber light.

### Example 15

### Preparation of Spray-Dried Dispersion Formulations of Compound of Formula (I)

The spray-dried dispersion (SDD) containing 25% a compound of Formula (I) (e.g. Compound 1, 2 or 3) and 75% PVP/VA 64, prepared as described above, is formulated as a suspension or a capsule.

### Suspension Preparation

An amber dosing bottle (30 mL) is tared on a balance. 200 mg SDD (50 mgA) 5% is then weighed into the dosing bottle. Using a 10-mL syringe, 5 mL of water (purified, USP) is added to the dosing bottle and the bottle is capped and shaken moderately for 30 seconds. The SDD suspension is stored in an amber vial at 2-8° C. prior to use, and dosed within 24 hours of preparation.

### Capsule Preparation

An empty hard gelatin capsule (Capsugel, Morristown, NJ), is placed on a balance and the weight was recorded. 200 mg SDD (50 mgA)+5% is then weighed onto weigh paper or an equivalent. All contents are transferred to the capsule using a ProFunnel device for Size 0 capsules. The filled capsule is placed on the balance and the weight is recorded. The weight of the empty capsule is subtracted from the filled weight, ensuring that the weight of the SDD within the capsule is 200 mg SDD+5%, or from 190.0 mg to 210.0 mg. The capsule is securely closed with the head, assuring it clicked into place. The capsules are stored in an amber vial at 2-8° C. prior to use, and dosed within 24 hours of preparation.

### Example 16

### Preparation of High-Dose Capsule Formulations of Compound of Formula (I)

### Capsule Preparation

An empty hard gelatin capsule (Capsugel, Morristown, NJ), is placed on a balance and the weight is recorded. 200 mg of a compound of Formula (I) (e.g. Compound 1, 2 or 3) is then weighed onto weigh paper or an equivalent. All contents are transferred to the capsule using a ProFunnel device for Size 0 capsules. The filled capsule is placed on the balance and the weight is recorded. The weight of the empty capsule is subtracted from the filled weight, ensuring that the weight of the compound of Formula (I) (e.g. Compound 1, 2 or 3) within the capsule was 200 mg±5%, or from 190 mg to 210 mg. The capsule is securely closed with the head, assuring it clicked into place. The capsules are stored in an amber vial at 2-8° C. prior to use, and dosed within 24 hours of preparation.

### Example 17

### CRF Receptor Binding Activity

Compounds of Formula (I) (e.g. Compound 1, 2 and 3) may be evaluated for binding activity to the CRF receptor by a standard radioligand binding assay as generally described by Grigoriadis et al. (see, e.g., Mol. Pharmacol vol 50, pp 679-686, 1996) and Hoare et al. (see, e.g., Mol. Pharmacol 63: 751-765, 2003.) By utilizing radiolabeled CRF ligands, the assay may be used to evaluate the binding activity of the compounds described herein with any CRF receptor subtype.

Briefly, the binding assay involves the displacement of a radiolabeled CRF ligand from the CRF receptor. More specifically, the binding assay is performed in 96-well assay plates using 1-10 μg cell membranes from cells stably transfected with human CRF receptors. Each well receives about 0.05 mL assay buffer (e.g., Dulbecco's phosphate buffered saline, 10 mM magnesium chloride, 2 mM EGTA) containing compound of interest or a reference ligand (for example, sauvagine, urocortin I, or CRF), 0.05 mL of [¹²⁵I] tyrosine-sauvagine (final concentration ˜150 pM or approximately the K_{D} as determined by Scatchard analysis) and 0.1 mL of a cell membrane suspension containing the CRF receptor. The mixture is incubated for 2 hours at 22° C. followed by separation of the bound and free radioligand by rapid filtration over glass fiber filters. Following three washes, the filters are dried and radioactivity (Auger electrons from ¹²⁵I) is counted using a scintillation counter. All radioligand binding data may be analyzed using the non-linear least-squares curve-fitting programs Prism (GraphPad Software Inc) or XLfit (ID Business Solutions Ltd).

### Example 18

### CRF1 Receptor Antagonist Activity

As reported in Fleck et al. (J. Pharmacology and Experimental Therapeutics, 341(2):518-531, 2012) (hereinafter “Fleck et al.” which is incorporated by reference in its entirety) the activity of a CRF₁ receptor antagonists may be expressed as the kinetically derived affinity (Kᵢ) calculated from the association (k₁) and dissociation (k₋₁) rate constants by the following equation:

Kᵢ=k₋₁/k₁

Compounds of Formula (I) (e.g. Compounds 1, 2 and 3) may be evaluated according to these procedures.

### Example 19

Dissociation Half-Life (t_{1/2}) of Representative Compounds

The dissociation half-life (t_{1/2}) of Compounds of Formula (I) (e.g. Compounds 1, 2 and 3) may be evaluated by the technique described in Fleck et al. As described therein, the dissociation rate constant for labeled and unlabeled ligands is denoted as k₋₁, while the half-life of drug dissociation from the receptor (t_{1/2}), which is equal to the median residence time, is calculated from the dissociation rate constant (k₋₁) by the following equation:

t_{1/2}=0.693/k₋₁.

Compounds of Formula (I) (e.g. Compounds 1, 2 and 3) may be evaluated according to these procedures.

### Example 20

Randomized, Double-Blind, Placebo-Controlled Study to Evaluate the Safety and Efficacy of a Compound of Formula (I) in Adult Subjects with Classic Congenital Adrenal Hyperplasia, Followed by Open-Label Treatment

### (I) Objectives

To evaluate the efficacy of Compound 1, 2, or 3, (150 mg twice daily [i.e., BID] based on the free base), compared with placebo, in reducing daily glucocorticoid dosage while maintaining adrenal androgen control.
To evaluate the efficacy of Compound 1, 2, or 3, compared with placebo, in reducing adrenal steroid levels following an initial 4-week treatment period.
To evaluate the effect of Compound 1, 2, or 3 compared with placebo, on clinical endpoints associated with supraphysiologic glucocorticoid dosing.
To evaluate plasma concentrations of Compound 1, 2, or 3 and metabolites.
To assess the safety and tolerability of Compound 1, 2, or 3.
To evaluate an alternate dosing regimen of Compound 1, 2, or 3 in subjects who have not reduced their glucocorticoid dose by Month 12.

### (II) Methodology:

This is a Phase 3, randomized, double-blind, placebo-controlled study to evaluate the efficacy, safety, and tolerability of Compound 1, 2, or 3, versus placebo administered BID with breakfast and the evening meal (doses separated by approximately 12 hours) for 24 weeks in approximately 165 adult subjects with classic CAH due to 21-hydroxylase deficiency. Eligible subjects will be randomly assigned in a 2:1 ratio (active:placebo) to 2 treatment groups: Compound 1, 2, or 3, 150 mg BID or placebo. After the 24-week randomized treatment period, there will be a 6 month, open-label treatment period, during which all subjects will receive Compound 1, 2, or 3 at 150 mg BID. At Month 12, subjects who have not reduced their glucocorticoid dose to ≤11 mg/m²/day will be re-randomized (2:1) to receive 150 mg every morning (qAM) and 250 mg every evening (qPM) or to continue 150 mg BID, in a blinded fashion. Subjects who have reduced their glucocorticoid dose to ≤11 mg/m²/day will continue to receive 150 mg BID in an open label fashion. A final study visit will be conducted approximately 4 weeks after the Month 18 visit.

(A) Screening Period (Weeks −4 Up to Day −1)

All subjects must provide signed and witnessed informed consent prior to the conduct of any study-related procedures. Subjects will undergo screening for up to 4 weeks (Weeks −4 to Day −1) to determine eligibility. There will be a second visit (optional at home) during the screening period to collect a blood sample (for hormone measurements). Subjects must be on a supraphysiologic glucocorticoid regimen defined as >14 mg/m²/day in hydrocortisone dose equivalents adjusted for body surface area (BSA) that has been stable at least 1 month leading up to screening. The glucocorticoid regimen should be optimized by the treating physician to achieve control of adrenal androgen levels and minimization of glucocorticoid dosage to the extent appropriate for the subject's individual medical needs and treatment goals.

Rescreening is permitted if a subject does not meet all eligibility requirements and returns to be rescreened. A subject that has failed screening twice may not be rescreened again without prior permission.

(B) Randomized, Double-Blind, Placebo-Controlled Treatment Period (Day 1 Up to Week 24)

(a) 4-Week Glucocorticoid Stable Period (Day 1 Up to Week 4)

During the first 4 weeks of the study, subjects should maintain their stable glucocorticoid regimen, except for sick-day guidelines (e.g., based on guidance provided by the investigator or their treating physician).

On Day 1 (baseline), subjects will collect a urine sample (all voids from midnight the night before the study visit to the first morning void after awakening for the day) at home in the morning and bring it to the site for measurement of androgen metabolite levels. They will hold their morning glucocorticoid dose and bring it with them to the study site so that a blood sample can be obtained prior to taking the morning glucocorticoid dose; subjects will then take their morning dose of glucocorticoid at the study site, and another blood sample will be taken approximately 2 hours post-dose in order to establish the baseline pre- and post-glucocorticoid hormone levels. Subjects should be fasting from the night before so that fasting blood tests and an oral glucose tolerance test can be performed, but should be encouraged to drink water to avoid any hypovolemic status.

Subjects will be randomized on Day 1 in a 2:1 ratio (active:placebo). Randomization will be stratified by total daily glucocorticoid dose, glucocorticoid type, and sex. Beginning on Day 1 (baseline), the study drug or placebo in the form of one or more capsules will be administered at home with the subject's evening meal; thereafter, the capsule(s) will be administered BID with the subject's breakfast and evening meal (doses separated by approximately 12 hours).

(b) 8-Week Glucocorticoid Reduction Period (Week 4 Up to Week 12)

During this period, subjects will undergo a down-titration (in 4 or fewer steps) of their glucocorticoid dose with the goal to reach a target dose of 8 to 10 mg/m²/day (hydrocortisone equivalents adjusted for body surface area (BSA)) by Week 12, unless the subject has any signs or symptoms suggestive of clinically relevant glucocorticoid insufficiency or unacceptable symptoms of hyperandrogenism.

At the week 4 visit, a similar procedure will be followed as for Day 1 to obtain a more detailed assessment of androgen status, with collection of a urine sample at home and collection of blood samples prior to and approximately 2 hours after dosing of morning glucocorticoid and capsule(s) at the study site. At this visit, the investigator will instruct the subject on the first step of the glucocorticoid dose reduction and arrange to contact the subject by telephone within a week of the study visit to assess how the subject is tolerating the glucocorticoid dose reduction. During the follow-up telephone contact, if the investigator feels that a clinical assessment and/or laboratory tests are needed, these can be performed as an unscheduled visit.

Subjects will have study visits at Weeks 6 (optional at home), 9 (optional at home), and 12 for study assessments, including collection of blood samples to assess hormone levels and routine safety assessments.

At the Week 6 visit, the investigator will instruct the subject on the second step of the glucocorticoid dose reduction and will arrange to contact the subject by telephone within a week of the study visit to assess how the subject is tolerating the glucocorticoid dose reduction. The investigator will contact the subject at approximately Week 8 to advise on the third step of glucocorticoid dose reduction (if applicable).

At the Week 9 study visit, the investigator will assess whether the subject is tolerating the third glucocorticoid dose reduction. The investigator will contact the subject at approximately Week 10 to advise on the fourth step of glucocorticoid dose reduction (if applicable).

If the subject experiences any of the following signs or symptoms at any time during the glucocorticoid dose reduction process, the glucocorticoid dose should NOT be reduced further but returned to the previous dose that was tolerated. However, before the glucocorticoid dose reduction is stopped for symptoms or signs of orthostatic hypotension, volume status should be optimized (e.g., with additional dietary salt, salt tablets, intravenous saline).
Unexplained hyponatremia (serum sodium<135 mmol/L)
Orthostatic hypotension with decrease in systolic blood pressure>20 mmHg or in diastolic blood pressure>10 mmHg after standing (from a seated position) after approximately 2 minutes, or severe symptoms of dizziness or lightheadedness upon standing
Severe nausea, food aversion, vomiting
Unacceptable symptoms of hyperandrogenism (e.g., hirsutism, acne, amenorrhea)

Glucocorticoid dose reductions during Weeks 4 to 12 should proceed even if androstenedione levels increase transiently, provided that the increase is asymptomatic and tolerated by the subject.

At the Week 12 visit, based on review of the subject's hormone levels collected up to that visit as well as based on clinical assessment, the investigator will determine the appropriate dose of glucocorticoid to continue past Week 12 (the reduced dose if tolerated, or a prior [higher] dose) in order to achieve adequate control of androgen levels (i.e., androstenedione≤120% of the subject's baseline or ≤upper limit of normal [ULN] for age and sex).

(c) 12-Week Glucocorticoid Optimization Period (Week 12 Up to Week 24)

Subjects will continue on the glucocorticoid regimen as instructed by the investigator at Week 12 and return to the study site at Week 16 (optional at home), Week 20 (optional at home), and Week 24 during the glucocorticoid optimization period. At these visits, the investigator will review the laboratory results from the preceding study visit and determine if the glucocorticoid regimen requires adjustment in order to achieve adequate control of androgen levels (i.e., androstenedione≤120% of the subject's baseline or ≤ULN for age and sex).

At the Week 24 visit, subjects will follow a similar procedure as Day 1 for additional androgen assessments with collection of a urine sample at home and collection of blood samples prior to and approximately 2 hours after dosing of morning glucocorticoid and study drug at the study site. Subjects should be fasting from the night before, but should be encouraged to drink water to avoid any hypovolemic status, and a glucose tolerance test will be performed (with study drug taken with the glucose load rather than a meal).

(C) Open-Label Treatment Period (Week 24 Up to Month 12)

For the purpose of this study, months are defined as 4 week intervals.

Starting the evening of the Week 24 visit (after all Week 24 assessments have been performed), all subjects will receive capsule(s) comprising study drug (Compound 1, 2, or 3) at 150 mg BID with breakfast and evening meals. Subjects should continue the glucocorticoid regimen specified by the investigator at Week 24. Subjects and investigators will remain blinded to subjects' treatment group assignment from the double-blind period.

(a) 1-Month Glucocorticoid Stable Period (Week 24 Up to Month 7)

During the first month of open-label treatment with Compound 1, 2, or 3, subjects should maintain a stable glucocorticoid regimen (except for sick-day guidelines).

(b) 3-Month Glucocorticoid Reduction Period (Month 7 Up to Month 10)

At Months 7 (optional at home), 8, and 9 (optional at home), investigators will decrease glucocorticoid doses in those subjects whose glucocorticoid dose is still greater than 11 mg/m²/day at Month 7 (unless there is a safety concern with regard to glucocorticoid insufficiency), with the goal to achieve a target physiologic dose of 8 to 10 mg/m²/day by Month 10. The glucocorticoid dose should be reduced by approximately 10% to 20% at each visit (Months 7, 8, and 9), as long as androstenedione levels are within control (i.e., androstenedione≤120% of the subject's baseline or ≤ULN for age and sex) and the subject is not experiencing any signs or symptoms suggestive of clinically relevant glucocorticoid insufficiency or unacceptable symptoms of hyperandrogenism. The glucocorticoid dose reduction will not require dose reduction below 8 mg/m²/day hydrocortisone equivalents. After each of the glucocorticoid dose reduction steps, the site should contact the subject by telephone (within a week) to assess how the subject is tolerating the glucocorticoid dose reduction. Subjects will have study visits at Months 8, 9, and 10 for study assessments including collection of blood samples for hormone levels.

(c) 2-Month Glucocorticoid Maintenance Period (Month 10 Up to Month 12)

Subjects will return to the study site at Months 10 and 12 for study assessments as outlined in the Schedule of Assessments. During this period, the goal should be to maintain stable glucocorticoid doses; however, the dose can be adjusted according to standard of care (e.g., to achieve the control of androgen levels appropriate to the treatment targets for each subject).

At the Month 12 visit, subjects will have additional androgen assessments with collection of a urine sample at home and blood sample collection before and approximately 2 hours after dosing of morning glucocorticoid and study drug at the study site. Subjects should be fasting from the night before (subjects should be encouraged to drink water to avoid any hypovolemic status). A glucose tolerance test will be performed (with capsule(s) taken with the glucose load rather than a meal) at the Month 12 visit.

(D) Open-Label or Double-Blind Active-Controlled Treatment (Month 12 to Month 18)

(a) 6-Month Glucocorticoid Maintenance Period (Month 12 to Month 18) for Subjects with Month 12 Glucocorticoid Dose 11 mg/m²/Day

Subjects with glucocorticoid dose 11 mg/m²/day at Month 12 will continue on active study drug at 150 mg BID until Month 18 with study visits at Months 14, 16, and 18. The goal during this period is to maintain stable glucocorticoid doses while androstenedione levels are within control (i.e., androstenedione≤120% of the subject's baseline or ≤ULN for age and sex), although the dose can be adjusted according to standard of care.

At the Month 18 visit, subjects will have additional androgen assessments with collection of a urine sample at home and blood sample collection before and approximately 2 hours after dosing of morning glucocorticoid and capsule(s) administration at the study site. Subjects should be fasting from the night before (subjects should be encouraged to drink water to avoid any hypovolemic status).

(E) Follow-Up Period (Month 19)

A final post-treatment visit will be conducted at Month 19, 1 month after subjects' final dose of capsule(s).

(F) Study Assessments and Study Visit Scheduling

Efficacy, safety, and PK will be assessed at scheduled times throughout the study. As much as possible, all study visits (including baseline and follow-up) should occur at approximately the same time in the morning to standardize time of day for assessment of efficacy, safety, and drug exposure.

In the double-blind, placebo-controlled portion of the study, all visits during the glucocorticoid stable period and glucocorticoid reduction period have a visit window of +5 days, and all visits during the glucocorticoid optimization period have a visit window of ±5 days. In the open-label treatment period, visits from Month 7 to Month 10 have a visit window of ±5 days and visits from Month 12 to Month 19 will have a visit window of±7 days. If a subject's glucocorticoid regimen is adjusted due to sick-day guidelines, the subject should resume their glucocorticoid dosing regimen for at least 3 days before their next scheduled hormone panel assessment, and this 3-day window supersedes all other visit windows. An independent Data and Safety Monitoring Board (DSMB) will periodically review ongoing clinical safety data to ensure the safety and well-being of study subjects.

### (III) Study Population

Approximately 165 female and male subjects, at least 18 years of age, with a documented medical diagnosis of classic CAH due to 21-hydroxylase deficiency will be enrolled into this study.

To participate in this study, subjects must meet the following criteria:
1. Subjects must provide written informed consent.
2. Be a female or male at least 18 years of age.
3. Have a medically confirmed diagnosis of classic 21-hydroxylase deficiency CAH based on standard medically accepted criteria such as elevated 17-OHP level, confirmed CYP21A2 genotype, positive newborn screening with confirmatory second-tier testing, or cosyntropin stimulation.
4. Be on a stable, supraphysiologic glucocorticoid dose regimen (defined as >14 mg/m²/day in hydrocortisone dose equivalents) that has been stable for at least 1 month prior to screening, is intended for chronic use, and consists of 1 or more of the following glucocorticoids: hydrocortisone (except sustained release), prednisone, prednisolone, methylprednisolone, or dexamethasone. Subjects who are on dexamethasone alone must be receiving≥0.5 mg/day.
5. If treated with fludrocortisone, dose should be stable for at least 1 month prior to screening with an upright plasma renin activity (PRA) during screening within the normal range on the subject's usual sodium intake. If PRA is not within the normal range, the subject must have systolic blood pressure>100 mmHg, without orthostatic hypotension, and with serum sodium and potassium in the normal range.
6. Female subjects of childbearing potential must agree to use contraception consistently from screening until the final study visit or 30 days after the last dose of study drug, whichever is longer. A female who is not of childbearing potential must meet 1 of the following:
   Postmenopausal, defined as no menses for 12 months without an alternative medical cause and confirmed by elevated follicle-stimulating hormone (FSH) consistent with a postmenopausal range
   Permanent sterilization procedure, such as hysterectomy, bilateral salpingectomy, or bilateral oophorectomy
7. Male subjects must agree to use contraception consistently from screening until 90 days after the last dose of study drug. The acceptable method of contraception for male subjects is condom with spermicide (cream, spray, foam, gel, suppository, or polymer film).

### (IV) Investigational Product, Dosage and Mode of Administration:

Compound 1, 2, or 3 will be administered at 150 mg BID (300 mg total daily dose), based on the free base, in oral capsule or tablet form with subjects' breakfast and evening meal (doses separated by approximately 12 hours). The dose may be adjusted to 150 mg qAM and 250 mg qPM at Month 12. Each administration will comprise three or more capsules or tablets containing 50 mg of Compound 1, 2, or 3.

Subjects will take the capsule(s) or tablets(s) by mouth beginning with the evening meal on Day 1, and then with breakfast and the evening meal (doses separated by approximately 12 hours) for the remainder of the treatment period. Each meal should be completed within 30 minutes of taking the capsule(s) or tablets(s).

If a subject forgets or is unable to take the capsule(s) or tablets(s), the subject should take his or her dose of study drug as soon as possible, as long as the subject's next dose will be at least 8 hours later. The subject will need to skip the dose if he or she is unable to take the study drug at least 8 hours prior to the next dose.

### (V) Criteria for Evaluation:

(A) Efficacy:

Daily glucocorticoid regimen expressed in hydrocortisone equivalents adjusted for body surface area (BSA) (mg/m²/day).

Hormone measurements: 17-hydroxyprogesterone (17-OHP) (serum; ng/dL), androstenedione (serum; ng/dL), testosterone (serum; ng/dL), adrenocorticotropic hormone (ACTH) (plasma; pg/mL), cortisol (serum; g/dL), luteinizing hormone (LH) (serum; IU/L), follicle stimulating hormone (FSH; IU/L), progesterone (serum; ng/mL), plasma renin activity (measured upright) (ng/mL/hr).

Urine androgen metabolite levels (androsterone and etiocholanolone).

Metabolic assessments (fasting lipid panel, homeostatic model assessment of insulin resistance [HOMA-IR] based on fasting glucose and insulin levels, glycated hemoglobin [HbA1c], glucose tolerance test).

Dual-energy X-ray absorptiometry (DXA) scan (bone mineral density and body composition). Blood pressure.

Hirsutism and Acne Scales (female subjects only).

Testicular ultrasounds (to detect adrenal rest tissue) (male subjects only).

Menstrual Cycle Questionnaire (only in female subjects of childbearing potential who are not on hormonal or intrauterine device contraceptives).

Bone markers: serum osteocalcin, serum bone-specific alkaline phosphatase, serum C-terminal telopeptide, urine N-terminal telopeptide.

(B) Patient-Reported Outcomes:

36-Item Short Form Health Survey (SF-36), EuroQol 5 Dimensions 5 Levels (EQ-5D-5L), Multidimensional Assessment of Fatigue (MAF), Psychological General Well-Being Index (PGWBI), and Medical Outcomes Study 12-Item Sleep Scale (MOS-12).

(C) Pharmacokinetics:

Blood samples to evaluate plasma concentrations of Compound 1, 2, or 3 and metabolites will be collected throughout the study.

(D) Safety:

Safety and tolerability will be monitored throughout the study and will include the following assessments:
Adverse events (including glucocorticoid-related events)
Clinical laboratory tests
Vital signs
Weight/body mass index (BMI), and waist circumference
Physical examinations
12-lead electrocardiograms
Brief Psychiatric Rating Scale (BPRS)
Columbia-Suicide Severity Rating Scale (C-SSRS)

### (VI) Study Endpoints:

The primary endpoint is the percent change from baseline in glucocorticoid daily dose (in hydrocortisone equivalents adjusted for BSA [mg/m²/day]) at Week 24, while Week 24 androstenedione is adequately controlled at ≤120% of baseline or ≤upper limit of normal for age and sex. The primary analysis of the primary endpoint will be performed using an analysis of covariance (ANCOVA) model.

The first key secondary endpoint is the change from baseline in serum androstenedione at Week 4, which will be analyzed using an ANCOVA model.

The second key secondary endpoint is the achievement of a reduction in glucocorticoid daily dose to physiologic levels (≤11 mg/m²/day in hydrocortisone equivalent adjusted for BSA) at Week 24 while maintaining androstenedione levels (as defined above in the primary endpoint), which will be analyzed using a Cochran-Mantel-Haenszel (CMH) test.

Additional key secondary endpoints are the changes from baseline in HOMA-IR, weight, and fat mass at Week 24, which will be analyzed using an ANCOVA model.

### Example 21

A Randomized, Double-Blind, Placebo-Controlled Study to Evaluate the Safety and Efficacy of a Compound of Formula (I) in Pediatric Subjects with Classic Congenital Adrenal Hyperplasia, Followed by Open-Label Treatment

### (I) Objectives

To evaluate the efficacy of Compound 1, 2, or 3 compared with placebo, in reducing adrenal androgen and precursor levels during a glucocorticoid-stable period.
To evaluate the efficacy of Compound 1, 2, or 3 compared with placebo, in reducing daily glucocorticoid dosage while maintaining adrenal androgen control.
To evaluate plasma concentrations of Compound 1, 2, or 3 and metabolites.
To assess the safety and tolerability of Compound 1, 2, or 3.

### (II) Methodology

This is a Phase 3, randomized, double-blind, placebo-controlled study to evaluate the efficacy, safety, and tolerability of Compound 1, 2, or 3 versus placebo administered twice daily (BID) with breakfast and evening meals for 28 weeks in approximately 81 pediatric subjects with classic CAH due to 21-hydroxylase deficiency. Eligible subjects will be randomly assigned in a 2:1 ratio (active:placebo) to either Compound 1, 2, or 3 (50 mg QD oral liquid for subjects 10 to <20 kg, 50 mg BID oral liquid for subjects 20 to <55 kg, or 100 mg BID oral capsule(s) or tablet(s) for subjects≥55 kg) or matching placebo (oral liquid placebo for subjects<55 kg and oral capsule(s) or tablet(s) placebo for subjects≥55 kg). After the 28-week placebo-controlled treatment period, there will be a 24-week, open-label treatment period, during which all subjects will receive Compound 1, 2, or 3 at the same doses as administered during the placebo-controlled treatment period. A final study visit will be conducted approximately 4 weeks after the Week 52 visit.

(A) Screening Period (Weeks −4 Up to Day −1)

Parental or legal guardian informed consent with signed and witnessed study subject assent (as required by the governing institutional review board or ethics committee and according to local laws and regulations) will be obtained prior to any study-related procedures. Subjects will undergo screening for up to 4 weeks (Weeks −4 to Day −1) to determine eligibility. Rescreening is permitted if a subject does not meet all eligibility requirements and returns to be rescreened. A subject that has failed screening twice may not be rescreened again without prior permission.

(B) Randomized, Double-Blind, Placebo-Controlled Treatment Period (Day 1 Up to Week 28)

(a) Glucocorticoid-Stable Period

One Day 1, subjects≥12 years of age should be fasting after midnight the night before until the first blood sample is collected at the site, after which they will be provided breakfast. They should be encouraged to drink water during the fasting period to avoid any hypovolemic status. Subjects<12 years of age do not need to fast.

On Day 1 (baseline), subjects≥6 years of age and >20 kg body weight will hold their morning glucocorticoid dose and bring it with them to the study site, arriving to the site by approximately 0800 hours. Blood samples will be obtained serially over approximately 3.5 hours (at 0830, 0900, 1000, 1100, and 1200 hours), with the morning glucocorticoid dose administered after the 0900 hour sample is collected. Subjects<6 years of age or <20 kg body weight will take their morning glucocorticoid dose at home and have a single blood sample collected at the site, timed to be approximately 2 hours after the morning glucocorticoid dose. Salivary samples for adrenal androgens and precursors will also be collected.

Subjects will be randomized on Day 1 in a 2:1 ratio (active:placebo). Randomization will be stratified by pubertal stage (Tanner stage 1 or 2 vs. 3, 4 or 5) and sex within each dose group. Beginning on Day 1 (baseline), the oral liquid or capsule(s) will be administered at home with the subject's evening meal; thereafter, the oral liquid or capsule(s) will be administered BID with the subject's breakfast and evening meals (doses separated by approximately 12 hours).

From Day 1 until Week 4, subjects should maintain a stable glucocorticoid regimen to the extent possible, except for sick-day guidelines. Sick-day dosing may follow alternate guidelines or can be based on guidance provided by the investigator or the subject's treating physician.

(b) Glucocorticoid Adjustment Period

At the Week 4 visit, subjects≥6 years of age and >20 kg body weight will hold their morning glucocorticoid and oral liquid or capsule(s) and bring it with them to the study site, arriving to the site by approximately 0800 hours. Blood samples will be obtained serially over approximately 6.5 hours (at 0830, 0900, 1000, 1100, 1200, 1300, and 1500 hours). The morning glucocorticoid dose and oral liquid or capsule(s) will be administered after the 0900 hours sample is collected. Subjects<6 years of age or <20 kg body weight will take their morning glucocorticoid dose at home (at approximately the same time as on Day 1) but hold their morning oral liquid or capsule(s) and have a single blood sample collected at the site, timed to be approximately 2 hours after the morning glucocorticoid dose.

Salivary samples for adrenal androgens and precursors will also be collected.

From Week 4 until Week 28, the subject's glucocorticoid dose should be adjusted according to their androstenedione levels, with the goal to reach a dose of approximately 8 to 10 mg/m²/day at Week 28, if androstenedione can be maintained≤baseline levels. Glucocorticoid dose adjustments can occur in as few as 1 or up to 4 steps, depending on the starting and target glucocorticoid doses and the amount of dose adjustment at each step. The target glucocorticoid dose should be within the range of 8 to 10 mg/m²/day to the extent possible, but could be lower than this range depending on practical issues considered in clinical practice related to available dosage strengths. Before any glucocorticoid dose reduction is implemented, the investigator will evaluate the subject for any symptoms suggestive of glucocorticoid insufficiency using a standardized checklist and will arrange for follow-up if needed after the dose reduction.

The first glucocorticoid dose adjustment step should be guided by the change in androstenedione (A4) at Week 4 from baseline. A suggested guideline is provided in the table below, but the exact amount adjusted may differ from this guideline based on practical issues considered in clinical practice related to available dosage strengths. The investigator should contact the subject once the Week 4 lab results are available in order to provide guidance on the amount of the first glucocorticoid dose adjustment.

TABLE 14

| Percent Change from Baseline | GC Dose Adjustment Step #1 |
|---|---|
| in Androstenedione at Week 4 | (approximately Week 6) |
| No change or increase | Consider whether GC dose needs to |
| | be increased |
| Decrease of >0 to ≤20% | 1 to 2 mg/m²/day GC dose decrease |
| Decrease of >20% to ≤40% | 2 to 3 mg/m²/day GC dose decrease |
| Decrease of >40% | 3 to 4 mg/m²/day GC dose decrease |

A follow-up blood test should be arranged approximately 2 weeks later at Week 8 (at home or the study site).

For all blood tests after the Week 4 visit, subjects should take their morning glucocorticoid dose at home with blood sample collection timed approximately 2 hours after the glucocorticoid dose.

If needed, subsequent glucocorticoid dose adjustment steps should occur when lab results are available (at approximately Week 10, Week 14, and Week 18) with follow-up blood tests at Week 12 (at home or the study site, and only if the glucocorticoid dose is modified at Week 10), Week 16 (at the study site), and Week 20 (at home or the study site).

The target amount of glucocorticoid dose reduction at each step is approximately 1 to 4 mg/m²/day but should be guided by the androstenedione level at the preceding blood test as well as on practical issues considered in clinical practice related to available dosage strengths.

TABLE 15

| Blood Test | Glucocorticoid Dose Adjustment Step |
|---|---|
| Week 8 (at home | Step 2 (if needed) at approximately |
| or the study site) | Week 10 (or when Week 8 labs available) |
| Week 12 (if Step 2 | Step 3 (if needed) at approximately |
| needed, at home or | Week 14 (or when Week 12 labs available) |
| the study site) | |
| Week 16 (at the | Step 4 (if needed) at approximately |
| study site) | Week 18 (or when Week 16 labs available) |
| Week 20 (at home | If androstenedione not ≤ baseline, further |
| or the study site) | glucocorticoid dose adjustment may be needed |

Subjects will return to the study site at Week 16 and Week 28 for assessments as outlined in the Schedule of Assessments. Prior to the Week 16 and Week 28 visits, subjects will hold their morning oral liquid or capsule(s) and bring it with them to the study site, but will take their morning glucocorticoid dose at home, with blood sample collection timed to be approximately 2 hours later.

For the Week 28 visit, subjects≥12 years of age should be fasting after midnight the night before until the first blood sample is collected at the site, after which they will be provided breakfast. Subjects should be encouraged to drink water during the fasting period to avoid hypovolemic status. Subjects<12 years of age do not need to fast.

(C) Open-Label Treatment Period (Week 28 to Week 52)

Starting the evening of the Week 28 visit (after all Week 28 assessments have been performed), all subjects will receive Compound 1, 2, or 3 (Compound 1, 2, or 3; 50 mg QD oral liquid for subjects 10 to <20 kg, 50 mg BID oral liquid for subjects 20 to <55 kg, or 100 mg BID oral capsule(s) or tablet(s) for subjects≥55 kg) with breakfast and evening meals. Subjects and investigators will remain blinded to subjects' treatment group assignment during the placebo-controlled treatment period.

For subjects who are still on greater than 10 mg/m²/day glucocorticoid dose at Week 28, further adjustments in glucocorticoid dose should be made following the guidelines used during the placebo-controlled period, and a blood sample will be collected at Week 32 (at home or the study site).

The first glucocorticoid dose adjustment step (if done) should be guided by the androstenedione change at Week 32 (compared with Week 28), after all subjects have been on open-label active study drug for 4 weeks. A suggested guideline is provided below but the exact amount adjusted may differ from this guideline based on practical issues considered in clinical practice related to available dosage strengths. The investigator should contact the subject once the Week 32 lab results are available in order to provide guidance on the amount of the first glucocorticoid dose adjustment (if needed) during the open-label period.

TABLE 16

| Percent Change from Week 28 | GC Dose Adjustment Step #1 |
|---|---|
| in Androstenedione at Week 32 | (approximately Week 34) |
| No change or increase | Consider whether GC dose needs to |
| | be increased) |
| Decrease of >0 to ≤20% | 1 to 2 mg/m²/day GC dose reduction |
| Decrease of >20% to ≤40% | 2 to 3 mg/m²/day GC dose reduction |
| Decrease of >40% | 3 to 4 mg/m²/day GC dose reduction |

If the glucocorticoid dose is modified at approximately Week 34, a follow-up blood test should be arranged approximately 2 weeks later at Week 36 (at home or the study site).

If needed, subsequent glucocorticoid dose adjustments should occur at approximately Week 38 and Week 42 (or when lab results are available) with follow-up blood tests at Week 40 (at the study site) and Week 44 (at home or the study site, and only if the glucocorticoid dose is modified at Week 42). The target amount of glucocorticoid dose reduction at each step is approximately 1 to 4 mg/m²/day but should be guided by the androstenedione level at the preceding blood test as well as practical issues considered in clinical practice related to available dosage strengths.

TABLE 17

| Blood Test | GC dose adjustment step |
|---|---|
| Week 36 (at home or at | Step 2 (if needed) at approximatel |
| the study site) | Week 38 (or when Week 36 A4 result |
| | is available) |
| Week 40 (at the study | Step 3 (if needed) at approximately |
| site) | Week 42 (or when Week 40 A4 result |
| | is available) |
| Week 44 (if Step 3 needed, | If A4 not < baseline, further GC |
| at home or the study site) | dose adjustment may be needed |

Subjects will return to the study site at Week 40 and Week 52 for assessments as outlined in the Schedule of assessments. Prior to the Week 40 and Week 52 visits, subjects will hold their morning oral liquid or capsule(s) and bring it with them to the study site, but will take their morning glucocorticoid dose at home, with blood sample collection timed to be approximately 2 hours later.

For the Week 52 visit, subjects≥12 years of age should be fasting after midnight the night before until the first blood sample is collected at the site, after which they will be provided breakfast. Subjects should be encouraged to drink water during the fasting period to avoid any hypovolemic status. Subjects<12 years of age do not need to fast.

(D) Study Assessments and Study Visit Scheduling

Efficacy, safety, and PK will be assessed at scheduled times throughout the study. As much as possible, all study visits (including baseline, during the study, and follow-up) should occur at approximately the same time in the morning to standardize time of day for assessment of efficacy, safety, and drug exposure.

The Week 4 visit will have a visit window of 5 days, and subsequent visits will have a visit window of 7 days. If a subject's glucocorticoid regimen is adjusted due to sick-day guidelines, the subject should resume their glucocorticoid dosing regimen for at least 3 days before their next scheduled lab test, and this 3-day window supersedes all other visit windows.

An independent Data Monitoring Committee will periodically review unblinded study data to ensure the safety and well-being of study subjects and to confirm observed exposures are consistent with expected target exposures.

### (III) Study Population

Approximately 81 female and male subjects, 2 to 17 years of age, with a documented medical diagnosis of classic CAH due to 21-hydroxylase deficiency will be enrolled into this study.

To participate in this study, subjects must meet the following criteria:
1. Have documentation of witnessed written or oral pediatric assent from the subject deemed capable of providing assent, and written informed consent from the subject's parent(s) or legal guardian in accordance with the governing institutional review board or ethics committee and according to local laws and regulations.
2. Be a female or male at least 2 years of age and less than 18 years of age and a body weight of at least 10 kg.
3. Have a medically confirmed diagnosis of classic 21-hydroxylase deficiency CAH based on standard medically accepted criteria such as elevated 17-OHP level, confirmed CYP21A2 genotype, positive newborn screening with confirmatory second-tier testing, or cosyntropin stimulation.
4. Be on a supraphysiologic glucocorticoid dose regimen (defined as >12 mg/m²/day in hydrocortisone dose equivalents) that has been above this threshold for at least 6 months and at a stable dose for at least 1 month prior to screening, is intended for chronic use, and consists of 1 or more of the following glucocorticoids: hydrocortisone (except sustained release), prednisone, prednisolone, methylprednisolone, or dexamethasone. Subjects must be on a morning dose of glucocorticoid.
5. Have an androstenedione level (prior to the morning glucocorticoid dose) greater than upper limit of normal (according to age, sex, and/or pubertal stage).
6. Have a 17-hydroxyprogesterone level (prior to the morning glucocorticoid dose) greater than 800 ng/dL.
7. If treated with fludrocortisone, dose should be stable for at least 1 month prior to screening with an upright plasma renin activity (PRA) during screening within the normal range on the subject's usual sodium intake. If PRA is not within the normal range, the subject must have systolic blood pressure>100 mmHg, without orthostatic hypotension, and with serum sodium and potassium in the normal range.
8. Female subjects of childbearing potential who are sexually active must agree to use contraception consistently from screening until the final study visit or 30 days after the last dose of study drug, whichever is longer. A female subject of childbearing potential is defined as a female capable of becoming pregnant, which includes subjects who have had their first menstrual cycle (i.e., menarche) and are not surgically sterile (i.e., bilateral oophorectomy, hysterectomy or bilateral tubal ligation for at least 3 months prior to screening). A male subject of childbearing potential is defined as a subject who has reached spermarche and has not been vasectomized for at least 3 months prior to screening. Male subjects of childbearing potential who are sexually active must agree to use effective barrier contraception consistently from screening until 90 days after the last dose of study drug. The acceptable method of contraception for male subjects is condom with spermicide (cream, spray, foam, gel, suppository, or polymer film).

### (IV) Investigational Product, Dosage and Mode of Administration

Compound 1, 2, or 3 (50 mg QD oral liquid for subjects 10 to <20 kg, 50 mg BID oral liquid for subjects 20 to <55 kg, or 100 mg BID oral capsule(s) or tablet(s) for subjects≥55 kg) will be administered with subjects' breakfast and evening meals (doses separated by approximately 12 hours) for BID or administered with subjects' evening meal for QD. Each oral capsule or tablet contains 50 mg Compound 1, 2, or 3 (free base). The oral liquid contains 50 mg of Compound 1, 2, or 3 (free base) per 1 mL and will be administered via a calibrated oral dosing syringe.

(V) Criteria for evaluation

(A) Efficacy
Hormone measurements: Androstenedione (A4; serum and saliva), 17-hydroxyprogesterone (17-OHP; serum and saliva), adrenocorticotropic hormone (ACTH; plasma), luteinizing hormone (LH; serum), testosterone (serum), plasma renin activity (measured upright).
Daily glucocorticoid regimen expressed in hydrocortisone equivalents adjusted for body surface area (BSA) (mg/m²/day).
Body weight and body mass index.
Growth (assessed as height velocity).
Bone age based on X-ray (only for subjects not at adult height and not with fused phalangeal epiphyses on X-ray).
Metabolic assessments (only in subjects≥12 years of age; fasting lipid panel and homeostatic model assessment of insulin resistance [HOMA-IR] based on fasting glucose and insulin levels).
Menstrual cycle questionnaire (only in female subjects who have undergone menarche and are not on hormonal or intrauterine device contraceptives).
Hirsutism (only for female subjects) and acne scales.
Testicular ultrasounds (to detect adrenal rest tissue; only in male subjects).

(B) Patient and Caregiver Reported Outcomes
EuroQol (European Quality of Life)-5 Dimensions-Youth (EQ-5D-Y)
Pediatric Quality of Life Instrument (Peds-QL)
Peds-QL Family Impact

(C) Pharmacokinetics
Blood samples to evaluate plasma concentrations of Compound 1, 2, or 3 and metabolites will be collected throughout the study.

(D) Other
Palatability assessment

(E) Safety
Adverse events (including glucocorticoid-related events)
Clinical laboratory tests (chemistry, hematology, coagulation, urinalysis)
Vital signs
Physical examinations, including height, weight, and Tanner stage
6- or 12-lead electrocardiograms
Brief Psychiatric Rating Scale, Children's Version (BPRS-C)
Columbia-Suicide Severity Rating Scale (C-SSRS) Children's Version (only for subjects≥6 years of age)

### (VI) Study Endpoints and Statistical Analysis

The primary endpoint is the change from baseline to Week 4 in serum androstenedione (average across all values obtained from 0830 to 1200 hours). The first key secondary endpoint is the change from baseline to Week 4 in serum 17-OHP (average across all values obtained from 0830 to 1200 hours). The second key secondary endpoint is the percent change from baseline to Week 28 in glucocorticoid daily dose (in hydrocortisone equivalents adjusted for BSA [mg/m²/day]), while Week 28 androstenedione is less than or equal to the baseline value.

One secondary endpoint is the achievement of a reduction in glucocorticoid daily dose to physiologic levels (≤11 mg/m²/day in hydrocortisone equivalent adjusted for BSA) at Week 28, while Week 28 androstenedione is less than or equal to the baseline value. Additional secondary endpoints include change from baseline in body mass index standard deviation score (SDS) at Week 28 and change in height velocity at Week 28 (restricted to subset of subjects not at adult height).

The continuous endpoints will be analyzed using an analysis of covariance (ANCOVA) model and will include treatment group (Compound 1, 2, or 3 v. placebo), stratification factors used in the randomization and, as appropriate, baseline value. The binary endpoint will be compared by treatment group (Compound 1, 2, or 3 vs. placebo) using a Cochran-Mantel-Haenszel (CMH) test stratified by factors used in the randomization. The overall Type I error of 0.05 will be controlled by testing the primary, first key secondary, and second key secondary endpoints sequentially in this order.

### Example 22

### A Phase 1 Study to Evaluate Tolerability and Pharmacokinetics of Compound 1, 2, or 3 in Healthy Subjects

### (I) Objectives

To assess the safety and tolerability of Compound 1, 2, or 3 in healthy subjects at total daily doses of 250 mg or 300 mg.
To assess the pharmacokinetics (PK) of Compound 1, 2, or 3 and metabolites in healthy subjects at total daily doses of 250 mg or 300 mg.

### (II) Methodology

This will be a Phase 1, multiple-dose, randomized, double-blind, placebo-controlled study designed to assess the safety, tolerability, and PK of Compound 1, 2, or 3 at 2 dose levels in healthy subjects. Approximately 30 subjects (male or female) will be enrolled and randomized 1:1:1 to Compound 1, 2, or 3 250 mg, Compound 1, 2, or 3 300 mg, or placebo taken as described in the table below. Randomization will occur on Day 1, and each subject will receive a 28-day regimen of Compound 1, 2, or 3 or placebo in a blinded fashion. Doses will be administered with breakfast and evening meals, approximately 12 hours apart.

TABLE 18
Dosing Regimen for Each Treatment Group:

| Treatment | Type/Timing of Dose | |
|---|---|---|
| Group | Morning (AM) Dose | Evening (PM) Dose |
| A | Compound 1, 2, or 3 100 mg | Compound 1, 2, or 3 150 mg |
| (N = 10) | (2 × 50 mg capsules | (3 × 50 mg capsules or |
| | or tablets + 1 × placebo | tablets) |
| | capsule or tablet) | |
| B | Compound 1, 2, or 3 150 mg | Compound 1, 2, or 3 150 mg |
| (N = 10) | (3 × 50 mg capsules or | (3 × 50 mg capsules or |
| | tablets) | tablets) |
| C | Placebo | Placebo |
| (N = 10) | (3 × placebo capsules or | (3 × placebo capsules or |
| | tablets) | tablets) |

Subjects will be screened for eligibility within 42 days prior to the initiation of dosing on Day 1. Subjects will be admitted to the study site no later than Day −1 at the time indicated by the study site. Subjects will be confined to the study center for the duration of dosing.

Confinement will end after collection of the PM blood sample and completion of scheduled study procedures on Day 28. Follow-up visits will be conducted at Weeks 5, 6, and 8 (i.e., days 35, 42, and 56) during a wash-out period after the final dose.

Blood samples for PK analysis of Compound 1, 2, or 3 and metabolites will be collected within 30 minutes prior to the first dose on Day 1, predose for both AM and PM doses daily on Days 7 through 14 (inclusive), Day 21, Day 28, and at follow-up visits at Weeks 5, 6, and 8. Additionally, blood samples for PK analysis of Compound 1, 2, or 3 and metabolites will be collected at approximately 0.5, 1, 2, 3, 4, 5, 6, 7, 8, 9, 10, and 12 hours following both the AM and PM doses on Day 1 and Day 14 (i.e., the final sample will be collected prior to the AM dose on the following day, Day 2 or Day 15, respectively).

Safety and tolerability assessments including adverse events (AEs), clinical laboratory tests (including chemistry, hematology, coagulation, and urinalysis), morning cortisol levels, body weight, vital signs measurements, physical examinations, and 12-lead electrocardiogram (ECG) will be monitored during the study.

### (III) Study Population

A total of approximately 30 healthy subjects (male or female), 18 to 55 years old inclusive, will be enrolled.

### (IV) Investigational Product, Dosage and Mode of Administration:

Compound 1, 2, or 3 will be supplied as capsules or tablets containing 50 mg of free base for oral administration, with breakfast and evening meals (approximately 12 hours apart).

### (V) Criteria for Evaluation:

(A) Pharmacokinetics:

Blood samples for plasma concentrations of Compound 1, 2, or 3 and metabolites will be collected predose on Day 1 (within 15 minutes prior to first dose), on Days 7, 8, 9, 10, 11, 12, 13, 14, 21, and 28 (within 15 minutes prior to each AM and PM dose), and at follow up visits at Weeks 5, 6, and 8 (Days 35, 42, and 56).

Blood samples to determine plasma concentrations for the PK profile of Compound 1, 2, or 3 and metabolites will be collected approximately 0.5, 1, 2, 3, 4, 5, 6, 7, 8, 9, 10, and 12 hours following both the AM and PM doses on Day 1 and Day 14. The following plasma PK parameters will be calculated for Compound 1, 2, or 3 and metabolites:
Area under the plasma concentration versus time curve (AUC) over the dosing interval (AUC₀₋ₜₐᵤ)
Area under the plasma concentration versus time curve from 0 to 24 hours (AUC₀₋₂₄)
Maximum plasma concentration (Cₘₐₓ)
Time to maximum plasma concentration (tₘₐₓ)
Molar AUC ratio of metabolites to the parent drug Compound 1, 2, or 3
Apparent systemic clearance after oral administration clearance (CL/F) (Compound 1, 2, or 3 only)
Accumulation ratio (R_{ac})

(B) Safety
AEs
Clinical laboratory tests (chemistry, hematology, coagulation, and urinalysis)
Morning cortisol levels
Vital signs (including orthostatic blood pressure and pulse) and body weight
Physical examinations
12-lead ECGs

### OTHER EMBODIMENTS

It is to be understood that the foregoing description is intended to illustrate and not limit the scope of the disclosure, which is defined by the scope of the appended claims. Other aspects, advantages, and modifications are within the scope of the following claims.

We claim the following:

1. A compound of Formula (I): 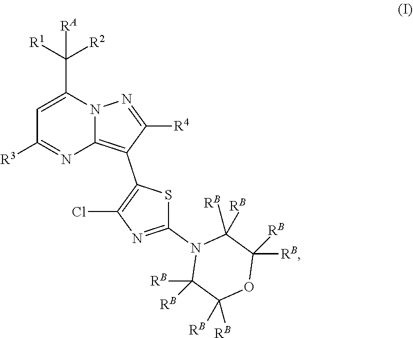
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
for use in a method of treating congenital adrenal hyperplasia in a subject,
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

2. The compound of claim 1, wherein the compound of Formula (I), or a pharmaceutically acceptable salt thereof, is administered at a frequency of twice daily.

3. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:100.

4. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:50.

5. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:10.

6. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:5.

7. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:3.

8. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.5.

9. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.

10. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:1.5.

11. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.

12. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.5.

13. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration about 1:3.

14. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:3.5.

15. The compound of claim 2, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:4.

16. The compound of any one of claims 1 to 15, wherein the amount of the compound of Formula (I), or a pharmaceutically acceptable salt thereof, administered daily is less than or equal to about 1000 mg based on the weight of the free base.

17. The compound of any one of claims 1 to 15, wherein the amount of the compound of Formula (I), or a pharmaceutically acceptable salt thereof, administered daily is from about 50 mg to about 1000 mg based on the weight of the free base.

18. The compound of any one of claims 1 to 15, wherein the amount of the compound of Formula (I), or a pharmaceutically acceptable salt thereof, administered daily is from about 100 mg to about 1000 mg based on the weight of the free base.

19. The compound of any one of claims 1 to 15, wherein the amount of the compound of Formula (I), or a pharmaceutically acceptable salt thereof, administered daily is from about 100 mg to about 500 mg based on the weight of the free base.

20. The compound of any one of claims 1 to 15, wherein the amount of the compound of Formula (I), or a pharmaceutically acceptable salt thereof, administered daily is from about 200 mg to about 500 mg based on the weight of the free base.

21. The compound of any one of claims 1 to 15, wherein the amount of the compound of Formula (I), or a pharmaceutically acceptable salt thereof, administered daily is from about 200 mg to about 450 mg based on the weight of the free base.

22. The compound of any one of claims 1 to 15, wherein the amount of the compound of Formula (I), or a pharmaceutically acceptable salt thereof, administered daily is about 250 mg based on the weight of the free base.

23. The compound of claim 22, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base.

24. The compound of any one of claims 1 to 15, wherein the amount of the compound of Formula (I), or a pharmaceutically acceptable salt thereof, administered daily is about 300 mg based on the weight of the free base.

25. The compound of claim 24, wherein the first administration of the compound of Formula (I), or a pharmaceutically acceptable salt thereof is about 100 mg and the second administration of the compound of Formula (I), or a pharmaceutically acceptable salt thereof is about 200 mg based on the weight of the free base.

26. The compound of any one of claims 1 to 15, wherein the amount of the compound of Formula (I), or a pharmaceutically acceptable salt thereof, administered daily is about 350 mg based on the weight of the free base.

27. The compound of claim 26, wherein the first administration of the compound of Formula (I), or a pharmaceutically acceptable salt thereof is about 150 mg based on the weight of the free base and the second administration of the compound of Formula (I), or a pharmaceutically acceptable salt thereof is about 200 mg based on the weight of the free base.

28. The compound of any one of claims 1 to 27, wherein the subject weighs greater than or equal to about 55 kg.

29. The compound of any one of claims 1 to 15, wherein the amount of the compound of Formula (I), or a pharmaceutically acceptable salt thereof, administered daily is about 100 mg based on the weight of the free base.

30. The compound of claim 29, wherein the subject weighs from about 10 kg to about 20 kg.

31. The compound of any one of claims 1 to 15, wherein the amount of the compound of Formula (I), or a pharmaceutically acceptable salt thereof, administered daily is about 250 mg based on the weight of the free base.

32. The compound of claim 31, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base.

33. The compound of claim 31 or 32, wherein the subject weighs from about 20 kg to about 55 kg.

34. A compound of Formula (I): 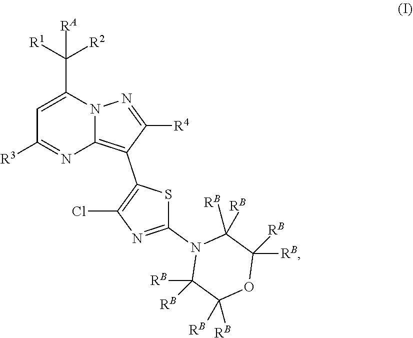
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
for use in a method of treating congenital adrenal hyperplasia in a subject,
wherein the compound of Formula (I), or a pharmaceutically acceptable salt thereof, is administered at a frequency of not less than twice daily; and
wherein the amount of the compound of Formula (I), or a pharmaceutically acceptable salt thereof, administered daily is less than 400 mg based on the weight of the free base.

35. The compound of claim 34, wherein the amount of the compound of Formula (I), or a pharmaceutically acceptable salt thereof, is administered at a frequency of twice daily.

36. The compound of claim 34 or 35, wherein the amount of the compound of Formula (I), or a pharmaceutically acceptable salt thereof, administered daily is from 100 mg to about 375 mg based on the weight of the free base.

37. The compound of claim 34 or 35, wherein the amount of the compound of Formula (I), or a pharmaceutically acceptable salt thereof, administered daily is from 100 mg to about 350 mg based on the weight of the free base.

38. The compound of claim 34 or 35, wherein the amount of the compound of Formula (I), or a pharmaceutically acceptable salt thereof, administered daily is from 100 mg to about 300 mg based on the weight of the free base.

39. The compound of claim 34 or 35, wherein the amount of the compound of Formula (I), or a pharmaceutically acceptable salt thereof, administered daily is from 100 mg to about 275 mg based on the weight of the free base.

40. The compound of claim 34 or 35, wherein the amount of the compound of Formula (I), or a pharmaceutically acceptable salt thereof, administered daily is from 100 mg to about 250 mg based on the weight of the free base.

41. The compound of claim 34 or 35, wherein the amount of the compound of Formula (I), or a pharmaceutically acceptable salt thereof, administered daily is about 250 mg based on the weight of the free base.

42. The compound of any one of claims 34 to 41, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base.

43. The compound of claim 34 or 35, wherein the amount of the compound of Formula (I), or a pharmaceutically acceptable salt thereof, administered daily is about 225 mg based on the weight of the free base.

44. The compound of any one of claims 34 to 40 and 43, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 125 mg based on the weight of the free base.

45. The compound of any one of claims 1 to 44, wherein the compound of Formula (I), or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject.

46. The compound of claim 45, wherein the reduction in level of any of biomarkers is determined by comparing the level of the biomarker as measured during the circadian release during a time period prior to administering the compound of Formula (I), or a pharmaceutically acceptable salt thereof and the level of the biomarker as measured during the circadian release on a day after administering the compound of Formula (I), or a pharmaceutically acceptable salt thereof.

47. The compound of claim 46, wherein the circadian release occurs between the hours of 2 a.m. and 10 a.m.

48. The compound of any one of claims 45 to 47, wherein the level of 17-hydroxyprogesterone is reduced by at least 25%.

49. The compound of any one of claims 45 to 47, wherein the level of 17-hydroxyprogesterone is reduced by at least 50%.

50. The compound of any one of claims 45 to 47, wherein the level of adrenocorticotropic hormone is reduced by at least 25%.

51. The compound of any one of claims 45 to 50, wherein the level of adrenocorticotropic hormone is reduced by at least 40%.

52. The compound of any one of claims 45 to 50, wherein the level of adrenocorticotropic hormone is reduced by at least 50%.

53. The compound of any one of claims 45 to 50, wherein the level of androstenedione is reduced by at least 25%.

54. The compound of any one of claims 45 to 50, wherein the level of androstenedione is reduced by at least 30%.

55. The compound of any one of claims 45 to 50, wherein the level of androstenedione is reduced by at least 50%.

56. The compound of any one of claims 45 to 55, wherein the level of 17-hydroxyprogesterone is reduced by at least 50% and the level of androstenedione is reduced by at least 50%.

57. A compound of Formula (I): 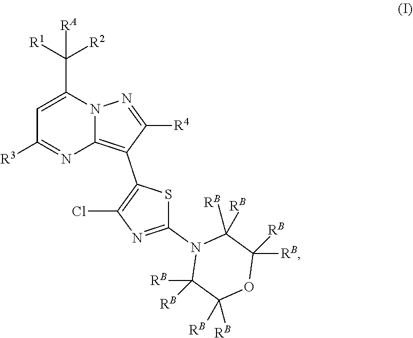
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
for use in a method of reducing the dosage of corticosteroid administered to a subject having congenital adrenal hyperplasia for controlling congenital adrenal hyperplasia,
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

58. The compound of claim 57, wherein the corticosteroid is a glucocorticoid.

59. A compound of Formula (I): 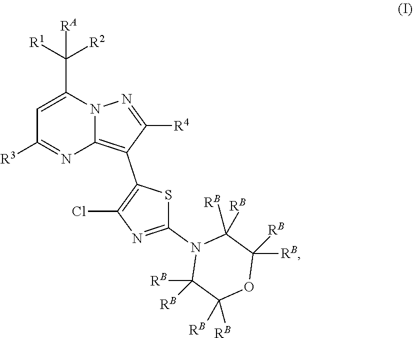
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium,
for use in a method of reducing the severity of one or more side effects of glucocorticoid treatment in a subject having congenital adrenal hyperplasia,
wherein the compound of Formula (I), or a pharmaceutically acceptable salt thereof, is administered at a frequency of not less than twice daily;
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations; and
wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, and reactivation of latent infections.

60. The compound of any one of claims 57 to 59, wherein the compound of Formula (I), or a pharmaceutically acceptable salt thereof, is administered at an amount sufficient to reduce the level of 17-hydroxyprogesterone (17-OHP) by at least 50% as compared to the level prior to administration.

61. The compound of any one of claims 57 to 60, wherein the compound of Formula (I), or a pharmaceutically acceptable salt thereof is administered at an amount sufficient to reduce the level of androstenedione by at least 30% as compared to the level prior to administration.

62. The compound of any one of claims 57 to 60, wherein the compound of Formula (I), or a pharmaceutically acceptable salt thereof is administered at an amount sufficient to (a) reduce the level of 17-hydroxyprogesterone (17-OHP) by at least 50% as compared to the level prior to administration; and (b) reduce the level of androstenedione by at least 30% as compared to the level prior to administration.

63. The compound of any one of claims 57 to 62, wherein the compound of Formula (I), or a pharmaceutically acceptable salt thereof, is administered twice daily at an amount from about 25 mg to about 250 mg based on the weight of the free base.

64. The compound of any one of claims 57 to 63, wherein the compound of Formula (I), or a pharmaceutically acceptable salt thereof, is administered twice daily as:
(a) a first administration of about 50 mg based on the weight of the free base and a second administration of about 150 mg based on the weight of the free base; or
(b) a first administration of about 100 mg based on the weight of the free base and a second administration of about 200 mg based on the weight of the free base; or
(c) a first administration of about 100 mg based on the weight of the free base and a second administration of about 250 mg based on the weight of the free base; or
(d) a first administration of about 150 mg based on the weight of the free base and a second administration of about 250 mg based on the weight of the free base.

65. The compound of any one of claims 1 to 64, wherein the compound of Formula (I) is administered in the free base form.

66. The compound of any one of claims 1 to 65, wherein the subject is in a fed state.

67. The compound of claim 66, wherein the compound of Formula (I), or a pharmaceutically acceptable salt thereof, is administered to the subject with a nutritional composition.

68. The compound of claim 67, wherein the nutritional composition is a liquid dietary supplement comprising about 1000 to about 2000 calories per liter with a fat content greater than about 30%.

69. The compound of claim 67, wherein the nutritional composition is a liquid dietary supplement comprising 1500 calories per liter with a caloric distribution of 14.7% protein, 32% fat and 53.3% carbohydrate.

70. The compound of any one of claims 67 to 69, wherein the nutritional composition is administered in an amount of about 6 to about 12 fluid ounces.

71. The compound of claim 70, wherein the nutritional composition is administered in an amount of about 8 fluid ounces.

72. The compound of any one of claims 67 to 71, wherein the nutritional composition is administered within 30 minutes of each administration of the compound of Formula (I), or a pharmaceutically acceptable salt thereof.

73. The compound of any one of claims 1 to 66, wherein the compound of Formula (I), or a pharmaceutically acceptable salt thereof, is administered to the subject with a meal.

74. The compound of claim 73, wherein the meal is a high fat meal.

75. The compound of claim 73, wherein the meal is a low fat meal.

76. The compound of any one of claims 73 to 75, wherein the meal is completed within about 30 minutes after administration of the compound of Formula (I), or a pharmaceutically acceptable salt thereof.

77. The compound of any one of claims 1 to 66 and 73 to 76, wherein the first administration of the compound of Formula (I), or a pharmaceutically acceptable salt thereof is with a morning meal.

78. The compound of any one of claims 1 to 66 and 73 to 77, wherein the second administration of the compound of Formula (I), or a pharmaceutically acceptable salt thereof is with an evening meal.

79. The compound of any one of claims 1 to 66 and 73 to 77, wherein the first administration of the compound of Formula (I), or a pharmaceutically acceptable salt thereof, is with a morning meal and the second administration of the compound of Formula (I), or a pharmaceutically acceptable salt thereof is with an evening meal.

80. The compound of any one of claims 1 to 79, wherein there are about 6 to about 14 hours between the first and second administrations of the compound of Formula (I), or a pharmaceutically acceptable salt thereof.

81. The compound of any one of claims 1 to 79, wherein there are about 8 to about 14 hours between the first and second administrations of the compound of Formula (I), or a pharmaceutically acceptable salt thereof.

82. The compound of any one of claims 1 to 79, wherein there are about 11 to about 13 hours between the first and second administrations of the compound of Formula (I), or a pharmaceutically acceptable salt thereof.

83. The compound of any one of claims 1 to 79, wherein there are about 12 hours between the first and second administrations of the compound of Formula (I), or a pharmaceutically acceptable salt thereof.

84. The compound of any one of claims 1 to 83, wherein the subject is concurrently receiving a dose of a glucocorticoid.

85. The compound of claim 84, wherein the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate.

86. The compound of claim 84 or 85, wherein the glucocorticoid is cortisol (hydrocortisone).

87. The compound of claim 84 or 85, wherein the glucocorticoid is cortisone.

88. The compound of claim 84 or 85, wherein the glucocorticoid is prednisone.

89. The compound of any one of claims 84 to 88, wherein the glucocorticoid dose is measured in hydrocortisone equivalents.

90. The compound of any one of claims 84 to 88, wherein the glucocorticoid dose is measured as a multiple of the upper limit of normal of physiologic dosing in hydrocortisone equivalents.

91. The compound of any one of claims 84 to 88, wherein the glucocorticoid dose is a physiologic dose as measured after a time period of administration of the compound of Formula (I), or a pharmaceutically acceptable salt thereof.

92. The compound of any one of claims 84 to 88, wherein the glucocorticoid dose is a physiologic dose of about 4 to about 12 mg/m²/day as measured after a time period of administration of the compound of Formula (I), or a pharmaceutically acceptable salt thereof.

93. The compound of any one of claims 84 to 88, wherein the glucocorticoid dose is a physiologic dose of about 4 to about 9 mg/m²/day as measured after a time period of administration of the compound of Formula (I), or a pharmaceutically acceptable salt thereof.

94. The compound of any one of claims 84 to 88, wherein the glucocorticoid dose is a physiologic dose that is less than about 8 mg/m²/day as measured after a time period of administration of the compound of Formula (I), or a pharmaceutically acceptable salt thereof.

95. The compound of any one of claims 84 to 94, wherein the glucocorticoid dose of the subject is reduced by about 10% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

96. The compound of any one of claims 84 to 94, wherein the glucocorticoid dose of the subject is reduced by about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

97. The compound of any one of claims 84 to 94, wherein the glucocorticoid dose of the subject is reduced by about 30% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

98. The compound of any one of claims 84 to 94, wherein the glucocorticoid dose of the subject is reduced by about 40% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

99. The compound of any one of claims 84 to 94, wherein the glucocorticoid dose of the subject is reduced by about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

100. The compound of any one of claims 84 to 94, wherein the glucocorticoid dose of the subject is reduced by about 60% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

101. The compound of any one of claims 84 to 94, wherein the glucocorticoid dose of the subject is reduced by about 70% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

102. The compound of any one of claims 84 to 94, wherein the glucocorticoid dose of the subject is reduced by less than about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

103. The compound of any one of claims 84 to 94, wherein the glucocorticoid dose of the subject is reduced by about 20% to about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

104. The compound of any one of claims 84 to 94, wherein the glucocorticoid dose of the subject is reduced by greater than about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

105. The compound of any one of claims 1 to 104, wherein the level of 17-hydroxyprogesterone is less than about 1.5 times the upper limit of normal after a time period of administration of the compound of Formula (I), or a pharmaceutically acceptable salt thereof.

106. The compound of any one of claims 1 to 105, wherein the level of 17-hydroxyprogesterone is within normal limits after a time period of administration of the compound of Formula (I), or a pharmaceutically acceptable salt thereof.

107. The compound of any one of claims 1 to 106, wherein the level of adrenocorticotropic hormone is less than about 1.5 times the upper limit of normal after a time period of administration of the compound of Formula (I), or a pharmaceutically acceptable salt thereof.

108. The compound of any one of claims 1 to 107, wherein the level of adrenocorticotropic hormone is within normal limits after a time period of administration of the compound of Formula (I), or a pharmaceutically acceptable salt thereof.

109. The compound of any one of claims 1 to 108, wherein the level of androstenedione is less than about 1.5 times the upper limit of normal after a time period of administration of the compound of Formula (I), or a pharmaceutically acceptable salt thereof.

110. The compound of any one of claims 1 to 109, wherein the level of androstenedione is within normal limits after a time period of administration of the compound of Formula (I), or a pharmaceutically acceptable salt thereof.

111. The compound of any one of claims 1 to 110, wherein the level of testosterone is reduced by at least about 25% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

112. The compound of any one of claims 1 to 111, wherein the level of testosterone is reduced by at least about 30% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

113. The compound of any one of claims 1 to 111, wherein the level of testosterone is reduced by at least about 50% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

114. The compound of any one of claims 1 to 113, wherein the level of testosterone is less than about 1.5 times the upper limit of normal after a time period of administration of the compound of Formula (I), or a pharmaceutically acceptable salt thereof.

115. The compound of any one of claims 1 to 114, wherein the level of testosterone is within normal limits after a time period of administration of the pharmaceutical composition comprising the compound of Formula (I), or a pharmaceutically acceptable salt thereof.

116. The compound of any one of claims 85 to 115, wherein the subject exhibits a decrease in glucocorticoid burden after a time period of administration of the compound of Formula (I), or a pharmaceutically acceptable salt thereof, wherein the decrease in glucocorticoid burden is relative to the glucocorticoid burden prior to administration of the compound of Formula (I), or a pharmaceutically acceptable salt thereof.

117. The compound of claim 116, wherein one or more symptoms of glucocorticoid burden selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor, control of ovarian adrenal rest tumor, and fertility, is improved after a time period of administration of the compound of Formula (I), or a pharmaceutically acceptable salt thereof, wherein the improvement in the one or more symptoms is relative to the status of the one or more symptoms prior to administration of the compound of Formula (I), or a pharmaceutically acceptable salt thereof.

118. The compound of any one of claims 91 to 117, wherein the time period of administration is at least about 4 weeks.

119. The compound of any one of claims 91 to 117, wherein the time period of administration is at least about 24 weeks.

120. The compound of any one of claims 91 to 117, wherein the time period of administration is at least about one year.

121. A compound of Formula (I): 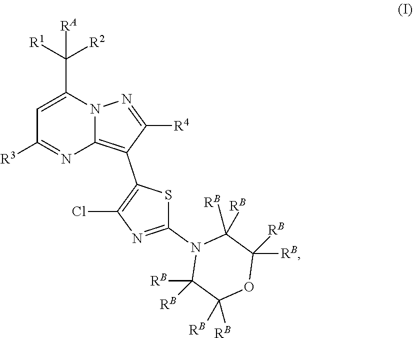
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium,
for use in a method of treating congenital adrenal hyperplasia in a subject, the method comprising:
(a) selecting a subject who has a glucocorticoid dose of greater than 11 mg/m²/day after a time period of being administered a compound of Formula (I), or a pharmaceutically acceptable thereof, at an amount of about 200 mg once daily based on the weight of the free base;
and
(b) administering to the subject the compound of Formula (I), or a pharmaceutically acceptable salt thereof, at a frequency of twice daily;
wherein the amount of the compound of Formula (I), or a pharmaceutically acceptable salt thereof, in the first administration is less than the amount of the compound of Formula (I), or a pharmaceutically acceptable salt thereof, in the second administration;
and wherein the amount of the compound of Formula (I), or a pharmaceutically acceptable salt thereof, administered daily is less than 400 mg based on the weight of the free base.

122. The compound of claim 121, wherein the time period of administration in step (a) is at least about 4 weeks.

123. The compound of claim 121, wherein the time period of administration in step (a) is at least about 24 weeks.

124. The compound of claim 121, wherein the time period of administration in step (a) is at least about one year.

125. The compound of any one of claims 1 to 124, wherein the subject is female.

126. The compound of any one of claims 1 to 124, wherein the subject is male.

127. The compound of any one of claims 1 to 126, wherein the compound of Formula (I), or a pharmaceutically acceptable salt thereof is administered as a pharmaceutical composition comprising: (a) the compound of Formula (I), or a pharmaceutically acceptable salt thereof; and (b) one or more of a lubricant, a diluent, a binder, and a glidant.

128. The compound of claim 127, wherein the pharmaceutical composition comprises about 70 wt % to about 97 wt % of the compound of Formula (I), or a pharmaceutically acceptable salt thereof, based on the weight of the free base.

129. The compound of claim 127, wherein the pharmaceutical composition comprises about 80 wt % to about 96 wt % of the compound of Formula (I), or a pharmaceutically acceptable salt thereof, based on the weight of the free base.

130. The compound of claim 127, wherein the pharmaceutical composition comprises about 85 wt % to about 90 wt % of the compound of Formula (I), or a pharmaceutically acceptable salt thereof, based on the weight of the free base.

131. The compound of any one of claims 127 to 130, wherein the pharmaceutical composition comprises a lubricant.

132. The compound of any one of claims 127 to 131, wherein the pharmaceutical composition comprises a diluent.

133. The compound of any one of claims 127 to 132, wherein the pharmaceutical composition comprises a binder.

134. The compound of any one of claims 127 to 133, wherein the pharmaceutical composition comprises a glidant.

135. The compound of any one of claims 1 to 134, wherein the pharmaceutical composition comprises the compound of Formula (I), or pharmaceutically acceptable salt thereof, in crystalline form.

136. The compound of any one of claims 1 to 135, wherein the pharmaceutical composition comprises the compound of Formula (I) as a free base.

137. The compound of any one of claims 1 to 136, wherein the pharmaceutical composition is formulated in unit dosage form, wherein the compound of Formula (I), or a pharmaceutically acceptable salt thereof, is present in an amount of about 25 mg to about 400 mg, based on the weight of the free base.

138. The compound of claim 137, wherein the compound of Formula (I), or a pharmaceutically acceptable salt thereof, is present in the unit dosage form in an amount of about 50 mg to about 400 mg, based on the weight of the free base.

139. The compound of claim 137, wherein the compound of Formula (I), or a pharmaceutically acceptable salt thereof, is present in the unit dosage form in an amount of about 50 mg, about 100 mg, about 150 mg, about 200 mg, about 250 mg, about 300 mg, about 350 mg, or about 400 mg, based on the weight of the free base.

140. The compound of claim 137, wherein the compound of Formula (I), or a pharmaceutically acceptable salt thereof, is present in the unit dosage form in an amount of about 75 mg, about 125 mg, about 175 mg, about 225 mg, about 275 mg, about 325 mg, or about 375 mg, based on the weight of the free base.

141. The compound of claim 137, wherein the compound of Formula (I), or a pharmaceutically acceptable salt thereof, is present in the unit dosage form in an amount of about 200 mg, based on the weight of the free base.

142. The compound of claim 137, wherein the compound of Formula (I), or a pharmaceutically acceptable salt thereof, is present in the unit dosage form in an amount of about 400 mg, based on the weight of the free base.

143. The compound of any one of claims 1 to 142, wherein the pharmaceutical composition is in the form of a tablet, capsule, sachet, powder, granules, coated particle, coated tablet, enterocoated tablet, enterocoated capsule, melting strip, or melting film.

144. The compound of claim 143, wherein the pharmaceutical composition is in tablet form.

145. The compound of claim 143, wherein the pharmaceutical composition is in capsule form.

146. The compound of any one of claims 144 or 145, wherein the tablet form or capsule form is coated.

147. The compound of any one of claims 1 to 126, wherein the compound of Formula (I) is administered as a pharmaceutical composition in oral solution dosage form comprising:
(a) the compound of Formula (I), or a pharmaceutically acceptable salt thereof;
(b) one or more of a sweetener, an anti-oxidant, and a flavor; and
(c) a liquid vehicle.

148. The compound of claim 147, wherein the pharmaceutical composition comprises:
(a) the compound of Formula (I), or a pharmaceutically acceptable salt thereof;
(b) a sweetener;
(c) an anti-oxidant;
(d) a flavor; and
(e) a liquid vehicle.

149. The compound of claim 148, wherein the pharmaceutical composition further comprises a surfactant.

150. The compound of claim 147, wherein the pharmaceutical composition comprises:
(a) a compound of Formula (I), or a pharmaceutically acceptable salt thereof,
(b) saccharin;
(c) butylated hydroxytoluene;
(d) FONA orange flavor; and
(e) medium-chain triglycerides.

151. The compound of claim 150, wherein the pharmaceutical composition further comprises oleoyl polyoxyl-6 glycerides.

152. The compound of any one of claims 147 to 151, wherein the pharmaceutical composition comprises the compound of Formula (I) as a free base.

153. The compound of any one of claims 147 to 152, wherein the pharmaceutical composition is formulated in unit dosage form, wherein the compound of Formula (I), or a pharmaceutically acceptable salt thereof, is present in an amount of about 5 mg/mL to about 200 mg/mL, based on the weight of the free base.

154. The compound of claim 153, wherein the unit dosage form comprises the compound of Formula (I), or a pharmaceutically acceptable salt thereof, in an amount of about 50 mg/mL, based on the weight of the free base.

155. The compound of any one of claims 1 to 126, wherein the compound of Formula (I), or a pharmaceutically acceptable salt thereof, is administered in a pharmaceutical composition comprising a spray-dried dispersion comprising:
a compound of Formula (I), or a pharmaceutically acceptable salt thereof; and
a polymer selected from a neutral polymer, an enteric polymer, and a pyrrolidone polymer;
wherein the weight ratio of the compound of Formula (I) to the polymer is from about 1:1 to about 1:9.

156. The compound of claim 155, wherein the spray-dried dispersion comprises:
a compound of Formula (I), or a pharmaceutically acceptable salt thereof; and
a polymer that is a copolymer of 1-vinyl-2-pyrrolidone and vinyl acetate having the structure: 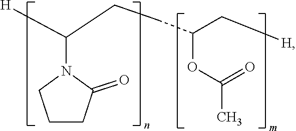
wherein the value of n is about 1 to about 2 times the value of m and the copolymer comprises 1-vinyl-2-pyrrolidone and vinyl acetate at a ratio of about 60:40 by weight; and
wherein the weight ratio of the compound of Formula (I) to the copolymer is from about 1:1 to about 1:9.

157. A method of treating congenital adrenal hyperplasia in a subject in need thereof comprising administering to the subject a compound of Formula (I): 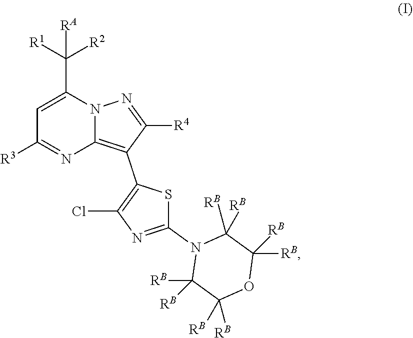
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

158. The method of claim 157, wherein the compound of Formula (I), or a pharmaceutically acceptable salt thereof, is administered at a frequency of twice daily.

159. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:100.

160. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:50.

161. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:10.

162. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:5.

163. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:3.

164. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.5.

165. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is from 1:1.1 to 1:2.

166. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:1.5.

167. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.

168. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:2.5.

169. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration about 1:3.

170. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:3.5.

171. The method of claim 157 or 158, wherein the ratio of the amount of the compound of Formula (I), or a pharmaceutically acceptable salt thereof, in the first administration to the amount of the compound of Formula (I), or a pharmaceutically acceptable salt thereof, in the second administration is about 1:4.

172. The method of any one of claims 157 to 171, wherein the amount of the compound of Formula (I), or a pharmaceutically acceptable salt thereof, administered daily is less than or equal to about 1000 mg based on the weight of the free base.

173. The method of any one of claims 157 to 171, wherein the amount of the compound of Formula (I), or a pharmaceutically acceptable salt thereof, administered daily is from about 50 mg to about 1000 mg based on the weight of the free base.

174. The method of any one of claims 157 to 171, wherein the amount of the compound of Formula (I), or a pharmaceutically acceptable salt thereof, administered daily is from about 100 mg to about 1000 mg based on the weight of the free base.

175. The method of any one of claims 157 to 171, wherein the amount of the compound of Formula (I), or a pharmaceutically acceptable salt thereof, administered daily is from about 100 mg to about 500 mg based on the weight of the free base.

176. The method of any one of claims 157 to 171, wherein the amount of the compound of Formula (I), or a pharmaceutically acceptable salt thereof, administered daily is from about 200 mg to about 500 mg based on the weight of the free base.

177. The method of any one of claims 157 to 171, wherein the amount of the compound of Formula (I), or a pharmaceutically acceptable salt thereof, administered daily is from about 200 mg to about 450 mg based on the weight of the free base.

178. The method of any one of claims 157 to 171, wherein the amount of the compound of Formula (I), or a pharmaceutically acceptable salt thereof, administered daily is about 250 mg based on the weight of the free base.

179. The method of claim 178, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base.

180. The method of any one of claims 157 to 171, wherein the amount of the compound of Formula (I), or a pharmaceutically acceptable salt thereof, administered daily is about 300 mg based on the weight of the free base.

181. The method of claim 180, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 200 mg based on the weight of the free base.

182. The method of any one of claims 157 to 171, wherein the amount of the compound of Formula (I), or a pharmaceutically acceptable salt thereof, administered daily is about 350 mg based on the weight of the free base.

183. The method of claim 182, wherein the first administration of the compound of Formula (I), or a pharmaceutically acceptable salt thereof is about 150 mg based on the weight of the free base and the second administration of the compound of Formula (I), or a pharmaceutically acceptable salt thereof is about 200 mg based on the weight of the free base.

184. The method of any one of claims 157 to 183, wherein the subject weighs greater than or equal to about 55 kg.

185. The method of any one of claims 157 to 171, wherein the amount of the compound of Formula (I), or a pharmaceutically acceptable salt thereof, administered daily is about 100 mg based on the weight of the free base.

186. The method of claim 185, wherein the subject weighs from about 10 kg to about 20 kg.

187. The method of any one of claims 157 to 171, wherein the amount of the compound of Formula (I), or a pharmaceutically acceptable salt thereof, administered daily is about 250 mg based on the weight of the free base.

188. The method of claim 187, wherein the wherein the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base.

189. The method of claim 187 or 188, wherein the subject weighs from about 20 kg to about 55 kg.

190. A method of treating congenital adrenal hyperplasia in a subject in need thereof comprising administering to the subject a compound of Formula (I): 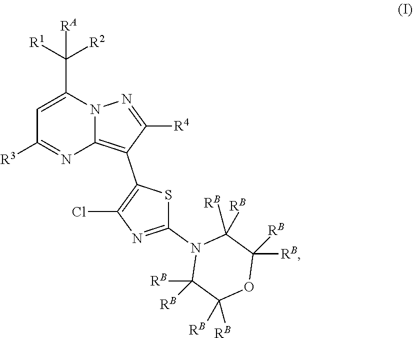
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
wherein the compound of Formula (I), or a pharmaceutically acceptable salt thereof, is administered at a frequency of not less than twice daily; and
wherein the amount of the compound of Formula (I), or a pharmaceutically acceptable salt thereof, administered daily is less than 400 mg based on the weight of the free base.

191. The method of claim 190, wherein the amount of the compound of Formula (I), or a pharmaceutically acceptable salt thereof, is administered at a frequency of twice daily.

192. The method of claim 190 or 191, wherein the amount of the compound of Formula (I), or a pharmaceutically acceptable salt thereof, administered daily is from 100 mg to about 375 mg based on the weight of the free base.

193. The method of claim 190 or 191, wherein the amount of the compound of Formula (I), or a pharmaceutically acceptable salt thereof, administered daily is from 100 mg to about 350 mg based on the weight of the free base.

194. The method of claim 190 or 191, wherein the amount of the compound of Formula (I), or a pharmaceutically acceptable salt thereof, administered daily is from 100 mg to about 300 mg based on the weight of the free base.

195. The method of claim 190 or 191, wherein the amount of the compound of Formula (I), or a pharmaceutically acceptable salt thereof, administered daily is from 100 mg to about 275 mg based on the weight of the free base.

196. The method of claim 190 or 191, wherein the amount of the compound of Formula (I), or a pharmaceutically acceptable salt thereof, administered daily is from about 100 mg to about 250 mg based on the weight of the free base.

197. The method of claim 190 or 191, wherein the amount of the compound of Formula (I), or a pharmaceutically acceptable salt thereof, administered daily is about 250 mg.

198. The method of claim 190 or 191, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 150 mg based on the weight of the free base.

199. The method of claim 190 or 191, wherein the amount of the compound of Formula (I), or a pharmaceutically acceptable salt thereof, administered daily is about 225 mg based on the weight of the free base.

200. The method of claim 199, wherein the first administration of the compound of Formula (I), or a pharmaceutically acceptable salt thereof is about 100 mg based on the weight of the free base and the second administration of the compound of Formula (I), or a pharmaceutically acceptable salt thereof is about 125 mg based on the weight of the free base.

201. The method of any one of claims 157 to 200, wherein the compound of Formula (I), or a pharmaceutically acceptable salt thereof, is administered in an amount sufficient to reduce the level of one or more biomarkers selected from (a) 17-hydroxyprogesterone (17-OHP); (b) adrenocorticotropic hormone (ACTH); and (c) androstenedione in the subject.

202. The method of claim 201, wherein the reduction in level of any of biomarkers is determined by comparing the level of the biomarker as measured during the circadian release during a time period prior to administering the compound of Formula (I), or a pharmaceutically acceptable salt thereof and the level of the biomarker as measured during the circadian release on a day after administering the compound of Formula (I), or a pharmaceutically acceptable salt thereof.

203. The method of claim 202, wherein the circadian release occurs between the hours of 2 a.m. and 10 a.m.

204. The method of any one of claims 201 to 203, wherein the level of 17-hydroxyprogesterone is reduced by at least 25%.

205. The method of any one of claims 201 to 203, wherein the level of 17-hydroxyprogesterone is reduced by at least 50%.

206. The method of any one of claims 201 to 203, wherein the level of adrenocorticotropic hormone is reduced by at least 25%.

207. The method of any one of claims 201 to 203, wherein the level of adrenocorticotropic hormone is reduced by at least 40%.

208. The method of any one of claims 201 to 203, wherein the level of adrenocorticotropic hormone is reduced by at least 50%.

209. The method of any one of claims 201 to 208, wherein the level of androstenedione is reduced by at least 25%.

210. The method of any one of claims 201 to 208, wherein the level of androstenedione is reduced by at least 30%.

211. The method of any one of claims 201 to 208, wherein the level of androstenedione is reduced by at least 50%.

212. The method of any one of claims 201 to 211, wherein the level of 17-hydroxyprogesterone is reduced by at least 50% and the level of androstenedione is reduced by at least 50%.

213. A method for reducing the severity of one or more symptoms selected from hirsutism, precocious puberty, fertility problems, acne, and growth impairment in a subject having classic congenital adrenal hyperplasia,
comprising administering to the subject a compound of Formula (I): 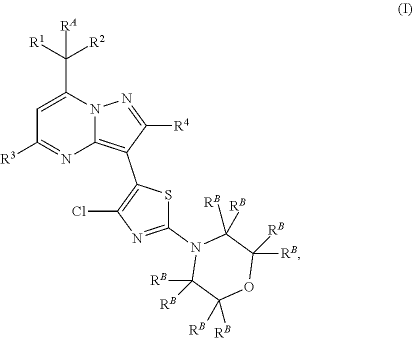
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

214. The method of claim 213, wherein the total daily amount of the compound of Formula (I) is sufficient to reduce the level of androstenedione in the subject.

215. The method of claim 213 or 214, wherein the growth impairment is selected from one or more of accelerated height velocity, accelerated weight velocity, or accelerated bone age.

216. The method of claim 213 or 214, wherein the androstenedione is reduced by at least 25%.

217. The method of claim 213 or 214, wherein the androstenedione is reduced by at least 30%.

218. The method of claim 213 or 214, wherein the androstenedione is reduced by at least 50%.

219. A method of reducing the level of one or more biomarkers of congenital adrenal hyperplasia in a subject having congenital adrenal hyperplasia comprising administering to the subject a compound of Formula (I): 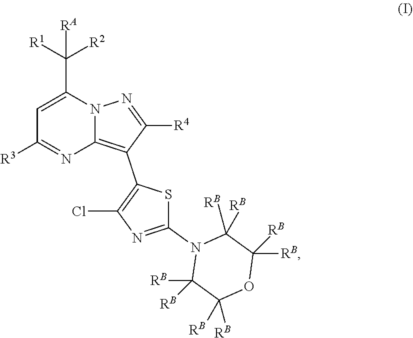
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

220. The method of claim 219, wherein the one or more biomarkers of congenital adrenal hyperplasia are selected from (a) 17-hydroxyprogesterone (17-OHP); (b) adrenocorticotropic hormone (ACTH); and (c) androstenedione.

221. A method of reducing the dosage of corticosteroid administered to a subject having congenital adrenal hyperplasia for controlling congenital adrenal hyperplasia comprising administering to the subject a compound of Formula (I): 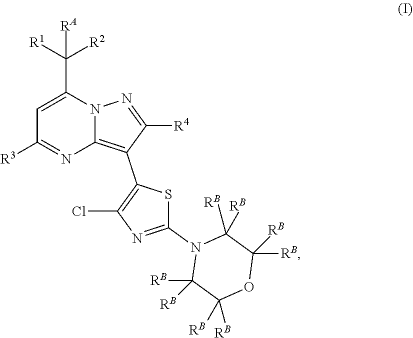
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium;
wherein the compound of Formula (I), or a pharmaceutically acceptable salt thereof, is administered at a frequency of not less than twice daily; and
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations.

222. The method of claim 221, wherein the corticosteroid is a glucocorticoid.

223. A method of reducing the severity of one or more side effects of glucocorticoid treatment in a subject having congenital adrenal hyperplasia comprising administering to the subject a compound of Formula (I): 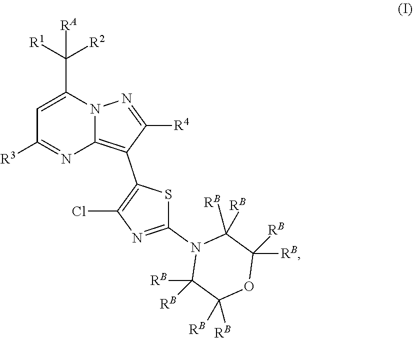
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium,
wherein the compound of Formula (I), or a pharmaceutically acceptable salt thereof, is administered at a frequency of not less than twice daily;
wherein the amount of the compound of Formula (I), or a pharmaceutically acceptable salt thereof, in the first administration is less than the amount of the compound of Formula (I), or a pharmaceutically acceptable salt thereof, in the second and any subsequent administrations; and
wherein the side effect is selected from osteoporosis, avascular necrosis of bone, myopathy, hyperglycemia, diabetes mellitus, dyslipidemia, weight gain, Cushing syndrome, Cushingoid features, growth suppression, adrenal suppression, gastritis, peptic ulcer, gastrointestinal bleeding, visceral perforation, hepatic steatosis, pancreatitis, hypertension, coronary heart disease, ischemic heart disease, heart failure, dermatoprosis, skin atrophy, ecchymosis, purpura, erosions, striae, delayed wound healing, easy bruising, acne, hirsutism, hair loss, mood changes, depression, euphoria, mood lability, irritability, akathisia, anxiety, cognitive impairment, psychosis, dementia, delirium, cataract, glaucoma, ptosis, mydriasis, opportunistic ocular infections, central serous chorioretinopathy, suppression of cell-mediated immunity, predisposition to infections, and reactivation of latent infections.

224. The method of any one of claims 219 to 223, wherein the compound of Formula (I), or a pharmaceutically acceptable salt thereof, is administered at an amount sufficient to reduce the level of 17-hydroxyprogesterone (17-OHP) by at least 50% as compared to the level prior to administration.

225. The method of any one of claims 219 to 224, wherein the compound of Formula (I), or a pharmaceutically acceptable salt thereof is administered at an amount sufficient to reduce the level of androstenedione by at least 30% as compared to the level prior to administration.

226. The method of any one of claims 219 to 224, wherein the compound of Formula (I), or a pharmaceutically acceptable salt thereof is administered at an amount sufficient to (a) reduce the level of 17-hydroxyprogesterone (17-OHP) by at least 50% as compared to the level prior to administration; and (b) reduce the level of androstenedione by at least 30% as compared to the level prior to administration.

227. The method of any one of claims 219 to 226, wherein the compound of Formula (I), or a pharmaceutically acceptable salt thereof, is administered twice daily at an amount from about 25 mg to about 250 mg based on the weight of the free base.

228. The method of any one of claims 219 to 227, wherein the compound of Formula (I), or a pharmaceutically acceptable salt thereof, is administered twice daily as:
(a) a first administration of about 50 mg based on the weight of the free base and a second administration of about 150 mg based on the weight of the free base; or
(b) a first administration of about 100 mg based on the weight of the free base and a second administration of about 200 mg based on the weight of the free base; or
(c) a first administration of about 100 mg based on the weight of the free base and a second administration of about 250 mg based on the weight of the free base; or
(d) a first administration of about 150 mg based on the weight of the free base and a second administration of about 250 mg based on the weight of the free base.

229. The method of any one of claims 219 to 228, wherein the compound of Formula (I) is administered in the free base form.

230. The method of any one of claims 219 to 229, wherein the subject is in a fed state.

231. The method of claim 230, wherein the compound of Formula (I), or a pharmaceutically acceptable salt thereof, is administered to the subject with a nutritional composition.

232. The method of claim 231, wherein the nutritional composition is a liquid dietary supplement comprising about 1000 to about 2000 calories per liter with a fat content greater than about 30%.

233. The method of claim 232, wherein the nutritional composition is a liquid dietary supplement comprising 1500 calories per liter with a caloric distribution of 14.7% protein, 32% fat and 53.3% carbohydrate.

234. The method of any one of claims 231 to 233, wherein the nutritional composition is administered in an amount of about 6 to about 12 fluid ounces.

235. The method any one of claims 231 to 233, wherein the nutritional composition is administered in an amount of about 8 fluid ounces.

236. The method of any one of claims 231 to 235, wherein the nutritional composition is administered within 30 minutes of each administration of the compound of Formula (I), or a pharmaceutically acceptable salt thereof.

237. The method of any one of claims 157 to 230, wherein the compound of Formula (I), or a pharmaceutically acceptable salt thereof, is administered to the subject with a meal.

238. The method of claim 237, wherein the meal is a high fat meal.

239. The method of claim 237, wherein the meal is a low fat meal.

240. The method of any one of claims 237 to 239, wherein the meal is completed within about 30 minutes after administration of the compound of Formula (I), or a pharmaceutically acceptable salt thereof.

241. The method of any one of claims 157 to 230 and 237 to 240, wherein the first administration of the compound of Formula (I), or a pharmaceutically acceptable salt thereof is with a morning meal.

242. The method of any one of claims 157 to 230 and 237 to 241, wherein the second administration of the compound of Formula (I), or a pharmaceutically acceptable salt thereof is with an evening meal.

243. The method of any one of claims 157 to 230 and 237 to 242, wherein the first administration of the compound of Formula (I), or a pharmaceutically acceptable salt thereof, is with a morning meal and the second administration of the compound of Formula (I), or a pharmaceutically acceptable salt thereof is with an evening meal.

244. The method of any one of claims 157 to 243, wherein there are about 6 to about 14 hours between the first and second administrations of the compound of Formula (I), or a pharmaceutically acceptable salt thereof.

245. The method of any one of claims 157 to 244, wherein there are about 8 to about 14 hours between the first and second administrations of the compound of Formula (I), or a pharmaceutically acceptable salt thereof.

246. The method of any one of claims 157 to 243, wherein there are about 11 to about 13 hours between the first and second administrations of the compound of Formula (I), or a pharmaceutically acceptable salt thereof.

247. The method of any one of claims 157 to 243, wherein there are about 12 hours between the first and second administrations of the compound of Formula (I), or a pharmaceutically acceptable salt thereof.

248. The method of any one of claims 157 to 247, wherein the subject is concurrently receiving a dose of a glucocorticoid.

249. The method of claim 248, wherein the glucocorticoid is selected from cortisol (hydrocortisone), cortisone, prednisone, prednisolone, methylprednisolone, dexamethasone, betamethasone, triamcinolone, fludrocortisone acetate, and deoxycorticosterone acetate.

250. The method of claim 248 or 249, wherein the glucocorticoid is cortisol (hydrocortisone).

251. The method of claim 248 or 249, wherein the glucocorticoid is cortisone.

252. The method of claim 248 or 249, wherein the glucocorticoid is prednisone.

253. The method of any one of claims 248 to 252, wherein the glucocorticoid dose is measured in hydrocortisone equivalents.

254. The method of any one of claims 248 to 253, wherein the glucocorticoid dose is measured as a multiple of the upper limit of normal of physiologic dosing in hydrocortisone equivalents.

255. The method of any one of claims 248 to 253, wherein the glucocorticoid dose is a physiologic dose as measured after a time period of administration of the compound of Formula (I), or a pharmaceutically acceptable salt thereof.

256. The method of any one of claims 248 to 253, wherein the glucocorticoid dose is a physiologic dose of about 4 to about 12 mg/m²/day as measured after a time period of administration of the compound of Formula (I), or a pharmaceutically acceptable salt thereof.

257. The method of any one of claims 248 to 253, wherein the glucocorticoid dose is a physiologic dose of about 4 to about 9 mg/m²/day as measured after a time period of administration of the compound of Formula (I), or a pharmaceutically acceptable salt thereof.

258. The method of any one of claims 248 to 253, wherein the glucocorticoid dose is a physiologic dose that is less than about 8 mg/m²/day as measured after a time period of administration of the compound of Formula (I), or a pharmaceutically acceptable salt thereof.

259. The method of any one of claims 248 to 258, wherein the glucocorticoid dose of the subject is reduced by about 10% after a time period of administration of the compound of Formula (I), or a pharmaceutically acceptable salt thereof, wherein the reduction of the glucocorticoid dose is relative to the glucocorticoid dose prior to administration of the compound of Formula (I).

260. The method of any one of claims 248 to 258, wherein the glucocorticoid dose of the subject is reduced by about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

261. The method of any one of claims 248 to 258, wherein the glucocorticoid dose of the subject is reduced by about 30% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

262. The method of any one of claims 248 to 258, wherein the glucocorticoid dose of the subject is reduced by about 40% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

263. The method of any one of claims 248 to 258, wherein the glucocorticoid dose of the subject is reduced by about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

264. The method of any one of claims 248 to 258, wherein the glucocorticoid dose of the subject is reduced by about 60% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

265. The method of any one of claims 248 to 258, wherein the glucocorticoid dose of the subject is reduced by about 70% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

266. The method of any one of claims 248 to 258, wherein the glucocorticoid dose of the subject is reduced by less than about 20% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

267. The method of any one of claims 248 to 258, wherein the glucocorticoid dose of the subject is reduced by about 20% to about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

268. The method of any one of claims 248 to 258, wherein the glucocorticoid dose of the subject is reduced by greater than about 50% after a time period of administration of the compound of Formula (I), or a pharmaceutically acceptable salt thereof, wherein the reduction of the glucocorticoid dose is relative to the glucocorticoid dose prior to administration of the compound of Formula (I), or a pharmaceutically acceptable salt thereof.

269. The method of any one of claims 157 to 268, wherein the level of 17-hydroxyprogesterone is less than about 1.5 times the upper limit of normal after a time period of administration of the compound of Formula (I), or a pharmaceutically acceptable salt thereof.

270. The method of any one of claims 157 to 269, wherein the level of 17-hydroxyprogesterone is within normal limits after a time period of administration of the compound of Formula (I), or a pharmaceutically acceptable salt thereof.

271. The method of any one of claims 157 to 270, wherein the level of adrenocorticotropic hormone is less than about 1.5 times the upper limit of normal after a time period of administration of the compound of Formula (I), or a pharmaceutically acceptable salt thereof.

272. The method of any one of claims 157 to 271, wherein the level of adrenocorticotropic hormone is within normal limits after a time period of administration of the pharmaceutical composition.

273. The method of any one of claims 157 to 272, wherein the level of androstenedione is less than about 1.5 times the upper limit of normal after a time period of administration of the compound of Formula (I), or a pharmaceutically acceptable salt thereof.

274. The method of any one of claims 157 to 273, wherein the level of androstenedione is within normal limits after a time period of administration of the compound of Formula (I), or a pharmaceutically acceptable salt thereof.

275. The method of any one of claims 157 to 274, wherein the level of testosterone is reduced by at least about 25% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

276. The method of any one of claims 157 to 275, wherein the level of testosterone is reduced by at least about 30% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

277. The method of any one of claims 157 to 276, wherein the level of testosterone is reduced by at least about 50% after a time period of administration of the compound of Formula (I), or a pharmaceutically acceptable salt thereof, wherein the reduction of the level of testosterone is relative to the level of testosterone prior to administration of the compound of Formula (I), or a pharmaceutically acceptable salt thereof.

278. The method of any one of claims 157 to 277, wherein the level of testosterone is less than about 1.5 times the upper limit of normal after a time period of administration of the pharmaceutical composition.

279. The method of any one of claims 157 to 278, wherein the level of testosterone is within normal limits after a time period of administration of the pharmaceutical composition comprising the compound of Formula (I), or a pharmaceutically acceptable salt thereof.

280. The method of any one of claims 248 to 279, wherein the subject exhibits a decrease in glucocorticoid burden after a time period of administration of the compound of Formula (I), or a pharmaceutically acceptable salt thereof, wherein the decrease in glucocorticoid burden is relative to the glucocorticoid burden prior to administration of the compound of Formula (I), or a pharmaceutically acceptable salt thereof.

281. The method of claim 280, wherein one or more symptoms of glucocorticoid burden selected from quality of life, fatigue, sleep, insulin resistance, glucose tolerance, glucose control, dyslipidemia, hyperlipidemia, bone mineral density, bone turnover, fat mass, weight, central obesity, blood pressure, hirsutism severity, menstrual cyclicity, control of testicular adrenal rest tumor, control of ovarian adrenal rest tumor, and fertility, is improved after a time period of administration of the compound of Formula (I), or a pharmaceutically acceptable salt thereof, wherein the improvement in the one or more symptoms is relative to the status of the one or more symptoms prior to administration of the compound of Formula (I), or a pharmaceutically acceptable salt thereof.

282. The method of any one of claims 256 to 281, wherein the time period of administration is at least about 4 weeks.

283. The method of any one of claims 256 to 281, wherein the time period of administration is at least about 24 weeks.

284. The method of any one of claims 256 to 281, wherein the time period of administration is at least about one year.

285. A method of treating congenital adrenal hyperplasia in a subject, the method comprising:
(a) selecting a subject who has a glucocorticoid dose of greater than 11 mg/m²/day after a time period of being administered a compound of Formula (I): 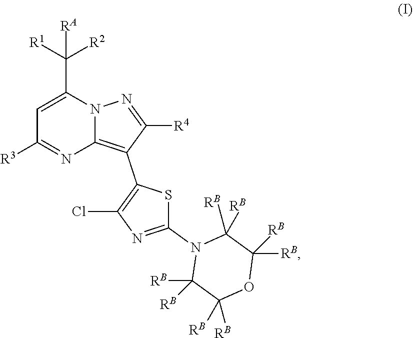
or a pharmaceutically acceptable salt thereof, wherein:
R¹ is —C(R^{A})₂C(R^{A})₃;
R² is —C(R^{A})₂C(R^{A})₃;
R³ is —C(R^{A})₃;
R⁴ is —C(R^{A})₃;
each R^{A} is independently hydrogen or deuterium; and
each R^{B} is independently hydrogen or deuterium, or a pharmaceutically acceptable thereof, at an amount of about 200 mg once daily based on the weight of the free base;
and
(b) administering to the subject the compound of Formula (I), or a pharmaceutically acceptable salt thereof, at a frequency of twice daily;
wherein the amount of the compound of Formula (I), or a pharmaceutically acceptable salt thereof, in the first administration is less than the amount of the compound of Formula (I), or a pharmaceutically acceptable salt thereof, in the second administration;
and wherein the amount of the compound of Formula (I), or a pharmaceutically acceptable salt thereof, administered daily is less than or equal to about 400 mg based on the weight of the free base.

286. The method of claim 285, wherein the time period of administration in step (a) is at least about 4 weeks.

287. The method of claim 285, wherein the time period of administration in step (a) is at least about 24 weeks.

288. The method of claim 285, wherein the time period of administration in step (a) is at least about one year.

289. The method of any one of claims 157 to 288, wherein the subject is female.

290. The method of any one of claims 157 to 288, wherein the subject is male.

291. The method of any one of claims 157 to 290, wherein the compound of Formula (I) is administered as a pharmaceutical composition comprising: (a) the compound of Formula (I), or a pharmaceutically acceptable salt thereof; and (b) one or more of a lubricant, a diluent, a binder, and a glidant.

292. The method of claim 291, wherein the pharmaceutical composition comprises about 70 wt % to about 97 wt % of the compound of Formula (I), or a pharmaceutically acceptable salt thereof, based on the weight of the free base.

293. The method of claim 291, wherein the pharmaceutical composition comprises about 80 wt % to about 96 wt % of the compound of Formula (I), or a pharmaceutically acceptable salt thereof, based on the weight of the free base.

294. The method of claim 291, wherein the pharmaceutical composition comprises about 85 wt % to about 90 wt % of the compound of Formula (I), or a pharmaceutically acceptable salt thereof, based on the weight of the free base.

295. The method of any one of claims 291 to 294, wherein the pharmaceutical composition comprises a lubricant.

296. The method of any one of claims 291 to 295, wherein the pharmaceutical composition comprises a diluent.

297. The method of any one of claims 291 to 296, wherein the pharmaceutical composition comprises a binder.

298. The method of any one of claims 291 to 297, wherein the pharmaceutical composition comprises a glidant.

299. The method of any one of claims 157 to 298, wherein the pharmaceutical composition comprises the compound of Formula (I), or pharmaceutically acceptable salt thereof, in crystalline form.

300. The method of any one of claims 157 to 299 wherein the pharmaceutical composition comprises the compound of Formula (I) as a free base.

301. The method of any one of claims 157 to 300, wherein the pharmaceutical composition comprises the compound of Formula (I), or pharmaceutically acceptable salt thereof, in crystalline form.

302. The method of any one of claims 291 to 301, wherein the pharmaceutical composition is formulated in unit dosage form, wherein the compound of Formula (I), or a pharmaceutically acceptable salt thereof, is present in an amount of about 50 mg, about 100 mg, about 150 mg, about 200 mg, about 250 mg, about 300 mg, about 350 mg, or about 400 mg, based on the weight of the free base.

303. The method of claim 302, wherein the compound of Formula (I), or a pharmaceutically acceptable salt thereof, is present in the unit dosage form in an amount of about 75 mg, about 125 mg, about 175 mg, about 225 mg, about 275 mg, about 325 mg, or about 375 mg, based on the weight of the free base.

304. The method of claim 302, wherein the compound of Formula (I), or a pharmaceutically acceptable salt thereof, is present in the unit dosage form in an amount of about 200 mg, based on the weight of the free base.

305. The method of claim 302, wherein the compound of Formula (I), or a pharmaceutically acceptable salt thereof, is present in the unit dosage form in an amount of about 300 mg, based on the weight of the free base.

306. The method of claim 302, wherein the compound of Formula (I), or a pharmaceutically acceptable salt thereof, is present in the unit dosage form in an amount of about 400 mg, based on the weight of the free base.

307. The method of any one of claims 157 to 306, that is in the form of a tablet, capsule, sachet, powder, granules, coated particle, coated tablet, enterocoated tablet, enterocoated capsule, melting strip, or melting film.

308. The method of claim 307, wherein the pharmaceutical composition is in tablet form.

309. The method of claim 307, wherein the pharmaceutical composition is in capsule form.

310. The method of any one of claims 157 to 306, wherein the compound of Formula (I) is administered as a pharmaceutical composition in oral solution dosage form comprising:
(a) the compound of Formula (I),
or a pharmaceutically acceptable salt thereof;
(b) one or more of a sweetener, an anti-oxidant, and a flavor; and
(c) a liquid vehicle.

311. The method of claim 310, wherein the pharmaceutical composition comprises:
(a) the compound of Formula (I), or a pharmaceutically acceptable salt thereof,
(b) a sweetener;
(c) an anti-oxidant;
(d) a flavor; and
(e) a liquid vehicle.

312. The method of claim 310, wherein the pharmaceutical composition further comprises a surfactant.

313. The method of claim 310, wherein the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of a sweetener;
(c) about 0.1 w/v % to about 0.2 w/v % of an anti-oxidant;
(d) about 0.05 w/v % to about 0.2 w/v % of a flavor;
(e) about 15 w/v % to about 25 w/v % of a surfactant; and
(f) about 70 w/v % to about 80 w/v % of a liquid vehicle.

314. The method of claim 310, wherein the pharmaceutical composition comprises:
(a) about 5 w/v % of the compound of Formula (I), or a pharmaceutically acceptable salt thereof, based on the weight of the free base;
(b) about 0.15 w/v % of a sweetener;
(c) about 0.17 w/v % of an anti-oxidant;
(d) about 0.1 w/v % of a flavor;
(e) about 20 w/v % of a surfactant; and
(f) about 75 w/v % of a liquid vehicle.

315. The method of claim 310, wherein the pharmaceutical composition comprises:
(a) a compound of Formula (I), or a pharmaceutically acceptable salt thereof,
(b) saccharin;
(c) butylated hydroxytoluene;
(d) FONA orange flavor; and
(e) medium-chain triglycerides.

316. The method of claim 315 wherein the pharmaceutical composition further comprises oleoyl polyoxyl-6 glycerides.

317. The method claim 310, wherein the pharmaceutical composition comprises:
(a) about 4 w/v % to about 6 w/v % of the compound of Formula (I), or a pharmaceutically acceptable salt thereof, based on the weight of the free base;
(b) about 0.1 w/v % to about 0.2 w/v % of saccharin;
(c) about 0.1 w/v % to about 0.2 w/v % of butylated hydroxytoluene;
(d) about 0.05 w/v % to about 0.2 w/v % of FONA orange flavor;
(e) about 15 w/v % to about 25 w/v % of oleoyl polyoxyl-6 glycerides; and
(f) about 70 w/v % to about 80 w/v % of medium-chain triglycerides.

318. The method of claim 310, wherein the pharmaceutical composition comprises:
(a) about 5 w/v % of the compound of Formula (I), or a pharmaceutically acceptable salt thereof, based on the weight of the free base;
(b) about 0.15 w/v % of saccharin;
(c) about 0.17 w/v % of butylated hydroxytoluene;
(d) about 0.1 w/v % of FONA orange flavor;
(e) about 20 w/v % of oleoyl polyoxyl-6 glycerides; and
(f) about 75 w/v % of medium-chain triglycerides.

319. The method of any one of claims 310 to 318, wherein the pharmaceutical composition comprises the compound of Formula (I) as a free base.

320. The method of any one of claims 310 to 319, wherein the pharmaceutical composition is formulated in unit dosage form, wherein the compound of Formula (I), or a pharmaceutically acceptable salt thereof, is present in an amount of about 5 mg/mL to about 200 mg/mL, based on the weight of the free base.

321. The method of claim 320, wherein the unit dosage form comprises the compound of Formula (I), or a pharmaceutically acceptable salt thereof, in an amount of about 50 mg/mL, based on the weight of the free base.

322. The method of any one of claims 157 to 306, wherein the compound of Formula (I) is administered in a pharmaceutical composition comprising a spray-dried dispersion comprising:
a compound of Formula (I), or a pharmaceutically acceptable salt thereof; and
a polymer selected from a neutral polymer, an enteric polymer, and a pyrrolidone polymer;
wherein the weight ratio of the compound of Formula (I) to the polymer is from about 1:1 to about 1:9.

323. The method of claim 322, wherein the spray-dried dispersion comprises:
a compound of Formula (I), or a pharmaceutically acceptable salt thereof; and
a polymer that is a copolymer of 1-vinyl-2-pyrrolidone and vinyl acetate having the structure: 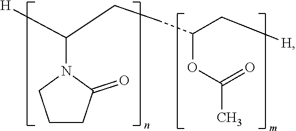
wherein the value of n is about 1 to about 2 times the value of m and the copolymer comprises 1-vinyl-2-pyrrolidone and vinyl acetate at a ratio of about 60:40 by weight; and
wherein the weight ratio of the compound of Formula (I) to the copolymer is from about 1:1 to about 1:9.

324. The method of claim 322, wherein the pharmaceutical composition comprises:
(a) the spray-dried dispersion of Example 3;
(b) calcium silicate;
(c) a combination of mannitol and microcrystalline cellulose; and
(d) croscarmellose sodium.

325. The method of claim 322, wherein the pharmaceutical composition comprises:
(a) about 1 w/w % to about 20 w/w % of the spray-dried dispersion of Example 3;
(b) about 0.1 w/w % to about 1 w/w % of calcium silicate;
(c) about 50 w/w % to about 60 w/w % of mannitol and about 10 w/w % to about 30 w/w % of microcrystalline cellulose; and
(d) about 5 w/w % to about 0.2 w/w % of croscarmellose sodium.

326. The method of claim 322, wherein the pharmaceutical composition comprises:
(a) about 13 w/w % of the spray-dried dispersion of Example 3;
(b) about 0.67 w/w % of calcium silicate;
(c) about 56 w/w % of mannitol and about 20 w/w % of microcrystalline cellulose; and
(d) about 10 w/w % of croscarmellose sodium.

327. The method of any one of claims 322 to 326, wherein the pharmaceutical composition is formulated as a tablet, capsule, sachet, powder, granules, coated particle, coated tablet, enterocoated tablet, enterocoated capsule, melting strip, or melting film.

328. The compound or method of any one of claims 1-327, wherein at least one of R^{A} or R^{B} is deuterium.

329. The compound of claim 328, wherein R¹ is —CH₂CD₃, or R² is —CH₂CD₃.

330. The compound of claim 328, wherein R¹ is —CH₂CD₃, and R² is —CH₂CD₃ or R¹ is —CH₂CH₃, and R² is —CH₂CH₃.

331. The compound of claim 328, wherein R¹ is —CD₂CD₃, or R² is —CD₂CD₃.

332. The compound of claim 331, wherein R¹ is —CD₂CD₃, and R² is —CD₂CD₃.

333. The compound of claim 328, wherein R³ is CD₃; or R⁴ is CD₃.

334. The compound of claim 328, wherein each R^{A} is hydrogen.

335. The compound of claim 328, wherein each R^{B} is hydrogen.
